(19) United States
(12) Patent Application Publication
(10) Pub. No.: US 2004/0097544 A1
(43) Pub. Date: May 20, 2004

(54) Use of an inhibitor of the Na+/H+ exchanger for the production of a medicament for the treatment or prophylaxis of disturbances of the central nervous system

(75) Inventors: Hans Jochen Lang, Hofheim (DE); Klaus Wirth, Kriftel (DE); Dieter Bingmann, Essen (DE); Udo Bonnet, Dortmund (DE); Martin Wiemann, Munster (DE)

Correspondence Address: FINNEGAN, HENDERSON, FARABOW, GARRETT & DUNNER, LLP, 1300 I STREET, NW, WASHINGTON, DC, 20005, US

(73) Assignee: Hoechst Marion Roussel Deutschland GMBH

(21) Appl. No.: 10/424,107

(22) Filed: Apr. 28, 2003

Related U.S. Application Data
(63) Continuation of application No. 09/685,339, filed on Oct. 11, 2000, now abandoned.
Application No. 09/685,339 is a continuation of application No. 09/159,126, filed on Sep. 23, 1998, now abandoned.

(30) Foreign Application Priority Data
Sep. 24, 1997 (DE) 19742096.6
Nov. 14, 1997 (DE) 19750498.1

(51) Int. Cl. A61K 31/47; A61K 31/404; A61K 31/255; A61K 31/165

(52) U.S. Cl. 514/309; 514/312; 514/418; 514/517; 514/617

(57) ABSTRACT

The use of an inhibitor of the Na⁺/H⁺ exchanger for the production of a medicament for the treatment or prophylaxis of disorders of the central nervous system. Inhibitors of the Na⁺/H⁺ exchanger are outstandingly suitable for the production of a medicament for the treatment or prophylaxis of disorders of the central nervous system; derivatives of guanidine are particularly highly suitable; cariporide (HOE 642) is especially suitable.

The use of an inhibitor of the Na⁺/H⁺ exchanger for the production of a medicament for the treatment or prophylaxis of disturbances of the central nervous system

The invention describes the use of inhibitors of the cellular sodium-hydrogen exchanger for the production of a medicament for the therapy and prophylaxis of disorders and disturbances which are caused by hyperexcitability of the central nervous system, in particular for the treatment of disorders of the epileptic type, centrally induced clonic and tonic spasms, anxiety disorders and psychoses.

Inhibitors of the sodium/hydrogen exchanger (NHE) have recently been characterized in numerous preclinical studies as substances which in the case of restricted circulation of the heart are suitable in a superior manner to protect endangered heart tissue from death. The protection of the heart tissue by NHE inhibitors includes all expressions of the damage caused by the inadequate circulation, starting with cardiac arrhythmias via hypercontraction of the heart muscle and temporary loss of function up to death of the heart tissue and permanent damage associated therewith.

The mechanism of action of the NHE inhibitors is that they decrease the increased sodium ion influx which arises in inadequately supplied tissues due to activation of the NHE as a result of intracellular acidification. The situation of sodium overloading of the tissue is thereby delayed. Since sodium and calcium ion transport are coupled to one another in the heart tissue, the life-threatening calcium overloading of the heart cells is thus prevented.

In the same manner as in the heart, a protection of the central nervous system under the influence of inhibitors of the NHE has likewise already been described in most of the patents cited here, active compounds of this type protecting the CNS similarly to the heart against ischemic conditions. These conditions are caused by an inadequate circulation and thus by an inadequate supply of nutrients, oxygen, minerals etc. Ischemic damage to the CNS of this type is particularly marked in the case of central nervous system infarcts, such as stroke.

In the case of normal healthy circulation, as expected, it was therefore, of course, also not possible to observe any protective effects of NHE inhibitors, since no ischemic tissue damage to the heart or to the CNS occurred.

It was therefore surprising that NHE inhibitors, beside the protective effects which only have an influence under conditions of inadequate circulation, also still have direct therapeutically utilizable actions against disorders and disturbances of the CNS which are independent of inadequate circulation states and occur in normal, nonischemic conditions. These pathological, nonischemically induced symptoms, which recently have been made accessible to treatment with NHE inhibitors, are disorders and disturbances which are substantially caused by a hyperexcitability of neurons of the central nervous system. NHE inhibitors act on disorders and disturbances of this type by a damping of the hyperactivity of CNS neurons.

Disturbances in which an inhibition of the excitability of the hyperactive neurons plays a dominant part include those of the epileptic type, for example grand mal, petit mal etc.

Neuronal hyperactivity, however, is not only observed in epilepsy. In other centrally induced functional disturbances such as in various psychoses, neuronal hyperactivity in various brain regions is also suspected as a cause. Since the described principle of action of the inhibition of the NHE can damp the neuronal hyperactivity independently of the trigger or disease, it is suitable not only for the therapy of epilepsies, but also for the treatment of affective psychoses and anxiety disturbances. The indications resulting therefrom thus include the treatment of epilepsies and affective psychoses and anxiety disturbances.

The NHE inhibitors employed antiepileptically and antipsychotically can be used on their own. The unique mechanism of action of the NHE inhibitors also makes it possible, however, to combine NHE inhibitors with other active compounds which are based on another mechanism of action. Thus, for example, combinations of NHE inhibitors with other substances having antiepileptic action or antipsychotic active compounds, or carboanhydrase inhibitors (e.g. acetazolamide) allow favorable therapy schemes and treatment results which are not attainable using the individual components of the combination.

In the model of CA3 neurons in hippocampus tissue slices, NHE inhibitors with their antiepileptic and antipsychotic therapy potential inhibit epileptiform discharges. Hyperexcitability of neurons in the form of so-called epileptiform discharges was triggered by the action of bicuculline, caffeine or low magnesium concentrations. HOE 642, a blocker of the Na⁺/H⁺ isoform1, showed a marked inhibition of the epileptiform discharges caused by the excitatory action of these substances.

Experimental demonstration of action of the damping of neuronal excitation states by NHE inhibitors:

The decrease in neuronal excitability was tested on slice preparations of CA3 neurons of the hippocampus (guinea-pigs). The excitation-inhibiting action of cariporide (HOE 642) was investigated in 8 experiments. The neuronal excitability was assessed with intracellular leads with the aid of the spontaneous bioelectric activity and the pharmacologically produced epileptiform activity which had been induced by bicuculline, caffeine or absence of magnesium. Both the frequency of the spontaneous action potentials and the frequency of so-called periodic paroxysmal depolarization was clearly reversibly reduced by 30-80% for more than 45 minutes after a wash-in time of 0.1-0.5 mM cariporide.

A relationship between neuronal excitability and inhibition of the Na⁺/H⁺ exchanger is already indicated in “Suppressive Action of propionate and inhibitors of cellular acid-extrusion mechanisms (IAE) on epileptiform activity of CA3 neurons, Eur. J. Physiol. R 153, P-562, 1997; however, the action of the guanidines and similar compounds is neither anticipated nor suggested by this.

The active compounds known and identified as NHE inhibitors are guanidine derivatives, preferably acylguanidines, inter alia as described in the following publications and patent disclosures: Edward J. Cragoe, Jr., “DIURETICS, Chemistry, Pharmacology and Medicine”, J. WILEY & Sons (1983), 303-341, additionally compounds of the following formulae:

I. (HOE 89/F 288—U.S. Pat. No. 5,292,755)

a) benzoylguanidines of the formula I

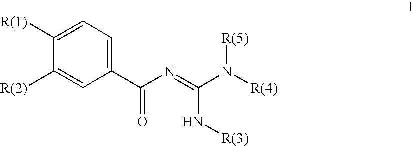

in which:

R(1) or R(2)

is R(6)—S(O)ₙ— or R(7)R(8)N—O₂S—;

and the other substituent R(1) or R(2) in each case

is H, F, Cl, Br, (C₁-C₄)-alkyl, (C₁-C₄)-alkoxy or phenoxy,

which is unsubstituted or substituted by 1-3 substituents selected from the group consisting of fluorine, chlorine, methyl and methoxy;

or the other substituent R(1) or R(2) in each case

is R(6)—S(O)ₙ or R(7)R(8)N—;

n is zero, 1 or 2;

R(6) is (C₁-C₆)-alkyl, (C₅-C₇)-cycloalkyl, cyclopentylmethyl, cyclohexylmethyl or phenyl,

which is unsubstituted or substituted by 1-3 substituents selected from the group consisting of fluorine, chlorine, methyl and methoxy;

R(7) and R(8)

identically or differently are H or (C₁-C₆)-alkyl;

or

R(7) is phenyl-(CH₂)ₘ;

m is 1-4;

or

R(7) is phenyl,

which is unsubstituted or substituted by 1-2 substituents selected from the group consisting of fluorine, chlorine, methyl and methoxy;

or

R(7) and R(8)

together are a straight-chain or branched (C₄-C₇)-chain, where the chain can additionally be interrupted by O, S or NR(9);

R(9) is H or methyl;

or

R(7) and R(8)

together with the nitrogen atom to which they are bonded, are a dihydroindole, tetrahydroquinoline or tetrahydroisoquinoline system;

R(3), R(4) and R(5)

independently of one another are H or (C₁-C₂)-alkyl,

or

R(3) and R(4)

together are a (C₂-C₄)-alkylene chain;

or

R(4) and R(5)

together are a (C₄-C₇)-alkylene chain;

and their pharmaceutically tolerable salts;

(HOE 92/F 34—U.S. Pat No. 5,373,924)

b) benzoylguanidines of the formula I

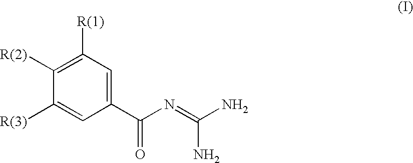

in which:

R(1) is R(4)—SOₘ or R(5)R(6)N—SO₂—;

m is zero, 1 or 2;

R(4) and R(5)

are C₁-C₈-alkyl, C₃-C₆-alkenyl or —CₙH₂ₙ—R(7);

n is zero, 1, 2, 3 or 4;

R(7) is C₅-C₇-cycloalkyl or phenyl,

which is unsubstituted or substituted by 1-3 substituents selected from the group consisting of F, Cl, CF₃, methyl, methoxy and NR(8)R(9);

R(8) and R(9)

are H or C₁-C₄-alkyl;

or

R(5) is H;

R(6) is H or C₁-C4-alkyl,

or

R(5) and R(6)

together are 4 or 5 methylene groups, of which one CH₂ group can be replaced by an O, S, NH, N—CH₃ or N-benzyl;

R(2) is hydrogen, F, Cl, Br, (C₁-C₄)-alkyl-, O—(CH₂)ₘCₚF₂ₚ₊₁ or —X—R(10);

m is zero or 1;

p is 1, 2 or 3;

X is O, S or NR(11);

R(10) is H, C₁-C₆-alkyl, C₅-C₇-cycloalkyl, cyclohexylmethyl, cyclopentylmethyl or —CₙH₂ₙ—R(12);

n is zero, 1, 2, 3 or 4;

R(12) is phenyl,

which is unsubstituted or substituted by 1-3 substituents selected from the group consisting of F, Cl, CF₃, methyl, methoxy and NR(8)R(9); R(8) and R(9)

are H or C₁-C₄-alkyl;

R(11) is hydrogen or C₁-C₃-alkyl;

or

R(10) and R(11)

together are 4 or 5 methylene groups, of which one CH₂ group can be replaced by O, S, NH, N—CH₃ or N-benzyl;

R(3) is defined as R(1), or is C₁-C₆-alkyl, nitro, cyano, trifluoromethyl, F,

Cl, Br, I or —X—R(10);

X is O, S or NR(11);

R(10) is H, C₁-C₆-alkyl, C₅-C7-cycloalkyl, cyclohexylmethyl, cyclopentylmethyl or —CₙH₂ₙ—R(12);

n is zero to 4;

R(12) is phenyl,

which is unsubstituted or substituted by 1-3 substituents selected from the group consisting of F, Cl, CF₃, methyl, methoxy and NR(8)R(9);

R(8) and R(9)

are H or C₁-C₄-alkyl;

R(11) is C₁-C₃-alkyl,

or

R(10) and R(11)

together are 4 or 5 methylene groups, of which one CH₂ group can be replaced by O, S, NH, N—CH₃ or N-benzyl;

and their pharmaceutically tolerable salts;

(HOE 92/F 035 EP-A 556 673)

c) ortho-substituted benzoylguanidines of the formula I

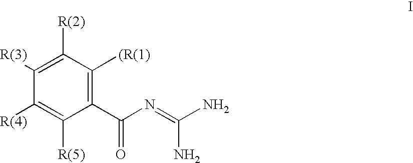

in which:

R(1) is F, Cl, Br, I, C₁-C₆-alkyl or —X—R(6);

X is O, S, NR(7) or Y—ZO;

Y is O or NR(7);

Z is C or SO;

R(6) is H, C₁-C₆-alkyl, C₅-C₇-cycloalkyl, cyclohexylmethyl, cyclopentylmethyl, —(CH₂)ₘCₚF₂ₚ₊₁ or —CₙH₂ₙ—R(8);

m is zero or 1;

p is 1-3;

n is zero to 4;

R(8) is phenyl,

which is unsubstituted or substituted by 1-3 substituents selected from the group consisting of the groups F, Cl, CF₃, methyl, methoxy and NR(9)R(10);

R(9) and R(10)

are H or C₁-C₄-alkyl;

R(7) is H or C₁-C₃-alkyl;

or

R(6) and R(7)

together are 4 or 5 methylene groups, of which one CH₂ group can be replaced by O, S, NH, N—CH₃ or N-benzyl;

R(3) is H or —X—R(6);

X is O, S, NR(7) or Y—ZO;

R(7) is H or C₁-C₃-alkyl;

Y is O or NR(7);

where Y is bonded to the phenyl radical of the formula I,

Z is C or SO;

R(6) is H, C₁-C₆-alkyl, C₅-C₇-cycloalkyl, cyclohexylmethyl, cyclopentylmethyl, —(CH₂)ₘCₚF₂ₚ₊₁ or —CₙH₂ₙ—R(8);

m is zero or 1;

p is 1-3;

n is zero to 4;

R(8) is phenyl,

which is unsubstituted or substituted by 1-3 substituents selected from the group consisting of F, Cl, CF₃, methyl, methoxy and NR(9)R(10);

R(9) and R(10)

are H or C₁-C₄-alkyl;

or

R(6) and R(7)

together are 4 or 5 methylene groups, of which one CH₂ group can be replaced by O, S, NH, N—CH₃ or N-benzyl;

R(2) and R(4)

identically or differently are R(11)—SO_{q}— or R(12)R(13)N—SO₂—;

q is zero-2;

R(11) is C₁-C₄-alkyl,

which is unsubstituted or carries phenyl as a substituent, where phenyl is unsubstituted or substituted by 1-3 substituents selected from the group consisting of F, Cl, CF₃, methyl, methoxy and NR(9)R(10);

R(9) and R(10)

are H or C₁-C₄-alkyl;

R(12) and R(13)

are defined as R(6) and R(7);

or one of the two radicals R(2) or R(4)

is hydrogen or is defined as R(1);

R(5) is H, methyl, F, Cl or methoxy,

and their pharmaceutically tolerable salts;

(HOE 92/F 036—U.S. Pat. No. 5,364,868)

d) benzoylguanidines of the formula I

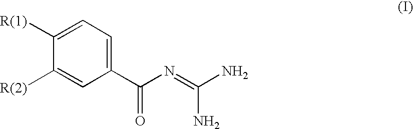

in which:

R(1) or R(2)

is an amino group —NR(3)R(4);

R(3) and R(4)

identically or differently are H, C₁-C₆-alkyl or C₃-C₇-cycloalkyl;

or

R(3) is phenyl-(CH₂)ₚ—;

p is 0, 1 , 2, 3 or 4;

or

R(3) is phenyl,

where the phenyl in each case is unsubstituted or carries one to two substituents selected from the group consisting of fluorine, chlorine, methyl and methoxy;

or

R(3) and R(4)

together can be a straight-chain or branched C₄-C₇-methylene chain, where one —CH₂— member of the methylene chain can be replaced by oxygen, S or NR(5);

R(5) is H or lower alkyl;

the other substituent R(1) or R(2) in each case

is H, F, Cl, C₁-C₄-alkyl, C₁-C₄-alkoxy, CF₃, CₘF₂ₘ₊₁—CH₂—, benzyl or phenoxy,

where the respective phenyl radical is unsubstituted or carries one to two substituents selected from the group consisting of methyl, methoxy, fluorine and chlorine;

m is 1, 2 or 3;

and their pharmaceutically tolerable salts;

(92/F 197 K—NZ 248 013)

e) benzoylguanidines of the formula I

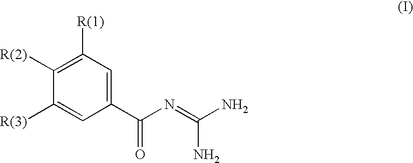

in which:

R(1) is R(4)—SOₘ or R(5)R(6)N—SO₂—;

m is zero, 1 or 2;

R(4) and R(5)

are C₁-C₈-alkyl, C₃-C₆-alkenyl or —CₙH₂ₙ—R(7);

n is zero, 1, 2, 3 or 4;

R(7) is C₅-C₇-cycloalkyl or phenyl,

which is unsubstituted or substituted by 1-3 substituents selected from the group consisting of F, Cl, CF₃, methyl, methoxy and NR(8)R(9);

R(8) and R(9)

are H or C₁-C₄-alkyl;

or

R(5) is H;

R(6) is H or C₁-C₄-alkyl;

or

R(5) and R(6)

together are 4 or 5 methylene groups, of which one CH₂ group can be replaced by an O, S, NH, N—CH₃ or N-benzyl;

R(2) is hydrogen, straight-chain or branched (C₅-C₈)-alkyl, —CR(13)═CHR(12) or —C≡CR(12);

R(12) is phenyl,

which is unsubstituted or substituted by 1-3 substituents selected from the group consisting of F, Cl, CF₃, methyl, methoxy and NR(14)R(15);

R(14) and R(15)

are H or (C₁-C₄)-alkyl;

or

R(12) is (C₁-C₉)-heteroaryl,

which is unsubstituted or substituted as phenyl,

or

R(12) is (C₁-C₆)-alkyl,

which is unsubstituted or substituted by 1-3 OH,

or

R(12) is (C₃-C₈)-cycloalkyl;

R(13) is hydrogen or methyl,

or

R(12) is (C₃-C₈)-cycloalkyl, (C₃-C₈)-cycloalkyl-(C₁-C₄)-alkyl, phenyl,

C₆H₅—(C₁-C₄)-alkyl, naphthyl, biphenylyl, 1,1-diphenyl-(C₁-C₄)-alkyl, cyclopentadienyl, pyridyl, pyrrolyl, furanyl, thienyl, thiazolyl, oxazolyl, indenyl, quinolyl, indolyl, benzofuranyl, benzothienyl, benzothiazolyl, benzoxazolyl, imidazolyl, pyrazolyl, triazolyl, tetrazolyl, isoxazolyl, isothiazolyl, pyrazinyl, pyrimidinyl, pyridazinyl, indazolyl, isoquinolyl, phthalazinyl, quinoxalinyl, quinazolinyl or cinnolinyl;

R(3) is defined as R(2);

and where the aromatic substituents R(2) and R(3) are unsubstituted or substituted by 1-3 substituents from the groups F, Cl, CF₃, (C₁-C₄)-alkyl or -alkoxy, or NR(10)R(11) with R(10) and R(11) being H or (C₁-C₄)-alkyl;

and their pharmaceutically tolerable salts;

(HOE 92/F 303 K—EP-A 589 336, NZ 248 703)

f) benzoylguanidines of the formula I

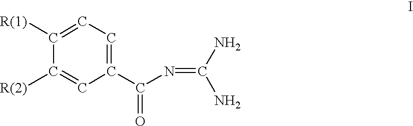

in which:

R(1) or R(2)

is R(3)—S(O)ₙ— or R(4)R(5)N—SO₂—

the other substituent R(1) or R(2) in each case

is H, OH, F, Cl, Br, I, C₁-C₄-alkyl, C₁-C₄-alkoxy, benzyloxy or phenoxy,

which is unsubstituted or carries one to three substituents selected from the group consisting of fluorine, chlorine, methyl, methoxy, hydroxyl or benzyloxy,

R(3)—S(O)ₙ, —NR(4)R(5) or 3,4-dehydropiperidine

R(3) is C₁-C₆-alkyl, C5-C₇-cycloalkyl, cyclopentylmethyl,

cyclohexylmethyl or phenyl,

which is unsubstituted or carries one to three substituents selected from the group consisting of fluorine, chlorine, methyl and methoxy;

R(4) and R(5)

identically or differently, are H or C₁-C₆-alkyl;

or

R(4) is phenyl-(CH₂)ₘ—;

m is 1, 2, 3 or 4;

or

R(4) is phenyl,

which is unsubstituted or carries one to two substituents selected from the group consisting of fluorine, chlorine, methyl and methoxy;

or

R(4) and R(5)

together are a straight-chain or branched C₄-C₇-chain, where the chain can additionally be interrupted by O, S or NR(6),

R(6) is H or methyl;

or

R(4) and R(5)

together with the nitrogen atom to which they are bonded, are a dihydroindole, tetrahydroquinoline or tetrahydroisoquinoline system;

n is zero, 1 or 2;

and their pharmaceutically tolerable salts;

(92/F 304—U.S. Pat. No. 5,416,094)

g) isoquinolines of the formula I

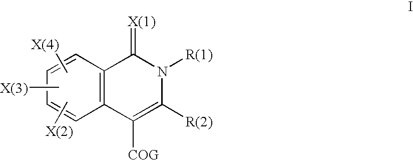

in which:

R(1) is hydrogen, alkyl, cycloalkyl, arylalkyl, alkenyl, substituted aminoalkyl or an aryl or heteroaryl ring;

where the rings are unsubstituted or substituted by 1-3 groups selected from the group consisting of halogen, nitro, amino, mono(lower alkyl)amino, di(lower alkyl)amino, lower alkyl, lower alkoxy, benzyloxy, phenoxy, hydroxyl, trifluoromethyl,

R(2) is hydrogen, halogen, alkyl or aryl;

which is unsubstituted or substituted by 1-3 groups selected from the group consisting of halogen, nitro, amino, mono(lower alkyl)amino, di(lower alkyl)amino, lower alkyl, lower alkoxy, benzyloxy, phenoxy, hydroxyl,

G is —N═C{[NR(3)R(4)][N(R5)R(6)]};

R(3), R(4), R(5) and R(6) are hydrogen,

X(2), X(3) and X(4)

independently of one another are hydrogen, halogen, nitro, amino, alkyl, sulfonamide, mono(lower alkyl)amino, di(lower alkyl)amino, lower alkyl, benzyloxy, hydroxyl;

X(1) is hydrogen, oxygen, sulfur or NR(7);

R(7) is hydrogen, alkyl, cycloalkyl, arylalkyl, alkenyl, substituted aminoalkyl or an aryl or a heteroaryl ring;

which rings are unsubstituted or substituted by 1-3 groups selected from the group consisting of halogen, nitro, amino, mono(lower alkyl)amino, di(lower alkyl)amino, lower alkyl, lower alkoxy, benzyloxy, phenoxy, hydroxyl and trifluoromethyl;

in which substituents each alkyl chain or alkenyl chain can be interrupted by oxygen, sulfur or NR(8);

R(8) is hydrogen, alkyl, cycloalkyl, arylalkyl, alkenyl, substituted aminoalkyl or an aryl or heteroaryl ring,

which rings are unsubstituted or substituted by 1-3 groups selected from the group consisting of halogen, nitro, amino, mono(lower alkyl)amino, di(lower alkyl)amino, lower alkyl, lower alkoxy, benzyloxy, phenoxy, hydroxyl and trifluoromethyl;

and their pharmaceutically acceptable salts;

(92/F 404—EP 602 522, NZ 250 438)

h) compounds of the formula I

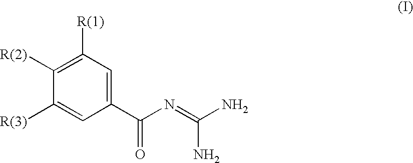

in which:

R(1) is hydrogen, F, Cl, Br, I, —NO₂, —C≡N, —CF₃, R(4)—SOₘ or

R(5)R(6)N—SO₂—;

m is zero, 1 or 2;

R(4) and R(5)

are (C₁-C₈)-alkyl, (C₃-C₆)-alkenyl, —CₙH₂ₙ—R(7) or CF₃;

n is zero, 1, 2, 3 or 4;

R(7) is (C₃-C₇)-cycloalkyl or phenyl,

which is not substituted or is substituted by 1-3 substituents selected from the group consisting of F, Cl, CF₃, methyl, methoxy and NR(8)R(9);

R(8) and R(9)

are H or C₁-C₄-alkyl;

or

R(5) is H;

R(6) is H or (C₁-C₄)-alkyl;

or

R(5) and R(6)

together are 4 or 5 methylene groups, of which one CH₂ group can be replaced by oxygen, S, NH, N—CH₃ or N-benzyl;

R(2) is —SR(10), —OR(10), —NHR(10), —NR(10)R(11), —CHR(10)R(12), —[CR(12)R(13)OR(13′)], —{C—[CH₂—OR(13′)]R(12)(R(13)} or —[CR(18)R(17)]ₚ—(CO)—[CR(19)R(20)]_{q}—R(14);

R(10) and R(11)

identically or differently are —[CHR(16)]ₛ—(CH₂)ₚ—(CHOH)_{q}—(CH₂)ᵣ—(CHOH)ₜ—R(21) or —(CH₂)ₚ—O—(CH₂—CH₂O)_{q}—R(21),

R(21) is hydrogen, methyl,

p, q, r identically or differently

are zero, 1, 2, 3 or 4;

s is zero or 1;

t is 1, 2, 3 or 4;

R(12) and R(13)

identically or differently are hydrogen, (C₁-C₆)-alkyl or, together with the carbon atom carrying them, are a (C₃-C₈)-cycloalkyl,

R(13′) is hydrogen or (C₁-C₄)-alkyl;

R(14) is H, (C₁-C₆)-alkyl, (C₃-C₈)-cycloalkyl or —CₐH₂ₐ—R(15);

a is zero, 1, 2, 3 or 4;

R(15) is phenyl,

which is unsubstituted or substituted by 1-3 substituents selected from the group consisting of F, Cl, CF₃, methyl, methoxy and NR(8)R(9);

R(8) and R(9)

are H or (C₁-C₄)-alkyl;

or

R(15) is (C₁-C₉)-heteroaryl,

which is unsubstituted or substituted as phenyl,

or

R(15) is (C₁-C₆)-alkyl,

which is unsubstituted or substituted by 1-3 OH;

R(16), R(17), R(18), R(19) and R(20)

are hydrogen or (C₁-C₃)-alkyl;

R(3) is defined as R(1),

or

R(3) is (C₁-C₆)-alkyl or —X—R(22);

X is oxygen, S or NR(16);

R(16) is H or (C₁-C₃)-alkyl;

or

R(22) and R(16)

together are 4 or 5 methylene groups, of which one CH₂ group can be replaced by oxygen, S, NH, N—CH₃ or N-benzyl;

R(22) is defined as R(14);

and their pharmaceutically tolerable salts;

(HOE 92/F 405—EP 602 523, NZ 250 437)

i) benzoylguanidines of the formula I

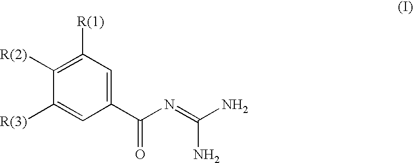

in which:

R(1) is hydrogen, F, Cl, Br, I, —NO₂, —C≡N, R(16—CₚH₂ₚ—O_{q}, R(4)—SOₘ or R(5)R(6)N—SO₂—;

m is zero, 1 or 2;

p is zero or 1;

q is zero, 1, 2 or 3;

R(16) is CᵣF₂ᵣ₊₁;

r is 1, 2 or 3;

R(4) and R(5)

are (C₁-C₈)-alkyl, (C₃-C₆)-alkenyl, —CₙH₂ₙ—R(7) or CF₃;

n is zero, 1, 2, 3 or 4;

R(7) is (C₃-C₇)-cycloalkyl or phenyl,

which is not substituted or is substituted by 1-3 substituents selected from the group consisting of F, Cl, CF₃, methyl, methoxy and NR(8)R(9);

R(8) and R(9)

are H or C₁-C₄-alkyl;

or

R(5) is H;

R(6) is H or (C₁-C₄)-alkyl;

or

R(5) and R(6)

together are 4 or 5 methylene groups, of which one CH₂ group can be replaced by oxygen, S, NH, N—CH₃ or N-benzyl,

R(2) is (C₁-C₉)-heteroaryl,

which is linked via C or N and which is unsubstituted or substituted by 1-3 substituents selected from the group consisting of F, Cl, CF₃, CH₃, methoxy, hydroxyl, amino, methylamino and dimethylamino;

or

R(2) is —SR(10), —OR(10), —NR(10)R(11), —CR(10)R(11)R(12);

R(10) is —CₐH₂ₐ—(C₁-C₉)-heteroaryl,

which is unsubstituted or substituted by 1-3 substituents selected from the group consisting of F, Cl, CF₃, CH₃, methoxy, hydroxyl, amino, methylamino and dimethylamino;

a is zero, 1 or 2;

R(11) and R(12)

independently of one another are defined as R(10) or are hydrogen or (C₁-C₄)-alkyl;

R(3) is defined as R(1), or is (C₁-C₆)-alkyl or —X—R(13);

X is oxygen, S, or NR(14);

R(14) is H or (C₁-C₃)-alkyl;

R(13) is H, (C₁-C₆)-alkyl, (C₃-C₈)-cycloalkyl or —C_{b}H_{2b}—R(15);

b is zero, 1, 2, 3 or 4;

or

R(13) and R(14)

together are 4 or 5 methylene groups, of which one CH₂ group can be replaced by oxygen, S, NH, N—CH₃ or N-benzyl;

R(15) is phenyl,

which is unsubstituted or substituted by 1-3 substituents selected from the group consisting of F, Cl, CF₃, methyl, methoxy and NR(8)R(9);

R(8) and R(9)

are H or (C₁-C₄)-alkyl;

and their pharmaceutically tolerable salts;

(HOE 92/F 411—NZ 250 450, EP 603 650)

k) benzoylguanidines of the formula I

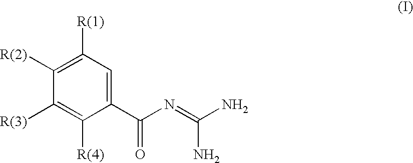

in which:

one of the substituents R(1), R(2), R(3) or R(4):

is an amino group —NR(5)[CₙH₂ₙ—R(6)];

R(5) is hydrogen or C₍₁₋₆₎-alkyl;

n is zero, 1, 2, 3 or 4;

R(6) is H or C₍₁₋₄₎-alkyl;

in which one CH₂ group can be replaced by 1 sulfur atom or a group NR(7);

R(7) is hydrogen, methyl or ethyl;

or

R(6) is C₍₃₋₈₎-cycloalkyl or phenyl,

which is unsubstituted or carries 1, 2 or 3 substituents selected from the group consisting of F, Cl, Br, methyl, methoxy, —NR(8)R(9);

R(8) and R(9)

are H, methyl or ethyl;

or

R(5) and R(6)

together with the nitrogen atom are a 5-, 6- or 7-membered ring, in which 1 carbon atom can be replaced by oxygen, S or NR(10);

R(10) is H, C₍₁₋₃₎-alkyl or benzyl;

and the other substituents R(1), R(2), R(3), R(4) in each case are:

hydrogen, F, Cl, Br, I, CN, CF₃, NO₂, CF₃—O—, CₘF₂ₘ₊₁—CH₂—O— or

R(11)—C_{q}H_{2q}—Xₚ—;

m is 1, 2 or 3;

q is zero, 1, 2, 3 or 4;

p is zero or 1;

X is oxygen or NR(12);

R(12) is H or C₍₁₋₃₎-alkyl;

R(11) is hydrogen, C₍₁₋₆₎-alkyl, C₍₃₋₈₎-cycloalkyl or phenyl,

which is unsubstituted or substituted by 1, 2 or 3 substituents selected from the group consisting of F, Cl, CH₃, CH₃—O—and NR(13)R(14);

R(13) and R(14)

are H, methyl or ethyl;

and their pharmaceutically tolerable salts;

(HOE 921F 422—EP 604 852)

l) benzoylguanidines of the formula I

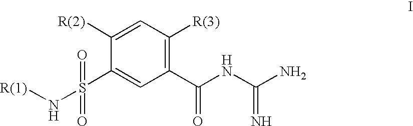

in which

R(1) is R(4)R(5)N—C(X)—;

X is oxygen, S or N—R(6);

R(4) and R(5)

identically or differently, are H, (C₁-C₈)-alkyl, (C₃-C₆)-alkenyl or —CₙH₂—R(7);

n is zero, 1, 2, 3 or 4;

R(7) is (C₅-C₇)-cycloalkyl or phenyl,

which is unsubstituted or substituted by 1-3 substituents selected from the group consisting of F, Cl, CF₃, methoxy and (C₁-C₄)-alkyl;

or

R(4) and R(5)

together are 4 or 5 methylene groups, of which one CH₂ group can be replaced by oxygen, S, NH, N—CH₃ or N-benzyl;

R(6) is defined as R(4) or is amidine;

R(2) is H, F, Cl, Br, I, (C₁-C₈)-alkyl, 1-alkenyl or 1-alkynyl, (C₃-C₈)-cycloalkyl, (C₃-C₈)-cycloalkyl-(C₁-C₄)-alkyl, phenyl, C₆H₅—(C₁-C₄)-alkyl, naphthyl, biphenylyl, 1,1-diphenyl-(C₁-C₄)-alkyl, cyclopentadienyl, pyridyl, thiopyridyl, pyrrolyl, furanyl, thienyl, thiazolyl, oxazolyl, indenyl, quinolyl, indolyl, benzofuranyl, benzothienyl, benzothiazolyl, benzoxazolyl or —W—R(8);

W is oxygen, S or NR(9);

R(8) is H, (C₁-C₆)-alkyl, (C₅-C₇)-cycloalkyl, cyclohexylmethyl, cyclopentylmethyl, —(CH₂)ₘCₚF₂ₚ₊₁ or —C_{q}H_{2q}—R(10);

m is zero or 1;

p is 1, 2 or 3;

q is zero, 1, 2, 3 or 4;

R(10) is phenyl,

which is unsubstituted or substituted by 1-3 substituents selected from the group consisting of F, Cl, CF₃, methyl, methoxy and NR(11)R(12);

R(11) and R(12)

are H or (C₁-C₄)-alkyl;

R(9) is H or (C₁-C₃)-alkyl;

or

R(8) and R(9)

together are 4 or 5 methylene groups, of which one CH₂ group can be replaced by oxygen, S, NH, N—CH₃ or N-benzyl;

R(3) is H, F, Cl, Br, I, (C₁-C₆)-alkyl or —W—R(8) as defined for R(2),

and their pharmaceutically acceptable salts;

(93/F 054—NZ 250 919, EP-A 612 723)

m) benzoylguanidines of the formula I

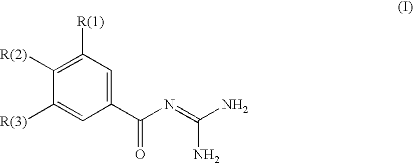

in which:

R(1), R(2), R(3)

are hydrogen, F, Cl, Br, I or (C₁-C₁₂)-alkyl;

one of the substituents R(1), R(2) or R(3)

is N₃, CN, OH or (C₁-C₁₀)-alkyloxy, if at least one of the remaining substituents R(1), R(2) or R(3) is a sufficiently lipophilic alkyl radical having 3 to 12 carbon atoms;

or

one of the substituents R(1), R(2) or R(3)

is R(4)—CₙH₂ₙ—Oₘ—;

m is zero or 1;

n is zero, 1, 2 or 3;

R(4) is CₚF₂ₚ₊₁;

p is 1, 2 or 3, if n is zero or 1;

or

R(4) is (C₃-C₁₂)-cycloalkyl, phenyl, pyridyl, quinolyl or isoquinolyl,

where the aromatic and heteroaromatic ring systems are unsubstituted or substituted by a substituent selected from the group consisting of F, Cl, CF₃, methyl, methoxy and

NR(5)R(6);

R(5) and R(6)

are hydrogen or (C₁-C₄)-alkyl;

or one of the substituents R(1), R(2) or R(3)

is —C≡CR(5) or —C[R(6)]═CR(5);

R(5) is phenyl,

which is unsubstituted or substituted by 1-3 substituents selected from the group consisting of F, Cl, CF₃, methyl, methoxy, hydroxyl, amino, methylamino and dimethylamino, (C₁-C₉)-heteroaryl,

which is unsubstituted or substituted as phenyl,

or

R(5) is (C₁-C6)-alkyl,

which is unsubstituted or substituted by 1-3 OH;

or

R(5) is (C₃-C₈)-cycloalkyl,

R(6) is hydrogen or methyl;

and their pharmacologically acceptable salts;

93/F 153—EP-A 627 413, NZ 260 660)

o) benzoylguanidines of the formula I

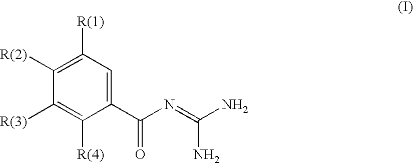

in which:

R(1) is hydrogen, F, Cl, Br, I, —NO₂, —C≡N, Xₒ—(CH₂)ₚ—(CF₂)_{q}—CF₃, R(5)—SOₘ, R(6)—CO— or R(6)R(7)N—SO₂—, where

X is oxygen, S or NR(14);

m is zero, 1 or 2;

o is zero or 1;

p is zero, 1 or 2;

q is zero, 1, 2, 3, 4, 5 or 6;

R(5) and R(6)

are (C₁-C₈)-alkyl, (C₃-C₆)-alkenyl, —CₙH₂ₙ—R(8) or CF₃;

n is zero, 1, 2, 3 or 4;

R(8) is (C₃-C₇)-cycloalkyl or phenyl,

which is not substituted or is substituted by 1-3 substituents selected from the group consisting of F, Cl, CF₃, methyl, methoxy and NR(9)R(10);

R(9) and R(10)

are H or C₁-C₄-alkyl;

or

R(6) is H;

R(7) is H or (C₁-C₄)-alkyl;

or

R(6) and R(7)

together are 4 or 5 methylene groups, of which one CH₂ group can be replaced by oxygen, S, NH, N—CH₃ or N-benzyl;

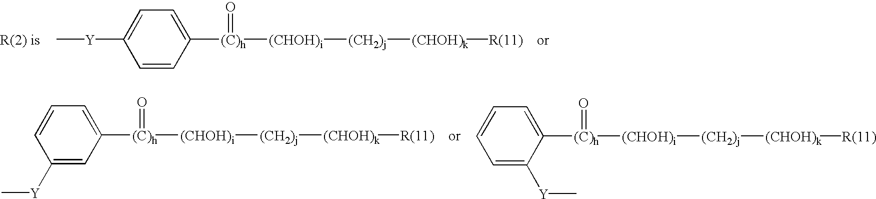

Y is oxygen, —S— or —NR(12)—;

R(11) and R(12)

are hydrogen or (C₁-C₃)-alkyl;

h is zero or 1;

i, j and k

independently are zero, 1, 2, 3 or 4;

but where h, i and k are not simultaneously zero,

R(3) is defined as R(1), or is (C₁-C₆)-alkyl or —X—R(13);

X is oxygen, S or NR(14);

R(14) is H or (C₁-C₃)-alkyl;

R(13) is H, (C₁-C₆)-alkyl, (C₃-C₈)-cycloalkyl or —C_{b}H_{2b}—R(15);

b is zero, 1, 2, 3 or 4;

or

R(13) and R(14)

together are 4 or 5 methylene groups, where one CH₂ group can be replaced by oxygen, S, NH, N—CH₃ or N-benzyl;

R(15) is phenyl,

which is unsubstituted or substituted by 1-3 substituents selected from the group consisting of F, Cl, CF₃, methyl, methoxy and NR(9)R(10);

R(9) and R(10)

are H or (C₁-C₄)-alkyl;

R(4) is hydrogen, —OR(16) or —NR(16)R(17);

R(16) and R(17)

independently are hydrogen or (C₁-C₃)-alkyl;

and their pharmaceutically tolerable salts;

(HOE 93/F 154—EP-A 628 543, NZ 260 681)

p) benzoylguanidines of the formula I

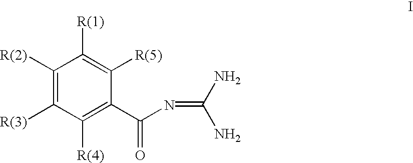

in which:

R(1) is R(6)—CO or R(7)R(8)N—CO;

R(6) is (C₁-C₈)-alkyl, (C₁-C₈)-perfluoroalkyl, (C₃-C₈)-alkenyl or —CₙH₂ₙ—R(9);

n is zero, 1, 2, 3 or 4;

R(9) is (C₃-C₈)-cycloalkyl, phenyl, biphenylyl or naphthyl,

where the aromatics are not substituted or are substituted by 1-3 substituents selected from the group consisting of F, Cl, CF₃, methyl, methoxy and NR(10)R(11);

R(10) and R(11)

are H, (C₁-C₄)-alkyl or (C₁-C₄)-perfluoroalkyl;

R(7) is H, (C₁-C₈)-alkyl, (C₁-C₈)-perfluoroalkyl, (C₃-C₈)-alkenyl or —CₙH₂ₙ—R(12);

n is zero, 1, 2, 3 or 4;

R(12) is (C₃-C₈)-cycloalkyl, phenyl, biphenylyl or naphthyl,

where the aromatics are not substituted or are substituted by 1-3 substituents selected from the group consisting of F, Cl, CF₃, methyl, methoxy and NR(13)R(14);

R(13) and R(14)

are H, (C₁-C₄)-alkyl or (C₁-C₄)-perfluoroalkyl;

R(8) is H, (C₁-C₄)-alkyl or (C₁-C₄)-perfluoroalkyl;

or

R(7) and R(8)

together are 4 or 5 methylene groups, of which one CH2 group can be replaced by oxygen, S, NH, N—CH₃ or N-benzyl;

R(2) is defined as R(1), or is H, F, Cl, Br, I, CN, NO₂, (C₁-C₈)-alkyl, (C₁-C₈)-perfluoroalkyl, (C3-C₈)-alkenyl or —CₙH₂ₙR(15);

n is zero, 1, 2, 3 or 4;

R(15) is (C₃-C₈)-cycloalkyl, phenyl, biphenylyl or naphthyl,

where the aromatics are not substituted or are substituted by 1-3 substituents selected from the group consisting of F, Cl, CF₃, methyl, methoxy and

NR(16)R(17);

R(16) and R(17)

are H, (C₁-C₄)-alkyl or (C₁-C₄)-perfluoroalkyl;

or

R(2) is (C₁-C₉)-heteroaryl,

which is linked via C or N and which is unsubstituted or substituted by 1-3 substituents selected from the group consisting of F, Cl, CF₃, CH₃, methoxy, hydroxyl, amino, methylamino and dimethylamino;

or

R(2) is SR(18), —OR(18), —NR(18)R(19), —CR(18)R(19)R(20);

R(18) is —CₐH₂ₐ—(C₁-C₉)-heteroaryl,

which is unsubstituted or substituted by 1-3 substituents selected from the group consisting of F, Cl, CF₃, CH₃, methoxy, hydroxyl, amino, methylamino and dimethylamino;

a is zero, 1 or 2;

R(19) and R(20)

independently of one another are defined as R(18) or are hydrogen, (C₁-C₄)-alkyl or (C₁-C₄)-perfluoroalkyl;

or

R(2) is R(21)—SOₘ or R(22)R(23)N—SO₂—;

m is 1 or 2;

R(21) is (C₁-C₈)-alkyl, (C₁-C₈)-perfluoroalkyl, (C₃-C₈)-alkenyl, —CₙH₂ₙ,—R(24),

n is zero, 1, 2, 3 or 4;

R(24) is (C₃-C₈)-cycloalkyl, phenyl, biphenylyl or naphthyl,

where the aromatics are not substituted or are substituted by 1-3 substituents selected from the group consisting of F, Cl, CF₃, methyl, methoxy and NR(27)R(28);

R(27) and R(28)

are H, (C₁-C₄)-alkyl or (C₁-C₄)-perfluoroalkyl;

R(22) is H, (C₁-C₈)-alkyl, (C₁-C₈)-perfluoroalkyl, (C₃-C₈)-alkenyl, —CₙH₂ₙ—R(29);

n is zero, 1, 2, 3 or 4;

R(29) is (C₃-C₈)-cycloalkyl, phenyl, biphenylyl or naphthyl,

where the aromatics are not substituted or are substituted by 1-3 substituents selected from the group consisting of F, Cl, CF₃, methyl, methoxy and NR(30)R(31);

R(30) and R(31)

are H, (C₁-C₄)-alkyl or (C₁-C₄)-perfluoroalkyl;

R(23) is H, (C₁-C₄)-alkyl or (C₁-C₄)-perfluoroalkyl;

or

R(22) and R(23)

together are 4 or 5 methylene groups, of which one CH₂ group can be replaced by oxygen, S, NH, N—CH₃ or N-benzyl;

or

R(2) is R(33)X—;

X is oxygen, S, NR(34), (D═O)A—, NR(34)C═MN^{(*)}R(35)—;

M is oxygen or S;

A is oxygen or NR(34);

D is C or SO;

R(33) is (C₁-C₈),alkyl (C₃-C₈)-alkenyl, (CH₂)_{b}C_{d}F_{2d+1}, —CₙH₂ₙ—R(36),

b is zero or 1;

d is 1, 2, 3, 4, 5, 6 or 7;

n is zero, 1, 2, 3 or 4;

R(36) is (C₃-C₈)-cycloalkyl, phenyl, biphenylyl or naphthyl,

where the aromatics are not substituted or are substituted by 1-3 substituents selected from the group consisting of F, Cl, CF₃, methyl, methoxy and NR(37)R(38);

R(37) and R(38)

are H, (C₁-C₄)-alkyl or (C₁-C₄)-perfluoroalkyl;

R(34) is H, (C₁-C₄)-alkyl or (C₁-C₄)-perfluoroalkyl;

R(35) is defined as R(33);

or

R(33) and R(34)

together are 4 or 5 methylene groups, of which one CH₂ group can be replaced by oxygen, S, NH, N—CH₃ or N-benzyl;

where A and N^{(*)} are bonded to the phenyl nucleus of the benzoylguanidine parent structure;

or

R(2) is —SR(40), —OR(40), —NHR(40), —NR(40)R(41), —CHR(40)R(42), —C[R(42)R(43)OH], —C≡CR(45), —CR(46)═CHR(45), —[CR(47)R(48)]ᵤ—(CO)—[CR(49)R(50)]ᵥ—R(44);

R(40), R(41)

identically or differently are —(CH₂)ₚ—(CHOH)_{q}—(CH₂)ᵣ—(CHOH)ₜ—R(51) or —(CH₂)ₚ—O—(CH₂—CH₂O)_{q}—R(51);

R(51) is hydrogen or methyl;

u is 1, 2, 3 or 4;

v is zero, 1, 2, 3 or 4;

p, q, r

identically or differently are zero, 1, 2, 3 or 4;

t is 1, 2, 3 or 4;

R(42) and R(43)

identically or differently are hydrogen or (C₁-C₆)-alkyl;

or

R(42) and R(43)

together with the carbon atom carrying them form a (C₃-C₈)-cycloalkyl;

R(44) is H, (C₁-C₆)-alkyl, (C₃-C₈)-cycloalkyl or —CₑH₂ₑ—R(45);

e is zero, 1, 2, 3 or 4;

R(45) is phenyl,

which is unsubstituted or substituted by 1-3 substituents from the group consisting of F, Cl, CF₃, methyl, methoxy and NR(52)R(53) where

R(52) and R(53) are H or (C₁-C₄)-alkyl, or

R(45) is (C₁-C₉)-heteroaryl,

which is unsubstituted or substituted as phenyl;

or

R(45) is (C₁-C₆)-alkyl,

which is unsubstituted or substituted by 1-3 OH;

R(46), R(47), R(48), R(49) and R(50)

are hydrogen or methyl;

or

R(2) is R(55)—NH—SO₂—;

R(55) is R(56)R(57)N—(C═Y)—;

Y is oxygen, S or N—R(58);

R(56) and R(57)

identically or differently are H, (C₁-C₈)-alkyl, (C₃-C₆)-alkenyl or —C_{f}H_{2f}—HR(59);

f is zero, 1, 2, 3 or 4;

R(59) is (C₅-C₇)-cycloalkyl or phenyl,

which is unsubstituted or substituted by 1-3 substituents selected from the group consisting of F, Cl, CF₃, methoxy and (C₁-C₄)-alkyl;

or

R(⁵⁶) and R(57)

together are 4 or 5 methylene groups, of which one CH₂ group can be replaced by oxygen, S, NH, N—CH₃ or N-benzyl;

R(58) is defined as R(56) or is amidine;

R(3), R(4) and R(5)

independently of one another are defined as R(1) or R(2);

and their pharmaceutically tolerable salts;

(HOE 93/F 220—EP-A 640 593, NZ 264 117)

q) benzoylguanidines of the formula I

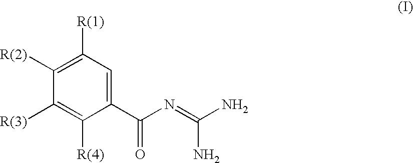

in which:

R(1) is hydrogen, F, Cl, Br, I, —NO², —C≡N, —Xₒ—(CH₂)ₚ—(CF₂)_{q}—CF₃,

R(5)—SOₘ—, R(6)—CO—, R(6)R(7)N—CO— or R(6)R(7)N—SO₂—;

X is oxygen, —S— or NR(14);

m is zero, 1 or 2;

o is zero or 1;

p is zero, 1 or 2;

q is zero, 1, 2, 3, 4, 5 or 6;

R(5) and R(6)

are (C₁-C₈)-alkyl, (C₃-C₆)-alkenyl, —CₙH₂ₙ—R(8) or CF₃;

n is zero, 1, 2, 3 or 4;

R(8) is (C₃-C₇)-cycloalkyl, phenyl,

which is not substituted or is substituted by 1 to 3 substituents selected from the group consisting of F, Cl, CF₃, methyl, methoxy and NR(9)R(10);

R(9) and R(10)

are H or (C₁-C₄)-alkyl;

or

R(6) is hydrogen;

R(7) is hydrogen or (C₁-C₄)-alkyl;

or

R(6) and R(7)

together are 4 or 5 methylene groups, of which one CH₂ group can be replaced by oxygen, S, NH, N—CH₃ or N-benzyl;

R(2) is

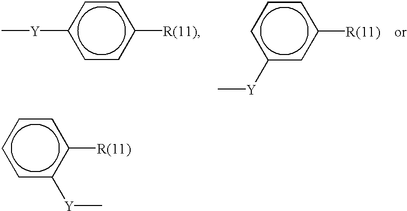

R(11) is (C₁-C₉)-heteroaryl,

which is linked via C or N and which is unsubstituted or substituted by 1 to 3 substituents selected from the group consisting of F, Cl, CF₃, CH₃, methoxy, hydroxyl, amino, methylamino, dimethylamino and benzyl;

Y is oxygen, —S— or NR(12);

R(12) is H or (C₁-C₄)-alkyl;

R(3) is defined as R(1);

or

R(3) is (C₁-C₆)-alkyl or —X—R(13);

X is oxygen, —S— or NR(14);

R(14) is H or (C₁-C₃)-alkyl;

R(13) is H, (C₁-C₆)-alkyl, (C₃-C₈)-cycloalkyl or —C_{b}H_{2b}—R(15);

b is zero, 1, 2, 3 or 4;

or

R(13) and R(14)

together are 4 or 5 methylene groups, of which one CH₂ group can be replaced by oxygen, S, NH, N—CH₃ or N-benzyl;

R(15) is phenyl,

which is unsubstituted or substituted by 1-3 substituents selected from the group consisting of F, Cl, CF₃, methyl, methoxy and NR(9)R(10);

R(9) and R(10)

are H or (C₁-C₄)-alkyl;

R(4) is hydrogen, —OR(16), —NR(16)R(17) or CᵣF₂ᵣ₊₁;

R(16) and R(17)

independently are hydrogen or (C₁-C₃)-alkyl;

r is 1, 2, 3 or 4;

and their pharmaceutically tolerable salts;

(HOE 93/F 223K—EP 639 573, NZ 264 130)

r) benzo-fused 5-membered ring heterocycles of the formula I

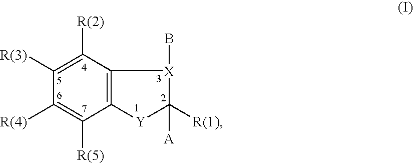

in which:

X is N or CR(6);

Y is oxygen, S or NR(7);

A, B together are a bond

or

A, B are both hydrogen, if X is CR(6) and Y is NR(7) simultaneously;

one of the substituents R(1) to R(6) is a —CO—N═C(NH₂)₂ group;

the other substituents R(1) to R(6) in each case

are hydrogen, F, Cl, Br, I or (C₁-C₆)-alkyl;

up to two of the other substituents R(1) to R(6)

are CN, NO₂, N₃, (C₁-C₄)-alkyloxy or CF₃;

up to one of the other substituents

is R(8)—CₙH₂ₙ—Z—;

n is zero to 10;

where the alkylene chain —CₙH₂ₙ— is straight-chain or branched and where one carbon atom can be replaced by an oxygen or sulfur atom or by a nitrogen atom;

R(8) is hydrogen, (C₂-C₆)-alkenyl or (C₃-C₁₀)-cycloalkyl,

which is unsubstituted or substituted by 1 to 4 methyl groups or an OH group, or can contain an ethylene group —CH═CH—, and in which one methylene group can be replaced by an oxygen or sulfur atom or by a nitrogen atom;

or

R(8) is phenyl,

which is unsubstituted or substituted by 1 to 3 substituents selected from the group consisting of F, Cl, Br, I, CF₃, CH₃—S(O)ₛ— or R(9)—W_{y}—;

s is zero, 1 or 2;

R(9) is H, methyl, ethyl,

W is oxygen or NR(10);

R(10) is H or methyl;

y is zero or 1;

or

R(8) is CₘF₂ₘ₊₁;

m is 1 to 3;

or

R(8) is 1- or 2-naphthyl, pyridyl, quinolyl or isoquinolyl;

Z is —CO—, —CH₂— or —[CR(11)(OH)]_{q}—;

q is 1, 2 or 3;

R(11) is H or methyl;

or

Z is oxygen or —NR(12)—;

R(12) is H or methyl;

or

Z is —S(O)ₛ—;

s is zero, 1 or 2;

or

Z is —SO₂—NR(13)—;

R(13) is H or (C₁-C₄)-alkyl;

R(7) is hydrogen, (C₁-C₁₀)-alkyl, (C₂-C₁₀)-alkenyl or R(8)—CₙH₂ₙ—;

and their pharmaceutically tolerable salts;

(HOE 93/F 236—EP-A 638 548, NZ 264 216)

s) benzoylguanidines of the formula I

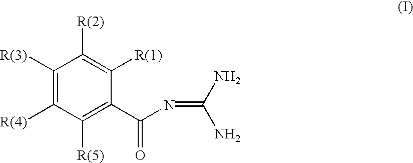

in which:

R(1), R(3) or R(4)

is —NR(6) C═X NR(7)R(8);

X is oxygen or S;

R(6) is hydrogen, (C₁-C₈)-alkyl, (C₁-C₈)-perfluoroalkyl, (C₃-C₈)-alkenyl or —CₙH₂ₙ—R(9);

n is zero, 1, 2, 3 or 4;

R(9) is (C₃-C₈)-cycloalkyl, phenyl, biphenylyl or naphthyl,

where the aromatics are not substituted or are substituted by 1-3 substituents selected from the group consisting of F, Cl, CF₃, methyl, methoxy and

NR(10)R(11);

R(10) and R(11)

are H, (C₁-C₄)-alkyl or (C₁-C₄)-perfluoroalkyl;

R(7) is hydrogen, (C₁-C₈)-alkyl, (C₁-C₈)-perfluoroalkyl, (C₃-C₈)-alkenyl or —CₒH₂ₒ—R(12);

o is zero, 1, 2, 3 or 4;

R(12) is (C₃-C₈)-cycloalkyl, phenyl, biphenylyl or naphthyl,

where the aromatics are not substituted or are substituted by 1-3 substituents selected from the group consisting of F, Cl, CF₃, methyl, methoxy and NR(13)R(14);

R(13) and R(14)

are H, (C₁-C₄)-alkyl or (C₁-C₄)-perfluoroalkyl;

R(8) is defined as R(7);

or

R(7) and R(8)

together are 4 or 5 methylene groups, of which one CH₂ group can be replaced by oxygen, S, NH, N—CH₃ or N-benzyl;

the remaining substituents R(2), R(3), R(4), R(5) or R(1), R(2), R(4), R(5) or R(1), R(2), R(3), R(5) in each case

independently of one another are hydrogen, F, Cl, Br, I, —Oₜₐ(C₁-C₈)-alkyl, —O_{tb}(C₃-C₈)-alkenyl,

—O_{tc}(CH₂)_{b}C_{d}F_{2d+1}, —O_{td}CₚH₂ₚR(18),

or up to 2 groups CN, NO₂, NR(16)R(17),

b is zero or 1;

d is 1, 2 , 3, 4, 5, 6 or 7;

ta is zero or 1;

tb is zero or 1;

tc is zero or 1;

td is zero or 1;

p is zero, 1, 2, 3 or 4;

R(18) is (C₃-C₈)-cycloalkyl, phenyl, biphenylyl or naphthyl,

where the aromatics are not substituted or are substituted by 1-3 substituents selected from the group consisting of F, Cl, CF₃, methyl, methoxy and NR(19)R(20);

R(19) and R(20)

are hydrogen or (C₁-C₄)-alkyl or (C₁-C₄)-perfluoroalkyl;

R(16) is hydrogen, (C₁-C₈)-alkyl, (C₁-C₈)-perfluoroalkyl, (C₃-C₈)-alkenyl, —C_{q}H_{2q}—R(21),

q is zero, 1, 2, 3 or 4;

R(21) is (C₃-C₈)-cycloalkyl, phenyl, biphenylyl or naphthyl,

where the aromatics are not substituted or are substituted by 1-3 substituents from the group F, Cl, CF₃, methyl, methoxy or NR(22)R(23),

R(22) and R(23) are hydrogen, (C₁-C₄)-alkyl or (C₁-C₄)-perfluoroalkyl;

R(17) is hydrogen, (C₁-C₈)-alkyl, (C₁-C₈)-perfluoroalkyl, (C₃-C₈)-alkenyl, —CᵣH₂ᵣ—R(24);

r is zero, 1, 2, 3 or 4;

R(24) is (C₃-C₈)-cycloalkyl, phenyl, biphenylyl or naphthyl,

where the aromatics are not substituted or are substituted by 1-3 substituents selected from the group consisting of F, Cl, CF₃, methyl, methoxy and NR(25)R(26);

R(25) and R(26)

are hydrogen, (C₁-C₄)-alkyl or (C₁-C₄)-perfluoroalkyl;

or

R(16) and R(17)

together are 4 or 5 methylene groups, of which one CH₂ group can be replaced by oxygen, S, NH, N—CH₃ or N-benzyl;

and their pharmaceutically tolerable salts;

(HOE 93/F 249—EP-A 640 587, NZ 264 282)

t) diacyl-substituted guanidines of the formula I

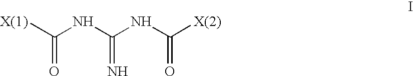

in which:

X(1) and X(2) are

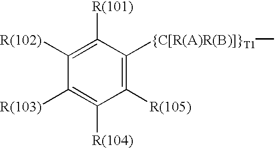

T1 is zero, 1, 2, 3 or 4;

R(A) and R(B)

independently of one another are hydrogen, F, Cl, Br, I, CN, OR(106), (C₁-C₈)-alkyl, (C₃-C₈)-cycloalkyl, O_{zk}(CH₂)_{zl}C_{zm}F_{2zm+1}, NR(107)R(108), phenyl or benzyl,

where the aromatics are not substituted or are substituted by 1-3 substituents selected from the group consisting of F, Cl, CF₃, methyl, methoxy and NR(109)R(110);

R(109) and R(110)

are hydrogen, (C₁-C₄)-alkyl or (C₁-C₄)-perfluoroalkyl;

zl is zero, 1, 2, 3 or 4;

zk is zero or 1;

zm is 1, 2, 3, 4, 5, 6, 7 or 8;

R(106)

is hydrogen, (C₁-C₈)-alkyl, (C₁-C₈)-perfluoroalkyl, (C₃-C₈)-alkenyl, (C₃-C₈)-cycloalkyl, phenyl or benzyl,

where the aromatics are not substituted or are substituted by 1-3 substituents selected from the group consisting of F, Cl, CF₃, methyl, methoxy and

NR(111)R(112);

R(111) and R(112)

are hydrogen, (C₁-C₄)-alkyl or (C₁-C₄)perfluoroalkyl;

R(107) and R(108)

independently of one another are defined as R(106),

or

R(107) and R(108)

together are 4 or 5 methylene groups, of which one CH₂ group can be replaced by oxygen, S, NH, N—CH₃ or N-benzyl;

or

X(1) and X(2) are

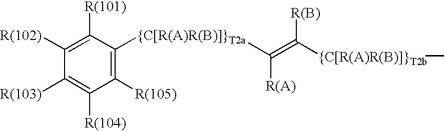

T2a and T2b

independently of one another are zero, 1 or 2;

where the double bond can have the (E)- or (Z)-configuration;

or

X(1) and X(2) are

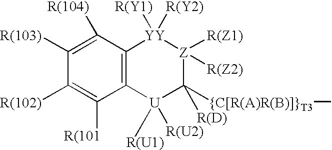

T3 is zero, 1 or 2;

U, YY and Z

independently of one another are C or N,

where U, YY, Z can carry the following number of substituents:

| | Bonded in the ring to | Number of permitted |
|---|---|---|
| U, YY or Z | a double bond | substituents |
| C | yes | 1 |
| C | no | 2 |
| N | yes | 0 |
| N | no | 1 |

R(D) is hydrogen, (C₁-C₈)-alkyl or (C₁-C₈)-perfluoroalkyl,

R(U1), R(U2), R(Y1), R(Y2), R(Z1), R(Z2)

independently of one another are hydrogen, F, Cl, Br, I, CN, OR(114), (C₁-C₈)-alkyl, (C₃-C8)-cycloalkyl, O_{zka}(CH₂)_{zla}C_{zma}F_{2zma+1},

NR(115)R(116), phenyl or benzyl,

where the aromatics are not substituted or are substituted by 1-3 substituents selected from the group consisting of F, Cl, CF₃, methyl, methoxy, NR(117)R(118),

R(117) and R(118)

are hydrogen, (C₁-C₄)-alkyl or (C₁-C₄)-perfluoroalkyl,

zka is zero or 1;

zla is zero, 1, 2, 3 or 4;

zma is 1, 2, 3, 4, 5, 6, 7 or 8;

R(114)

is hydrogen, (C₁-C₈)-alkyl, (C₁-C₈)-perfluoroalkyl, (C₃-C₈)-alkenyl, (C₃-C₈)-cycloalkyl, phenyl or benzyl,

where the aromatics are not substituted or are substituted by 1-3 substituents selected from the group consisting of F, Cl, CF₃, methyl, methoxy and

NR(119)R(120);

R(119) and R(120)

are hydrogen, (C₁-C₄)-alkyl or (C₁-C₄)-perfluoroalkyl;

R(115) and R(116) independently of one another are defined as R(114);

or

R(115) and R(116)

together are 4 or 5 methylene groups, of which one CH₂ group can be replaced by oxygen, S, NH, N—CH₃ or N-benzyl;

but where the constitution of U is nitrogen (N), YY is nitrogen (N) and Z is carbon (C) is excluded,

R(101), R(102), R(103), R(104) and R(105)

independently of one another are hydrogen, F, Cl, Br, I, —C≡N,

X_{zoa}—(CH₂)_{zpa}—(C_{zqa}F_{2zqa+1}), R(110a)—SO_{zbm}, R(110b)R(110c)N—CO,

R(111a)—CO— or R(112a)R(113a)N—SO₂—,

where the perfluoroalkyl group is straight-chain or branched,

X is oxygen, S or NR(114a);

R(114a)

is H or (C₁-C₃)-alkyl;

zoa is zero or 1;

zbm is zero, 1 or 2;

zpa is zero, 1, 2, 3 or 4;

zqa is 1, 2, 3, 4, 5, 6, 7 or 8;

R(110a), R(110b), R(111a) and R(112a)

independently of one another are (C₁-C₈)-alkyl, (C₃-C₈)-alkenyl, —CₙH_{2zn}—R(115a) or (C₁-C₈)-perfluoroalkyl;

zn is zero, 1, 2, 3 or 4;

R(115a)

is (C₃-C₈)-cycloalkyl, phenyl, biphenylyl or naphthyl,

where the aromatics are not substituted or are substituted by 1-3 substituents selected from the group consisting of F, Cl, CF₃, methyl, methoxy and NR(116a)R(117a);

R(116a) and R(117a)

are hydrogen, (C₁-C₄)-perfluoroalkyl or (C₁-C₄)-alkyl;

or

R(110b), R(111a) and R(112a)

are hydrogen;

R(110c) and R(113a)

independently are hydrogen, (C₁-C₄)-perfluoroalkyl or (C₁-C₄)-alkyl;

or

R(110b) and R(110c) and R(112a) and R(113a)

together are 4 or 5 methylene groups, of which one CH₂ group can be replaced by oxygen, sulfur, NH, N—CH₃ or N-benzyl;

or

R(101), R(102), R(103), R(104), R(105)

independently of one another are (C₁-C₈)-alkyl, —C_{zal}H_{2zal}R(118a) or (C₃-C₈)-alkenyl,

zal is zero, 1, 2, 3 or 4;

R(118a)

is (C₃-C₈)-cycloalkyl, phenyl, biphenylyl or naphthyl,

where the aromatics are not substituted or are substituted by 1-3 substituents from the group consisting of F, Cl, CF₃, methyl, methoxy or

NR(119a)R(119b);

R(119a) and R(119b)

are hydrogen, (C₁-C₄)-alkyl or (C₁-C₄)-perfluoroalkyl;

or

R(101), R(102), R(103), R(104), R(105)

independently of one another are (C₁-C₉)-heteroaryl,

which is linked via C or N and which is unsubstituted or substituted by 1-3 substituents from the group consisting of F, Cl, CF₃, CH₃, methoxy, hydroxyl, amino, methylamino and dimethylamino;

or

R(101), R(102), R(103), R(104), R(105)

independently of one another are —C≡C—R(193);

R(193)

is phenyl which is not substituted or is substituted by 1-3 substituents from the group consisting of F, Cl, CF₃, methyl, methoxy or NR(194)R(195);

R(194) and R(195)

are hydrogen or CH₃;

or

R(101), R(102), R(103), R(104), R(105)

independently of one another are

—Y-para-C₆H₄—(CO)_{zh}—(CHOH)_{zi}—(CH2)_{zj}—(CHOH)_{zk}—R(123),

—Y-meta-C₆H₄—(CO)_{zad}—(CHOH)_{zae}—(CH₂)_{zaf}—(CHOH)_{zag}—R(124)

or

—Y-ortho-C₆H₄—(CO)_{zah}—(CHOH)_{zao}—(CH₂)_{zap}—(CHOH)_{zak}—R(125);

Y is oxygen, —S— or —NR(122d)—;

zh, zad, zah

independently are zero or 1;

zi, zj, zk, zae, zaf, zag, zao, zap and zak

independently are zero, 1, 2, 3 or 4;

but where in each case

zh, zi and zk are not simultaneously zero,

zad, zae and zag are not simultaneously zero, and

zah, zao and zak are not simultaneously zero,

R(123), R(124), R(125) and R(122d)

independently are hydrogen or (C₁-C₃)-alkyl;

or

R(101), R(102), R(103), R(104) and R(105)

independently of one another are SR(129), —OR(130),

—NR(131)R(132) or —CR(133)R(134)R(135);

R(129), R(130), R(131) and R(133)

independently are —C_{zab}H_{2zab}—(C₁-C₉)-heteroaryl,

which is unsubstituted or substituted by 1-3 substituents from the group consisting of F, Cl, CF₃, CH₃, methoxy, hydroxyl, amino, methylamino and dimethylamino;

zab is zero, 1 or 2;

R(132), R(134) and R(135)

independently of one another are defined as R(129) or are hydrogen, (C₁-C₄)-alkyl or (C₁-C₄)-perfluoroalkyl;

or

R(101), R(102), R(103), R(104) and R(105)

independently of one another are —W-para-(C₆H₄)—R(196), —W-meta-(C₆H₄)—R(197) or —W-ortho-(C₆H₄)—R(198);

R(196), R(197) and R(198)

independently are (C₁-C₉)-heteroaryl,

which is linked via C or N and which is unsubstituted or substituted by 1 to 3 substituents from the group consisting of F, Cl, CF₃, CH₃, methoxy, hydroxyl, amino, methylamino, dimethylamino and benzyl;

W is oxygen, S or NR(136)—;

R(136)

is hydrogen or (C₁-C₄)-alkyl;

or

R(101), R(102), R(103), R(104) and R(105)

independently of one another are R(146)X(1a)—;

X(1a)

is oxygen, S, NR(147), (D═O)A—, NR(148)C═MN^{(*)}R(149)—;

M is oxygen or sulfur;

A is oxygen or NR(150);

D is C or SO;

R(146)

is (C₁-C₈)-alkyl, (C₃-C₈)-alkenyl, (CH₂)_{zbz}C_{zdz}F_{2zdz+1} or —C_{zxa}H_{2zxa}—

R(151);

zbz is zero or 1;

zdz is 1, 2, 3, 4, 5, 6 or 7;

zxa is zero, 1, 2, 3 or 4;

R(151)

is (C₃-C₈)-cycloalkyl, phenyl, biphenylyl or naphthyl,

where the aromatics are not substituted or are substituted by 1-3 substituents from the group consisting of F, Cl, CF₃, methyl, methoxy and

NR(152)R(153);

R(152) and R(153)

are hydrogen, (C₁-C₄)-alkyl or (C₁-C₄)-perfluoroalkyl;

R(147), R(148) and R(150)

independently are hydrogen, (C₁-C₄)-alkyl, (C₁-C₄)-perfluoroalkyl;

R(149) is defined as R(146),

or

R(146) and R(147), or R(146) and R(148)

together are 4 or 5 methylene groups, of which one CH₂ group can be replaced by oxygen, sulfur, NH, N—CH₃ or N-benzyl;

where A and N^{(*)} are bonded to the phenyl nucleus of the alkanoyl parent structure;

or

R(101), R(102), R(103), R(104) and R(105)

independently of one another are —SR(164), —OR(165), —NHR(166),

—NR(167)R(168), —CHR(169)R(170), —CR(154)R(155)OH,

—C≡CR(156), —CR(158)═CR(157) or

—[CR(159)R(160)]_{zu}—(C═O)—[CR(161)R(162)]_{zv}—R(163);

R(164), R(165), R(166), R(167), R(169)

identically or differently are —(CH₂)_{zy}—(CHOH)_{zz}—(CH₂)_{zaa}—(CHOH)_{zt}—R(171) or —(CH₂)_{zab}—O—(CH₂—CH₂O)_{zac}—R(172);

R(171) and R(172)

are hydrogen or methyl;

zu is 1, 2, 3 or 4;

zv is zero, 1, 2, 3 or 4;

zy, zz, zaa, zab, zac

identically or differently are zero, 1, 2, 3 or 4;

zt is 1, 2, 3 or 4;

R(168), R(170), R(154), R(155)

identically or differently are hydrogen or (C₁-C₆)-alkyl,

or

R(169) and R(170), or R(154) and R(155) together with the carbon atom carrying them are a (C₃-C₈)-cycloalkyl;

R(163)

is hydrogen, (C₁-C₆)-alkyl, (C₃-C₈)-cycloalkyl or

—C_{zeb}H_{2zeb}—R(173);

zeb is zero, 1, 2, 3 or 4;

R(156), R(157) and R(173)

independently are phenyl which is unsubstituted or is substituted by 1-3 substituents from the group consisting of F, Cl, CF₃, methyl, methoxy and

NR(174)R(175);

R(174) and R(175)

are hydrogen or (C₁-C₄)-alkyl;

or

R(156), R(157) and R(173)

independently are (C₁-C₉)-heteroaryl,

which is unsubstituted or substituted as phenyl;

R(158), R(159), R(160), R(161) and R(162)

are hydrogen or methyl,

or

R(101), R(102), R(103), R(104), R(105)

independently of one another are R(176)—NH—SO₂—;

R(176)

is R(177)R(178)N—(C═Y′)—;

Y′ is oxygen, S or N—R(179);

R(177) and R(178)

identically or differently are hydrogen, (C₁-C₈)-alkyl, (C₃-C6)-alkenyl or —C_{zfa}H_{2zfa}—R(180);

zfa is zero, 1, 2, 3 or 4;

R(180)

is (C₅-C₇)-cycloalkyl or phenyl,

which is unsubstituted or substituted by 1-3 substituents selected from the group consisting of F, Cl, CF₃, methoxy

or (C₁-C₄)-alkyl;

or

R(177) and R(178)

together are 4 or 5 methylene groups, of which one CH₂ group can be replaced by oxygen, sulfur, NH, N—CH₃ or N-benzyl;

R(179)

is defined as R(177) or is amidine,

or

R(101), R(102), R(103), R(104), R(105)

independently of one another are NR(184a)R(185), OR(184b),

SR(184c) or —C_{znx}H_{2znx}—R(184d);

znx is zero, 1, 2, 3 or 4;

R(184d)

is (C₃-C₇)-cycloalkyl or phenyl,

which is not substituted or is substituted by 1-3 substituents from the group consisting of F, Cl, CF₃, methyl, methoxy and NR(116k)R(117k);

R(116k) and R(117k)

are hydrogen or C₁-C₄-alkyl;

R(184a), R(184b), R(184c), R(185)

independently of one another are hydrogen, (C₁-C₈)-alkyl, (C₁-C₈)-perfluoroalkyl or (CH₂)_{zao}—R(184g);

zao is zero, 1, 2, 3 or 4;

184g

is (C₃-C₇)-cycloalkyl or phenyl,

which is not substituted or is substituted by 1-3 substituents from the group consisting of F, Cl, CF₃, methyl, methoxy and NR(184u)R(184v);

R(184u) and R(184v)

are hydrogen or C₁-C₄-alkyl;

or

R(184a) and R(185)

together are 4 or 5 methylene groups, of which one CH₂ group can be replaced by oxygen, sulfur, NH, N—CH₃ or N-benzyl;

and their pharmaceutically tolerable salts;

(HOE 93/F 254—EP-A 640 588, NZ 264 307)

u) benzoylguanidines of the formula I

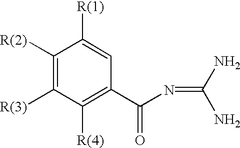

in which:

R(1) is H, F, Cl, Br, I, CN, NO₂, (C₁-C₈)-alkyl, (C₃-C₈)-cycloalkyl or

Xₐ—(CH₂)_{b}—(CF₂)_{c}—CF₃;

X is oxygen, S or NR(5);

a is zero or 1;

b is zero, 1 or 2;

c is zero, 1, 2 or 3;

R(5) is H, (C₁-C₄)-alkyl or —C_{d}H_{2d}R(6);

d is zero, 1, 2, 3 or 4;

R(6) is (C₃-C₈)-cycloalkyl, phenyl, biphenylyl or naphthyl,

where the aromatics are not substituted or are substituted by 1 to 3 substituents selected from the group consisting of F, Cl, CF₃, methyl, methoxy and NR(7)R(8);

R(7) and R(8)

independently are H or (C₁-C4)-alkyl;

or

R(1) is —SR(10), —OR(10) or —CR(10)R(11)R(12);

R(10) is —C_{f}H_{2f}—(C₃-C₈)-cycloalkyl, —(C₁-C₉)-heteroaryl or phenyl,

where the aromatic systems are unsubstituted or substituted by 1 to 3 substituents selected from the group consisting of F, Cl, CF₃, CH₃, methoxy, hydroxyl, amino, methylamino and dimethylamino;

f is zero, 1 or 2;

R(11) and R(12)

independently of one another are defined as R(10) or are hydrogen or (C₁-C₄)-alkyl;

or

R(1) is phenyl, naphthyl, biphenylyl or (C₁-C₉)-heteroaryl,

the latter linked via C or N,

and which are unsubstituted or substituted by 1 to 3 substituents selected from the group consisting of F, Cl, CF₃, CH₃, methoxy, hydroxyl, amino, methylamino and dimethylamino;

or

R(1) is —SR(13), —OR(13), —NHR(13), —NR(13)R(14), —CHR(13)R(15), —C[R(15)R(16)]OH, —C≡CR(18), —C[R(19)]═CR(18), —[CR(20)R(21)]ₖ—(CO)—[CR(22)R(23)R(24)]ₗ

R(13) and R(14)

identically or differently are —(CH₂)_{g}—(CHOH)ₕ—(CH₂)ᵢ—(CHOH)ⱼ—R(17),

R(17) is hydrogen or methyl;

—(CH₂)_{g}—O—(CH₂—CH₂O)ₕ—R(24),

g, h, i

identically or differently are zero, 1, 2, 3 or 4;

j is 1, 2, 3 or 4;

R(15) and R(16)

identically or differently are hydrogen, (C₁-C₆)-alkyl or together with the carbon atom carrying them are a (C₃-C₈)-cycloalkyl;

R(18) is phenyl,

which is unsubstituted or substituted by 1 to 3 substituents selected from the group consisting of F, Cl, CF₃, methyl, methoxy and NR(25)R(26);

R(25) and R(26)

are H or (C₁-C₄)-alkyl;

or

R(18) is (C₁-C₉)-heteroaryl,

which is unsubstituted or substituted as phenyl;

or

R(18) is (C₁-C₆)-alkyl,

which is unsubstituted or substituted by 1 to 3 OH;

or

R(18) is (C₃-C₈)-cycloalkyl;

R(19), R(20), R(21), R(22) and R(23)

are hydrogen or methyl;

k is zero, 1, 2, 3 or 4;

l is zero, 1, 2, 3or 4;

R(24) is H, (C₁-C₆)-alkyl, (C₃-C₈)-cycloalkyl or —CₘH₂ₘ—R(18);

m is 1, 2, 3 or 4;

R(2) and R(3)

independently of one another are defined as R(1);

R(4) is (C₁-C₃)-alkyl, F, Cl, Br, I, CN or —(CH₂)ₙ—(CF₂)ₒ—CF₃;

n is zero or 1;

o is zero, 1 or 2;

and their pharmaceutically tolerable salts;

(HOE 93/F 436—EP-A 659 748), NZ 270 264)

v) acylguanidines of the formula I

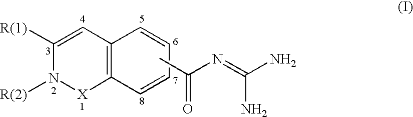

in which:

X is carbonyl, sulfonyl,

R(1) is H, (C₁-C₈)-alkyl,

unsubstituted or substituted by hydroxyl, (C₃-C₈)-cycloalkyl, phenyl,

which is unsubstituted or substituted by 1-3 substituents from the group F, Cl, CF₃, CH₃, methoxy, hydroxyl, amino, methylamino or dimethylamino,

R(2) is H, (C₁-C₄)-alkyl,

and their pharmaceutically tolerable salts;

(HOE 94/F 014 K—EP-A 666 252, NZ 270 370)

w) phenyl-substituted alkylcarboxylic acid guanidides, carrying perfluoroalkyl groups, of the formula I

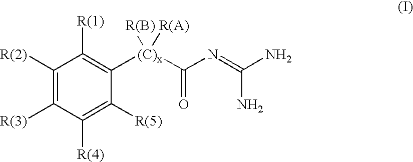

in which:

R(A) is hydrogen, F, Cl, Br, I, CN, OR(6), (C₁-C₈)-alkyl, (C₃-C₈)-cycloalkyl, Oᵣ(CH₂)ₐC_{b}F_{2b+1} or NR(7)R(8);

r is zero or 1;

a is zero, 1, 2, 3 or 4;

b is 1, 2, 3, 4, 5, 6, 7 or 8;

R(6) is hydrogen, (C₁-C₈)-alkyl, (C₃-C₈)-alkenyl, (C₃-C₈)-cycloalkyl, phenyl or benzyl,

where the aromatics are not substituted or are substituted by 1-3 substituents selected from the group consisting of F, Cl, CF₃, methyl, methoxy and

NR(9)R(10);

R(9) and R(10)

are H, (C₁-C₄)-alkyl or (C₁-C₄)-perfluoroalkyl;

R(7) and R(8)

independently of one another are defined as R(6);

R(B) independently is defined as R(A);

X is 1, 2 or 3;

R(1) is hydrogen, (C₁-C₈)-alkyl, (C₃-C₈)-cycloalkyl, —Oₜ(CH₂)_{d}CₑF₂ₑ₊₁, F, Cl, Br, I or CN;

t is zero or 1;

d is zero, 1, 2, 3 or 4;

e is 1, 2, 3, 4, 5, 6, 7 or 8;

R(2), R(3), R(4) and R(5)

independently of one another are defined as R(1);

but with the condition that at least one of the substituents R(1), R(2), R(3), R(4), R(5), R(A) and R(B) is an —Oₜ(CH₂)_{d}CₑF₂ₑ₊₁ or an Oᵣ(CH₂)ₐC_{b}F_{2b+1} group,

and their pharmaceutically tolerable salts;

(HOE 94/F 094—EP-A 676 395, NZ 270 894)

x) heteroaroylguanidines of the formula I

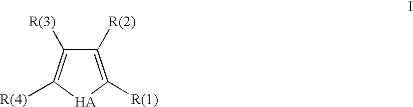

in which:

HA is SOₘ, O or NR(5);

m is zero, 1 or 2;

R(5) is hydrogen, (C₁-C₈)-alkyl or —CₐₘH₂ₐₘR(81);

am is zero, 1 or 2;

R(81) is (C₃-C₈)-cycloalkyl or phenyl,

which is not substituted or is substituted by 1-3 substituents selected from the group consisting of F, Cl, CF₃, methyl, methoxy and NR(82)R(83);

R(82) and R(83)

is H or CH₃;

or

R(81) is (C₁-C₉)-heteroaryl,

which is linked via C or N and which is unsubstituted or substituted by 1-3 substituents selected from the group consisting of F, Cl, CF₃, CH₃, methoxy, hydroxyl, amino, methylamino and dimethylamino;

one of the two substituents R(1) and R(2)

is —CO—N═C(NH₂)₂;

and the other in each case

is hydrogen, F, Cl, Br, I, (C₁-C₃)-alkyl, —OR(6), CᵣF₂ᵣ₊₁, —CO—N═C(NH₂)₂ or —NR(6)R(7);

R(6) and R(7)

independently are hydrogen or (C₁-C₃)-alkyl;

r is 1, 2, 3 or 4;

R(3) and R(4)

independently of one another are hydrogen, F, Cl, Br, I, —C≡N, X—(CH₂)ₚ—(C_{q}—F_{2q+1}), R(8)—SO_{bm}, R(9)R(10)N—CO, R(11)—CO — or

R(12)R(13)N—SO₂—,

where the perfluoroalkyl group is straight-chain or branched,

X is oxygen, S or NR(14);

R(14) is H or (C₁-C₃)-alkyl;

bm is zero, 1 or 2;

p is zero, 1 or 2;

q is zero, 1, 2, 3, 4, 5 or 6;

R(8), R(9), R(11) and R(12)

independently are (C₁-C₈)-alkyl, (C₃-C₆)-alkenyl, —CₙH₂ₙ—R(15), CF₃;

n is zero, 1, 2, 3 or 4;

R(15) is (C₃-C₇)-cycloalkyl or phenyl,

which is not substituted or is substituted by 1-3 substituents selected from the group consisting of F, Cl, CF₃, methyl, methoxy or NR(16)R(17);

R(16) and R(17)

are H or C₁-C₄-alkyl;

or

R(9), R(11) and R(12)

are H;

R(10) and R(13)

independently are H or (C₁-C₄)-alkyl;

or

R(9) and R(10), and R(12) and R(13)

together are 4 or 5 methylene groups, of which one CH₂ group can be replaced by oxygen, S, NH, N—CH₃ or N-benzyl,

or

R(3) and R(4)

independently of one another are (C₁-C₈)-alkyl or —CₐₗH₂ₐₗR(18);

al is zero, 1 or 2;

R(18) is (C₃-C₈)-cycloalkyl or phenyl;

which is not substituted or is substituted by 1-3 substituents selected from the group consisting of F, Cl, CF₃, methyl, methoxy and NR(19)R(20);

R(19) and R(20)

are H or CH₃;

or

R(3) and R(4)

independently of one another are (C₁-C₉)-heteroaryl,

which is linked via C or N and which is unsubstituted or substituted by 1-3 substituents selected from the group consisting of F, Cl, CF₃, CH₃, methoxy, hydroxyl, amino, methylamino and dimethylamino;

or

R(3) and R(4)

independently of one another are

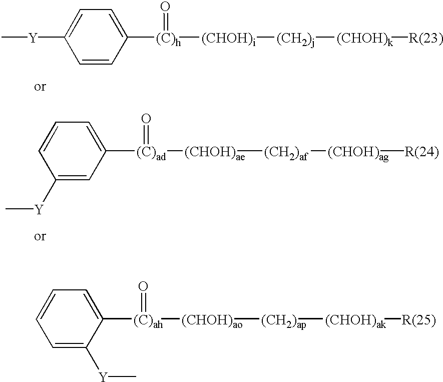

Y is oxygen, —S— or —NR(22)—;

h, ad, ah independently are zero or 1;

i, j, k, ae, af, ag, ao, ap and ak independently are zero, 1, 2, 3, 4,

but where in each case

h, i and k are not simultaneously zero,

ad, ae and ag are not simultaneously zero,

ah, ao and ak are not simultaneously zero,

R(23), R(24), R(25) and R(22)

independently are hydrogen or (C₁-C₃)-alkyl;

or

R(3) and R(4)

independently of one another are hydrogen, F, Cl, Br, I, CN, (C₁-C₈)-alkyl, (C₁-C₈)-perfluoroalkyl, (C₃-C₈)-alkenyl or —C_{g}H_{2g}R(26);

g is zero, 1, 2, 3 or 4;

R(26) is (C₃-C₈)-cycloalkyl, phenyl, biphenylyl or naphthyl,

where the aromatics are not substituted or are substituted by 1-3 substituents selected from the group, consisting of F, Cl, CF₃, methyl, methoxy and

NR(27)R(28);

R(27) and R(28)

are H, (C₁-C₄)-alkyl or (C₁-C₄)-pefluoroalkyl;

or

R(3) and R(4)

independently of one another are SR(29), —OR(30), —NR(31)R(32) or —CR(33)R(34)R(35);

R(29), R(30), R(31) and R(33)

independently are —CₐH₂ₐ—(C₁-C₉)-heteroaryl,

which is unsubstituted or substituted by 1-3 substituents selected from the group consisting of F, Cl, CF₃, CH₃, methoxy, hydroxyl, amino, methylamino and dimethylamino;

a is zero, 1 or 2;

R(32), R(34) and R(35)

independently of one another are defined as R(29) or are hydrogen, (C₁-C₄)-alkyl or (C₁-C₄)-perfluoroalkyl;

or

R(3) and R(4)

independently of one another are

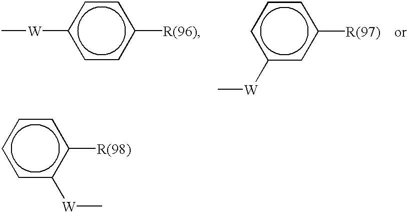

R(96), R(97) and R(98)

independently are (C₁-C₉)-heteroaryl,

which is linked via C or N and which is unsubstituted or substituted by 1 to 3 substituents selected from the group consisting of F, Cl, CF₃, CH₃, methoxy, hydroxyl, amino, methylamino, dimethylamino or benzyl;

W is oxygen, S or NR(36)—;

R(36) is H or (C₁-C₄)-alkyl;

or

R(3) and R(4)

independently of one another are R(37)—SO_{cm} or R(38)R(39)N—SO₂—;

cm is 1 or 2;

R(37) is (C₁-C₈)-alkyl, (C₁-C₈)-perfluoroalkyl, (C₃-C₈)-alkenyl or

—CₛH₂ₛR(40);

s is zero, 1, 2, 3 or 4;

R(40) is (C₃-C₈)-cycloalkyl, phenyl, biphenylyl or naphthyl,

where the aromatics are not substituted or are substituted by 1-3 substituents selected from the group consisting of F, Cl, CF₃, methyl, methoxy and NR(41)R(42);

R(41) and R(42)

are H, (C₁-C₄)-alkyl or (C₁-C₄)-perfluoroalkyl;

R(38) is H, (C₁-C₈)-alkyl, (C₁-C₈)-perfluoroalkyl, (C₃-C₈)-alkenyl or —C_{w}H_{2w}—R(43);

w is zero, 1, 2, 3 or 4;

R(43) is (C₃-C₈)-cycloalkyl, phenyl, biphenylyl or naphthyl,

where the aromatics are not substituted or are substituted by 1-3 substituents selected from the group consisting of F, Cl, CF₃, methyl, methoxy and NR(44)R(45);

R(44) and R(45)

are H, (C₁-C₄)-alkyl or (C₁-C₄)-perfluoroalkyl;

R(39) is H, (C₁-C₄)-alkyl or (C₁-C₄)-perfluoroalkyl;

or

R(38) and R(39)

together are 4 or 5 methylene groups, of which one CH₂ group can be replaced by oxygen, S, NH, N—CH₃ or N-benzyl;

or

R(3) and R(4)

independently of one another are R(46)X(1)—;

X(1) is oxygen, S, NR(47), (D═O)A—, NR(48)C═MN^{(*)}R(49)—,

M is oxygen or S;

A is oxygen or NR(50);

D is C or SO;

R(46) is (C₁-C₈)-alkyl, (C₃-C₈)-alkenyl, (CH₂)_{b}C_{d}F_{2d+1} or —CₓH₂ₓ—R(51);

b is zero or 1;

d is 1, 2, 3, 4, 5, 6 or 7;

x is zero, 1, 2, 3 or 4;

R(51) is (C₃-C₈)-cycloalkyl, phenyl, biphenylyl or naphthyl,

where the aromatics are not substituted or are substituted by 1-3 substituents selected from the group consisting of F, Cl, CF₃, methyl, methoxy and NR(52)R(53);

R(52) and R(53)

are H, (C₁-C₄)-alkyl or (C₁-C₄)-perfluoroalkyl;

R(47), R(48) and R(50)

independently are hydrogen, (C₁-C₄)-alkyl or (C₁-C₄)-pefluoroalkyl;

R(49) is defined as R(46);

or

R(46) and R(47), or R(46) and R(48)

together are 4 or 5 methylene groups, of which one CH₂ group can be replaced by oxygen, S, NH, N—CH₃ or N-benzyl,

where A and N^{(*)} are bonded to the phenyl nucleus of the benzoylguanidine parent structure;

or

R(3) and R(4)

independently of one another are —SR(64), —OR(65), —NHR(66), —NR(67)R(68), —CHR(69)R(70), —C(OH)R(54)R(55), —C≡CR(56), —CR(58)═CHR(57), —[CR(59)R(60)]ᵤ—(CO)—[CR(61)R(62)]ᵥ—R(63);

R(64), R(65), R(66), R(67) and R(69)

identically or differently are —(CH₂)_{y}—(CHOH)_{z}—(CH₂)ₐₐ—(CHOH)ₜ—R(71) or —(CH₂)_{ab}—O—(CH₂—CH₂O)_{ac}—R(72),

R(71) and R(72)

are hydrogen or methyl;

u is 1, 2, 3 or 4;

v is zero, 1, 2, 3 or 4;

y, z, aa, ab and ac

identically or differently are zero, 1, 2, 3 or 4;

t is 1, 2, 3 or 4;

R(68), R(70), R(54) and R(55)

identically or differently are hydrogen, (C₁-C₆)-alkyl;

or

R(69) and R(70), or R(54) and R(55)

together with the carbon atom carrying them are a (C3-C8)-cycloalkyl;

R(63)

is H, (C₁-C₆)-alkyl, (C₃-C₈)-cycloalkyl or —CₑH₂ₑ—R(73);

e is zero, 1, 2, 3 or 4;

R(56), R(57) and R(73)

independently are phenyl,

which is unsubstituted or substituted by 1-3 substituents selected from the group consisting of F, Cl, CF₃, methyl, methoxy and NR(74)R(75);

R(74) and R(75)

are H or (C₁-C₄)-alkyl;

or

R(56), R(57) and R(73)

independently are (C₁-C₉)-heteroaryl,

which is unsubstituted or substitued as phenyl;

R(58), R(59), R(60), R(61) and R(62)

are hydrogen or methyl,

or

R(3) and R(4)

independently of one another are R(76)—NH—SO₂—;

R(76) is R(77)R(78)N—(C═Y′)—;

Y′ is oxygen, S or N—R(79);

R(77) and R(78)

identically or differently are H, (C₁-C₈)-alkyl, (C₃-C₆)-alkenyl, —C_{f}H_{2f}R(80);

f is zero, 1, 2, 3 or 4;

R(80) is (C₅-C₇)-cycloalkyl or phenyl,

which is unsubstituted or substituted by 1-3 substituents selected from the group consisting of F, Cl, CF₃, methoxy and (C₁-C₄)-alkyl;

or

R(77) and R(78)

together are 4 or 5 methylene groups, of which one CH₂ group can be replaced by oxygen, S, NH, N—CH₃ or N-benzyl,

R(79)

is defined as R(77) or is amidine;

or

R(3) and R(4)

independently of one another are NR(84)R(85);

R(84) and R(85)

independently of one another are H, (C₁-C₄)-alkyl, or together are 4 or 5 methylene groups,

of which one CH₂ group can be replaced by oxygen, S, NH, N—CH₃ or N-benzyl;

and their pharmaceutically tolerable salts;

(HOE 94/F 123—EP-A 682 017, NZ 272 058)

y) bicyclic heteroaroylguanidines of the formula I

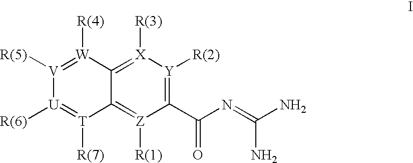

in which:

T, U, V, W, X, Y and Z

independently of one another are nitrogen or carbon;

but with the restriction that X and Z are not simultaneously nitrogen,

and that T, U, V, W, X, Y and Z carry no substituents if they are nitrogen,

and that no more than four of them are simultaneously nitrogen,

R(1) and R(2)

independently of one another are hydrogen, F, Cl, Br, I, (C₁-C₃)-alkyl, (C₁-C₃)-perfluoroalkyl, OR(8), NR(8)R(9) or C(═O)N═C(NH₂)₂;

R(8) and R(9)

independently of one another are hydrogen or (C₁-C₃)-alkyl,

or

R(8) and R(9)

together are 4 or 5 methylene groups, of which one CH₂ group can be replaced by oxygen, S, NH, N—CH₃ or N-benzyl;

R(3), R(4), R(5), R(6) and R(7)

independently of one another are hydrogen, F, Cl, Br, I, —C≡N,

Xₖ—(CH₂)ₚ—(C_{q}F_{2q+1}), R(10a)—SO_{bm}, R(10b)R(10c)N—CO, R(11)—CO— or

R(12)R(13)N—SO₂—,

where the perfluoroalkyl group is straight-chain or branched;

X is oxygen, S or NR(14);

R(14) is H or (C₁-C₃)-alkyl;

bm is zero, 1 or 2;

p is zero, 1 or 2;

k is zero or 1;

q 1, 2, 3, 4, 5 or 6;

R(10a), R(10b), R(11) and R(12)

independently of one another are (C₁-C₈)-alkyl, (C₃-C₆)-alkenyl, —CₙH₂ₙ—R(15) or (C₁-C₈)-perfluoroalkyl;

n is zero, 1, 2, 3 or 4;

R(15) is (C₃-C₇)-cycloalkyl or phenyl, which is not substituted

or is substituted by 1-3 substituents selected from the group consisting of F, Cl, CF₃, methyl, methoxy and

NR(16)R(17);

R(16) and R(17)

are H or C₁-C₄-alkyl;

or

R(10b), R(11) and R(12)

are hydrogen;

R(10c) and R(13)

independently are hydrogen or (C₁-C₄)-alkyl;

or

R(10b) and R(10c) and R(12) and R(13)

together are 4 or 5 methylene groups, of which one CH₂ group can be replaced by oxygen, sulfur, NH, N—CH₃ or N-benzyl;

or

R(3), R(4), R(5), R(6) and R(7)

independently of one another are (C₁-C₈)-alkyl, —CₐₗH₂ₐₗR(18) or (C₃-C₈)-alkenyl;

al is zero, 1 or 2;

R(18) is (C₃-C₈)-cycloalkyl, phenyl, biphenylyl or naphthyl, where the

aromatics are not substituted or are substituted by 1-3 substituents selected from the group consisting of F, Cl, CF₃, methyl, methoxy and NR(19a)R(19b);

R(19a) and R(19b)

are hydrogen, (C₁-C₄)-alkyl or (C₁-C₄)-perfluoroalkyl;

or

R(3), R(4), R(5), R(6) and R(7)

independently of one another are (C₁-C₉)-heteroaryl, which is linked via C or N and which is unsubstituted or substituted by 1-3 substituents from the group consisting of F, Cl, CF₃, CH₃, methoxy, hydroxyl, amino, methylamino or dimethylamino;

or

R(3), R(4), R(5), R(6) and R(7)

independently of one another are

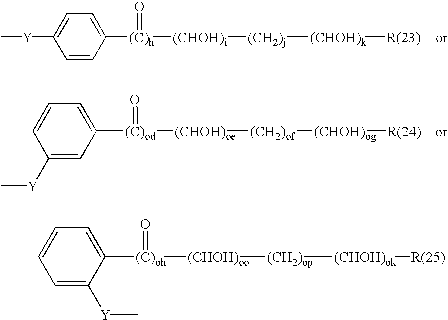

Y is oxygen, —S— or —NR(22)—;

h, ad, ah

independently of one another are zero or 1;

i, j, k, ae, af, ag, ao, ap and ak

independently of one another are zero, 1, 2, 3 or 4;

but where in each case

h, i and k are not simultaneously zero,

ad, ae and ag are not simultaneously zero, and

ah, ao and ak are not simultaneously zero,

R(23), R(24), R(25) and R(22)

independently of one another are hydrogen or (C₁-C₃)-alkyl;

or

R(3), R(4), R(5), R(6) and R(7)

independently of one another are SR(29), —OR(30), —NR(31)R(32) or

—CR(33)R(34)R(35);

R(29), R(30), R(31) and R(33)

independently of one another are —CₐH₂ₐ—(C₁-C₉)-heteroaryl, which is unsubstituted or substituted by 1-3 substituents selected from the group consisting of F, Cl, CF₃, CH₃, methoxy, hydroxyl, amino, methylamino and dimethylamino;

a is zero, 1 or 2;

R(32), R(34) and R(35)

independently of one another are defined as R(29) or are hydrogen, (C₁-C₄)-alkyl or (C₁-C₄)-perfluoroalkyl;

or

R(3), R(4), R(5), R(6) and R(7)

independently of one another are

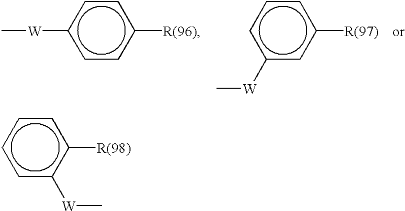

R(96), R(97) and R(98)

independently of one another are (C₁-C₉)-heteroaryl, which is linked via C or N and which is unsubstitued or substituted by 1 to 3 substituents from the group consisting of F, Cl, CF₃, CH₃, methoxy, hydroxyl, amino, methylamino, dimethylamino or benzyl;

W is oxygen, S or NR(36)—;

R(36) is H or (C₁-C₄)-alkyl;

or

R(3), R(4), R(5), R(6) and R(7)

independently of one another are R(46)X(1)—;

X(1) is oxygen, S, NR(47), (D═O)A— or NR(48)C═MN^{(*)}R(49)—;

M is oxygen or sulfur;

A is oxygen or NR(50);

D is C or SO;

R(46) is (C₁-C₈)-alkyl, (C₃-C₈)-alkenyl, (CH₂)_{b}C_{d}F_{2d+1} or —CₓH₂ₓ—R(51);

b is zero or 1;

d is 1, 2, 3, 4, 5, 6 or 7;

x is zero, 1, 2, 3 or 4;

R(51) is (C₃-C₈)-cycloalkyl, phenyl, biphenylyl or naphthyl,

where the aromatics are not substituted or are substituted by 1-3 substituents selected from the group consisting of F, Cl, CF₃, methyl, methoxy and NR(52)R(53);

R(52) and R(53)

are hydrogen, (C₁-C₄)-alkyl or (C₁-C₄)-perfluoroalkyl;

R(47), R(48) and R(50) independently

are hydrogen, (C₁-C₄)-alkyl or (C₁-C₄)-perfluoroalkyl;

R(49) is defined as R(46);

or

R(46) and R(47), or R(46) and R(48)

together are 4 or 5 methylene groups, of which one CH₂ group can be replaced by oxygen, sulfur, NH, N—CH₃ or N-benzyl;

where A and N^{(*)} are bonded to the phenyl nucleus of the heteroaroylguanidine parent structure;

or

R(3), R(4), R(5), R(6) and R(7)

independently of one another are -SR(64), —OR(65), —NHR(66), —NR(67)R(68), —CHR(69)R(70) or —CR(54)R(55)OH, —C≡CR(56), —CR(58)═CR(57) or —[CR(59)R(60)]ᵤ—CO—[CR(61)R(62)]ᵥ—R(63);

R(64), R(65), R(66), R(67) and R(69)

identically or differently are

—(CH₂)_{y}—(CHOH)_{z}—(CH₂)ₐₐ—(CHOH)ₜ—R(71) or

—(CH₂)_{ab}—O—(CH₂—CH₂O)_{ac}—R(72);

R(71) and R(72)

independently of one another are hydrogen or methyl;

u is 1, 2, 3 or 4;

v is zero, 1, 2, 3 or 4;

y, z, aa, ab and ac identically or differently

are zero, 1, 2, 3 or 4;

t is 1, 2, 3 or 4;

R(68), R(70), R(54) and R(55)

identically or differently are hydrogen or (C₁-C₆)-alkyl;

or

R(69) and R(70), or R(54) and R(55)

together with the carbon atom carrying them are (C3-C8)-cycloalkyl;

R(63)

is hydrogen, (C₁-C₆)-alkyl, (C₃-C₈)-cycloalkyl or —CₑH₂ₑ—R(73);

e is zero, 1, 2, 3 or 4;

R(56), R(57) and R(73) independently

are phenyl, which is unsubstituted or substituted by 1-3 substituents selected from the group consisting of F, Cl, CF₃, methyl, methoxy and NR(74)R(75);

R(74) and R(75)

are hydrogen or (C₁-C₄)-alkyl;

or

R(56), R(57) and R(73) independently

are (C₁-C₉)-heteroaryl, which is unsubstituted or substituted as phenyl;

R(58), R(59), R(60), R(61) and R(62)

are hydrogen or methyl;

or

R(3), R(4), R(5), R(6) and R(7)

independently of one another are R(76)—NH—SO₂—;

R(76) is R(77)R(78)N—(C═Y′)—;

Y′ is oxygen, S or N—R(79);

R(77) and R(78)

identically or differently are hydrogen, (C₁-C₈)-alkyl, (C₃-C₆)-alkenyl or —C_{f}H_{2f}—R(80);

f is zero, 1, 2, 3 or 4;

R(80) is (C₅-C₇)-cycloalkyl or phenyl, which is

unsubstituted or substituted by 1-3 substituents selected from the group consisting of F, Cl, CF₃, methoxy and (C₁-C₄)-alkyl;

or

R(77) and R(78)

together are 4 or 5 methylene groups, of which one CH₂ group can be replaced by oxygen, sulfur, NH, N—CH₃ or N-benzyl;

R(79) is defined as R(77) or is amidine;

or

R(3), R(4), R(5), R(6) and R(7)

independently of one another are NR(84a)R(85), OR(84b), SR(84c) or —CₙH₂ₙ—R(84d);

n is zero, 1, 2, 3 or 4;

R(84d) is (C₃-C₇)-cycloalkyl or phenyl, which is not substituted

or is substituted by 1-3 substituents from the group consisting of F, Cl, CF₃, methyl, methoxy and NR(16)R(17);

R(16) and R(17)

are hydrogen, or C₁-C₄-alkyl;

R(84a), R(84b), R(84c) and R(85)

independently of one another are hydrogen, (C₁-C₈)-alkyl, (C₁-C₈)-perfluoroalkyl or (CH₂)ₐₓ—R(84g);

ax is zero, 1, 2, 3 or 4;

R(84g) is (C₃-C₇)-cycloalkyl or phenyl, which is not

substituted or is substituted by 1-3 substituents selected from the group consisting of F, Cl, CF₃, methyl, methoxy and NR(84u)R(84v);

R(84u) and R(84v)

are hydrogen or C₁-C₄-alkyl;

or

R(84a) and R(85)

together are 4 or 5 methylene groups, of which one CH₂ group can be replaced by oxygen, sulfur, NH, N—CH₃ or N-benzyl,

and their pharmaceutically tolerable salts;

(HOE 94/F 134—EP-A 686 627, NZ 272 103)

z) benzoylguanidines of the formula I

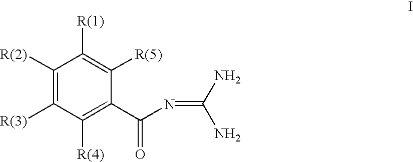

in which:

R(1) is R(6)—SOₘ;

m is zero, 1 or 2;

R(6) is perfluoroalkyl having 1, 2, 3, 4, 5 or 6 carbon atoms, which

is straight-chain or branched;

R(2) and R(3)

independently of one another are hydrogen, F, Cl, Br, I, alkyl having 1, 2, 3 or 4 carbon atoms, alkoxy having 1, 2, 3 or 4 carbon atoms or phenoxy,

which is unsubstituted or substituted by 1-3 substituents selected from the group consisting of F, Cl, methyl and methoxy; methoxy;

or

R(2) and R(3)

independently of one another are pyrrol-1-yl, pyrrol-2-yl- or pyrrol-3-yl,

which is not substituted or is substituted by 1 to 4 substituents selected from the group consisting of F, Cl, Br, I, CN, alkanoyl having 2, 3, 4, 5, 6, 7 or 8 carbon atoms, alkoxycarbonyl having 2, 3, 4, 5, 6, 7 or 8 carbon atoms, formyl, carboxyl, CF₃, methyl and methoxy;

R(4) and R(5)

independently of one another are hydrogen, alkyl having 1, 2 or 3 carbon atoms, F, Cl, Br, I, CN, OR(7), NR(8)R(9) or

—(CH₂)ₙ—(CF₂)ₒ—CF₃;

R(7), R(8) and R(9)

independently of one another are hydrogen or alkyl having 1, 2, 3 or 4 carbon atoms;

n is zero or 1;

o is zero, 1 or 2;

and their pharmacologically acceptable salts;

(HOE 94/F 168—EP-A 690 048, NZ 272 373)

ab) phenyl-substituted alkenylcarboxylic acid guanidides, carrying perfluoroalkyl groups, of the formula I

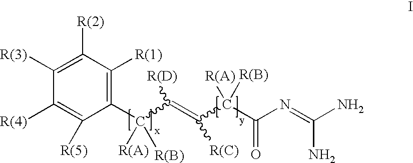

in which:

R(A) is hydrogen, F, Cl, Br, I, CN, OH, OR(6), (C₁-C₈)-alkyl,

Oᵣ(CH₂)ₐC_{b}F_{2b+1}, (C₃-C₈)-cycloalkyl or NR(7)R(8);

r is zero or 1;

a is zero, 1, 2, 3 or 4;

b is 1, 2, 3, 4, 5, 6, 7 or 8;

R(6) is (C₁-C₈)-alkyl, (C₁-C₄)-perfluoroalkyl, (C₃-C₈)-alkenyl, (C₃-C₈)-cycloalkyl, phenyl or benzyl;

where the aromatics are not substituted or are substituted by 1-3 substituents from the group consisting of F, Cl, CF₃, methyl, methoxy and

NR(9)R(10);

R(9) and R(10)

are H, (C₁-C₄)-alkyl or (C₁-C₄)-perfluoroalkyl;

R(7) and R(8)

independently of one another are defined as R(6);

or

R(7) and R(8)

together are 4 or 5 methylene groups, of which one CH₂ group can be replaced by oxygen, sulfur, NH, N—CH₃ or N-benzyl;

R(B) independently is defined as R(A);

X is zero, 1 or 2;

Y is zero, 1 or 2;

R(C) is hydrogen, F, Cl, Br, I, CN, OR(12), (C₁-C₈)-alkyl, Oₚ(CH₂)_{f}C_{g}F_{2g+1}

or (C₃-C₈)-cycloalkyl;

p is zero or 1;

f is zero, 1, 2, 3 or 4;

g is 1, 2, 3, 4, 5, 6, 7 or 8;

R(12)

is (C₁-C₈)-alkyl, (C₁-C₄)-perfluoroalkyl, (C₃-C₈)-alkenyl, (C₃-C₈)-cycloalkyl, phenyl or benzyl;

where the aromatics phenyl or benzyl are not substituted or are substituted by 1-3 substituents from the group consisting of F, Cl, CF₃, methyl, methoxy and NR(13)R(14);

R(13) and R(14)

independently of one another are H, (C₁-C₄)-alkyl or (C₁-C₄)-perfluoroalkyl;

R(D) independently is defined as R(C),

R(1) is hydrogen, (C₁-C₈)-alkyl, —Oₜ(CH₂)_{d}CₑF₂ₑ₊₁, (C₃-C₈)-cycloalkyl, F, Cl, Br, I or CN;

t is zero or 1;

d is zero, 1, 2, 3 or 4;

e is 1, 2, 3, 4, 5, 6, 7 or 8;

R(2), R(3), R(4) and R(5)

independently of one another are defined as R(1);

but with the condition that at least one of the substituents R(A), R(B), R(C), R(D), R(1), R(2), R(4) or R(5) is an Oᵣ(CH₂)ₐC_{b}F_{2b+1}, Oₚ(CH₂)_{f}C_{g}F_{2g+1} or Oₜ(CH₂)_{d}CₑF₂ₑ₊₁ group and R(3) is not an Oₜ(CH₂)_{d}CₑF₂ₑ₊₁ group;

and their pharmaceutically tolerable salts;

(HOE 94/F 182—EP-A 690 048, NZ 272 449)

ac) ortho-amino-substituted benzoylguanidines of the formula I

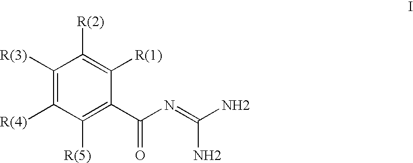

in which:

R(1) is NR(50)R(6),

R(50) and R(6)

independently of one another are hydrogen, (C₁-C₈)-alkyl or (C₁-C₈)-perfluoroalkyl;

R(2), R(3), R(4) and R(5)

independently of one another are R(10)—SOₐ—, R(11)R(12)N—CO—, R(13)—CO— or R(14)R(15)N—SO₂—;

a is zero, 1 or 2,

R(10), R(11), R(12), R(13), R(14) and R(15)

independently of one another are (C₁-C₈)-alkyl, (C₁-C₈)-perfluoroalkyl, (C₃-C₆)-alkenyl or —C_{ab}H_{2ab}—R(16);

ab is zero, 1, 2, 3 or 4;

R(16) is (C₃-C₇)-cycloalkyl, phenyl,

which is not substituted or is substituted by 1-3 substituents selected from the group consisting of F, Cl, CF₃, methyl, methoxy or NR(,17)R(18);

R(17) and R(18)

independently of one another are H, CF₃ or (C₁-C₄)-alkyl;

or

R(11), R(12), and also R(14) and R(15)

together are 4 or 5 methylene groups, of which one CH₂ group can be replaced by oxygen, S, NH, N—CH₃ or N-benzyl;

or

R(11), R(12), R(14) and R(15)

independently of one another are hydrogen;

or

R(2), R(3), R(4) and R(5)

independently of one another are SR(21), —OR(22), —NR(23)R(24) or —CR(25)R(26)R(27);

R(21), R(22), R(23) and R(25)

independently of one another are —C_{b}H_{2b}—(C₁-C₉)-heteroaryl, which is unsubstituted or substituted by 1-3 substituents selected from the group consisting of F, Cl, CF₃, CH₃, methoxy, hydroxyl, amino, methylamino and dimethylamino;

b is zero, 1 or 2;

R(24), R(26) and R(27)

independently of one another are hydrogen, (C₁-C₄)-alkyl or (C₁-C₄)-perfluoroalkyl;

or

R(2), R(3), R(4) and R(5)

independently of one another are hydrogen, F, Cl, Br, I, CN,

—(Xa)_{dg}—C_{da}H_{2da+1}, —(Xb)_{dh}—(CH₂)_{db}—C_{de}F_{2de+1}, (C₃-C₈)-alkenyl or

—C_{df}H_{2df}R(30);

(Xa) is O, S or NR(33);

R(33)

is H, (C₁-C₄)-alkyl or (C₁-C₄)-perfluoroalkyl;

dg is zero or 1;

(Xb) is O, S or NR(34);

R(34)

is H, (C₁-C₄)-alkyl or (C₁-C₄)-perfluoroalkyl;

dh is zero or 1;

da is zero, 1, 2, 3, 4, 5, 6, 7, 8;

db is zero, 1, 2, 3, 4;

de is zero, 1, 2, 3, 4, 5, 6, 7;

df is zero, 1, 2, 3, 4;

R(30)

is (C₃-C₈)-cycloalkyl, phenyl, biphenylyl or naphthyl, where the aromatics phenyl, biphenylyl or naphthyl are not substituted or are substituted by 1-3 substituents selected from the group consisting of F, Cl, CF₃, methyl, methoxy and

NR(31)R(32);

R(31) and R(32)

are H, (C₁-C₄)-alkyl or (C₁-C₄)-perfluoroalkyl;

or

R(2), R(3), R(4) and R(5)

independently of one another are NR(40)R(41) or

—(Xe)—(CH₂)_{eb}R(45);

R(40) and R(41)

independently of one another are hydrogen, (C₁-C₈)-alkyl, (C₁-C₈)-perfluoroalkyl or (CH₂)ₑ—R(42);

e is zero, 1, 2, 3 or 4;

R(42)

is (C₃-C₇)-cycloalkyl, phenyl, which is not substituted or is substituted by 1-3 substituents selected from the group consisting of F, Cl, CF₃, methyl, methoxy and

NR(43)R(44);

R(43) and R(44)

independently of one another are H, CF₃ or (C₁-C₄)-alkyl;

or

R(40) and R(41)

together are 4 or 5 methylene groups, of which one CH₂ group can be replaced by oxygen, sulfur, NH, N—CH₃ or N-benzyl;

(Xe) is O, S or NR(47);

R(47)

is H, (C₁-C₄)-alkyl or (C₁-C₄)-perfluoroalkyl;

eb is zero, 1, 2, 3 or 4;

R(45) is (C₃-C₇)-cycloalkyl, phenyl, which is not substituted or is

substituted by 1-3 substituents selected from the group consisting of F, Cl, CF₃, methyl, methoxy, NR(50)R(51) and

—(Xfa)—(CH₂)_{ed}—(Xfb)R(46);

Xfa is CH₂, O, S or NR(48);

Xfb is O, S or NR(49);

ed is 1, 2, 3 or 4;

R(46)

is H, (C₁-C₄)-alkyl or (C₁-C₄)-perfluoroalkyl;

R(48), R(49), R(50) and R(51)

independently of one another are H or (C₁-C₄)-alkyl or (C₁-C₄)-perfluoroalkyl;

where R(3) and R(4), however, cannot be hydrogen,

and their pharmaceutically tolerable salts;

(HOE 94/F 265—NZ 272 946, EP-A 700 904)

ad) benzoylguanidines of the formula I

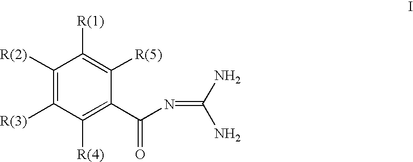

in which:

one of the three substituents R(1), R(2) and R(3)

is (C₁-C₉)-heteroaryl-N—oxide,

which is linked via C or N and which is unsubstituted or substituted by 1-3 substituents selected from the group consisting of F, Cl, CF₃, CH₃, methoxy, hydroxyl, amino, methylamino and dimethylamino;

or

one of the three substituents R(1), R(2) and R(3)

is —SR(10), —OR(10), —NR(10)R(11) or —CR(10)R(11)R(12);

R(10)

is —CₐH₂ₐ—(C₁-C₉)-heteroaryl-N-oxide,

which is unsubstituted or substituted by 1-3 substituents selected from the group consisting of F, Cl, CF₃, CH₃, methoxy, hydroxyl, amino, methylamino and dimethylamino;

a is zero, 1 or 2;

R(11) and R(12)

independently of one another are defined as R(10), are hydrogen or (C₁-C₄)-alkyl;

and the other substituents R(1), R(2) and R(3) in each case

independently of one another are (C₁-C₈)-alkyl, (C₂-C₈)-alkenyl or

—CₘH₂ₘR(14);

m is zero, 1 or 2;

R(14) is (C₃-C₈)-cycloalkyl or phenyl,

which is not substituted or is substituted by 1-3 substituents selected from the group consisting of F, Cl, CF₃₁ methyl, methoxy and

NR(15)R(16),

R(15) and R(16)

are hydrogen or CH₃;

or

the other substituents R(1), R(2) and R(3) in each case

independently of one another are hydrogenq, F, Cl, Br, I, —C≡N,

X—(CH₂)ₚ—(C_{q}F_{2q+1}), R(22)—SOᵤ, R(23)R(24)N—CO, R(25)—CO— or

R(26)R(27)N—SO₂—,

where the perfluoroalkyl group is straight-chain or branched;

X is a bond, oxygen, S or NR(28);

u is zero, 1 or 2;

is zero, 1 or 2;

is zero, 1, 2, 3, 4, 5 or 6;

R(22), R(23), R(25) and R(26)

independently are (C₁-C₈)-alkyl, (C₂-C₆)-alkenyl, —CₙH₂ₙ—R(29)

or CF₃;

n is zero, 1, 2, 3 or 4;

R(28) is hydrogen or (C₁-C₃)-alkyl;

R(29) is (C₃-C7)-cycloalkyl or phenyl;

which is not substituted or is substituted by 1-3 substituents selected from the group consisting of F, Cl, CF₃, methyl, methoxy and

NR(30)R(31);

R(30) and R(31)

are hydrogen or C₁-C₄-alkyl,

or

R(23), R(25) and R(26)

are also hydrogen;

R(24) and R(27)

independently of one another are hydrogen or (C₁-C₄)-alkyl;

or

R(23) and R(24), and also R(26) and R(27) together are 4 or 5 methylene groups, of which one CH₂ group can be replaced by oxygen, S, NH, N—CH₃ or N-benzyl;

or

the other substituents R(1), R(2) and R(3) in each case

independently of one another are OR(35) or NR(35)R(36);

R(35) and R(36)

independently of one another are hydrogen or (C₁-C₆)-alkyl;

or

R(35) and R(36)

together are 4-7 methylene groups, of which one CH₂ group can be replaced by oxygen, S, NH, N—CH₃ or N-benzyl,

R(4) and R(5)

independently of one another are hydrogen, (C₁-C₄)-alkyl, F, Cl,

—OR(32), —NR(33)R(34) or CᵣF₂ᵣ₊₁;

R(32), R(33) and R(34)

independently of one another are hydrogen or (C₁-C₃)-alkyl;

r is 1, 2, 3 or 4;

and their pharmaceutically tolerable salts;

(HOE 94/F 266—EP-A 702 001, NZ 272 948)

ae) benzoylguanidines of the formula I

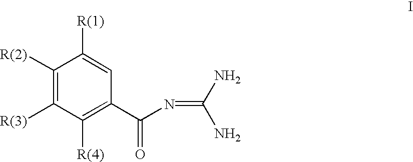

in which:

R(1) is hydrogen, F, Cl, Br, I, CN, NO₂, OH, (C₁-C₈)-alkyl, (C₃-C₈)-cycloalkyl, Oₐ—(CH₂)_{b}—(CF₂)_{c}—CF₃;

a is zero or 1;

b is zero, 1 or 2;

c is zero, 1, 2 or 3;

or

R(1) is R(5)—SOₘ or R(6)R(7)N—SO₂—;

m is zero, 1 or 2;

R(5) and R(6) independently of one another

are (C₁-C₈)-alkyl, (C₃-C₆)-alkenyl, CF₃ or —CₙH₂ₙ—R(8);

n is zero, 1, 2, 3 or 4;

R(7) is hydrogen or (C₁-C₄)-alkyl;

R(8) is (C₃-C₇)-cycloalkyl or phenyl,

which is not substituted or is substituted by 1-3 substituents selected from the group consisting of F, Cl, CF₃, methyl, methoxy and NR(9)R(10);

R(9) and R(10) independently of one another

are hydrogen or (C₁-C₄)-alkyl;

or

R(6) is H;

or R(6) and R(7)

together are 4 or 5 methylene groups, of which one CH₂ group can be replaced by oxygen, S, NH, N—CH₃ or N-benzyl,

or

R(1) is —SR(11), —OR(11) or —CR(11)R(12)R(13);

R(11) is —CₚH₂ₚ—(C₃-C₈)-cycloalkyl, —(C₁-C₉)-heteroaryl or phenyl,

where the aromatic systems are unsubstituted or substituted by 1-3 substituents selected from the group consisting of F, Cl, CF₃, CH₃, methoxy, hydroxyl, amino, methylamino and dimethylamino;

R(12), R(13) independently of one another

are defined as R(11) or are hydrogen or (C₁-C₄)-alkyl;

p is zero, 1 or 2;

or

R(1) is phenyl, naphthyl, biphenylyl or (C₁-C₉)-heteroaryl, the latter linked via C or N,

which are unsubstituted or substituted by 1-3 substituents selected from the group consisting of F, Cl, CF₃, CH₃, methoxy, hydroxyl, amino, methylamino and dimethylamino;

R(2) is —CF₂R(14), —CF[R(15)][R(16)], —CF[(CF₂)_{q}—CF₃)][R(15)], —C[(CF₂)ᵣ—CF₃]═CR(15)R(16);

R(14) is (C₁-C₄)-alkyl or (C₃-C₆)-cycloalkyl;

R(15) and R(16) independently of one another

are hydrogen or (C₁-C₄)-alkyl;

q is zero, 1 or 2;

r is zero, 1 or 2;

R(3) is defined as R(1);

R(4) is hydrogen, (C₁-C₃)-alkyl, F, Cl, Br, I, CN, —(CH₂)ₛ—(CF₂)ₜ—CF₃;

s is zero or 1;

t is zero, 1 or 2;

and their pharmaceutically tolerable salts;

(HOE 94/F 267—EP-A 700 899, NZ 272 947)

af) benzoylguanidines of the formula I

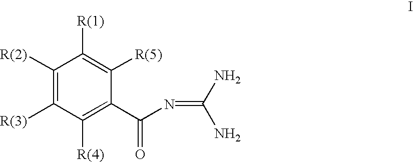

in which:

one of the three substituents R(1), R(2) and R(3)

is —Y-4—[(CH₂)ₖ—CHR(7)—(C═O)R(8)]-phenyl,

—Y-3—(CH₂)ₖ—CHR(7)—(C═O)R(8)]-phenyl or

—Y-2—[(CH₂)ₖ—CHR(7)—(C═O)R(8)]-phenyl,

where the phenyl in each case is unsubstituted or substituted by 1-2 substituents from the group F, Cl, —CF₃, methyl, hydroxyl, methoxy, or —NR(37)R(38);

R(37) and R(38)

independently of one another are hydrogen or —CH3;

Y is a bond, oxygen, —S— or —NR(9);

R(9) is hydrogen or —(C₁-C₄)-alkyl;

R(7) is —OR(10) or —NR(10)R(11);

R(10) and R(11)

independently of one another are hydrogen, —(C₁-C₈)-alkyl, —(C₁-C₈)-alkanoyl, —(C₁-C₈)-alkoxycarbonyl, benzyl, benzyloxycarbonyl;

or

R(10) is trityl;

R(8) is —OR(12) or —NR(12)R(13);

R(12) and R(13)

independently of one another are hydrogen, —(C₁-C₈)-alkyl or benzyl;

k is zero, 1, 2, 3 or 4;

and the other radicals R(1), R(2) and R(3) in each case

independently of one another are —(C₁-C₈)-alkyl, —(C₂-C₈)-alkenyl or

—(CH₂)ₘR(14);

m is zero, 1 or 2;

R(14) is —(C₃-C₈)-cycloalkyl or phenyl,

which is not substituted or is substituted by 1-3 substituents selected from the group consisting of F, Cl, —CF₃, methyl, methoxy and —N R(15)R(16);

R(15) and R(16)

are hydrogen or —CH₃;

or

the other radicals R(1), R(2) and R(3) in each case

independently of one another are R(18)R(19)N—(C═Y′)—NH—SO₂—;

Y′ is oxygen, —S— or —N—R(20);

R(18) and R(19)

independently of one another are hydrogen, —(C₁-C₈)-alkyl, —(C₃-C₆)-alkenyl or —(CH₂)ₜ—R(21);

t is zero, 1, 2, 3 or 4;

R(21) is —(C₅-C₇)-cycloalkyl or phenyl,

which is unsubstituted or substituted by 1-3 substituents selected from the group consisting of F, Cl, —CF₃, methoxy and —(C₁-C₄)-alkyl;

or

R(18) and R(19)

together are 4 or 5 methylene groups, of which one CH₂ group can be replaced by oxygen, —S—, —NH—, —N—CH₃ or —N-benzyl;

R(20)

is defined as R(18) or is amidine;

or

the other radicals R(1), R(2) and R(3) in each case

independently of one another are hydrogen, F, Cl, Br, I, —C≡N, X—(CH₂)ₚ—(C_{q}F_{2q+1}), R(22)—SOᵤ—, R(23)R(24)N—CO—, R(25)—CO— or R(26)R(27)N—SO₂—, where the perfluoroalkyl group is straight-chain or branched;

X is a bond, oxygen, —S— or —NR(28);

u is zero, 1 or 2;

is zero, 1 or 2;

q is 1, 2, 3, 4, 5 or 6;

R(22), R(23), R(25) and R(26)

independently of one another are —(C₁-C₈)-alkyl, —(C₃-C₆)-alkenyl, —(CH₂)ₙ—R(29) or —CF₃;

n is zero, 1, 2, 3 or 4;

R(28) is hydrogen or —(C₁-C₃)-alkyl;

R(29) is —(C₃-C₇)-cycloalkyl or phenyl,

which is not substituted or is substituted by 1-3 substituents selected from the group consisting of F, Cl, —CF₃, methyl, methoxy and —NR(30)R(31);

R(30) and R(31)

are hydrogen or —(C₁-C₄)-alkyl;

or

R(23), R(25) and R(26)

are hydrogen;

R(24) and R(27)

independently of one another are hydrogen or —(C₁-C₄)-alkyl;

or

R(23) and R(24), and also R(26) and R(27)

together are 4 or 5 methylene groups, of which one CH₂ group can be replaced by oxygen, —S—, —NH—, —N—CH₃ or —N-benzyl;

or

the other radicals R(1), R(2) and R(3) in each case

independently of one another are —OR(35) or —NR(35)R(36);

R(35) and R(36)

independently of one another are hydrogen or —(C₁-C₆)-alkyl;

or

R(35) and R(36)

together are 4-7 methylene groups, of which one CH₂ group

can be replaced by oxygen, —S—, —NH—, —N—CH₃ or —N-benzyl;

R(4) and R(5)

independently of one another are hydrogen, —(C₁-C₄)-alkyl, F, Cl, —OR(32), —NR(33)R(34) or —CᵣF₂ᵣ₊₁;

R(32), R(33) and R(34)

independently of one another are hydrogen or —(C₁-C₃)-alkyl;

r is 1, 2, 3 or 4;

and their pharmaceutically tolerable salts;

(HOE 94/F 352—EP-A 713 684, NZ 280 517)

ag) benzoylguanidines of the formula I

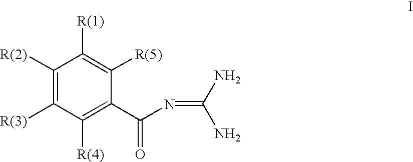

in which:

R(1) is R(6)—CO or R(7)R(8)N—CO;

R(6) is (C₁-C₈)-alkyl, (C₁-C₈)-perfluoroalkyl, (C₃-C₈)-alkenyl

or —CₙH₂ₙ—R(9),

n is zero, 1, 2, 3 or 4;

R(9) is (C₃-C₈)-cycloalkyl, phenyl, biphenylyl or naphthyl,

where the aromatics are not substituted or are substituted by 1-3 substituents selected from the group consisting of F, Cl, CF₃, methyl, methoxy and

NR(10)R(11),

R(10) and R(11)

are H, (C₁-C₄)-alkyl or (C₁-C₄)-perfluoroalkyl;

R(7) is H, (C₁-C₈)-alkyl, (C₁-C₈)-perfluoroalkyl, (C₃-C₈)-alkenyl or —CₙH₂ₙ—R(12);

n is zero, 1, 2, 3 or 4;

R(12) is (C₃-C₈)-cycloalkyl, phenyl, biphenylyl or naphthyl,

where the aromatics are not substituted or are substituted by 1-3 substituents selected from the group consisting of F, Cl, CF₃, methyl, methoxy and NR(13)R(14);

R(13) and R(14)

are H, (C₁-C₄)-alkyl or (C₁-C₄)-perfluoroalkyl;

R(8) is H, (C₁-C₄)-alkyl or (C₁-C₄)-perfluoroalkyl;

or

R(7) and R(8) together

are 4 or 5 methylene groups, of which one CH₂ group can be replaced by oxygen, S, NH, N—CH₃ or N-benzyl;

R(2) is defined as R(1), or is H, OH, F, Cl, Br, I, CN, NO₂, (C₁-C₈)-alkyl, (C₁-C₈)-perfluoroalkyl, (C₃-C₈)-alkenyl or —CₙH₂ₙR(15);

n is zero, 1, 2, 3 or 4;

R(15) is (C₃-C₈)-cycloalkyl, phenyl, biphenylyl or naphthyl,

where the aromatics are not substituted or are substituted by 1-3 substituents selected from the group consisting of F, Cl, CF₃, methyl, methoxy and NR(16)R(17);

R(16) and R(17)

are H, (C₁-C₄)-alkyl or (C₁-C₄)-perfluoroalkyl;

or

R(2) is (C₁-C₉)-heteroaryl,

which is linked via C or N and which is unsubstituted or substituted by 1-3 substituents selected from the group consisting of F, Cl, CF₃, CH₃, methoxy, hydroxyl, amino, methylamino and dimethylamino;

or

R(2) is SR(18), —OR(18), —NR(18)R(19) or —CR(18)R(19)R(20);

R(18) is —CₐH₂ₐ—(C₁-C₉)-heteroaryl,

which is unsubstituted or substituted by 1-3 substituents selected from the group consisting of F, Cl, CF₃, CH₃, methoxy, hydroxyl, amino, methylamino, dimethylamino;

a is zero, 1 or 2;

R(19) and R(20)

independently of one another are defined as R(18) or are hydrogen, (C₁-C₄)-alkyl or (C₁-C₄)-perfluoroalkyl;

or

R(2) is R(21)—SOₘ or R(22)R(23)N—SO₂—;

m is 1 or 2;

R(21) is (C₁-C₈)-alkyl, (C₁-C₈)-perfluoroalkyl, (C₃-C₈)-alkenyl or —CₙH₂ₙ—R(24);

n is zero, 1, 2, 3 or 4;

R(24) is (C₃-C₈)-cycloalkyl, phenyl, biphenylyl or naphthyl,

where the aromatics are not substituted or are substituted by 1-3 substituents selected from the group consisting of F, Cl, CF₃, methyl, methoxy and NR(27)R(28);

R(27) and R(28)

are H, (C₁-C₄)-alkyl or (C₁-C₄)-perfluoroalkyl;

R(22) is H, (C₁-C₈)-alkyl, (C₁-C₈)-perfluoroalkyl, (C₃-C₈)-alkenyl or —CₙH₂ₙ—R(29);

n is zero, 1, 2, 3 or 4;

R(29) is (C₃-C₈)-cycloalkyl, phenyl, biphenylyl or naphthyl,

where the aromatics are not substituted or are substituted by 1-3 substituents selected from the group consisting of F, Cl, CF₃, methyl, methoxy and NR(30)R(31);

R(30) and R(31)

are H, (C₁-C₄)-alkyl or (C₁-C₄)-perfluoroalkyl;

R(23) is hydrogen, (C₁-C₄)-alkyl or (C₁-C₄)-perfluoroalkyl;

or

R(22) and R(23)

together are 4 or 5 methylene groups, of which one CH₂ group can be replaced by oxygen, S, NH, N—CH₃or N-benzyl;

or

R(2) is R(33)X—;

X is oxygen, S, NR(34), (D═O)A— or NR(34)C═MN^{(*)}R(35)—;

M is oxygen or S;

A is oxygen or NR(34);

D is C or SO;

R(33) is (C₁-C₈)-alkyl, (C₃-C₈)-alkenyl, (CH₂)_{b}C_{d}F_{2d+1} or —CₙH₂ₙ—R(36);

b is zero or 1;

d is 1, 2, 3, 4, 5, 6 or 7;

n is zero, 1, 2, 3, or 4;

R(36) is (C₃-C₈)-cycloalkyl, phenyl, biphenylyl or naphthyl,

where the aromatics are not substituted or are substituted by 1-3 substituents selected from the group consisting of F, Cl, CF₃, methyl, methoxy and NR(37)R(38);

R(37) and R(38)

are H, (C₁-C₄)-alkyl or (C₁-C₄)-perfluoroalkyl;

R(34) is hydrogen, (C₁-C₄)-alkyl or (C₁-C₄)-perfluoroalkyl;

R(35) is defined as R(33);

or

R(33) and R(34)

together are 4 or 5 methylene groups, of which one CH₂ group can be replaced by oxygen, S, NH, N—CH₃ or N-benzyl;

where A and N^{(*)} are bonded to the phenyl nucleus of the benzoylguanidine parent structure;

or

R(2) is —SR(40), —OR(40), —NHR(40), —NR(40)R(41), —CHR(40)R(42), —CR(42)R(43)OH, —C≡CR(45), —CR(46)═CR(45) or —[CR(47)R(48)]ᵤ—CO—[C(R49)R(50)]ᵥ—R(44);

R(40) and R(41)

independently of one another are —(CH₂)ₚ—(CHOH)_{q}—(CH₂)ᵣ—(CHOH)ₜ—R(51) or —(CH₂)ₚ—O—(CH₂—CH₂O)_{q}—R(51);

R(51) is hydrogen or methyl;

u is 1, 2, 3 or 4;

v is zero, 1, 2, 3 or 4;

p, q and r

independently of one another are zero, 1, 2, 3 or 4;

t is 1, 2, 3 or 4;

R(42) and R(43)

independently of one another are hydrogen or (C₁-C₆)-alkyl;

or

R(42) and R(43)

together with the carbon atom carrying them are a (C₃-C₈)-cycloalkyl;

R(44) is hydrogen, (C₁-C₆)-alkyl, (C₃-C₈)-cycloalkyl, —CₑH₂ₑ—R(45);

e is zero, 1, 2, 3 or 4;

R(45) is phenyl,

which is unsubstituted or substituted by 1-3 substituents selected from the group consisting of F, Cl, CF₃, methyl, methoxy and NR(52)R(53);

R(52) and R(53)

are H or (C₁-C₄)-alkyl;

or

R(45) is (C₁-C₉)-heteroaryl,

which is unsubstituted or substituted as phenyl;

or

R(45) is (C₁-C₆)-alkyl,

which is unsubstituted or substituted by 1-3 OH;

R(46), R(47), R(48), R(49) and R(50)

independently of one another are hydrogen or methyl;

or

R(2) is R(55)—NH—SO₂—;

R(55) is R(56)R(57)N—(C═Y)—;

Y is oxygen, S or N—R(58);

R(56) and R(57)

independently of one another are hydrogen, (C₁-C₈)-alkyl, (C₃-C₆)-alkenyl or —C_{f}H_{2f}—R(59);

f is zero, 1, 2, 3 or 4;

R(59) is (C₅-C₇)-cycloalkyl, phenyl,

which is unsubstituted or substituted by 1-3 substituents selected from the group consisting of F, Cl, CF₃, methoxy and (C₁-C₄)-alkyl;

or

R(56) and R(57)

together are 4 or 5 methylene groups, of which one CH₂ group can be replaced by oxygen, S, NH, N—CH₃ or N-benzyl;

R(58)

is defined as R(56) or is amidine;

R(3), R(4) and R(5) are independently of one another defined as R(1) or R(2), but where at least one of the substituents R(2), R(3), R(4) and R(5) must be OH;

and their pharmaceutically tolerable salts;

(HOE 95/F 007 K—EP-A 723 956, NZ 280 887)

ah) benzoylguanidines of the formula I

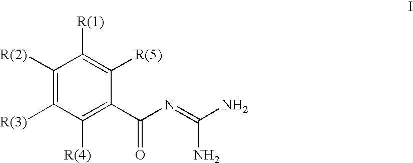

in which:

one of the three substituents R(1), R(2) and R(3)

is R(6)—A—B—D—;

R(6) is a basic protonatable radical, i.e. an amino group —NR(7)R(8), an amidino group R(7)R(8)N—C[═N—R(9)]— or a guanidino group

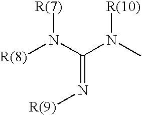

R(7), R(8), R(9) and R(10)

independently of one another are hydrogen or alkyl having 1, 2, 3 or 4 carbon atoms;

or

R(7) and R(8)

together are CₐH₂ₐ;

a is 4, 5, 6 or 7;

where if a =5, 6 or 7 a methylene group of the group CₐH₂ₐ can be replaced by a heteroatom group O, SOₘ or NR(11),

or

R(8) and R(9) or R(9) and R(10) or R(7) and R(10)

are a group CₐH₂ₐ;

a is 2, 3, 4 or 5;

where if a=3, 4 or 5 a methylene group of the group CₐH₂ₐ can be replaced by a heteroatom group O, SOₘ or NR(11);

m is zero, 1 or 2;

R(11) is hydrogen or methyl;

or

R(6) is a basic heteroaromatic ring system having 1-9 carbon atoms;

A is C_{b}H_{2b};

b is 1, 2, 3, 4, 5, 6, 7, 8, 9 or 10;

where in the group C_{b}H_{2b} one or two methylene groups can be replaced by one of the groupings selected from the group consisting of —O—, —CO—, —CH[OR(20)]—, —SOₘ—, —NR(20)—, —NR(20)—CO—, —NR(20)—CO—NH—, —NR(20)—CO—NH—SO₂—,

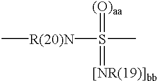

and —SOₐₐ[NR(19)]_{bb}—;

and where in the group C_{b}H_{2b} a methylene group can be replaced by —CH—R(99), where R(99) together with R(7) forms a pyrrolidine or piperidine ring;

aa is 1 or 2;

bb is 0 or 1;

aa+bb=2;

R(19) is hydrogen or alkyl having 1, 2, 3 or 4 carbon atoms;

R(20) is hydrogen or methyl;

B is a phenylene or naphthylene radical

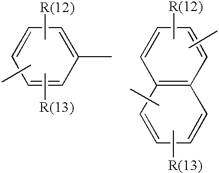

R(12) and R(13)

independently of one another are hydrogen, methyl, F, Cl, Br, I CF₃ or —SO_{w}—R(14);

R(14) is methyl or NR(15)R(16);

R(15) and R(16)

independently of one another are hydrogen or alkyl having 1, 2, 3 or 4 carbon atoms;

w is zero, 1 or 2;

D is —C_{d}H_{2d}—X_{f}—;

d is zero, 1, 2, 3 or 4;

X is —O—, —CO—, —CH[OR(21)]—, —SOₘ— or —NR(21)—;

f is zero or 1;

R(21) is hydrogen or methyl;

m is zero, 1 or 2;

and the other substituents R(1) and R(2) and R(3) in each case

independently of one another are hydrogen, F, Cl, Br, I, —CN, —(C₁-C₈)-alkyl, —(C₂-C₈)-alkenyl, —NR(35)R(36) or R(17)—C_{g}H_{2g}—Zₕ—;

g is zero, 1, 2, 3 or 4;

h is zero or 1;

R(35) and R(36)

independently of one another are hydrogen or alkyl having 1, 2, 3, 4, 5 or 6 carbon atoms;

or

R(35) and R(36)

together are 4-7 methylene groups, of which one CH₂ group can be replaced by oxygen, —S—, —NH—, —NCH₃ or —N-benzyl;

Z is —O—, —CO—, —SOᵥ—,—NR(18)—, —NR(18)—CO—, —NR(18)—CO—NH— or —NR(18)—SO₂—;

R(18) is hydrogen or methyl;

v is zero, 1 or 2;

R(17) is hydrogen, cycloalkyl having 3, 5 or 6 carbon atoms or CₖF₂ₖ₊₁—;

k is 1, 2 or 3,

or

R(17) is pyrrol-1-yl, pyrrol-2-yl or pyrrol-3-yl,

which is not substituted or is substituted by 1-4 substituents selected from the group consisting of F, Cl, Br, I, —CN, (C₂-C₈)-alkanoyl, (C₂-C₈)-alkoxycarbonyl, formyl, carboxyl, —CF₃, methyl and methoxy;

or

R(17) is (C₃-C₈)-cycloalkyl or phenyl,

which is not substituted or is substituted by 1-3 substituents selected from the group consisting of F and Cl, —CF₃, methyl, hydroxyl, methoxy, —NR(37)R(38), CH₃SO₂— and H₂NO₂S—;

R(37) and R(38)

are hydrogen or —CH₃;

R(4) and R(5)

independently of one another are hydrogen, alkyl having 1, 2, 3 or 4 carbon atoms, F, Cl, —OR(32), —NR(33)R(34) or —CᵣF₂ᵣ₊₁;

R(32), R(33) and R(34)

independently of one another are hydrogen or alkyl having 1, 2 or 3 carbon atoms;

r is 1, 2, 3 or 4;

and their pharmacologically tolerable salts;

(HOE 95/F 072—EP-A 738 712, NZ 286 380)

ai) indenoylguanidines of the formula I

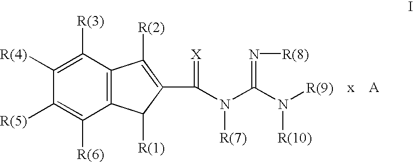

in which:

R(1) and R(2)

independently of one another are hydrogen, alkyl having 1, 2, 3, 4, 5, 6, 7, 8, 9 or 10 carbon atoms, cycloalkyl having 3, 4, 5 or 6 carbon atoms, O-alkyl having 1, 2, 3 or 4 carbon atoms, O—C(═O)-alkyl having 1, 2, 3 or 4 carbon atoms or CₘH₂ₘ—NR(12)R(13);

R(12) and R(13)

independently of one another are hydrogen or alkyl having 1, 2, 3 or 4 carbon atoms;

m is zero, 1, 2, 3 or 4;

NH—C(═O)—NH₂, C(═O)—O-alkyl having 1, 2, 3 or 4 carbon atoms, C(═O)—NH₂, C(═O)—NH-alkyl having 1, 2, 3 or 4 carbon atoms, C(═O)—N(alkyl)₂ having 1, 2, 3 or 4 carbon atoms in each alkyl group, alkenyl having 2, 3, 4, 5, 6, 7, 8, 9 or 10 carbon atoms, alkynyl having 2, 3, 4, 5, 6, 7, 8, 9 or 10 carbon atoms, alkylaryl having 1, 2, 3 or 4 carbon atoms in the alkyl group, alkenylaryl having 2, 3, 4, 5, 6, 7, 8, 9 or 10 carbon atoms in the alkenyl group, alkynylaryl having 2, 3, 4, 5, 6, 7, 8, 9 or 10 carbon atoms in the alkynyl group, C₁-C₄-alkyl-substituted aryl, C₁-C₄-alkylheteroaryl, C₁-C₄-alkenylheteroaryl, aminoalkylaryl having 1, 2, 3 or 4 carbon atoms in the alkyl group, substituted aryl, heteroaryl and substituted heteroaryl;

R(3), R(4), R(5) and R(6)

independently of one another are hydrogen, alkyl having 1, 2, 3, 4, 5, 6, 7, 8, 9 or 10 carbon atoms, O-alkyl having 1, 2, 3, 4, 5, 6, 7, 8, 9 or 10 carbon atoms, halogen, (such as F, Cl, Br, I), OH, aryl, substituted aryl, heteroaryl, substituted heteroaryl, O-lower alkyl, O-aryl, O-lower alkylaryl, O-substituted aryl, O-lower alkyl-substituted aryl, O—C(═O)—C₁-C₄-alkylaryl, O—C(═O)—N H—C₁-C₄-alkyl, O—C(═O)—N(C₁-C₄-alkyl)₂, NO₂, CN, CF₃, NH₂, NH—C(═O)—C₁-C₄-alkyl, NH—C(═O)—NH₂, COOH, C(═O)—O—C₁-C₄-alkyl, C(═O)—NH₂, C(—O)—NH—C₁-C₄-alkyl, C(═O)—N(C₁-C₄-alkyl)₂, C₁-C₄—COOH, C₁-C₄-alkyl-C (═O)—O—C₁-C₄-alkyl, SO₃H, SO₂-alkyl, SO₂-alkylaryl, SO₂—N-(alkyl)₂, SO₂—N(alkyl)(alkylaryl), C(═O)—R(11), C₁-C₁₀-alkyl-C(═O)—R(11), C₂-C₁₀-alkenyl-C(═O)—R(11), C₂-C₁₀-alkynyl-C(═O)—R(11), NH—C(═O)—C₁-C₁₀-alkyl-C(═O)—R(11), O—C₁-C₁₁-alkyl-C(═O)—R(11);

R(11) is C₁-C₄-alkyl, C₁-C₄-alkynyl, aryl, substituted aryl, NH₂, NH—C₁-C₄-alkyl, N—(C₁-C₄-alkyl)₂, SO₃H, SO₂-alkyl, SO₂-alkylaryl, SO₂—N-(alkyl)₂, SO₂—N(alkyl)(alkylaryl);

X is O, S or NH;

R(7), R(8), R(9) and R(10)

independently of one another are hydrogen, alkyl, cycloalkyl, aryl, alkylaryl;

or

R(8) and R(9)

together are part of a 5-, 6- or 7-membered heterocyclic ring;

A is absent or is a nontoxic organic or inorganic acid;

(HOE 95/F 109—EP 748 795, NZ 286 583)

ak) benzyloxycarbonylguanidines of the formula I

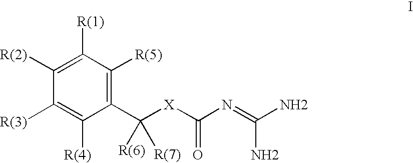

in which:

R(1), R(2) and R(3)

independently of one another are —Y-[4-R(8)-phenyl], —Y-[3—R(8)-phenyl] or —Y-[2—R(8)-phenyl],

where the phenyl is in each case unsubstituted or substituted by 1-2 substituents from the group consisting of F, Cl, —CF₃, methyl, hydroxyl, methoxy and —NR(96)R(97);

R(96) and R(97)

independently of one another are hydrogen or —CH3;

Y is a bond, CH₂, oxygen, —S— or —NR(9);

R(9) is hydrogen or alkyl having 1, 2, 3 or 4 carbon atoms;

R(8) is SOₐ[NR(98)]_{b}NR(99)R(10);

a is 1 or 2;

b is 0 or 1;

a+b=2;

R(98), R(99) and R(10)

independently of one another are hydrogen, —(C₁-C₈)-alkyl, benzyl, —(C₂-C₈)-alkylene-NR(11)R(12), (C₂-C₈)-alkylene-NR(13)—(C₂-C₈)-alkylene-NR(37)R(38) or (C₀-C₈)-alkylene-CR(39)R(40)CR(41)R(42)(C₀-C₈)-alkylene-NR(43)R(44);

R(11), R(12), R(13), R(37), R(38), R(43) and R(44)

independently of one another are hydrogen, —(C₁-C₈)-alkyl or benzyl;

R(39), R(40), R(41) and R(42)

independently of one another are hydrogen, —(C₁-C₈)-alkyl or —(C₀-C₃)-alkylenephenyl,

where the phenyl is not substituted or is substituted by 1-3 substituents selected from the group consisting of F, Cl, —CF₃, methyl and methoxy;

or

R(99) and R(10)

together are 4-6 methylene groups, of which one CH₂ group can be replaced by oxygen, —S—, —NH—, —N—CH₃ or —N-benzyl;

or

R(8) is SOₐ[NR(98)]_{b}NR(95)—C[═N—R(94)]—NR(93)R(92);

R(92), R(93), R(94) and R(95)

independently of one another are hydrogen or alkyl having 1, 2, 3 or 4 carbon atoms;

or

R(1), R(2) and R(3)

independently of one another are pyrrol-1-yl, pyrrol-2-yl or pyrrol-3-yl,

which is not substituted or is substituted by 1-4 substituents selected from the group consisting of F., Cl, Br, I, —CN, (C₂-C₈)-alkanoyl, (C₂-C₈)-alkoxycarbonyl, formyl, carboxyl, —CF₃, methyl, methoxy;

or

R(1), R(2) and R(3)

independently of one another are hydrogen, —(C₁-C₈)-alkyl, —(C₂-C₈)-alkenyl or —(CH₂)ₘR(14);

m is zero, 1 or 2;

R(14) is —(C₃-C₈)-cycloalkyl or phenyl,

which is not substituted or is substituted by 1-3 substituents selected from the group consisting of F and Cl, —CF₃, methyl, methoxy and —NR(15)R(16);

R(15) and R(16)

are hydrogen or —CH₃;

or

R(1), R(2) and R(3)

independently of one another are —Q-4-[(CH₂)ₖ—CHR(17)—(C═O)R(20)]-phenyl, —Q-3-(CH₂)ₖ—CHR(17)—(C═O)R(20)]-phenyl or —Q-2-[(CH₂)ₖ—CHR(17)—(C═O)R(20)]-phenyl,

where the phenyl in each case is unsubstituted or substituted by 1-2 substituents from the group F, Cl, —CF₃, methyl, hydroxyl, methoxy and —NR(35)R(36);

R(35) and R(36)

independently of one another are hydrogen or —CH3;

Q is a bond, oxygen, —S— or —NR(18);

R(18) is hydrogen or —(C₁-C₄)-alkyl;

R(17) is —OR(21) or —NR(21)R(22);

R(21) and R(22)

independently of one another are hydrogen, —(C₁-C₈)-alkyl, —(C₁-C₈)-alkanoyl, —(C₁-C₈)-alkoxycarbonyl, benzyl, benzyloxycarbonyl;

or

R(21) is trityl;

R(20) is —OR(23) or —NR(23)R(24);

R(23), R(24) independently of one another are

hydrogen, —(C₁-C₈)-alkyl or benzyl;

k is zero, 1, 2, 3 or 4;

or

R(1), R(2) and R(3)

independently of one another are (C₁-C₉)-heteroaryl,

which is linked via C or N and which is unsubstituted or substituted by 1-3 substituents from the group F, Cl, CF₃, CH₃, methoxy, hydroxyl, amino, methylamino and dimethylamino;

or

R(1), R(2) and R(3)

are —SR(25), —OR(25), —NR(25)R(26), —CR(25)R(26)R(27);

R(25) is —C_{f}H_{2f}—(C₁-C₉)-heteroaryl,

which is unsubstituted or substituted by 1-3 substituents from the group F, Cl, CF₃, CH₃, methoxy, hydroxyl, amino, methylamino and dimethylamino;

f is zero, 1 or 2;

R(26) and R(27)

independently of one another are defined as R(25) or are hydrogen or (C₁-C₄)-alkyl,

or

R(1), R(2) and R(3)

independently of one another are (C₁-C₉)-heteroaryl-N-oxide,

which is linked via C or N and which is unsubstituted or substituted by 1-3 substituents selected from the group consisting of F, Cl, CF₃, CH₃, methoxy, hydroxyl, amino, methylamino and dimethylamino;

or

R(1), R(2) and R(3)

independently of one another are -SR(28), —OR(28), —NR(28)R(29) or —CR(28)R(29)R(30);

R(28) is —C_{g}H_{2g}—(C₁-C₉)-heteroaryl-N-oxide,

which is unsubstituted or substituted by 1-3 substituents selected from the group consisting of F, Cl, CF₃, CH₃, methoxy, hydroxyl, amino, methylamino and dimethylamino;

g is zero, 1 or 2;

R(29), R(30)

independently of one another are defined as R(28), hydrogen or (C₁-C₄)-alkyl;

or

R(1), R(2) and R(3)

independently of one another are hydrogen, F, Cl, Br, I, —C≡N, T—(CH₂)ₕ—(CᵢF₂ᵢ₊₁), R(31)SOₗ—R(32)R(33)N—CO—, R(34)—CO— or

R(45)R(46)N—SO₂, where the perfluoroalkyl group is straight-chain or branched;

T is a bond, oxygen, —S— or —NR(47);

l is zero, 1 or 2;

h is zero, 1 or 2;

i is 1, 2, 3, 4, 5 or 6;

R(31), R(32), R(34) and R(45)

independently of one another are —(C₁-C₈)-alkyl, —(C₃-C₆)-alkenyl, (CH₂)ₙR(48) or —CF₃;

n is zero, 1, 2, 3 or 4;

R(47) is hydrogen or alkyl with 1, 2 or 3 carbon atoms;

R(48) is —(C₃-C₇)-cycloalkyl or phenyl,

which is not substituted or is substituted by 1-3 substituents selected from the group consisting of F, Cl, —CF₃, methyl, methoxy and —NR(49)R(50);

R(49) and R(50)

are hydrogen or alkyl having 1, 2, 3 or 4 carbon atoms;

or

R(32), R(34) and R(45)

are hydrogen;

R(33) and R(46)

independently of one another are hydrogen or alkyl having 1, 2, 3 or 4 carbon atoms;

or

R(32) and R(33), and R(45) and R(46)

together are 5 or 6 methylene groups, of which one CH₂ group can be replaced by oxygen, —S—, —NH—, —NCH₃ or —N-benzyl;

or

R(1), R(2) and R(3)

independently of one another are R(51)—A—G—D—;

R(51) is a basic protonatable radical, i.e. an amino group —NR(52)R(53), an amidino group R(52)R(53)N—C[═N—R(54)]— or a guanidino group R(52)R(53)N—C[═N—R(54)]—NR(55)—;

R(52), R(53), R(54) and R(55)

independently of one another are hydrogen or alkyl having 1, 2, 3 or 4 carbon atoms;

or

R(52) and R(53) are

a group C_{α}H_{2α};

α is 4, 5, 6 or 7;

where if α=5, 6 or 7 a carbon atom of the group C_{α}H_{2α} can be replaced by a heteroatom group O, SO_{d} or NR(56),

or

R(53) and R(54) or R(54) and R(55) or R(52) and R(55) are

a group C_{y}H_{2y};

y is 2, 3, 4 or 5;

where if y=3, 4 or 5 a carbon atom of the group C_{y}H_{2y} can be replaced by a heteroatom group O, SO_{d} or NR(56);

d is zero, 1 or 2;

R(56) is hydrogen or methyl;

or

R(51) is a basic heteroaromatic ring system having 1-9 carbon atoms;

A is a group CₑH₂ₑ;

e is zero, 1, 2, 3, 4, 5, 6, 7, 8, 9 or 10;

where in the group CₑH₂ₑ a carbon atom can be replaced by one of the groupings —O—, —CO—, or —SOᵣ—;

r is zero, 1 or 2;

G is a phenylene radical

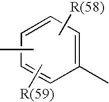

R(58) and R(59)

independently of one another are hydrogen, methyl, methoxy, F, Cl, Br, I, CF₃ or —Soₛ—R(60);

s zero 1 or 2;

R(60) is methyl or NR(61)R(62);

R(61) and R(62)

independently of one another are hydrogen or alkyl having 1, 2, 3 or 4 carbon atoms;

D is —CᵥH₂ᵥ—E_{w}—;

v is zero, 1, 2, 3 or 4;

E is —O—, —CO—, —CH[OR(63)]—, —SOₐₐ— or —NR(63)—;

w is zero or 1;

aa is zero, 1 or 2

R(63) is hydrogen or methyl,

or

R(1), R(2) and R(3)

independently of one another are —CF₂R(64), —CF[R(65)][R(66)], —CF[(CF₂)_{q}—CF₃)][R(65)], —C[(CF₂)ₚ—CF₃]═CR(65)R(66);

R(64) is alkyl having 1, 2, 3 or 4 carbon atoms or cycloalkyl having 3, 4, 5, 6 or 7 carbon atoms;

R(65) and R(66) independently of one another are hydrogen or alkyl having 1, 2, 3 or 4 carbon atoms;

q is zero, 1 or 2;

p is zero, 1 or 2;

or

R(1), R(2) and R(3)

independently of one another are —OR(67) or —NR(67)R(68);

R(67) and R(68)

independently of one another are hydrogen or alkyl having 1, 2, 3, 4, 5 or 6 carbon atoms;

or

R(67) and R(68)

together are 4, 5, 6 or 7 methylene groups, of which one CH₂ group can be replaced by oxygen, —S—, SO₂, —NH—, —NCH₃ or —N-benzyl;

R(4) and R(5)

independently of one another are hydrogen, alkyl having 1, 2, 3 or 4 carbon atoms, F, Cl, —OR(69), —NR(70)R(71) or —C_{z}F_{2z+1};

R(69), R(70) and R(71)

independently of one another are hydrogen or alkyl having 1, 2 or 3 carbon atoms;

z is 1, 2, 3 or 4;

R(6) and R(7)

independently of one another are hydrogen or alkyl having 1, 2, 3 or 4 carbon atoms;

X is oxygen or NR(72);

R(72) is hydrogen or alkyl having 1, 2, 3 or 4 carbon atoms;

and their pharmaceutically tolerable salts;

(HOE 95/F 115—EP 744 397, NZ 286 622)

al) alkenylcarboxylic acid guanidides, carrying fluorophenyl groups, of the Formula I

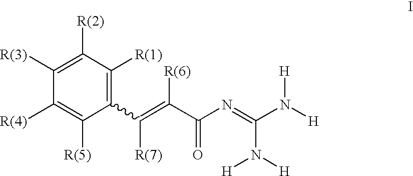

in which:

R(6) is hydrogen, (C₁-C₈)-alkyl, (C₃-C₈)-cycloalkyl or phenyl,

where the phenyl group is not substituted or is substituted by 1-3 substituents selected from the group consisting of F, Cl, CF₃, methyl, methoxy and NR(9)R(10);

R(9) and R(10)

are hydrogen, (C₁-C₄)-alkyl or (C₁-C₄)-perfluoroalkyl;

R(7) independently is defined as R(6);

R(1), R(2), R(3), R(4) and R(5)

independently of one another are hydrogen or F;

where, however, at least one of the radicals R(1), R(2), R(3), R(4) and R(5) must be fluorine;

and their pharmaceutically tolerable salts;

(HOE 95/F 167—NZ 299 015)

am) benzoylguanidines of the formula I

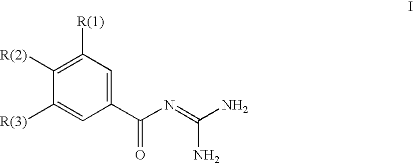

in which:

R(1) is R(4)—SOₘ or R(5)R(6)N—SO₂—;

m is 1 or 2;

R(4) and R(5)

independently of one another are alkyl having 1, 2, 3, 4, 5, 6, 7 or 8 carbon atoms, alkenyl having 3, 4, 5 or 6 carbon atoms, CF₃ or —CₙH₂ₙ—R(7);

n is zero, 1, 2, 3 or 4;

R(6) is H or alkyl having 1, 2, 3 or 4 carbon atoms;

R(7) is cycloalkyl having 3, 4, 5, 6, 7 or 8 carbon atoms or phenyl,

which is not substituted or is substituted by 1-3 substituents selected from the group consisting of F, Cl, CF₃, methyl, methoxy and NR(8)R(9);

R(8) and R(9)

are H or alkyl having 1, 2, 3 or 4 carbon atoms;

or

R(5) is also hydrogen;

or

R(5) and R(6)

together are 4 or 5 methylene groups, of which a CH₂ group can be replaced by oxygen, S, NH, N—CH₃ or N-benzyl;

or

R(1) is —Oₚ—(CH₂)_{q}—(CF₂)ᵣ—CF₃;

p is zero or 1;

q is zero, 1 or 2;

r is zero, 1, 2 or 3;

or

R(1) is —SR(10), —OR(10) or —CR(10)R(11)R(12);

R(10), R(11) and R(12)

independently of one another are hydrogen, alkyl having 1, 2, 3, 4, 5, 6, 7 or 8 carbon atoms, —CₛH₂ₛ—(C₃-C₈)-cycloalkyl or an aromatic system selected from the group consisting of pyridyl, pyrrolyl, quinolyl, isoquinolyl, imidazolyl or phenyl;

s is zero, 1 or 2;

where the aromatic systems pyridyl, pyrrolyl, quinolyl, isoquinolyl, imidazolyl and phenyl are unsubstituted or substituted by 1-3 substituents selected from the group consisting of F, Cl, CF₃, CH₃, methoxy, hydroxyl, amino, methylamino and dimethylamino;

R(2) is —(CH₂)ᵤ—(CF₂)ₜ—CF₃;

t is zero, 1, 2 or 3;

u is zero or 1;

R(3) is hydrogen or independently is defined as R(1);

and their pharmaceutically tolerable salts;

(HOE 95/F 173—NZ 299 052)

an) substituted cinnamic acid guanidides of the formula I

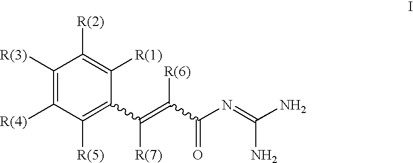

in which:

at least one of the substituents R(1), R(2), R(3), R(4) and R(5) is

—Xₐ—Y_{b}—Lₙ—U;

X is CR(16)R(17), O, S or NR(18);

R(16), R(17) and R(18)

independently of one another are H, alkyl having 1, 2, 3 or 4 carbon atoms or perfluoroalkyl having 1, 2, 3 or 4 carbon atoms;

a is zero or 1;

Y is alkylene having 1, 2, 3, 4, 5, 6, 7 or 8 carbon atoms, alkylene-T having 1, 2, 3, 4, 5, 6, 7 or 8 carbon atoms in the alkylene group, T, T-alkylene having 1, 2, 3, 4, 5, 6, 7 or 8 carbon atoms in the alkylene group;

T is NR(20), O, S or phenylene,

where the phenylene is not substituted or is substituted by 1-3 substituents selected from the group consisting of F, Cl, CF₃, methyl, methoxy and NR(21)R(22);

R(20), R(21) and R(22)

independently of one another are H, alkyl having 1, 2, 3 or 4 carbon atoms or perfluoroalkyl having 1, 2, 3 or 4 carbon atoms;

b is zero or 1;

L is O, S, NR(23) or CₖH₂ₖ;

k is 1, 2, 3, 4, 5, 6, 7 or 8;

n is zero or 1;

U is NR(24)R(25) or an N—containing heterocycle having 1, 2, 3, 4, 5, 6, 7, 8 or 9 carbon atoms;

R(24) and R(25)

independently of one another are hydrogen, alkyl having 1, 2, 3, 4, 5, 6, 7 or 8 carbon atoms or perfluoroalkyl having 1, 2, 3, 4, 5, 6, 7 or 8 carbon atoms;

or

R(24) and R(25)

together are 4 or 5 methylene groups, of which one CH₂ group can be replaced by oxygen, S, NH, N—CH₃ or N-benzyl;

where the N-containing heterocycles are N- or C-bridged and are not substituted or are substituted by 1-3 substituents selected from the group consisting of F, Cl, CF₃, methyl, methoxy and NR(27)R(28);

R(23), R(27) and R(28)

independently of one another are H, alkyl having 1, 2, 3 or 4 carbon atoms or perfluoroalkyl having 1, 2, 3 or 4 carbon atoms;

and the other substituents R(1), R(2), R(3), R(4) and R(5) in each case

independently of one another are H, F, Cl, Br, I, CN, —Oₙ—CₘH₂ₘ₊₁, —Oₚ—(CH₂)ₛ—C_{q}F_{2q+1} or —CᵣH₂ᵣR(10);

n is zero or 1;

m is zero, 1, 2, 3, 4, 5, 6, 7 or 8;

p is zero or 1;

q is 1, 2, 3, 4, 5, 6, 7 or 8;

s is zero, 1, 2, 3 or 4;

r is zero, 1, 2, 3 or 4;

R(10)

is cycloalkyl having 3, 4, 5, 6, 7 or 8 carbon atoms, or phenyl,

where the phenyl is not substituted or is substituted by 1-3 substituents selected from the group consisting of F, Cl, CF₃, methyl, methoxy and NR(11)R(12);

R(11) and R(12)

independently of one another are H, alkyl having 1, 2, 3 or 4 carbon atoms or perfluoroalkyl having 1, 2, 3 or 4 carbon atoms;

R(6) and R(7)

independently of one another are hydrogen, F, Cl, Br, I, CN, alkyl having 1, 2, 3, 4, 5, 6, 7 or 8 carbon atoms, perfluoroalkyl having 1, 2, 3, 4, 5, 6, 7 or 8 carbon atoms, cycloalkyl having 3, 4, 5, 6, 7 or 8 carbon atoms, or phenyl,

which is not substituted or is substituted by 1-3 substituents selected from the group consisting of F, Cl, CF₃, methyl, methoxy and NR(14)R(15);

R(14) and R(15)

independently of one another are H, alkyl having 1, 2, 3 or 4 carbon atoms or perfluoroalkyl having 1, 2, 3 or 4 carbon atoms;

and their pharmaceutically tolerable salts;

(HOE 95/F 220—NZ 299 052)

ao) benzoylguanidines of the formula I

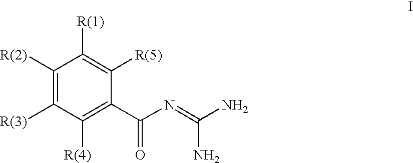

in which:

at least one of the substituents R(1), R(2) and R(3) is R(6)—C(OH)₂—;

R(6) is perfluoroalkyl having 1, 2 or 3 carbon atoms, which is straight-chain or branched;

and the other substituents R(1), R(2) and R(3)

independently of one another are hydrogen, OH, F, Cl, Br, I, alkyl having 1, 2, 3, 4, 5 or 6 carbon atoms, cycloalkyl having 3, 4, 5 or 6 carbon atoms, alkoxy having 1, 2, 3 or 4 carbon atoms, or phenoxy,

which is unsubstituted or is substituted by 1-3 substituents selected from the group consisting of F, Cl, methyl and methoxy;

or

the other substituents R(1), R(2) and R(3)

independently of one another are alkyl-SOₓ, —CR(7)═CR(8)R(9) or —C≡—CR(9);

x is zero, 1 or 2;

R(7) is hydrogen or methyl;

R(8) and R(9)

independently of one another are hydrogen, alkyl having 1, 2, 3 or 4 carbon atoms, cycloalkyl having 3, 4, 5, 6, 7 or 8 carbon atoms, or phenyl,

which is unsubstituted or is substituted by 1-3 substituents selected from the group consisting of F, Cl, CF₃, methyl and methoxy;

or

the other substituents R(1), R(2) and R(3)

independently of one another are phenyl, C₈H₅—(C₁-C₄)-alkyl, naphthyl, biphenylyl, quinolinyl, isoquinolinyl or imidazolyl,

where quinolinyl, isoquinolinyl or imidazolyl are bonded via C or N and where phenyl, C₆H₅-(C₁-C₄)-alkyl, naphthyl, biphenylyl, quinolinyl, isoquinolinyl and imidazolyl are unsubstituted or are substituted by 1-3 substituents selected from the group consisting of F, Cl, CF₃, CH₃, methoxy, hydroxyl, amino, methylamino and dimethylamino;

or

the other substituents R(1), R(2) and R(3)

independently of one another are SR(10), —OR(10), —CR(10)R(11)R(12);

R(10)

is —C_{f}H_{2f}—(C₃-C₈)-cycloalkyl, quinolinyl, isoquinolinyl, pyridinyl, imidazolyl or phenyl,

where the aromatic systems quinolinyl, isoquinolinyl, pyridinyl, imidazolyl and phenyl are unsubstituted or are substituted by 1-3 substituents selected from the group consisting of F, Cl, CF₃, CH₃, methoxy, hydroxyl, amino, methylamino and dimethylamino;

f is zero, ₁ or 2;

R(11) and R(12)

independently of one another are defined as R(10), hydrogen or alkyl having 1, 2, 3 or 4 carbon atoms;

R(4) and R(5)

independently of one another are hydrogen, alkyl having 1, 2 or 3 carbon atoms, F, Cl, Br, I, CN, OR(13), NR(14)R(15), —(CH₂)ₙ—(CF₂)ₒ—CF₃;

R(13), R(14) and R(15)

independently of one another are hydrogen or alkyl having 1, 2, 3 or 4 carbon atoms;

n is zero or 1;

o is zero, 1 or 2;

and their pharmacologically acceptable salts;

(HOE 95/F 253—NZ 299 682)

ap) sulfonimidamides of the formula I

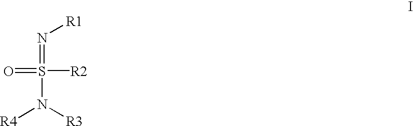

in which:

at least one of the three substituents R(1), R(2) and R(3) is a benzoylguanidine,

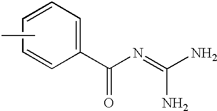

which is unsubstituted or substituted in the phenyl moiety by 1-4 radicals selected from the group consisting of alkyl having 1, 2, 3, 4, 5, 6, 7 or 8 carbon atoms, alkenyl having 2, 3, 4, 5, 6, 7 or 8 carbon atoms, —(CH₂)ₘ—R(14), F, Cl, Br, I, —C≡N, CF₃, R(22)SO₂—, R(23)R(24)N—CO—, R(25)—CO—,

R(26)R(27)N—SO₂, —OR(35), —SR(35) or —NR(35)R(36);

m is zero, 1 or 2;

R(14)

is —(C₃-C₈)-cycloalkyl or phenyl,

which is not substituted or is substituted by 1-3 substituents selected from the group consisting of F and Cl, —CF₃, methyl, methoxy and —NR(15)R(16);

R(15) and R(16)

independently of one another are hydrogen or —CH₃;

R(22), R(23), R(25) and R(26)

independently of one another are alkyl having 1, 2, 3, 4, 5, 6, 7 or 8 carbon atoms, alkenyl having 2, 3, 4, 5, 6, 7 or 8 carbon atoms, (CH₂)ₙR(29) or —CF₃;

n is zero, 1, 2, 3 or 4;

R(29) is —(C₃-C₇)-cycloalkyl or phenyl,

which is not substituted or is substituted by 1-3 substituents selected from the group consisting of F, Cl, —CF₃, methyl, methoxy and —NR(30)R(31);

R(30) and R(31)

are hydrogen or alkyl having 1, 2, 3 or 4 carbon atoms;

or

R(23), R(25) and R(26)

are hydrogen;

R(24) and R(27)

independently of one another are hydrogen or alkyl having 1, 2, 3 or 4 carbon atoms;

or

R(23) and R(24), and also R(26) and R(27)

together are 5 or 6 methylene groups, of which one CH₂ group can be replaced by oxygen, —S—, —NH—, —NCH₃ or —N-benzyl;

R(35) and R(36)

independently of one another are hydrogen or alkyl having 1, 2, 3, 4, 5 or 6 carbon atoms;

or

R(35) and R(36)

together are 4-7 methylene groups, of which one CH₂ group can be replaced by oxygen, —S—, —NH—, —NCH₃ or —N-benzyl;

or

R(35) is phenyl,

which is not substituted or is substituted by 1-3 substituents selected from the group consisting of F, Cl, —CF₃, methyl, methoxy, SO₂R(5), SO₂NR(6)R(7) and —NR(32)R(33);

R(5) is alkyl having 1, 2, 3, 4, 5 or 6 carbon atoms;

R(6) and R(7)

independently of one another are hydrogen or alkyl having 1, 2, 3 or 4 carbon atoms;

R(32) and R(33)

independently of one another are hydrogen or alkyl having 1, 2, 3 or 4 carbon atoms;

or

R(35) is C₁-C₉-heteroaryl,

which is unsubstituted or substituted by 1-3 substituents selected from the group consisting of F, Cl, CF₃, CH₃, methoxy, hydroxyl, amino, methylamino and dimethylamino;

and the other substituents R(1), R(2) and R(3) in each case

independently of one another are alkyl having 1, 2, 3, 4, 5, 6, 7 or 8 carbon atoms, (CH₂)ₚR(10);

p is zero, 1, 2, 3 or 4;

R(10) is phenyl,

which is not substituted or is substituted by 1-3 substituents selected from the group consisting of F, Cl, —CF₃, methyl, methoxy, —SO₂NR(17)R(8) and —SO₂R(9);

R(17) and R(8)

independently of one another are hydrogen or alkyl having 1, 2, 3 or 4 carbon atoms;

R(9) is alkyl having 1, 2, 3 or 4 carbon atoms;

or

the other radical R(1) or R(3) in each case

is hydrogen,

R(4) is hydrogen or alkyl having 1, 2, 3 or 4 carbon atoms;

and their pharmaceutically tolerable salts;

(HOE 95/F 265—NZ 299 739)

aq) benzoylguanidines of the formula I

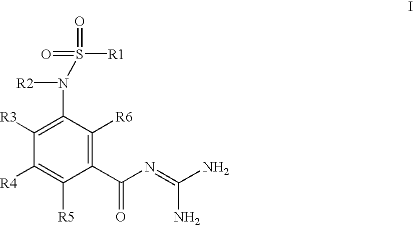

in which:

R(1) is alkyl having 1, 2, 3, 4, 5, 6, 7 or 8 carbon atoms or NR(7)R(8);

R(7) and R(8)

independently of one another are hydrogen or alkyl having 1, 2, 3, 4, 5, 6, 7 or 8 carbon atoms;

R(2) is hydrogen, alkyl having 1, 2, 3, 4, 5, 6, 7 or 8 carbon atoms or —SO₂R(9);

R(9) independently is defined as R(1);

R(3) is hydrogen, —SR(25), —OR(25), —NR(25)R(26) or —CR(25)R(26)R(27);

R(25) is hydrogen, alkyl having 1, 2, 3, 4, 5, 6, 7 or 8 carbon atoms, or phenyl,

which is unsubstituted or substituted by 1-3 substituents selected from the group consisting of F, Cl, CF₃, CH₃, methoxy, hydroxyl, amino, methylamino and dimethylamino;

or

R(25)

is —(C₁-C₉)-heteroaryl,

which is unsubstituted or substituted by 1-3 substituents selected from the group consisting of F, Cl, CF₃, CH₃, methoxy, hydroxyl, amino, methylamino and dimethylamino;

R(26) and R(27)

independently of one another are defined as R(25) or are hydrogen or alkyl having 1, 2, 3, 4, 5, 6, 7 or 8 carbon atoms;

R(4) is hydrogen, F, Cl, Br, I, OH, —C≡N, CF₃, alkyl having 1, 2, 3, 4, 5, 6, 7 or 8 carbon atoms, alkenyl having 2, 3, 4, 5, 6, 7 or 8 carbon atoms or —(CH₂)ₘR(14);

m is zero, 1 or 2;

R(14) is —(C₃-C₈)-cycloalkyl or phenyl,

which is not substituted or is substituted by 1-3 substituents selected from the group consisting of F and Cl, —CF₃, methyl, methoxy and —NR(15)R(16);

R(15) and R(16)

independently of one another are hydrogen or —CH₃;

R(5) and R(6)

independently of one another are hydrogen, alkyl having 1, 2, 3 or 4 carbon atoms, F, Cl, —OR(32), —NR(33)R(34) or CF₃;

R(32), R(33) and R(34)

independently of one another are hydrogen or alkyl having 1, 2, 3 or 4 carbon atoms;

and their pharmaceutically tolerable salts;

(HOE 95/F 269 K—EP-A 774 458)

ar) benzenedicarboxylic acid diguanidides of the formula I

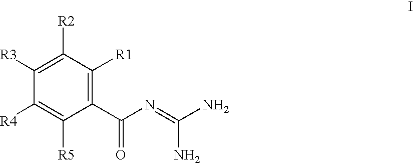

in which:

one of the radicals R(1), R(2), R(3) and R(4)

is —CO—N═C(NH₂)₂;

and of the other radicals R(1), R(2), R(3) and R(4) in each case:

R(1) is hydrogen, alkyl having 1, 2, 3 or 4 carbon atoms, F, Cl, Br, I, —OR(32), —NR(33)R(34) or CF₃;

R(32), R(33) and R(34)

independently of one another are hydrogen or alkyl having 1, 2, 3 or 4 carbon atoms;

R(2) and R(4)

independently of one another are hydrogen, F, Cl, Br, I, OH, —CN, CF₃, —CO—N═C(NH₂)₂, alkyl having 1, 2, 3, 4, 5, 6, 7 or 8 carbon atoms, alkenyl having 2, 3, 4, 5, 6, 7 or 8 carbon atoms or —(CH₂)ₘR(14);

m is zero, 1 or 2;

R(14) is —(C₃-C₈)-cycloalkyl or phenyl,

which is not substituted or is substituted by 1-3 substituents selected from the group consisting of F and Cl, —CF₃, methyl, methoxy and —NR(15)R(16);

R(15) and R(16)

are hydrogen or —CH₃;

or

R(2) and R(4)

independently of one another are pyrrol-1-yl, pyrrol-2-yl or pyrrol-3-yl,

which is not substituted or is substituted by 1-4 substituents selected from the group consisting of F, Cl, Br, 1, —CN, (C₂-C₈)-alkanoyl, (C₂-C₈)-alkoxycarbonyl, formyl, carboxyl, —CF₃, methyl, methoxy;

or

R(2) and R(4)

independently of one another are R(22)—SO₂—, R(23)R(24)N—CO—, R(28)—CO— or R(29)R(30)N—SO₂;

R(22) and R(28)

independently of one another are methyl or —CF₃;

R(23), R(24), R(29) and R(30)

independently of one another are hydrogen or methyl;

or

R(2) and R(4)

independently of one another are —OR(35) or —NR(35)R(36);

R(35) and R(36)

independently of one another are hydrogen or alkyl having 1, 2, 3, 4, 5 or 6 carbon atoms;

or

R(35) and R(36)

together are 4-7 methylene groups, of which one CH₂ group can be replaced by oxygen, —S—, —NH—, —NCH₃ or —N-benzyl;

R(3) is hydrogen, —SR(25), —OR(25), —NR(25)R(26), —CR(25)R(26)R(27);

R(25) is hydrogen, alkyl having 1, 2, 3, 4, 5, 6, 7 or 8 carbon atoms or phenyl,

which is unsubstituted or substituted by 1-3 substituents selected from the group consisting of F, Cl, CF₃, CH₃, methoxy, hydroxyl, amino, methylamino and dimethylamino;

or

R(25) is —(C₁-C₉)-heteroaryl,

which is unsubstituted or substituted by 1-3 substituents selected from the group consisting of F, Cl, CF₃, CH₃, methoxy, hydroxyl, amino, methylamino and dimethylamino;

R(26) and R(27)

independently of one another are defined as R(25) or are hydrogen or alkyl having 1, 2, 3, 4, 5, 6, 7 or 8 carbon atoms;

R(5) is alkyl having 1, 2, 3 or 4 carbon atoms, F, Cl, Br, I, X—(CH₂)_{y}—CF₃ or phenyl,

which is not substituted or is substituted by 1-3 substituents selected from the group consisting of F and Cl, —CF₃, methyl, methoxy and —NR(6)R(7);

R(6) and R(7)

independently of one another are hydrogen or —CH₃;

X is a bond or oxygen;

y is zero, 1 or 2;

and their pharmaceutically tolerable salts;

(HOE 95/F 269 BK—EP-A 774 457)

as) benzenedicarboxylic acid diguanidides of the formula I

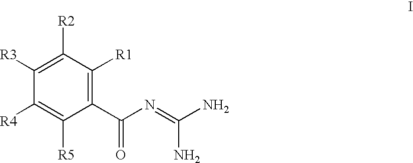

in which:

one of the radicals R(1), R(2), R(3) and R(5)

is —CO—N═C(NH₂)₂;

and of the other radicals R(1), R(2), R(3) and R(5) in each case:

R(1) and R(5)

independently of one another are hydrogen, alkyl having 1, 2, 3 or 4 carbon atoms, F, Cl, —OR(32), —NR(33)R(34) or CF₃;

R(32), R(33) and R(34)

independently of one another are hydrogen or alkyl having 1, 2, 3 or 4 carbon atoms;

R(2) is hydrogen, F, Cl, Br, I, OH, —CN, CF₃, —CO—N═C(NH₂)₂, alkyl having 1, 2, 3, 4, 5, 6, 7 or 8 carbon atoms, alkenyl having 2, 3, 4, 5, 6, 7 or 8 carbon atoms or —(CH₂)ₘR(14);

m is zero, 1 or 2;

R(14) is —(C₃-C₈)-cycloalkyl or phenyl,

which is not substituted or is substituted by 1-3 substituents selected from the group consisting of F and Cl, —CF₃, methyl, methoxy and —NR(15)R(16);

R(15) and R(16)

independently of one another are hydrogen or —CH₃;

or

R(2) is R(22)—SO₂—, R(23)R(24)N—CO—, R(28)—CO— or R(29)R(30)N—SO₂;

R(22) and R(28)

independently of one another are methyl or —CF₃;

R(23), R(24), R(29) and R(30)

independently of one another are hydrogen or methyl;

or

R(2) is —OR(35) or —NR(35)R(36);

R(35) and R(36)

independently of one another are hydrogen or alkyl having 1, 2, 3, 4, 5 or 6 carbon atoms;

or

R(35) and R(36)

together are 4-7 methylene groups, of which one CH₂ group can be replaced by oxygen, —S—, —NH—, —NCH₃ or —N-benzyl;

R(3) is hydrogen, —SR(25), —OR(25), —NR(25)R(26), —CR(25)R(26)R(27);

R(25) is hydrogen, alkyl having 1, 2, 3, 4, 5, 6, 7 or 8 carbon atoms, or phenyl,

which is unsubstituted or substituted by 1-3 substituents selected from the group consisting of F, Cl, CF₃, CH₃, methoxy, hydroxyl, amino, methylamino and dimethylamino;

or

R(25) is —(C₁-C₉)-heteroaryl,

which is unsubstituted or substituted by 1-3 substituents selected from the group consisting of F, Cl, CF₃, CH₃, methoxy, hydroxyl, amino, methylamino and dimethylamino;

R(26) and R(27)

independently of one another are defined as R(25) or are hydrogen or alkyl having 1, 2, 3, 4, 5, 6, 7 or 8 carbon atoms;

R(4) is CF₃, alkyl having 1, 2, 3, 4, 5, 6, 7 or 8 carbon atoms, alkenyl having 2, 3, 4, 5, 6, 7 or 8 carbon atoms, —(C₃-C₈)-cycloalkyl or —(CH₂)ₘR(14);

m is 1 or 2;

R(14) is —(C₃-C₈)-cycloalkyl or phenyl,

which is not substituted or is substituted by 1-3 substituents selected from the group consisting of F and Cl, —CF₃, methyl, methoxy and —NR(15)R(16);

R(15) and R(16)

independently of one another are hydrogen or —CH₃;

or

R(4) is phenyl,

which is substituted by 2, 3, 4 or five substituents selected from the group consisting of F, Cl, CF₃, methyl, methoxy and —NR(15)R(16);

R(15) and R(16)

independently of one another are hydrogen or CH₃;

and their pharmaceutically tolerable salts;

(HOE 96/F 013—EP-A 787 717)

at) diaryldicarboxylic acid diguanidides of the formula I

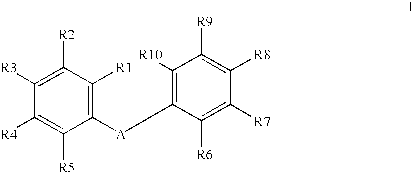

in which:

one of the radicals R(1), R(2), R(3), R(4) and R(5)

is —CO—N═C(NH₂)₂;

the other radicals R(1) and R(5) in each case

independently of one another are hydrogen, alkyl having 1, 2, 3 or 4 carbon atoms, F, Cl, —OR(32), —NR(33)R(34) or CF₃;

R(32), R(33) and R(34)

independently of one another are hydrogen or alkyl having 1, 2, 3 or 4 carbon atoms;

the other radicals R(2) and R(4) in each case

independently of one another are hydrogen, F, Cl, Br, I, OH, —CN, CF₃, —CO—N═C(NH₂)₂, alkyl having 1, 2, 3, 4, 5, 6, 7 or 8 carbon atoms, alkenyl having 2, 3, 4, 5, 6, 7 or 8 carbon atoms or —(CH₂)ₘR(14);

m is zero, 1 or 2;

R(14) is —(C₃-C₈)-cycloalkyl or phenyl,

which is not substituted or is substituted by 1-3 substituents selected from the group consisting of F and Cl, —CF₃, methyl, methoxy and —NR(15)R(16);

R(15) and R(16)

are hydrogen or —CH₃;

or

the other radicals R(2) and R(4) in each case

independently of one another are pyrrol-1-yl, pyrrol-2-yl or pyrrol-3-yl,

which is not substituted or is substituted by 1-4 substituents selected from the group consisting of F, Cl, Br, 1, —CN, (C₂-C₈)-alkanoyl, (C₂-C₈)-alkoxycarbonyl, formyl, carboxyl, —CF₃, methyl, methoxy;

or

the other radicals R(2) and R(4) in each case

are R(22)—SO₂—, R(23)R(24)N—CO—, R(28)—CO— or R(29)R(30)N—SO₂;

R(22) and R(28)

independently of one another are methyl or —CF₃;

R(23), R(24), R(29) and R(30)

independently of one another are hydrogen or methyl;

or

the other radicals R(2) and R(4) in each case

independently of one another are —OR(35) or —NR(35)R(36);

R(35) and R(36)

independently of one another are hydrogen or alkyl having 1, 2, 3, 4, 5 or 6 carbon atoms;

or

R(35) and R(36)

together are 4-7 methylene groups, of which one CH₂ group can be replaced by oxygen, —S—, —NH—, —NCH₃ or —N-benzyl;

the other radical R(3) in each case

is hydrogen, —SR(25), —OR(25), —NR(25)R(26), —CR(25)R(26)R(27);

R(25) is hydrogen, alkyl having 1, 2, 3, 4, 5, 6, 7 or 8 carbon atoms, or phenyl,

which is unsubstituted or substituted by 1-3 substituents selected from the group consisting of F, Cl, CF₃, CH₃, methoxy, hydroxyl, amino, methylamino and dimethylamino;

or

R(25) is —(C₁-C₉)-heteroaryl,

which is unsubstituted or substituted by 1-3 substituents selected from the group consisting of F. Cl, CF₃, CH₃, methoxy, hydroxyl, amino, methylamino and dimethylamino;

R(26) and R(27)

independently of one another are defined as R(25) or are hydrogen or alkyl having 1, 2, 3, 4, 5, 6, 7 or 8 carbon atoms;

one of the radicals R(6), R(7), R(8), R(9) and R(10)

is —CO—N═C(NH₂)₂;

the other radicals R(6) and R(10) in each case

independently of one another are hydrogen, alkyl having 1, 2, 3 or 4 carbon atoms, F, Cl, —OR(132), —NR(133)R(134) or CF₃;

R(132), R(133) and R(134)

independently of one another are hydrogen or alkyl having 1, 2, 3 or 4 carbon atoms;

the other radicals R(7) and R(9) in each case

independently of one another are hydrogen, F, Cl, Br, I, OH, —CN, CF₃, —CO—N═C(NH₂)₂, alkyl having 1, 2, 3, 4, 5, 6, 7 or 8 carbon atoms, alkenyl having 2, 3, 4, 5, 6, 7 or 8 carbon atoms or —(CH₂)ₘₘR(114);

mm is zero, 1 or 2;

R(114)

is —(C₃-C₈)-cycloalkyl or phenyl, which is not substituted or is substituted by 1-3 substituents selected from the group consisting of F and Cl, —CF₃, methyl, methoxy and —NR(115)R(116);

R(115) and R(116)

are hydrogen or —CH₃;

or

the other radicals R(7) and R(9) in each case

independently of one another are pyrrol-1-yl, pyrrol-2-yl or pyrrol-3-yl,

which is not substituted or is substituted by 1-4 substituents selected from the group consisting of F, Cl, Br, I, —CN, (C₂-C₈)-alkanoyl, (C₂-C₈)-alkoxycarbonyl, formyl, carboxyl, —CF₃, methyl and methoxy;

or

the other radicals R(7) and R(9) in each case

are R(122)-SO₂—, R(123)R(124)N—CO—, R(128)—CO— or

R(129)R(130)N—SO₂;

R(122) and R(128)

independently of one another are methyl or —CF₃;

R(123), R(124), R(129) and R(130)

independently of one another are hydrogen or methyl;

or

the other radicals R(7) and R(9) in each case

independently of one another are —OR(135) or —NR(135)R(136);

R(135) and R(136)

independently of one another are hydrogen or alkyl having 1, 2, 3, 4, 5 or 6 carbon atoms;

or

R(135) and R(136)

together are 4-7 methylene groups, of which one CH₂ group can be replaced by oxygen, —S—, —NH—, —NCH₃ or —N-benzyl;

the other radical R(8) in each case

is hydrogen, —SR(125), —OR(125), —NR(125)R(126) or —CR(125)R(126)R(127);

R(125)

is hydrogen, alkyl having 1, 2, 3, 4, 5, 6, 7 or 8 carbon atoms or phenyl,

which is unsubstituted or substituted by 1-3 substituents selected from the group consisting of F, Cl, CF₃, CH₃, methoxy, hydroxyl, amino, methylamino and dimethylamino;

or

R(125)

is —(C₁-C₉)-heteroaryl,

which is unsubstituted or substituted by 1-3 substituents selected from the group consisting of F, Cl, CF₃, CH₃, methoxy, hydroxyl, amino, methylamino and dimethylamino;

R(126) and R(127)

independently of one another are defined as R(125) or are hydrogen or alkyl having 1, 2, 3, 4, 5, 6, 7 or 8 carbon atoms;

A is absent or is —NR(11)—CO—, —NR(12)—CO—NR(13)—, —NR(17)—CO—NR(18)—SO₂—, —NR(19)—SO₂—, —SO₂—NR(19)—SO₂—, —SO₂—NR(19)—CO—, —O—CO—NR(19)—SO₂— or —CR(20)═CR(21)—;

R(11), R(12), R(13), R(17), R(18), R(19), R(20) and R(21)

independently of one another are hydrogen or alkyl having 1, 2, 3, 4, 5, 6, 7 or 8 carbon atoms

and their pharmaceutically tolerable salts;

(HOE 96/F 026—EP-A 790 245)

au) substituted thiophenylalkenylcarboxylic acid guanidides of the formula I

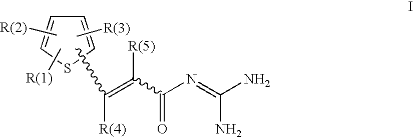

in which:

at least one of the substituents R(1), R(2) and R(3)

is —Oₚ—(CH₂)ₛ—C_{q}F_{2q+1}, R(40)CO— or R(³¹)SOₖ—;

p is zero or 1;

s is zero, 1, 2, 3 or 4;

q is 1, 2, 3, 4, 5, 6, 7 or 8;

k is zero, 1 or 2;

R(40) is alkyl having 1, 2, 3, 4, 5, 6, 7 or 8 carbon atoms,

perfluoroalkyl having 1, 2, 3, 4, 5, 6, 7 or 8 carbon atoms, cycloalkyl having 3, 4, 5, 6, 7 or 8 carbon atoms, or phenyl,

which is not substituted or is substituted by 1-3 substituents selected from the group consisting of F, Cl, CF₃, methyl and methoxy;

R(31) is alkyl having 1, 2, 3, 4, 5, 6, 7 or 8 carbon atoms, perfluoroalkyl having 1, 2, 3, 4, 5, 6, 7 or 8 carbon atoms, cycloalkyl having 3, 4, 5, 6, 7 or 8 carbon atoms, or phenyl,

which is not substituted or is substituted by 1-3 substituents selected from the group consisting of F, Cl, CF₃, methyl or methoxy;

or

R(31) is NR(41)R(42);

R(41) and R(42)

independently of one another are hydrogen, alkyl having 1, 2, 3 or 4 carbon atoms, perfluoroalkyl having 1, 2, 3 or 4 carbon atoms,

or

R(41) and R(42)

together are 4 or 5 methylene groups, of which one CH₂ group can be replaced by oxygen, S, NH, N—CH₃ or N-benzyl;

and the other substituents R(1), R(2) and R(3) in each case

independently of one another are H, F, Cl, Br, I, CN, —Oₙₐ—CₘₐH₂ₘₐ₊₁

or —O_{ga}CᵣₐH₂ᵣₐR(10);

na is zero or 1;

ma is zero, 1, 2, 3, 4, 5, 6, 7 or 8;

ga is zero or 1;

ra is zero, 1, 2, 3 or 4;

R(10) is cycloalkyl having 3, 4, 5, 6, 7 or 8 carbon atoms or phenyl,

where the phenyl is not substituted or is substituted by 1-3 substituents selected from the group consisting of F, Cl, CF₃, methyl and methoxy;

R(4) and R(5)

independently of one another are hydrogen, F, Cl, Br, I, CN, alkyl having 1, 2, 3, 4, 5, 6, 7 or 8 carbon atoms, perfluoroalkyl having 1, 2, 3, 4, 5, 6, 7 or 8 carbon atoms, cycloalkyl having 3, 4, 5, 6, 7 or 8 carbon atoms or phenyl,

which is not substituted or is substituted by 1-3 substituents selected from the group consisting of F, Cl, CF₃, methyl, methoxy and NR(14)R(15);

R(14) and R(15)

independently of one another are H, alkyl having 1, 2, 3 or 4 carbon atoms or perfl.uoroalkyl having 1, 2, 3 or 4 carbon atoms;

and their pharmaceutically tolerable salts;

(HOE 96/F 032—EP-A 791 557)

av) ortho-substituted benzoylguanidines of the formula I

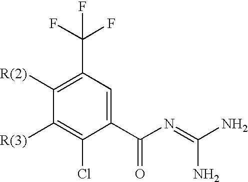

in which:

R(2) and R(3)

independently of one another are hydrogen, Cl, Br, I, (C₁-C₈)-alkyl, (C₃-C₈)-cycloalkyl or —OR(5);

R(5) is (C₁-C₈)-alkyl or —C_{d}H_{2d}—(C₃-C₈)-cycloalkyl;

d is zero, 1 or 2;

where one of the two substituents R(2) and R(3) is always hydrogen but both substituents R(2) and R(3) are not simultaneously hydrogen,

and their pharmaceutically tolerable salts;

(HOE 96/F 042—EP-A 794 171)

aw) benzoylguanidines of the formula I

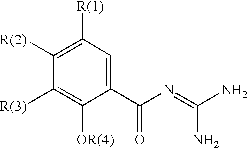

in which:

R(1) is H, F, Cl, Br, I, CN, NO₂, alkyl having 1, 2, 3, 4, 5, 6, 7 or 8 carbon atoms, alkoxy having 1, 2, 3, 4, 5, 6, 7 or 8 carbon atoms, cycloalkyl having 3, 4, 5, 6, 7 or 8 carbon atoms, cycloalkoxy having 3, 4, 5, 6, 7 or 8 carbon atoms or Xₐ—(CH₂)_{b}—(CF₂)_{c}—CF₃;

X is oxygen, S, NR(5),

a is zero or 1;

b is zero, 1 or 2;

c is zero, 1, 2 or 3;

R(5) is H, alkyl having 1, 2, 3 or 4 carbon atoms or —C_{d}H_{2d}R(6);

d is zero, 1, 2, 3 or 4;

R(6) is cycloalkyl having 3, 4, 5, 6, 7 or 8 carbon atoms, phenyl, biphenylyl or naphthyl,

where the aromatics phenyl, biphenylyl or naphthyl are not substituted or are substituted by 1-3 substituents selected from the group consisting of F, Cl, CF₃, methyl, methoxy and NR(7)R(8);

R(7) and R(8)

independently are H or alkyl having 1, 2, 3 or 4 carbon atoms;

or

R(1) is —SR(10), —OR(10) or —CR(10)R(11)R(12);

R(10) is —C_{f}H₂f-cycloalkyl having 3, 4, 5, 6, 7 or 8 carbon atoms in the cycloalkyl ring, or phenyl,

where phenyl is unsubstituted or substituted by 1-3 substituents selected from the group consisting of F, Cl, CF₃, CH₃, methoxy, hydroxyl, amino, methylamino and dimethylamino;

f is zero, 1 or 2;

R(11) and R(12)

independently of one another are defined as R(10) or are hydrogen or alkyl having 1, 2, 3 or 4 carbon atoms;

or

R(1) is phenyl, naphthyl, biphenylyl or heteroaryl having 1, 2, 3, 4, 5, 6, 7, 8 or 9 carbon atoms, with the latter being linked via a carbon atom or a nitrogen atom of the ring,

which are in each case unsubstituted or substituted by 1-3 substituents selected from the group consisting of F, CI, CF₃, CH₃, methoxy, hydroxyl, amino, methylamino and dimethylamino,

or

R(1) is —SR(13), —OR(13), —NHR(13), —NR(13)R(14), —CHR(13)R(15), —C[R(15)R(16)OH], —C≡CR(18), —C[R(19)]═CHR(18), —C[R(20)R(21)]ₖ—(CO)—[CR(22)R(23)]ₗ—R(24),

k is zero, 1, 2, 3 or 4;

l is zero, 1, 2, 3 or 4;

R(13) and R(14)

identically or differently are —(CH₂)_{g}—(CHOH)ₕ—(CH₂)ᵢ—(CHOH)ⱼ—R(17) or —(CH₂)_{g}—O—(CH₂—CH₂O)ₕ—R(24); R(17) is hydrogen or methyl,

g, h and i

identically or differently are zero, 1, 2, 3 or 4;

j is 1, 2, 3 or 4;

R(15) and R(16)

identically or differently are hydrogen, alkyl having 1, 2, 3, 4, 5 or 6 carbon atoms or, together with the carbon atom carrying them, are cycloalkyl having 3, 4, 5, 6, 7 or 8 carbon atoms;

R(18)

is phenyl,

which is unsubstituted or substituted by 1-3 substituents selected from the group consisting of F, Cl, CF₃, methyl, methoxy and NR(25)R(26);

R(25) and R(26)

are H or alkyl having 1, 2, 3 or 4 carbon atoms;

or

R(18) is heteroaryl having 1, 2, 3, 4, 5, 6, 7, 8 or 9 carbon atoms,

which is unsubstituted or substituted as phenyl;

or

R(18) is alkyl having 1, 2, 3, 4, 5 or 6 carbon atoms,

which is unsubstituted or substituted by 1-3 OH;

or

R(18)

is cycloalkyl having 3, 4, 5, 6, 7 or 8 carbon atoms;

R(19), R(20), R(21), R(22) and R(23)

identically or differently are hydrogen or methyl;

R(24) is H, alkyl having 1, 2, 3, 4, 5 or 6 carbon atoms, cycloalkyl having 3, 4, 5, 6, 7 or 8 carbon atoms or —CₘH₂m—R(18);

m is 1, 2, 3 or 4;

R(2) and R(3)

are defined as R(1);

R(4) is alkyl having 1, 2, 3 or 4 carbon atoms;

and their pharmaceutically tolerable salts;

(HOE 96/F 043—EP-A 794 172)

ax) ortho-substituted benzoylguanidines of the formula I

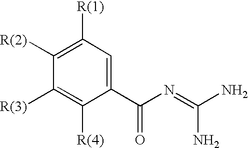

in which:

R(1) is H, F, Cl, Br, I, CN, NO₂, alkyl having 1, 2, 3, 4, 5, 6, 7 or 8 carbon atoms, alkoxy having 1, 2, 3, 4, 5, 6, 7 or 8 carbon atoms, cycloalkyl having 3, 4, 5, 6, 7 or 8 carbon atoms, cycloalkoxy having 3, 4, 5, 6, 7 or 8 carbon atoms or Xₐ—(CH₂)_{b}—(CF₂)_{c}—CF₃;

X is oxygen, S, NR(5),

a is zero or 1;

b is zero, 1 or 2;

c is zero, 1, 2 or 3;

R (5) is H, alkyl having 1, 2, 3 or 4 carbon atoms or —C_{d}H_{2d}R(6);

d is zero, 1, 2, 3 or 4;

R(6) is cycloalkyl having 3, 4, 5, 6, 7 or 8 carbon atoms, phenyl, biphenylyl or naphthyl,

where the aromatics phenyl, biphenylyl or naphthyl are not substituted or are substituted by 1-3 substituents selected from the group consisting of F, Cl, CF₃, methyl, methoxy and NR(7)R(8);

R(7) and R(8)

independently are H or alkyl having 1, 2, 3 or 4 carbon atoms;

or

R(1) is —SR(10), —OR(10) or —CR(10)R(11)R(12);

R(10) is —C_{f}H_{2f}-cycloalkyl having 3, 4, 5, 6, 7 or 8 carbon atoms in the cycloalkyl ring, or phenyl,

where phenyl is unsubstituted or substituted by 1-3 substituents selected from the group consisting of F, Cl, CF₃, CH₃, methoxy, hydroxyl, amino, methylamino and dimethylamino;

f is zero, 1 or 2;

R(11) and R(12)

independently of one another are defined as R(10), or are hydrogen or alkyl having 1, 2, 3 or 4 carbon atoms;

or

R(1) is phenyl, naphthyl, biphenylyl or heteroaryl having 1, 2, 3, 4, 5, 6, 7, 8 or 9 carbon atoms, with the latter being linked via a carbon atom or a nitrogen atom of the ring,

which are in each case unsubstituted or substituted by 1-3 substituents selected from the group consisting of F, Cl, CF₃, CH₃, methoxy, hydroxyl, amino, methylamino and dimethylarnino,

or

R(1) is —SR(13), —OR(13), —NHR(13), —NR(13)R(14), —CHR(13)R(15), —C[R(15)R(16)OH], —C≡CR(18), —C[R(19)]=CHR(18), —C[R(20)R(21)]ₖ—(CO)—[CR(22)R(23)]ₗ—R(24),

k is zero, 1, 2, 3 or 4;

l is zero, 1, 2, 3 or 4;

R(13) and R(14)

identically or differently are —(CH₂)_{g}—(CHOH)ₕ—(CH₂)ᵢ—(CHOH)ⱼ—R(17) or —(CH₂)_{g}—O—(CH₂—CH₂₀)ₕ—R(24);

R(17) is hydrogen or methyl,

g, h and i

identically or differently are zero, 1, 2, 3 or 4;

is 1, 2, 3 or 4;

R(15) and R(16)

identically or differently are hydrogen, alkyl having 1, 2, 3, 4, 5 or 6 carbon atoms or, together with the carbon atom carrying them, are cycloalkyl having 3, 4, 5, 6, 7 or 8 carbon atoms;

R(18)

is phenyl,

which is unsubstituted or substituted by 1-3 substituents selected from the group consisting of F, Cl, CF₃, methyl, methoxy and NR(25)R(26);

R(25) and R(26)

are H or alkyl having 1, 2, 3 or 4 carbon atoms;

or

R(18) is heteroaryl having 1, 2, 3, 4, 5, 6, 7, 8 or 9 carbon atoms,

which is unsubstituted or substituted as phenyl;

or

R(18) is alkyl having 1, 2, 3, 4, 5 or 6 carbon atoms,

which is unsubstituted or substituted by 1-3 OH;

or

R(18)

is cycloalkyl having 3, 4, 5, 6, 7 or 8 carbon atoms;

R(19), R(20), R(21), R(22) and R(23)

identically or differently are hydrogen or methyl;

R(24) is H, alkyl having 1, 2, 3, 4, 5 or 6 carbon atoms, cycloalkyl having 3, 4, 5, 6, 7 or 8 carbon atoms or —CₘH₂ₘ—R(18);

m is 1, 2, 3 or 4;

one of the two substituents R(2) and R(3)

is hydroxyl;

and

the other of the substituents R(2) and R(3) in each case

is defined as R(1);

R(4) is alkyl having 1, 2, 3 or 4 carbon atoms; alkoxy having 1, 2, 3 or 4 carbon atoms, F, Cl, Br, I or —(CH₂)ₙ—(CF₂)ₒ—CF₃;

n is zero or 1;

o is zero or 1;

and their pharmaceutically tolerable salts;

(HOE 96/F 135—EP-A 810 207)

ay) bis-ortho-substituted benzoylguanidines of the formula I

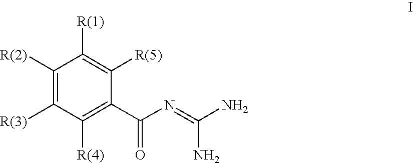

in which:

R(1), R(2) and R(3)

independently of one another are R(10)—SOₐ— or R(14)R(15)N—SO₂—;

a is zero, 1 or 2,

R(10), R(14) and R(15)

independently of one another are alkyl having 1, 2, 3, 4, 5, 6, 7 or 8 carbon atoms, perfluoroalkyl having 1, 2, 3, 4, 5, 6, 7 or 8 carbon atoms, alkenyl having 3, 4, 5 or 6 carbon atoms or —C_{ab}H_{2ab}—R(16);

ab is zero, 1, 2, 3 or 4;

R(16) is cycloalkyl having 3, 4, 5, 6 or 7 carbon atoms, or phenyl,

which is not substituted or is substituted by 1-3 substituents selected from the group consisting of F, Cl, CF₃₁ methyl, methoxy and NR(17)R(18);

R(17) and R(18)

independently of one another are hydrogen, CF₃ or alkyl having 1, 2, 3 or 4 carbon atoms;

or

R(14) and R(15)

together are 4 or 5 methylene groups, of which one CH₂ group can be replaced by oxygen, sulfur, NH, N—CH₃ or N-benzyl;

or

R(14) and R(15)

are hydrogen;

or

R(1), R(2) and R(3)

independently of one another are SR(21), —OR(22), —NR(23)R(24) or —CR(25)R(26)R(27);

R(21), R(22), R(23) and R(25)

independently of one another are —C_{b}H_{2b}—(C₁-C₉)-heteroaryl,

which is unsubstituted or substituted by 1-3 substituents selected from the group consisting of F, Cl, CF₃₁ CH₃, methoxy, hydroxyl, amino, methylamino and dimethylamino;

b is zero, 1 or 2;

R(24), R(26) and R(27)

independently of one another are hydrogen, alkyl having 1, 2, 3 or 4 carbon atoms or perfluoroalkyl having 1, 2, 3 or 4 carbon atoms;

or

R(1), R(2) and R(3)

independently of one another are hydrogen, F, Cl, Br, I, CN, —(Xa)_{dg}—C_{da}H_{2da+1}, —(Xb)_{dh}—(CH₂)_{db}—C_{de}F_{2de+1}, alkenyl having 3, 4, 5, 6, 7 or 8 carbon atoms or —C_{df}H_{2df}R(³⁰);

(Xa) is oxygen, sulfur or NR(33);

R(33) is hydrogen, alkyl having 1, 2, 3 or 4 carbon atoms or

perfluoroalkyl having 1, 2, 3 or 4 carbon atoms;

dg is zero or 1;

(Xb) is oxygen, sulfur or NR(34);

R(34) is hydrogen, alkyl having 1, 2, 3 or 4 carbon atoms or perfluoroalkyl having 1, 2, 3 or 4 carbon atoms;

dh is zero or 1;

da is zero, 1, 2, 3, 4, 5, 6, 7 or 8;

db is zero, 1, 2, 3 or 4;

de is zero, 1, 2, 3, 4, 5, 6 or 7;

df is zero, 1, 2, 3 or 4;

R(30) is cycloalkyl having 3, 4, 5, 6, 7 or 8 carbon atoms, phenyl, biphenylyl or naphthyl,

where the aromatics phenyl, biphenylyl and naphthyl are not substituted or are substituted by 1-3 substituents selected from the group consisting of F, Cl, CF₃, methyl, methoxy and NR(31)R(32);

R(31) and R(32)

are hydrogen, alkyl having 1, 2, 3 or 4 carbon atoms or perfluoroalkyl having 1, 2, 3 or 4 carbon atoms;

or

R(1), R(2) and R(3)

independently of one another are NR(40)R(41) or —(Xe)—(CH₂)_{eb}R(45);

R(40) and R(41)

independently of one another are hydrogen, alkyl having 1, 2, 3, 4, 5, 6, 7 or 8 carbon atoms, perfluoroalkyl having 1, 2, 3, 4, 5, 6, 7 or 8 carbon atoms or (CH₂)ₑ—R(42);

e is zero, 1, 2, 3 or 4;

R(42) is cycloalkyl having 3, 4, 5, 6 or 7 carbon atoms or phenyl,

which is not substituted or is substituted by 1-3 substituents selected from the group consisting of F, Cl, CF₃, methyl, methoxy and

NR(43)R(44);

R(43) and R(44)

independently of one another are hydrogen, CF₃ or alkyl having 1, 2, 3 or 4 carbon atoms;

or

R(40) and R(41)

together are 4 or 5 methylene groups, of which one CH₂ group can be replaced by oxygen, sulfur, NH, N—CH₃ or N-benzyl;

(Xe) is oxygen, sulfur or NR(47);

R(47) is hydrogen, alkyl having 1, 2, 3 or 4 carbon atoms or perfluoroalkyl having 1, 2, 3 or 4 carbon atoms;

eb is zero, 1, 2, 3 or 4;

R(45) is cycloalkyl having 3, 4, 5, 6 or 7 carbon atoms, or phenyl,

which is not substituted or is substituted by 1-3 substituents selected from the group consisting of F, Cl, CF₃, methyl, methoxy, NR(50)R(51) and —(Xfa)—(CH₂)_{ed}—(Xfb)R(46);

Xfa is CH₂, oxygen, sulfur or NR(48);

Xfb is oxygen, sulfur or NR(49);

R(48), R(49), R(50) and R(51)

independently of one another are hydrogen, alkyl having 1, 2, 3 or 4 carbon atoms or perfluoroalkyl having 1, 2, 3 or 4 carbon atoms;

ed is 1, 2, 3 or 4;

R(46) is hydrogen, alkyl having 1, 2, 3 or 4 carbon atoms or perfluoroalkyl having 1, 2, 3 or 4 carbon atoms;

or

R(1), R(2) and R(3)

independently of one another are —CHR(52)R(53);

R(52) is —(CH₂)_{g}—(CHOH)ₕ—(CH)ᵢ—(CHOH)ₖ—R(54) or —(CH₂)_{g}—O—(CH₂-CH₂O)ₕ—R(54);

R(54) is hydrogen or methyl;

g, h, i

identically or differently are zero, 1, 2, 3 or 4;

k is 1, 2, 3 or 4;

R(53) is hydrogen or alkyl having 1, 2, 3 or 4 carbon atoms;

or

R(1), R(2) and R(3)

independently of one another are —C(OH)R(55)R(56);

R(55) and R(56)

identically or differently are hydrogen or alkyl having 1, 2, 3 or 4 carbon atoms;

or

R(55) and R(56)

together are cycloalkyl having 3, 4, 5 or 6 carbon atoms;

or

R(55) is —CH₂OH;

and

R(4) and R(5)

independently of one another are alkyl having 1, 2, 3 or 4 carbon atoms, alkoxy having 1, 2, 3 or 4 carbon atoms, OH, F, Cl, Br, I, CN, —Oₙ—(CH₂)ₒ—(CF₂)ₚ—CF₃;

n is zero or 1;

o is zero, 1 or 2;

p is zero, 1 or 2;

and their pharmaceutically tolerable salts;

(96/F 136—EP-A 810 205)

az) substituted 1-naphthoylguanidines of the formula I

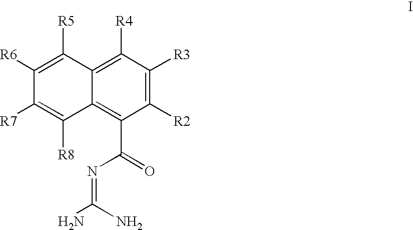

in which:

R2, R3, R4, R5, R6, R7 and R8

independently of one another are H, F, Cl, Br, I, CN, NO₂, CF₃, C₂F5 or XₐY_{b}Z;

X is O, S, NR(10), CR(11)R(12), C═O, C(═O)NR(10), C(═O)O, SO, SO₂, SO₂NR(10), OC═O, NR(10)C═O or NR(10)SO₂,

where the linkage with the naphthalene ring in each case takes place via the left atom;

R(10), R(11) and R(12)

independently of one another are H, alkyl having 1, 2, 3, 4, 5 or 6 carbon atoms, perfluoroalkyl having 1, 2, 3 or 4 carbon atoms or cycloalkyl having 3, 4, 5, 6 or 7 carbon atoms;

a is zero or 1;

Y is alkylene having 1, 2, 3, 4, 5, 6, 7 or 8 CH₂ groups,

where one of these CH₂ groups can be replaced by O, S, NR(13) or o-, p- or m-phenylene;

R(13) is H, alkyl having 1, 2, 3, 4, 5 or 6 carbon atoms, perfluoroalkyl having 1, 2, 3 or 4 carbon atoms or cycloalkyl having 3, 4, 5 or 6 carbon atoms;

b is zero or 1;

Z is H, alkyl having 1, 2, 3, 4, 5, 6 or 7 carbon atoms, cycloalkyl

having 3, 4, 5, 6 or 7 carbon atoms, C(═O)R(15), SO₂R(15),

NR(16)R(17) or phenyl,

which is not substituted or is substituted by 1-3 substituents selected from the group consisting of F, Cl, Br, CF₃, methyl, methoxy and NR(21)R(22);

R(21) and R(22)

independently of one another are H or alkyl having 1, 2, 3 or 4 carbon atoms or perfluoroalkyl having 1, 2, 3 or 4 carbon atoms;

R(15) is N═C(NH₂)₂, NR(18)R(19), N(CH₂)_{c}NR(18)R(19) or OR(20);

c is 2 or 3;

R(18) and R(19)

independently of one another are H, alkyl having 1, 2, 3, 4, 5, 6, 7 or 8 carbon atoms or perfluoroalkyl having 1, 2, 3 or 4 carbon atoms;

or

R(18) and R(19)

together are 4 or 5 methylene groups,

of which one CH₂ group can be replaced by oxygen, S, NH, N—CH₃, N-benzyl or N-(p-chlorophenyl);

R(20) is H, alkyl having 1, 2, 3, 4, 5 or 6 carbon atoms, perfluoroalkyl having 1, 2, 3 or 4 carbon atoms or cycloalkyl having 3, 4, 5, 6 or 7 carbon atoms;

R(16) and R(17)

independently of one another are H, alkyl having 1, 2, 3, 4, 5, 6, 7 or 8 carbon atoms or perfluoroalkyl having 1, 2, 3 or 4 carbon atoms;

or

R(16) and R(17)

together are 4 or 5 methylene groups, of which one CH₂ group can be replaced by oxygen, S, NH, N—CH₃, N-benzyl or N-(p-chlorophenyl);

or

Z is an N-containing heterocycle having 1, 2, 3, 4, 5, 6, 7, 8 or 9 carbon atoms,

where the N-containing heterocycle is linked via N or C and is not substituted or is substituted by 1-3 substituents selected from the group consisting of F, Cl, Br, CF₃, methyl, methoxy and NR(21)R(22);

but where, in the case where R(4) is an alkoxy radical, at least one of the substituents R(2), R(3), R(5), R(6), R(7) and R(8) is not hydrogen;

and their pharmaceutically tolerable salts;

(96/F 137—EP-A 810 206)

ba) substituted 2-naphthoylguanidines of the formula I

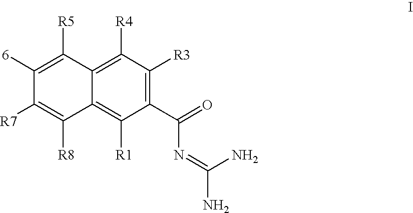

in which:

at least one of the substituents R1, R3, R4, R5, R6, R7 and R8 is

XYₐWZ or X′YₐWZ′;

X is O, S, NR(10) or CR(11)R(12);

R(10), R(11) and R(12)

independently of one another are H, alkyl having 1, 2, 3, 4, 5 or 6 carbon atoms, perfluoroalkyl having 1, 2, 3 or 4 carbon atoms or cycloalkyl having 3, 4, 5, 6 or 7 carbon atoms;

Y is alkylene having 1, 2, 3, 4, 5, 6, 7 or 8 CH₂ groups,

where one of these CH₂ groups can be replaced by O, S, NR(13) or o-, p- or m-phenylene;

R(13) is H, alkyl having 1, 2, 3, 4, 5 or 6 carbon atoms, perfluoroalkyl having 1, 2, 3 or 4 carbon atoms or cycloalkyl having 3, 4, 5 or 6 carbon atoms;

a is zero or 1;

W is CH₂, SO₂, S(═O)(═NH) or—if W does not immediately follow a heteroatom of the group XYₐ—alternatively O or NR(14);

R(14) is H, alkyl having 1, 2, 3, 4, 5 or —6 carbon atoms, perfluoroalkyl having 1, 2, 3 or 4 carbon atoms or cycloalkyl having 3, 4, 5 or 6 carbon atoms;

Z is C(═O)R(15), SO₂R(15) or—if W is not O or —NR(14)—alternatively NR(16)R(17);

R(15) is N═C(NH₂)₂, NR(18)R(19), N(CH₂)bNR(18)R(19) or OR(20);

b is 2 or 3;

R(18) and R(19)

independently of one another are H, alkyl having 1, 2, 3, 4, 5, 6, 7 or 8 carbon atoms or perfluoroalkyl having 1, 2, 3 or 4 carbon atoms;

or

R(18) and R(19)

together are 4 or 5 methylene groups,

of which one CH₂ group can be replaced by oxygen, S, NH, N—CH₃, N-benzyl or N-(p-chlorophenyl);

R(20) is

H, alkyl having 1, 2, 3, 4, 5 or 6 carbon atoms, perfluoroalkyl having 1, 2, 3 or 4 carbon atoms or cycloalkyl having 3, 4, 5, 6 or 7 carbon atoms;

R(16) and R(17)

independently of one another are H, alkyl having 1, 2, 3, 4, 5, 6, 7 or 8 carbon atoms or perfluoroalkyl having 1, 2, 3 or 4 carbon atoms;

or,

R(16) and R(17)

together are 4 or 5 methylene groups,

of which one CH₂ group can be replaced by oxygen, S, NH, N—CH₃, N-benzyl or N-(p-chlorophenyl);

X′ is C═O, C(═O)NR(30), C(═O)O, SO, SO₂, SO₂NR(30), OC═O, NR(30)C═O or NR(30)SO₂,

where the linkage with the naphthalene ring in each case takes place via the left atom;

R(30) is H, alkyl having 1, 2, 3, 4, 5 or 6 carbon atoms, perfluoroalkyl having 1, 2, 3 or 4 carbon atoms or cycloalkyl having 3, 4, 5, 6 or 7 carbon atoms;

Z′ is C(═O)R(15), SO₂R(15), an N-containing heterocycle having 1, 2, 3, 4, 5, 6, 7, 8 or 9 carbon atoms,

where the N-containing heterocycle is linked via N or C and is not substituted or is substituted by 1-3 substituents selected from the group consisting of F, Cl, Br, CF₃, methyl, methoxy and NR(21)R(22);

R(21) and R(22)

independently of one another are H, alkyl having 1, 2, 3 or 4 carbon atoms or perfluoroalkyl having 1, 2, 3 or 4 carbon atoms;

R(15) is N═C(NH₂)₂, NR(18)R(19), N(CH₂)_{b}NR(18)R(19) or OR(20);

R(18) and R(19)

independently of one another are H, alkyl having 1, 2, 3, 4, 5, 6, 7 or 8 carbon atoms or perfluoroalkyl having 1, 2, 3 or 4 carbon atoms;

or

R(18) and R(19)

together are 4 or 5 methylene groups,

of which one CH₂ group can be replaced by oxygen, S, NH, N—CH₃, N-benzyl or N-(p-chlorophenyl);

b is 2 or 3;

R(20) is H, alkyl having 1, 2, 3, 4, 5 or 6 carbon atoms, perfluoroalkyl having 1, 2, 3 or 4 carbon atoms or cycloalkyl having 3, 4, 5, 6 or 7 carbon atoms;

or

Z′—if W is not 0 or NR(14)—is NR(16)R(17);

R(16) and R(17)

independently of one another are H, alkyl having 1, 2, 3, 4, 5, 6, 7 or 8 carbon atoms or perfluoroalkyl having 1, 2, 3 or 4 carbon atoms;

or

R(16) and R(17)

together are 4 or 5 methylene groups,

of which one CH₂ group can be replaced by oxygen, S, NH, N—CH₃, N-benzyl or N-(p-chloro-phenyl);

and the other substituents R1, R3, R4, R5, R6, R7 and R8 in each case,

which are still not allocated by the definitions given above,

independently of one another are H, F, Cl, Br, I, CN, NO₂, CF₃, C₂F₅ or VₚQ_{q}U;

V is O, S, SO, SO₂, NR(60), OC═O, C═O, C(═O)NR(60), C(═O)O or CR(66)R(67);

R(60), R(66) and R(67)

independently of one another are H, alkyl having 1, 2, 3, 4, 5 or 6 carbon atoms, perfluoroalkyl having 1, 2, 3 or 4 carbon atoms or cycloalkyl having 3, 4, 5, 6 or 7 carbon atoms;

p is zero or 1;

Q is alkylene having 1, 2, 3, 4, 5, 6, 7 or 8 CH₂ groups, where one of these CH₂ groups can be replaced by O, S, NR(68) or o-, p- or m-phenylene;

R(68) is H, alkyl having 1, 2, 3, 4, 5 or 6 carbon atoms, perfluoroalkyl having 1, 2, 3 or 4 carbon atoms or cycloalkyl having 3, 4, 5 or 6 carbon atoms;

q is zero or 1;

U is H, alkyl having 1, 2, 3, 4, 5, 6 or 7 carbon atoms, cycloalkyl having 3, 4, 5, 6 or 7 carbon atoms, C(═O)R(65), SO₂R(65), NR(61)R(62) or phenyl,

which is not substituted or is substituted by 1-3 substituents selected from the group consisting of F, Cl, Br, CF₃, methyl, methoxy and NR(63)R(64);

R(63) and R(64)

independently of one another are H, alkyl having 1, 2, 3 or 4 carbon atoms or perfluoroalkyl having 1, 2, 3 or 4 carbon atoms;

R(65) is N═C(NH₂)₂, NR(61)R(62) or OR(60);

R(61) and R(62)

independently of one another are H, alkyl having 1, 2, 3, 4, 5, 6, 7 or 8 carbon atoms or perfluoroalkyl having 1, 2, 3 or 4 carbon atoms;

or

R(61) and R(62)

together are 4 or 5 methylene groups,

of which one CH₂ group can be replaced by oxygen, S, NH, N—CH₃, N-benzyl or N-(p-chlorophenyl);

or

U is an N-containing heterocycle having 1, 2, 3, 4, 5, 6, 7, 8 or 9 carbon atoms,

where the N-containing heterocycle is linked via N or C and is not substituted or is substituted by 1-3 substituents selected from the group consisting of F, Cl, Br, CF₃, methyl, methoxy and NR(63)R(64);

but where at least one of the substituents R5, R6, R7 and R8 is not

hydrogen; and their pharmaceutically tolerable salts;

(96/F 141—EP-A 811610)

bb) ortho-substituted benzoylguanidines of the formula I

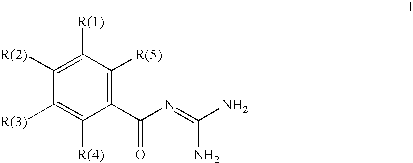

in which:

R(1) is H, F, Cl, Br, I, CN, NO₂, alkyl having 1, 2, 3, 4, 5, 6, 7 or 8 carbon atoms, alkoxy having 1, 2, 3, 4, 5, 6, 7 or 8 carbon atoms, cycloalkyl having 3, 4, 5, 6, 7 or 8 carbon atoms, cycloalkoxy having 3, 4, 5, 6, 7 or 8 carbon atoms or Xₐ—(CH₂)_{b}—(CF₂)_{b}—CF₃;

X is oxygen, sulfur or NR(9);

a is zero or 1;

b is zero, 1 or 2;

c is zero, 1, 2 or 3;

R(9) is H, alkyl having 1, 2, 3 or 4 carbon atoms or —C_{d}H_{2d}R(6);

d is zero, 1, 2, 3 or 4;

R(6) is cycloalkyl having 3, 4, 5, 6, 7 or 8 carbon atoms, phenyl, biphenylyl or naphthyl,

where the aromatics phenyl, biphenylyl or naphthyl are not substituted or are substituted by 1-3 substituents selected from the group consisting of F, Cl, CF₃, methyl, methoxy and NR(7)R(8);

R(7) and R(8) are

independently, H or alkyl having 1, 2, 3 or 4 carbon atoms;

or

R(1) is —SR(10), —OR(10) or —CR(10)R(11)R(12);

R(10) is —C_{f}H_{2f}-cycloalkyl having 3, 4, 5, 6, 7 or 8 carbon atoms in the cycloalkyl ring, heteroaryl having 1, 2, 3, 4, 5, 6, 7, 8 or 9 carbon atoms or phenyl,

where heteroaryl and phenyl are unsubstituted or are substituted by 1-3 substituents selected from the group consisting of F, Cl, CF₃, CH₃, methoxy, hydroxyl, amino, methylamino and dimethylamino;

f is zero, 1 or 2;

R(11) and (R12),

independently of each other, are defined as R(10) or are hydrogen or alkyl having 1, 2, 3 or 4 carbon atoms;

or

R(1) is phenyl, naphthyl, biphenylyi. or heteroaryl having 1, 2, 3, 4, 5, 6, 7, 10 8, or 9 carbon atoms, the latter linked via a carbon or nitrogen atom of the ring,

each of which is unsubstituted or is substituted by 1-3 substituents selected from the group consisting of F, Cl, CF₃, CH₃, methoxy, hydroxyl, amino, methylamino and dimethylamino,

or

(R1) is —SR(13), —OR(13), —NHR(13), —NR(13)R(14), —CHR(13)R(15), —C[R(15)R(16)OH], —C≡CR(18), —C[R(19)]═CHR(18), —C[R(20)R(21)]ₖ—(CO)—[CR(22)R(23)]ₗ—R(24),

k is zero, 1, 2, 3 or 4;

l is zero, 1, 2, 3 or 4;

R(13) and R(14),

identically or differently, are —(CH₂)_{g}—(CHOH)ₕ—(CH₂)ᵢ—(CHOH)ₖₖ—R(17) or —(CH₂)_{g}—O—(CH₂—CH₂O)ₕ—R(24);

R(17) is hydrogen or methyl,

g, h and i,

identically or differently, are zero, 1, 2, 3 or 4;

kk is 1, 2, 3 or 4;

R(15) and R(16),

identically or differently, are hydrogen, alkyl having 1, 2, 3, 4, 5 or 6 carbon atoms, or, together with the carbon atom carrying them, cycloalkyl having 3, 4, 5, 6, 7 or 8 carbon atoms;

R(18)

is phenyl,

which is unsubstituted or is substituted by 1-3 substituents selected from the group consisting of F, Cl, CF₃, methyl, methoxy and NR(25)R(26);

R(25) and R(26) are

H or alkyl having 1, 2, 3 or 4 carbon atoms;

or

R(18) is heteroaryl having 1, 2, 3, 4, 5, 6, 7, 8 or 9 carbon atoms,

which is unsubstituted or is substituted as phenyl;

or

R(18) is alkyl having 1, 2, 3, 4, 5 or 6 carbon atoms,

which is unsubstituted or is substituted by 1-3 OH;

or

R(18)

is cycloalkyl having 3, 4, 5, 6, 7 or 8 carbon atoms;

R(19), R(20), R(21), R(22) and R(23),

identically or differently, are hydrogen or methyl;

R(24) is H, alkyl having 1, 2, 3, 4, 5 or 6 carbon atoms, cycloalkyl

having 3, 4, 5, 6, 7 or 8 carbon atoms or —CₘH₂ₘ—R(18);

m is 1, 2, 3 or 4;

one of the two substituents R(2) and R(3) is —O—CO—R(27);

R(27) is alkyl having 1, 2, 3, 4, 5, 6, 7 or 8 carbon atoms, cycloalkyl having 3, 4, 5, 6, 7 or 8 carbon atoms, phenyl, biphenylyl, naphthyl, pyridyl or quinolinyl,

where phenyl, biphenylyl, naphthyl, pyridyl or quinolyl are unsubstituted or substituted by 1-3 substituents selected from the group consisting of F, Cl, CF₃, methyl, methoxy and NR(7)R(8);

R(7) and R(8)

independently of one another are hydrogen or alkyl having 1, 2, 3 or 4 carbon atoms;

where one of the substituents R(2) and R(3)

is always defined as R(1);

R(4) and R(5)

independently of one another are hydrogen, alkyl having 1, 2, 3 or 4 carbon atoms, alkoxy having 1, 2, 3 or 4 carbon atoms, F, Cl, Br, I, CN or —(CH₂)ₙ—(CF₂)ₒ—CF₃,

n is zeroor 1,

o is zero or 1,

and their pharmaceutically tolerable salts;

(96/F 154)

bc) benzoylguanidines of the formula I

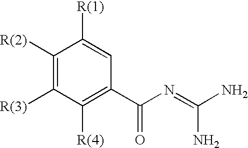

in which:

R(1) is R(13)—SOₘ or R (14)R(15)N—SO₂—;

m is 1 or 2;

R(13) is alkyl having 1, 2, 3, 4, 5, 6, 7 or 8 carbon atoms, perfluoroalkyl having 1, 2, 3, 4, 5, 6, 7 or 8 carbon atoms, alkenyl having 3, 4, 5, 6, 7 or 8 carbon atoms or —CₙH₂ₙ—R(16),

n is zero, 1, 2, 3 or 4;

R(16) is cycloalkyl having 3, 4, 5, 6, 7 or 8 carbon atoms, phenyl, biphenylyl or naphthyl,

where phenyl, biphenylyl and naphthyl are not substituted or are substituted by 1-3 substituents selected from the group consisting of F, Cl, CF₃, methyl, methoxy and

NR(25)R(26);

R(25) and R(26) are,

independently of each other, hydrogen, alkyl having 1, 2, 3 or 4 carbon atoms or perfluoroalkyl having 1, 2, 3 or 4 carbon atoms;

R(14) is hydrogen, alkyl having 1, 2, 3, 4, 5, 6, 7 or 8 carbon atoms, perfluoroalkyl having 1, 2, 3, 4, 5, 6, 7 or 8 carbon atoms, alkenyl having 3, 4, 5, 6, 7 or 8 carbon atoms or —CₙH₂ₙ—R(27),

n is zero, 1, 2, 3 or 4;

R(27) is cycloalkyl having 3, 4, 5, 6, 7 or 8 carbon atoms, phenyl, biphenylyl or naphthyl,

where phenyl, biphenylyl and naphthyl are not substituted or are substituted by 1-3 substituents selected from the group consisting of F, Cl, CF₃, methyl, methoxy and NR(28)R(29);

R(28) and R(29) are,

independently of each other, hydrogen, alkyl having 1, 2, 3 or 4 carbon atoms or perfluoroalkyl having 1, 2, 3 or 4 carbon atoms;

R(15) is hydrogen, alkyl having 1, 2, 3 or 4 carbon atoms or perfluoroalkyl having 1, 2, 3 or 4 carbon atoms;

or

R(14) and R(15) are,

together, 4 or 5 methylene groups of which one CH₂ group can be replaced by oxygen, S, NH, N—CH₃ or N-benzyl;

one of the substituents R(2) and R(3)

is hydrogen;

and the other of the substituents R(2) and R(3) in each case is —CHR(30)R(31);

R(30) is

—(CH₂)_{g}—(CHOH)ₕ—(CH₂)ᵣ—(CHOH)ₖ—R(32) or —(CH₂)_{g}—O-(CH₂-CH₂O )ₕ—R(24);

R(24) and R(32) are,

independently of each other, hydrogen or methyl;

g, h and i are,

identically or differently, zero, 1, 2, 3 or 4;

k is 1, 2, 3 or 4;

or the other of the substituents R(2) and R(3) in each case is

—C(OH)R(33)R(34);

R(31), R(33) and R(34) are,

identically or differently, hydrogen or alkyl having 1, 2, 3 or 4 carbon atoms,

or

R(33) and R(34) are,

together, cycloalkyl having 3, 4, 5 or 6 carbon atoms;

or

R(33) is —CH₂OH;

R(4) is alkyl having 1, 2, 3 or 4 carbon atoms, alkoxy having 1, 2, 3 or 4 carbon atoms, F, Cl, Br, I, CN or —(CH₂)ₙ—(CF₂)ₒ—CF₃;

n is zero or 1;

o is zero, 1 or 2;

and their pharmaceutically tolerable salts;

(96/F 202)

bd) indanylidineacetylguanidines of the formula I

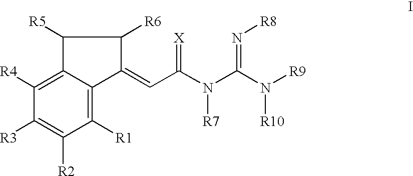

in which:

R1, R2, R3, R4, R5 and R6

independently of one another are H, C₁-C₁₀-alkyl; haloalkyl having 1-6 carbon atoms, O—C₁-C₁₀-alkyl, haloalkoxy having 1-6 carbon atoms, F, Cl, Br, I, aryl, substituted aryl, heteroaryl, substituted heteroaryl, OH, O-lower alkyl, O-aryl, O-lower alkylaryl, O-substituted aryl, O-lower alkyl-substituted aryl, O—C(═O)—C₁-C₄-alkylaryl, O—C(═O)—NH—C₁-C₄-alkyl, O—C(═O)—N(C₁-C₄-alkyl)₂, NO₂, CN, CF₃, NH₂, NH—C(═O)—C₁-C₄-alkyl, NH—C(═O)—NH₂, COOH, C(═O)—O—C₁-C₄-alkyl, C(═O)—NH₂, C(═O)—NH—C₁-C₄-alkyl, C(═O)—N(C₁-C₄-alkyl)₂, C₁-C₄—COOH, C₁-C₄-alkyl-C(═O)—O—C₁-C₄-alkyl, SO₃H, SO₂-alkyl; SO₂-alkylaryl, SO₂—N-(alkyl)₂, SO₂—N(alkyl)(alkylaryl), C(═O)—R11, C₁-C₁₀-alkyl-C(═O)-R11, C₂-C₁₀-alkenyl-C(═O)—R11, C₂-C₁₀-alkynyl-C(═O)—R11, NH—C(═O)—C₁-C₁₀-alkyl-C(═O)—R11 or O—C₁-C₁₁-alkyl-C(═O)-R11;

R11 is C₁-C₄-alkyl, C₁-C₄-alkynyl, aryl, substituted aryl, NH₂, NH—C₁-C₄-alkyl, N—(C₁-C₄-alkyl)₂, SO₃H, SO₂-alkyl, SO₂-alkylaryl, SO₂—N-(alkyl)₂ or SO₂—N(alkyl)(alkylaryl);

X is O, S or NH;

R7, R8, R9 and R10

independently of one another are H, alkyl, cycloalkyl, aryl, alkylaryl,

or

R8 and R9

together are part of a 5-, 6- or 7-membered heterocyclic ring;

or their pharmaceutically acceptable salts;

(96/F 226)

be) phenyl-substituted alkenylcarboxylic acid guanidides of the formula

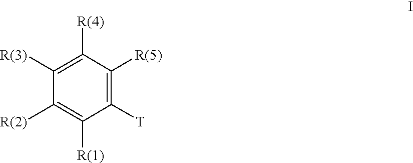

in which:

T is

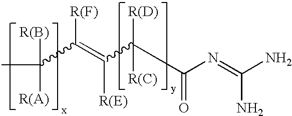

R(A) is hydrogen, F, Cl, Br, I, CN, OH, OR(6), (C₁-C₄)-alkyl, Oᵣ(CH₂)ₐC_{b}F_{2b+1}, (C₃-C₈)-cycloalkyl oder NR(7)R(8)

r is zero or 1;

a is zero, 1, 2, 3 or 4;

b is 1, 2, 3 or 4;

R(6) is (C₁-C₄)-alkyl, (C₁-C₄)-perfluoroalkyl, (C₃-C₆)-alkenyl, (C₃-C₈)-cycloalkyl, phenyl or benzyl,

the phenyl nucleus not being substituted or being substituted by 1-3 substituents selected from the group consisting of F, Cl, CF₃, methyl, methoxy and NR(9)R(10),

R(9) and R(10)

are H, (C₁-C₄)-alkyl or (C₁-C₉)-perfluoroalkyl;

R(7) and R(8)

independently of one another are defined as R(6);

or

R(7) and R(8)

together are 4 or 5 methylene groups, of which one CH₂ group can be replaced by oxygen, sulfur, NH, N—CH₃ or N-benzyl;

R(B), R(C) and R(D)

independently are defined as R(A);

x is zero, 1 or 2;

y is zero, 1 or 2;

R(F) is hydrogen, F, Cl, Br, I, CN, OR(12), (C₁-C₈)-alkyl, Oₚ(CH₂)_{f}C_{g}F_{2g+1}, (C₃-C₈)-cycloalkyl or (C₁-C₉)-heteroaryl;

p is zero or 1;

f is zero, 1, 2, 3 or 4;

g is 1, 2, 3, 4, 5, 6, 7 or 8;

R(12) is (C₁-C₈)-alkyl, (C₁-C₄)-perfluoroalkyl, (C₃-C₈)-alkenyl, (C₃-C₈)-cycloalkyl, phenyl or benzyl,

the phenyl nucleus not being substituted or being substituted by 1-3 substituents selected from the group consisting of F, Cl, CF₃, methyl, methoxy and NR(13)R(14);

R(13) and R(14)

are H, (C₁-C₄)-alkyl or (C₁-C₄)-perfluoroalkyl;

R(E) is defined independently as R(F);

R(1) is defined independently as T;

or

R(1) is hydrogen, —OₖCₘH₂ₘ₊₁, —Oₙ(CH₂)ₚC_{q}F_{2q+1}, F, Cl, Br, I, CN, —(C═O)—N═C(NH₂)₂, —SOᵣR(17), —SOᵣ₂NR(31)R(32), —Oᵤ(CH₂)ᵥC₆H₅, —Oᵤ₂—(C₁-C₉)-heteroaryl or —Sᵤ₂—(C₁-C₉)-heteroaryl;

k is zero or 1;

m is zero,1, 2, 3, 4, 5, 6, 7 or 8;

n is zero or 1;

p is zero, 1, 2, 3 or 4;

q is 1, 2, 3, 4, 5, 6, 7 or 8;

r is zero, 1 or 2;

r2 is zero, 1 or 2;

R(31) and R(32)

independently of one another are hydrogen, (C₁-C₈)-alkyl or (C₁-C₈)-perfluoroalkyl;

or

R(31) and R(32)

are, together, 4 or 5 methylene groups of which one CH₂ group can be replaced by oxygen, S, NH, N—CH₃ or N-benzyl;

R(17) is (C₁-C₈)-alkyl;

u is zero or 1;

u2 is zero or 1;

v is zero, 1, 2, 3 or 4;

where the phenyl nucleus is not substituted or is substituted by 1-3 substituents selected from the group consisting of F, Cl, CF₃, methyl, methoxy, —(CH₂)_{w}NR(21)R(22), NR(18)R(19) and (C₁-C₉)-heteroaryl;

R(18), R(19), R(21) and R(22)

independently of one another are (C₁-C₄)-alkyl or (C₁-C₄)-perfluoroalkyl;

w is 1, 2, 3 or 4;

where the heterocycle of the (C₁-C₉)-heteroaryl is unsubstituted or substituted by 1-3 substituents selected from the group consisting of F, Cl, CF₃, m-ethyl or methoxy;

R(2), R(3), R(4) and R(5)

independently of one another are defined as R(1),

or

R(1) and R(2) or R(2) and R(3)

in each case together are —CH—CH═CH—CH—,

which is not substituted or is substituted by 1-3 substituents selected from the group consisting of F, Cl, CF₃, methyl, methoxy, —(CH₂)_{w2}NR(24)R(25) and NR(26)R(27);

R(24), R(25), R(26) and R(27)

are H, (C₁-C₄)-alkyl or (C₁-C₄)-perfluoroalkyl;

w2 is 1, 2, 3 or 4;

the radical T being present in the molecule at least twice, but only three times at most;

and their pharmaceutically tolerable salts;

(97/F 082)

bf) benzoylguanidines of the formula I

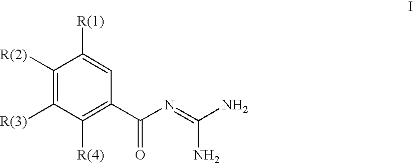

in which:

R(1) is CF₃;

one of the substituents R(2) and R(3)

is hydrogen;

and the other substituent R(2) or R(3) in each case

is —C(OH)(CH₃)—CH₂OH, —CH(CH₃)—CH₂OH or —C(OH)(CH₃)₂;

R(4) is methyl, methoxy, Cl or CF₃;

and their pharmaceutically tolerable salts.

(DE 195 02 895, DE 44 30212, EP 667 341, DE 44 04 183, EP 708 088, EP 723 963, EP 0 694 537, DE 44 21 495, EP 699 660, EP 699 663, EP 699 666, DE4337611, EP 0719766, WO94/26709, WO 9604 241, EP 726 254, U.S. Pat. No. 4,251,545, DE 35-02 629, WO 84/00875, Kumamoto et al., Pharm. Bull. [1966], 7-13; U.S. Pat. No. 3,780,027, JP 8225513; EP 743 301)

II. Also Suitable are Compounds of the Formula

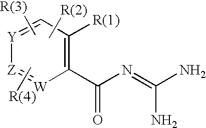

in which:

W, Y and Z

are a nitrogen atom or a carbon atom substituted by R(2) or R(3) or R(4);

R(1) is hydrogen, A, Hal, —CF₃, —CH₂F, —CHF₂, —CH₂CF₃, —C₂F₅, —CN, —NO₂, -ethynyl, or an X—R′;

A is alkyl having I to 6 carbon atoms;

Hal is F, Cl, Br or I;

X is oxygen, S or NR″;

R″ is hydrogen, A or a cyclic methylene chain having 3 to 7 carbon atoms;

R′ is H, A, HO—A—, HOOC—A—, (C₃-C₇)-cycloalkyl, (C₆-C₈)-cycloalkylalkyl, CF₃, CH₂F, CHF₂, CH₂—CF₃, Ph, —CH₂—Ph or Het;

Ph is phenyl, naphthyl or biphenylyl which is unsubstituted or mono-, di- or trisubstituted by A, OA, NR′R″, Hal, CF₃;

Het is a mono- or binuclear saturated, unsaturated or aromatic heterocycle having 1 to 4 nitrogen, oxygen and/or sulfur atoms,

which is unsubstituted or mono-, di- or trisubstituted by Hal, CF₃, A, OH, OA, —X—R′, —CN, —NO₂, and/or carbonyl oxygen,

where Het is bonded via N or an alkylene chain CₘH₂ₘ where m=zero to 6;

or

R′ and R′″

together are alkylene having 4-5 carbon atoms, in which one CH₂ group can also be replaced by oxygen, S, NH, N—A, N—Ph and N—CH₂—Ph;

R(2) and R(3)

independently of one another are hydrogen, Hal, A, HO—A—, X—R′, —C(═N—OH)—A, A—O—CO—(C₁-C₄)-alkyl-, CN, NO₂, COOH, halogen-substituted A, in particular CF₃, CH₂F, CHF₂, C₂F₅, CH₂CF₃, or S(O)ₙR′″;

R′″ is A, Ph or -Het;

n is zero, 1 or 2;

or

R(2) and R(3)

independently of one another are SO₂NR′R″, Ph or —O—Ph, —O—CH₂—Ph, —CO—A, —CHO, —COOA, —CSNR′R″, CONR′R″, —CH═CH—COOH, —CH═CH—COOA, indenyl, indanyl, decahydronaphthyl, cyclopentenyl, dihydrothienyl, dihydrofuryl, heterobicyclyl, alkylthienyl, halothienyl, haloalkylthienyl, acylthienyl, halofuryl, haloalkylfuryl or pyrrolyl;

or

R(2) and R(3)

independently of one another are R(5)—O—;

R(5) is hydrogen, A, (C₁-C₆)-alkenyl or (C₃-C₇)-cycloalkyl;

R(4) is Ph, Het, —O-Het; CF₃, S(O)ₙR′″, —SO₂NR′R″, alk;

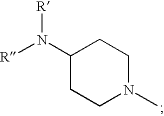

or

two of the substituents R(1) to R(4)

together are a group —O—CR(6)R(7)—CO—NR(8)—,

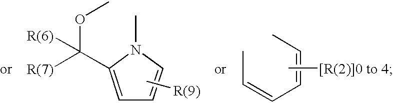

where R(2) has the meaning indicated;

R(6), R(7), R(8) and R(9)

independently of one another are H or A;

or

R(8) is (C₅-C₇)-cycloalkyl;

or

R(9) is cyano;

alk is straight-chain or branched (C₁-C₈)-alkyl or (C₃-C₈)-cycloalkyl, which is unsubstituted or mono-, di- or trisubstituted by A;

or

alk is an ethenyl or ethynyl radical which is substituted by H, A, Ph or Het.

(DE 4127 026, DE 43 37 609, JP 07025768, Edward J. Cragoe, Jr., DIURETICS [Chemistry, Pharmacology and Medicine), J. Wiley & Sons (1983), 303-341]

III. Compounds of the Formula

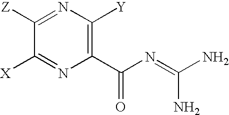

in which:

X is H, Hal, (Hal)₃C—, (C₁-C₆)-alkyl, (C₃-C₆)-cycloalkyl, substituted phenyl, (C₁-C₅)-alkyl-S— or (C₁-C₅)-alkyl-SO₂—;

Y is NH₂ or substituted amino;

or

X and Z

together are a —(CH₂)₄— or a 1,3-butadienylene chain;

or

Z is H, Hal, OH, HS, (C₁-C₅)-alkyl, (C₃-C₆)-cycloalkyl, substituted phenyl;

or

Z is an amino group —NR(1)R(2);

R(1) is H, straight- and branched-chain, optionally substituted (C₁-C₈)-alkyl,

which can be interrupted by oxygen;

or

R(1) is (C₃-C₈)-alkenyl, (C₃-C₈)-alkynyl, (C₃-C₇)-cycloalkyl or OH-substituted phenyl or OH-substituted phenyl-(C₁-C₄)-alkyl or OH-substituted (C₃-C₇)-cycloalkyl;

R(2) is 1-morpholino, hydrogen or a straight or branched (C₁-C₈)-alkyl chain,

which can be interrupted by oxygen or an amino group,

which straight or branched (C₁-C₈)-alkyl chain is unsubstituted or substituted by

a substituted or unsubstituted mono- or polynuclear heterocycle which contains nitrogen, oxygen or sulfur atoms;

or

which alkyl chain is substituted by phenyl,

optionally mono- or polysubstituted by (C₁-C₄) alkoxy, optionally substituted by OH, alkylamino, alkyl or phenyl;

or

by an aminocarbonyl group

or

by hydroxyl or (C₁-C₄)-alkoxy groups,

or

R(2) is phenyl,

unsubstituted or substituted by alkyl, alkoxy, an amino group, which as substituents carries:

H, a mono- or polynuclear heterocycle which contains nitrogen, oxygen or sulfur atoms, which is unsubstituted or substituted by H, Hal or (C₁-C₄)-alkyl;

a phenyl radical,

unsubstituted or substituted by a substituent selected from the group consisting of (C₁-C₄)-alkyl, (C₁-C₄)-alkoxy, Hal and OH;

or

R(2) is 1-piperidino,

unsubstituted or substituted in the 4-position by an acyl radical of an aliphatic, alicyclic, aromatic or heteroaromatic carboxylic acid, (C₁-C₈) alkyl, which for its part can be substituted by OH or (C₁-C₄)-alkoxy or a (C₁-C₄)-alkoxy-substituted phenyl radical;

or

R(2) is amidino,

which is unsubstituted or substituted by phenyl,

which is unsubstituted or substituted by Hal or alkyl;

or

R(2) is an acyl radical of an aliphatic, alicyclic, aromatic or heteroaromatic carboxylic acid,

or

R(2) is a (C₁-C₈) alkyl chain, which can be substituted by a phenyl radical carrying OH, alkoxy or alkyl radicals.

or

R(1) and R(2)

together with the nitrogen atom to which they are bonded, are a piperazine ring,

which is unsubstituted or, via a (C₁-C₆)-methylene chain, carries a mono- or polynuclear heterocycle,

which contains nitrogen, oxygen or sulfur (DE 4127 026 and DE 43 37 609).

Hal is F, Cl, Br or I.

(EP 708 091, EP 622 356, JP 5-125085)

IV. Likewise suitable are indoloylguanidine derivatives of the formula

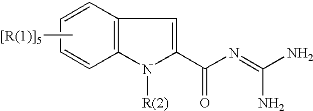

in which

R(2) is hydrogen, unsubstituted or substituted (C₁-C₈)-alkyl, (C₃-C₇)-cycloalkyl, OH, (C₁-C₆)-alkyl-O—, an aromatic radical or a group —CH₂—R(20);

R(20) is (C₂-C₆)-alkenyl or (C₂-C₆)-alkynyl;

R(1) is 1 to 5 identical or different substituents, which are:

hydrogen, unsubstituted or substituted (C₁-C₈)-alkyl, (C₂-C₆)-alkenyl, (C₂-C₆)-alkynyl, (C₃-C₇)-cycloalkyl, halogen, —NO₂, (C₂-C₈)-alkanoyl, arylalkanoyl having up to 10 carbon atoms, aroyl having up to 11 carbon atoms, —COOH, (C₂-C₆)-alkoxycarbonyl, an aromatic group or one of the following mentioned groups: —OR(3), —NR(6)R(7) or —S(O)nR(40);

R(3) is hydrogen, (C₁-C₈)-alkyl, substituted (C₁-C₈)-alkyl, (C₃-C₇)-cycloalkyl, an aromatic radical or a group —CH₂-R(30); R(30) is alkenyl or alkynyl;

R(6) and R(7)

independently of one another are hydrogen, unsubstituted or substituted (C₁-C₈)-alkyl, (C₃-C₇)-cycloalkyl, (C₂-C₈)-alkanoyl, an arylalkanoyl group having up to 10 carbon atoms, an aroyl group having up to 11 carbon atoms, an aromatic group or —CH₂—R(60);

R(60) is (C₂-C₆)-alkenyl or (C₂-C₆)-alkynyl;

or

R(6) and R(7)

together with the nitrogen atom are a 5-7-membered cyclic amine, which can additionally contain further heteroatoms in the ring;

n is zero, 1 or 2;

R(40) is unsubstituted or substituted (C₁-C₈)-alkyl, or an aromatic group, or a group

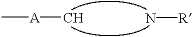

A is oxygen , —S(O)ₙ— or —N(R50)—;

R(50) is hydrogen or (C₁-C₈)-alkyl;

R′ is hydrogen, unsubstituted or substituted (C₁-C₈)-alkyl, in which the ring represents a saturated 3-8-membered heterocycle having a nitrogen atom,

said substituted alkyl carries one or more groups selected from the group consisting of halogen, —OH, (C₁-C₆)-alkoxy, —CN, —COOH, (C₂-C₆)-alkoxycarbonyl, (C₂-C₈)-alkanoyl, arylalkanoyl having up to 10 carbon atoms, aroyl having up to 11 carbon atoms, an aromatic group, —CONR(4)(R5),

R(4) and R(5)

identically or differently are hydrogen or (C₁-C₈)-alkyl;

or

R(4) and R(5)

are connected to one another and together form a 5-7-membered cyclic amine which can additionally contain further heteroatoms in the ring,

or said substituted alkyl carries a group

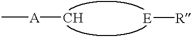

in which:

E is a nitrogen atom or a CH group;

R″ is hydrogen, (C₁-C₈)-alkyl which is unsubstituted or

substituted by OH or substituted (C₁-C₈)-alkyl, (C₁-C₆)-alkoxy, —CN, —COOH, (C₂-C₆)-alkoxycarbonyl, (C₂-C₈)-alkanoyl, aralkanoyl having up to 10 carbon atoms, aroyl having up to 11 carbon atoms, an aromatic group, —NR(6)R(7), —CONR(4)R(5);

R(4) and R(5)

independently of one another are hydrogen or (C₁-C₈)-alkyl;

where the cyclic system of the formula

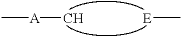

is a 3-8-membered saturated aliphatic or heterocyclic ring system having a nitrogen atom,

and where the aromatic groups mentioned are an aryl radical having up to 10 carbon atoms, a 5- or 6-membered heteroaryl radical having 1-4 nitrogen atoms, a 5- or 6-membered heteroaryl group containing 1 or 2 nitrogen atoms and a heteroatom which is oxygen or sulfur, or furyl, and where the aryl radicals mentioned can be unsubstituted or substituted by unsubstituted (C₁-C₈)-alkyl or substituted (C₁-C₈)-alkyl, halogen, —NO₂, (C₂-C₆)-alkoxycarbonyl, COOH, —OR(3), NR(6)R(7), —CONR(4)R(5), —SO₂NR(6)R(7) or S(O)ₙR(40),

where R(1) and the guanidinocarbonyl radical can be in any desired position of the 5- or 6-membered ring of the indole system, and the appropriate pharmaceutically tolerable salts.

(WO 95 04052)

V. Additionally suitable are heterocyclic guanidine derivatives of the formula

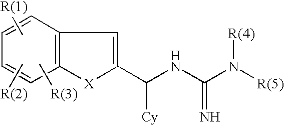

in which:

X is —O—, —S—, —NH—, —N[(C₁-C₄)-alkyl]- or —N(phenyl)—;

R(1), R(2) and R(3)

are hydrogen, halogen, (C₁-C₄)-alkyl, (C₁-C₄)-alkyl-O—, phenyl, benzyl;

or

two of the substituents R(1), R(2) and R(3)

together with one side of the benzo system are a 4-6-membered carbocyclic ring;

R(4) and R(5)

independently of one another are hydrogen, (C₁-C₁₂)-alkyl, benzhydryl, aralkyl,

which is unsubstituted or substituted by one or more substituents from the groups halogen, (C₁-C₄)-alkyl, (C₁-C₄)-alkyl-O— or —CF₃, —(CH₂)ₘ—CH₂—T,

m is zero to 3;

T is —CO—O—T(1);

T(1) is hydrogen or (C₁-C₄)-alkyl;

Cy is a benzo-fused unsaturated or dihydro-5-membered ring heterocycle

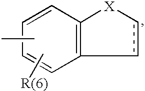

a pyrazole or imidazole ring of the formula

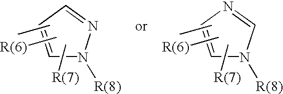

a naphthyl radical or a dihydro- or tetrahydronaphthyl radical

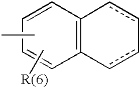

a 2-, 3- or 4-pyridyl radical

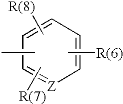

z is N— or CH; a thienyl radical

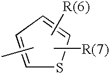

R(6) is hydrogen, halogen, hydroxyl, (C₁-C₁₀)-alkyl, (C₁-C₁₀)-alkyl-O—, phenoxy, (C₁-C₁₀)-alkyloxymethyloxy- or —(O)ₙS—R(9);

n is zero, 1 or 2;

R(9) is (C₁-C₁₀)-alkyl, thienyl, pyridyl, thiazolyl, thiadiazolyl, imidazolyl, pyrazolyl or phenyl,

each of which is unsubstituted or mono- or disubstituted by halogen, (C₁-C₄)-alkyl or (C₁-C₄)-alkyl-O—;

R(7) and R(8)

are hydrogen, halogen, hydroxyl, (C₁-C₁₀)-alkyl, (C₁-C₁₀)-alkyl-O—, phenyl, phenoxy or (C₁-C₁₀)-alkoxymethyloxy;

or

Cy is phenyl,

which is unsubstituted or is mono- or disubstituted by halogen, (C₁-C₄)-alkyl or (C₁-C₄)-alkyl-O—;

or

Cy is —Gr—Am;

Gr is —R(13)—R(12)—(CH₂)_{q}—C[W][W(1)]—(CH₂)_{q}—; R(13)R(14)— or —R(15)—;

R(12) is a single bond, —O—, —(O)ₙS—, —CO— or —CONH—;

n is zero, 1 or 2;

R(13) is a single bond, phenyl, thienyl, pyridyl, thiazolyl, thiadiazolyl, imidazolyl or pyrazolyl;

R(14) is a single bond or SO₂—;

R(15) is (C₂-C₁₀)-alkenyl or (C₂-C₁₀)-alkynyl;

W and W(1)

independently of one another are hydrogen, (C₁-C₄)-alkyl;

or

W and W(1)

cyclically connected to one another are a (C₃-C₈)-hydrocarbon ring;

q and q′

are zero to 9;

Am is —NR(10)R(11);

R(10) is hydrogen, (C₁-C₄)-alkyl or benzyl,

R(11) is (C₁-C₄)-alkyl, phenyl or benzyl;

or

R(10) and R(11)

together are a (C₃-C₁₀)-alkylene group,

which is unsubstituted or substituted by —COOH, (C₁-C₅)-alkoxycarbonyl, (C₂-C₄)-hydroxylalkylene or benzyl;

or

Am is pyrrolyl, pyridyl, pyrazolyl, morpholinyl, dihydropyridyl, tetrahydropyridyl, quinuclidinyl, imidazolyl, 3-azabicyclo[3.2.1]octyl,

which is unsubstituted or substituted by (C₁-C₄)-alkyl,

or

Am is azabicyclo[3.2.2]nonyl;

or

Am is a piperazine group of the formula

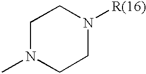

R(16) is hydrogen, (C₁-C₄)-alkyl, (C₃-C₆)-cycloalkyl, phenyl, tolyl, methoxyphenyl, halophenyl, diphenylmethylene, benzyl or pyridyl;

or

Am is an azido group —(O)ₜ—(CH₂)_{q}—C[W][W(1)]—(CH₂)_{q}′—N₃;

t is zero or 1;

where W and W(1) have the previously indicated meaning;

and the optical enantiomers and the pharmacologically tolerable salts.

VI. Additionally suitable are the guanidine compounds such as are described in EP-743 301 (DE 195 17 848), EP 758 644 (DE 195 29 612), EP 760 365 (DE 195 31138)

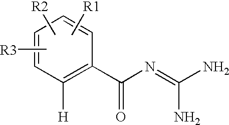

where R1=R2 is H, halo, alkyl, CN, NO₂, perfluoroalkyl, SOₙCF₃; R3 is CH═CH₂, CH₂—CH=CH₂, CH₂—CH₂—CH═CH₂, cycloalkenyl, cycloalkenylalkyl;

n is zero, 1 or 2;

R4 is alkyl, (substituted) phenyl,

or as described in DE 195 48 708, WO 97 25 310, WO 97 27 183, DE 196 01303, EP 787 728, JP 82 25 513, JP 090 59 245, JP 090 67 332, JP 090 67 340, WO 97 11055 and EP 743 301.

The disclosures of all the documents cited in this application are specifically incorporated by reference herein. The disclosures of German Priority Applications Nos. 19742096.6 (Sep. 24, 1997) and 19750498.1 (Nov. 14, 1997) are also specifically incorporated by reference herein.

What is claimed is:

1. A method for the treatment or prophylaxis of a disturbance of the central nervous system which comprises administering to a host in need of such treatment or prophylaxis an effective amount of an inhibitor of the Na⁺/H⁺ exchanger.

2. A method as claimed in claim 1, wherein the inhibitor of the Na⁺/H⁺ exchanger is:
a) a benzoylguanidine of the formula 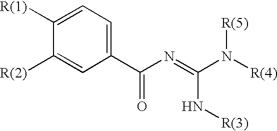
in which:
R(1) or R(2)
is R(6)—S(O)ₙ— or R(7)R(8)N—O₂S—;
and the other substituent R(1) or R(2) in each case
is H, F, Cl, Br, (C₁-C₄)-alkyl, (C₁-C₄)-alkoxy or phenoxy,
which is unsubstituted or substituted by 1-3 substituents selected from the group consisting of fluorine, chlorine, methyl and methoxy;
or the other substituent R(1) or R(2) in each case
is R(6)—S(O)ₙ or R(7)R(8)N—;
n is zero, 1 or 2;
R(6) is (C₁-C₆)-alkyl, (C₅-C₇)-cycloalkyl, cyclopentylmethyl, cyclohexylmethyl or phenyl,
which is unsubstituted or substituted by 1-3 substituents selected from the group consisting of fluorine, chlorine, methyl and methoxy;
R(7) and R(8)
identically or differently are H or (C₁-C₆)-alkyl;
or
R(7) is phenyl-(CH₂)ₘ;
m is 1-4;
or
R(7) is phenyl,
which is unsubstituted or substituted by 1-2 substituents selected from the group consisting of fluorine, chlorine, methyl and methoxy;
or
R(7) and R(8)
together are a straight-chain or branched (C₄-C₇)-chain, where the chain can additionally be interrupted by O, S or NR(9);
R(9) is H or methyl;
or
R(7) and R(8)
together with the nitrogen atom to which they are bonded, are a dihydroindole, tetrahydroquinoline or tetrahydroisoquinoline system;
R(3), R(4) and R(5)
independently of one another are H or (C₁-C₂)-alkyl,
or
R(3) and R(4)
together are a (C₂-C₄)-alkylene chain;
or
R(4) and R(5)
together are a (C₄-C₇)-alkylene chain;
or a pharmaceutically tolerable salt thereof;
b) a benzoylguanidine of the formula 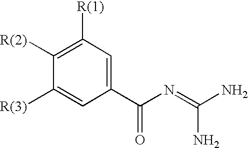
in which:
R(1) is R(4)—SOₘ or R(5)R(6)N—SO₂—;
m is zero, 1 or 2;
R(4) and R(5)
are C₁-C₈-alkyl, C₃-C₆-alkenyl or —CₙH₂ₙ—R(7);
n is zero, 1, 2, 3 or 4;
R(7) is C₅-C₇-cycloalkyl or phenyl,
which is unsubstituted or substituted by 1-3 substituents selected from the group consisting of F, Cl, CF₃, methyl, methoxy and NR(8)R(9);
R(8) and R(9)
are H or C₁-C₄-alkyl;
or
R(5) is H;
R(6) is H or C₁-C₄-alkyl,
or
R(5) and R(6)
together are 4 or 5 methylene groups, of which one CH₂ group can be replaced by an O, S, NH, N—CH₃ or N-benzyl;
R(2) is hydrogen, F, Cl, Br, (C₁-C₄)-alkyl-, O—(CH₂)ₘCₚF₂ₚ₊₁ or —X—R(10);
m is zero or 1;
p is 1, 2 or 3;
X is O, S or NR(11);
R(10) is H, C₁-C₆-alkyl, C₅-C₇-cycloalkyl, cyclohexylmethyl, cyclopentylmethyl or —CₙH₂ₙ—R(12);
n is zero, 1, 2, 3 or 4;
R(12) is phenyl,
which is unsubstituted or substituted by 1-3 substituents selected from the group consisting of F, Cl, CF₃, methyl, methoxy and NR(8)R(9);
R(8) and R(9)
are H or C₁-C₄-alkyl;
R(11) is hydrogen or C₁-C₃-alkyl;
or
R(10) and R(11)
together are 4 or 5 methylene groups, of which one CH₂ group can be replaced by O, S, NH, N—CH₃ or N-benzyl;
R(3) is defined as R(1), or is C₁-C₆-alkyl, nitro, cyano, trifluoromethyl, F, Cl, Br, I or —X—R(10);
X is O, S or NR(11);
R(10) is H, C₁-C₆-alkyl, C₅-C₇-cycloalkyl, cyclohexylmethyl, cyclopentylmethyl or —CₙH₂ₙ—R(12);
n is zero to 4;
R(12) is phenyl,
which is unsubstituted or substituted by 1-3 substituents selected from the group consisting of F, Cl, CF₃, methyl, methoxy und NR(8)R(9);
R(8) and R(9)
are H or C₁-C₄-alkyl;
R(11) is C₁-C₃-alkyl,
or
R(10) and R(11)
together are 4 or 5 methylene groups, of which one CH₂ group can be replaced by O, S, NH, N—CH₃ or N-benzyl;
or a pharmaceutically tolerable salt thereof;
c) an ortho-substituted benzoylguanidine of the formula 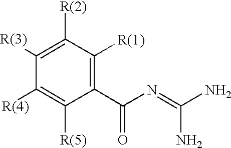
in which:
R(1) is F, Cl, Br, I, C₁-C₆-alkyl or —X—R(6);
X is O, S, NR(7) or Y—ZO;
Y is O or NR(7);
Z is C or SO;
R(6) is H, C₁-C₆-alkyl, C₅-C₇-cycloalkyl, cyclohexylmethyl, cyclopentylmethyl, —(CH₂)ₘCₚF₂ₚ₊₁ or —CₙH₂ₙ—R(8);
m is zero or 1;
p is 1-3;
n is zero to 4;
R(8) is phenyl,
which is unsubstituted or substituted by 1-3 substituents selected from the group consisting of F, Cl, CF₃, methyl, methoxy and NR(9)R(10);
R(9) and R(10)
are H or C₁-C₄-alkyl;
R(7) is H or C₁-C₃-alkyl;
or
R(6) and R(7)
together are 4 or 5 methylene groups, of which one CH₂ group can be replaced by O, S, NH, N—CH₃ or N-benzyl;
R(3) is H or —X—R(6);
X is O, S, NR(7) or Y—ZO;
R(7) is H or C₁-C₃-alkyl;
Y is O or NR(7);
where Y is bonded to the phenyl radical of the formula I,
Z is C or SO;
R(6) is H, C₁-C₆-alkyl, C₅-C₇-cycloalkyl, cyclohexylmethyl, cyclopentylmethyl, —(CH₂)ₘCₚF₂ₚ₊₁ or —CₙH₂ₙ—R(8);
m is zero or 1;
p is 1-3;
n is zero to 4;
R(8) is phenyl,
which is unsubstituted or substituted by 1-3 substituents selected from the group consisting of F, Cl, CF₃, methyl, methoxy and NR(9)R(10);
R(9) and R(10)
are H or C₁-C₄-alkyl;
or
R(6) and R(7)
together are 4 or 5 methylene groups, of which one CH₂ group can be replaced by O, S, NH, N—CH₃ or N-benzyl;
R(2) and R(4)
identically or differently are R(11)—SO_{q}— or R(12)R(13)N—SO₂—;
q is zero -2;
R(11) is C₁-C₄-alkyl,
which is unsubstituted or carries phenyl as a substituent, where phenyl is unsubstituted or substituted by 1-3 substituents selected from the group consisting of F, Cl, CF₃, methyl, methoxy and NR(9)R(10);
R(9) and R(10)
are H or C₁-C₄-alkyl;
R(12) and R(13)
are defined as R(6) and R(7);
or one of the two radicals R(2) or R(4)
is hydrogen or is defined as R(1);
R(5) is H, methyl, F, Cl or methoxy,
or a pharmaceutically tolerable salt thereof;
d) a benzoylguanidine of the formula 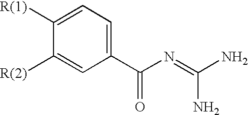
in which:
R(1) or R(2)
is an amino group —NR(3)R(4);
R(3) and R(4)
identically or differently are H, C₁-C₆-alkyl or C₃-C₇-cycloalkyl;
or
R(3) is phenyl-(CH₂)ₚ—;
p is 0, 1, 2, 3 or 4;
or
R(3) is phenyl,
where the phenyl in each case is unsubstituted or carries one to two substituents selected from the group consisting of fluorine, chlorine, methyl and methoxy;
or
R(3) and R(4)
together can be a straight-chain or branched C₄-C₇-methylene chain, where one —CH₂— member of the methylene chain can be replaced by oxygen, S or NR(5); R(5) is H or lower alkyl;
the other substituent R(1) or R(2) in each case
is H, F, Cl, C₁-C₄-alkyl, C₁-C₄-alkoxy, CF₃, CₘF₂ₘ₊₁—CH₂—, benzyl or phenoxy,
where the respective phenyl radical is unsubstituted or carries one to two substituents selected from the group consisting of methyl, methoxy, fluorine and chlorine;
m is 1, 2 or 3;
or a pharmaceutically tolerable salt thereof;
e) a benzoylguanidine of the formula 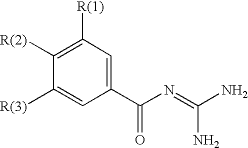
in which:
R(1) is R(4)—SOₘ or R(5)R(6)N—SO₂—;
m is zero, 1 or 2;
R(4) and R(5)
are C₁-C₈-alkyl, C₃-C₆-alkenyl or —CₙH₂ₙ—R(7);
n is zero,1, 2, 3 or 4;
R(7) is C₅-C₇-cycloalkyl or phenyl,
which is unsubstituted or substituted by 1-3 substituents selected from the group consisting of F, Cl, CF₃, methyl, methoxy and NR(8)R(9);
R(8) and R(9)
are H or C₁-C₁-alkyl;
or
R(5) is H;
R(6) is H or C₁-C₄-alkyl;
or
R(5) and R(6)
together are 4 or 5 methylene groups, of which one CH₂ group can be replaced by an O, S, NH, N—CH₃ or N-benzyl;
R(2) is hydrogen, straight-chain or branched (C₅-C₈)-alkyl, —CR(13)═CHR(12) or —C≡CR(12);
R(12) is phenyl,
which is unsubstituted or substituted by 1-3 substituents selected from the group consisting of F, Cl, CF₃, methyl, methoxy and NR(14)R(15);
R(14) and R(15)
are H or (C₁-C₄)-alkyl;
or
R(12) is (C₁-C₉)-heteroaryl,
which is unsubstituted or substituted as phenyl,
or
R(12) is (C₁-C₆)-alkyl,
which is unsubstituted or substituted by 1-3 OH,
or
R(12) is (C₃-C₈)-cycloalkyl;
R(13) is hydrogen or methyl,
or
R(12) is (C₃-C₈)-cycloalkyl, (C₃-C₈)-cycloalkyl-(C₁-C₄)-alkyl, phenyl, C₆H₅—(C₁-C₄)-alkyl, naphthyl, biphenylyl, 1,1-diphenyl-(C₁-C₄)-alkyl, cyclopentadienyl, pyridyl, pyrrolyl, furanyl, thienyl, thiazolyl, oxazolyl, indenyl, quinolyl, indolyl, benzofuranyl, benzothienyl, benzothiazolyl, benzoxazolyl, imidazolyl, pyrazolyl, triazolyl, tetrazolyl, isoxazolyl, isothiazolyl, pyrazinyl, pyrimidinyl, pyridazinyl, indazolyl, isoquinolyl, phthalazinyl, quinoxalinyl, quinazolinyl or cinnolinyl;
R(3) is defined as R(2);
and where the aromatic substituents R(2) and R(3) are unsubstituted or substituted by 1-3 substituents selected from F, Cl, CF₃, (C₁-C₄)-alkyl and -alkoxy, and NR(10)R(11) with R(10) and R(11) being H or (C₁-C₄)-alkyl;
or a pharmaceutically tolerable salt thereof;
f) a benzoylguanidine of the formula 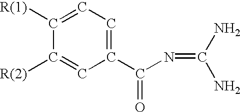
in which:
R(1) or R(2)
is R(3)—S(O)ₙ— or R(4)R(5)N—SO₂—;
the other substituent R(1) or R(2) in each case
is H, OH, F, Cl, Br, I, C₁-C₄-alkyl, C₁-C₄-alkoxy, benzyloxy or phenoxy,
which is unsubstituted or carries one to three substituents selected from the group consisting of fluorine, chlorine, methyl, methoxy, hydroxyl and benzyloxy,
R(3)—S(O)ₙ, —NR(4)R(5) or 3,4-dehydropiperidine;
R(3) is C₁-C₆-alkyl, C₅-C₇-cycloalkyl, cyclopentylmethyl, cyclohexylmethyl or phenyl,
which is unsubstituted or carries one to three substituents selected from the group consisting of fluorine, chlorine, methyl and methoxy;
R(4) and R(5)
identically or differently, are H or C₁-C₆-alkyl;
or
R(4) is phenyl-(CH₂)ₘ—;
m is 1, 2, 3 or 4;
or
R(4) is phenyl,
which is unsubstituted or carries one to two substituents selected from the group consisting of fluorine, chlorine, methyl and methoxy;
or
R(4) and R(5)
together are a straight-chain or branched C₄-C₇-chain, where the chain can additionally be interrupted by O, S or NR(6),
R(6) is H or methyl;
or
R(4) and R(5)
together with the nitrogen atom to which they are bonded, are a dihydroindole, tetrahydroquinoline or tetrahydroisoquinoline system;
n is zero, 1 or 2;
or a pharmaceutically tolerable salt thereof;
g) an isoquinoline of the formula 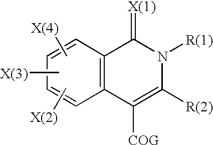
in which:
R(1) is hydrogen, alkyl, cycloalkyl, arylalkyl, alkenyl, substituted aminoalkyl or an aryl or heteroaryl ring;
where the rings are unsubstituted or substituted by 1-3 substituents selected from the group consisting of halogen, nitro, amino, mono(lower alkyl)amino, di(lower alkyl)amino, lower alkyl, lower alkoxy, benzyloxy, phenoxy, hydroxyl, and trifluoromethyl,
R(2) is hydrogen, halogen, alkyl or aryl;
which is unsubstituted or substituted by 1-3 substituents selected from the group consisting of halogen, nitro, amino, mono(lower alkyl)amino, di(lower alkyl)amino, lower alkyl, lower alkoxy, benzyloxy, phenoxy, and hydroxyl,
G is —N═C{[NR(3)R(4)][N(R5)R(6)]};
R(3), R(4), R(5) and R(6) are hydrogen,
X(2), X(3) and X(4)
independently of one another are hydrogen, halogen, nitro, amino, alkyl, sulfonamide, mono(lower alkyl)amino, di(lower alkyl)amino, lower alkyl, benzyloxy, hydroxyl;
X(1) is hydrogen, oxygen, sulfur or NR(7);
R(7) is hydrogen, alkyl, cycloalkyl, arylalkyl, alkenyl, substituted aminoalkyl or an aryl or a heteroaryl ring;
which rings are unsubstituted or substituted by 1-3 substituents selected from the group consisting of halogen, nitro, amino, mono(lower alkyl)amino, di(lower alkyl)amino, lower alkyl, lower alkoxy, benzyloxy, phenoxy, hydroxyl and trifluoromethyl;
in which substituents each alkyl chain or alkenyl chain can be interrupted by oxygen, sulfur or NR(8);
R(8) is hydrogen, alkyl, cycloalkyl, arylalkyl, alkenyl, substituted aminoalkyl or an aryl or heteroaryl ring,
which rings are unsubstituted or substituted by 1-3 substituents selected from the group consisting of halogen, nitro, amino, mono(lower alkyl)amino, di(lower alkyl)amino, lower alkyl, lower alkoxy, benzyloxy, phenoxy, hydroxyl and trifluoromethyl;
or a pharmaceutically acceptable salt thereof;
h) a compound of the formula 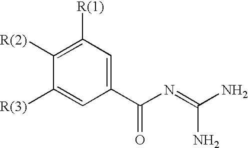
in which:
R(1) is hydrogen, F, Cl, Br, I, —NO₂, —C≡N, —CF₃, R(4)—SOₘ or R(5)R(6)N—SO₂—;
m is zero, 1 or 2;
R(4) and R(5)
are (C₁-C₈)-alkyl, (C₃-C₆)-alkenyl, —CₙH₂ₙ—R(7) or CF₃;
n is zero, 1, 2, 3 or 4;
R(7) is (C₃-C₇)-cycloalkyl or phenyl,
which is not substituted or is substituted by 1-3 substituents selected from the group consisting of F, Cl, CF₃, methyl, methoxy and NR(8)R(9);
R(8) and R(9)
are H or C₁-C₄-alkyl;
or
R(5) is H;
R(6) is H or (C₁-C₄)-alkyl;
or
R(5) and R(6)
together are 4 or 5 methylene groups, of which one CH₂ group can be replaced by oxygen, S, NH, N—CH₃ or N-benzyl;
R(2) is —SR(10), —OR(10), —NHR(10), —NR(10)R(11), —CHR(10)R(12), —[CR(12)R(13)OR(13′)], —{C—[CH₂—OR(13′)]R(12)(R(13)} or —[CR(18)R(17)]ₚ—(CO)—[CR(19)R(20)]_{q}—R(14);
R(10) and R(11)
identically or differently
are —[CHR(16)]ₛ—(CH₂)ₚ—(CHOH)_{q}—(CH₂)ᵣ—(CHOH)ₜ—R(21) or —(CH₂)ₚ—O—(CH₂—CH₂O)_{q}—R(21),
R(21) is hydrogen, methyl,
p, q, r identically or differently
are zero, 1, 2, 3 or 4;
s is zero or 1;
t is 1, 2, 3 or 4;
R(12) and R(13)
identically or differently are hydrogen, (C₁-C₆)-alkyl or, together with the carbon atom carrying them, are a (C₃-C₈)-cycloalkyl,
R(13′) is hydrogen or (C₁-C₄)-alkyl;
R(14) is H, (C₁-C₆)-alkyl, (C₃-C₈)-cycloalkyl or —CₐH₂ₐ—R(15);
a is zero, 1, 2, 3 or 4;
R(15) is phenyl,
which is unsubstituted or substituted by 1-3 substituents selected from the group consisting of F, Cl, CF₃, methyl, methoxy and NR(8)R(9);
R(8) and R(9)
are H or (C₁-C₄)-alkyl;
or
R(15) is (C₁-C₉)-heteroaryl,
which is unsubstituted or substituted as phenyl,
or
R(15) is (C₁-C₆)-alkyl,
which is unsubstituted or substituted by 1-3 OH;
R(16), R(17), R(18), R(19) and R(20)
are hydrogen or (C₁-C₃)-alkyl;
R(3) is defined as R(1),
or
R(3) is (C₁-C₆)-alkyl or —X—R(22);
X is oxygen, S or NR(16);
R(16) is H or (C₁-C₃)-alkyl;
or
R(22) and R(16)
together are 4 or 5 methylene groups, of which one CH₂ group can be replaced by oxygen, S, NH, N—CH₃ or N-benzyl; or
R(22) is defined as R(14);
or a pharmaceutically tolerable salt thereof;
i) a benzoylguanidine of the formula 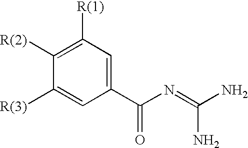
in which:
R(1) is hydrogen, F, Cl, Br, I, —NO₂, —C≡N, R(16)—CₚH₂ₚ—O_{q}, R(4)—SOₘ or R(5)R(6)N—SO₂—;
m is zero, 1 or 2;
p is, zero or 1;
q is zero, 1, 2 or 3;
R(16) is CᵣF₂ᵣ₊₁;
r is 1, 2 or 3;
R(4) and R(5)
are (C₁-C₈)-alkyl, (C₃-C₆)-alkenyl, —CₙH₂ₙ—R(7) or CF₃;
n is zero, 1, 2, 3 or 4;
R(7) is (C₃-C₇)-cycloalkyl or phenyl,
which is not substituted or is substituted by 1-3 substituents selected from the group consisting of F, Cl, CF₃, methyl, methoxy and NR(8)R(9);
R(8) and R(9)
are H or C₁-C₄-alkyl;
or
R(5) is H;
R(6) is H or (C₁-C₄)-alkyl;
or
R(5) and R(6)
together are 4 or 5 methylene groups, of which one CH₂ group can be replaced by oxygen, S, NH, N—CH₃ or N-benzyl,
R(2) is (C₁-C₉)-heteroaryl,
which is linked via C or N and which is unsubstituted or substituted by 1-3 substituents selected from the group consisting of F, Cl, CF₃, CH₃, methoxy, hydroxyl, amino, methylamino and dimethylamino;
or
R(2) is —SR(10), —OR(10), —NR(10)R(11), —CR(10)R(11)R(12);
R(10) is —CₐH₂ₐ—(C₁-C₉)-heteroaryl,
which is unsubstituted or substituted by 1-3 substituents selected from the group consisting of F, Cl, CF₃, CH₃, methoxy, hydroxyl, amino, methylamino and dimethylamino;
a is zero, 1 or 2;
R(11) and R(12)
independently of one another are defined as R(10) or are hydrogen or (C₁-C₄)-alkyl;
R(3) is defined as R(1), or is (C₁-C₆)-alkyl or —X—R(13);
X is oxygen, S, or NR(14);
R(14) is H or (C₁-C₃)-alkyl;
R(13) is H, (C₁-C₆)-alkyl, (C₃-C₈)-cycloalkyl or —C_{b}H_{2b}—R(15);
b is zero, 1, 2, 3 or 4;
or
R(13) and R(14)
together are 4 or 5 methylene groups, of which one CH₂ group can be replaced by oxygen, S, NH, N—CH₃ or N-benzyl;
R(15) is phenyl,
which is unsubstituted or substituted by 1-3 substituents selected from the group consisting of F, Cl, CF₃, methyl, methoxy and NR(8)R(9);
R(8) and R(9)
are H or (C₁-C₄)-alkyl;
or a pharmaceutically tolerable salt thereof;
k) a benzoylguanidine of the formula 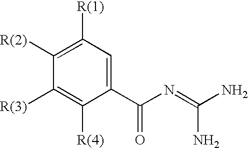
in which:
one of the substituents R(1), R(2), R(3) or R(4):
is an amino group —NR(5)[CₙH₂ₙ—R(6)];
R(5) is hydrogen or C₍₁₋₆₎-alkyl;
n is zero, 1, 2, 3 or 4;
R(6) is H or C₍₁₋₄₎-alkyl;
in which one CH₂ group can be replaced by 1 sulfur atom or a group
NR(7);
R(7) is hydrogen, methyl or ethyl;
or
R(6) is C₍₃₋₈)-cycloalkyl or phenyl,
which is unsubstituted or carries 1, 2 or 3 substituents selected from the group consisting of F, Cl, Br, methyl, methoxy, and —NR(8)R(9);
R(8) and R(9)
are H, methyl or ethyl;
or
R(5) and R(6)
together with the nitrogen atom are a 5-, 6- or 7-membered ring, in which 1 carbon atom can be replaced by oxygen, S or NR(10);
R(10) is H, C₍₁₋₃₎-alkyl or benzyl;
and the other substituents R(1), R(2), R(3), R(4) in each case are:
hydrogen, F, Cl, Br, I, CN, CF₃, NO₂, CF₃—O—, CₘF₂ₘ₊₁—CH₂—O— or
R(11)—C_{q}H_{2q}—Xₚ—;
m is 1, 2 or 3;
q is zero, 1, 2, 3 or 4;
p is zero or 1;
X is oxygen or NR(12);
R(12) is H or C₍₁₋₃₎-alkyl;
R(11) is hydrogen, C₍₁₋₆₎-alkyl, C₍₃₋₈₎-cycloalkyl or phenyl,
which is unsubstituted or substituted by 1, 2 or 3 substituents selected from the group consisting of F, Cl, CH₃, CH₃—O— and NR(13)R(14);
R(13) and R(14)
are H, methyl or ethyl;
or a pharmaceutically tolerable salt thereof;
I) a benzoylguanidine of the formula 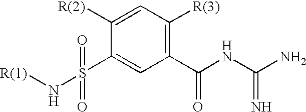
in which
R(1) is R(4)R(5)N—C(X)—;
X is oxygen, S or N—R(6);
R(4) and R(5)
identically or differently, are H, (C₁-C₈)-alkyl, (C₃-C₆)-alkenyl or —CₙH₂ₙ—R(7);
n is zero, 1, 2, 3 or 4;
R(7) is (C₅-C₇)-cycloalkyl or phenyl,
which is unsubstituted or substituted by 1-3 substituents selected from the group consisting of F, Cl, CF₃, methoxy and (C₁-C₄)-alkyl;
or
R(4) and R(5)
together are 4 or 5 methylene groups, of which one CH₂ group can be replaced by oxygen, S, NH, N—CH₃ or N-benzyl;
R(6) is defined as R(4) or is amidine;
R(2) is H, F, Cl, Br, I, (C₁-C₈)-alkyl, 1-alkenyl or 1-alkynyl, (C₃-C₈)-cycloalkyl, (C₃-C₈)-cycloalkyl-(C₁-C₄)-alkyl, phenyl, C₆H₅—(C₁-C₄)-alkyl, naphthyl, biphenylyl, 1,1-diphenyl-(C₁-C₄)-alkyl, cyclopentadienyl, pyridyl, thiopyridyl, pyrrolyl, furanyl, thienyl, thiazolyl, oxazolyl, indenyl, quinolyl, indolyl, benzofuranyl, benzothienyl, benzothiazolyl, benzoxazolyl or —W—R(8);
W is oxygen, S or NR(9);
R(8) is H, (C₁-C₆)-alkyl, (C₅-C₇)-cycloalkyl, cyclohexylmethyl, cyclopentylmethyl, —(CH₂)ₘCₚF₂ₚ₊₁ or —C_{q}H_{2q}—R(10);
m is zero or 1;
p is 1, 2 or 3;
q is zero, 1, 2, 3 or 4;
R(10) is phenyl,
which is unsubstituted or substituted by 1-3 substituents selected from the group consisting of F, Cl, CF₃, methyl, methoxy and NR(11)R(12);
R(11) and R(12)
are H or (C₁-C₄)-alkyl;
R(9) is H or (C₁-C₃)-alkyl;
or
R(8) and R(9)
together are 4 or 5 methylene groups, of which one CH₂ group can be replaced by oxygen, S, NH, N—CH₃ or N-benzyl;
R(3) is H, F, Cl, Br, I, (C₁-C₆)-alkyl or —W—R(8) as defined for R(2),
or a pharmaceutically acceptable salt thereof;
m) a benzoylguanidine of the formula 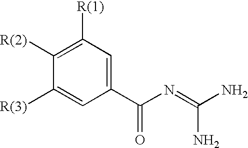
in which:
R(1), R(2), R(3)
are hydrogen, F, Cl, Br, I or (C₁-C₁₂)-alkyl;
one of the substituents R(1), R(2) or R(3)
is N₃, CN, OH or (C₁-C₁₀)-alkyloxy, if at least one of the remaining substituents R(1), R(2) or R(3) is a sufficiently lipophilic alkyl radical having 3 to 12 carbon atoms;
or
one of the substituents R(1), R(2) or R(3)
is R(4)—CₙH₂ₙ—Oₘ—,
m is zero or 1;
n is zero, 1, 2 or 3;
R(4) is CₚF₂ₚ₊₁;
p is 1, 2 or 3, if n is zero or 1;
or
R(4) is (C₃-C₁₂)-cycloalkyl, phenyl, pyridyl, quinolyl or isoquinolyl,
where the aromatic and heteroaromatic ring systems are unsubstituted or substituted by a substituent selected from the group consisting of F, Cl, CF₃, methyl, methoxy and
NR(5)R(6);
R(5) and R(6)
are hydrogen or (C₁-C₄)-alkyl;
or one of the substituents R(1), R(2) or R(3)
is —C≡CR(5) or —C[R(6)]=CR(5);
R(5) is phenyl,
which is unsubstituted or substituted by 1-3 substituents selected from the group consisting of F, Cl, CF₃, methyl, methoxy, hydroxyl, amino, methylamino and dimethylamino, and (C₁-C₉)-heteroaryl,
which is unsubstituted or substituted as phenyl,
or
R(5) is (C₁-C₆)-alkyl,
which is unsubstituted or substituted by 1-3 OH;
or
R(5) is (C₃-C₈)-cycloalkyl,
R(6) is hydrogen or methyl;
or a pharmacologically acceptable salt thereof;
o) a benzoylguanidine of the formula 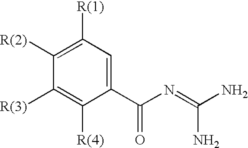
in which:
R(1) is hydrogen, F, Cl, Br, I, —NO₂, —C≡N, Xₒ—(CH₂)ₚ—(CF₂)_{q}—CF₃, R(5)—SOₘ, R(6)—CO— or R(6)R(7)N—SO₂—, where
X is oxygen, S or NR(14);
m is zero, 1 or 2;
o is zero or 1;
p is zero, 1 or 2;
q is zero, 1, 2, 3, 4, 5 or 6;
R(5) and R(6)
are (C₁-C₈)-alkyl, (C₃-C₆)-alkenyl, —CₙH₂ₙ—R(8) or CF₃;
n is zero, 1, 2, 3 or 4;
R(8) is (C₃-C₇)-cycloalkyl or phenyl,
which is not substituted or is substituted by 1-3 substituents selected from the group consisting of F, Cl, CF₃, methyl, methoxy and NR(9)R(10);
R(9) and R(10)
are H or C₁-C₄-alkyl;
or
R(6) is H;
R(7) is H or (C₁-C₄)-alkyl;
or
R(6) and R(7)
together are 4 or 5 methylene groups, of which one CH₂ group can be replaced by oxygen, S, NH, N—CH₃ or N-benzyl; 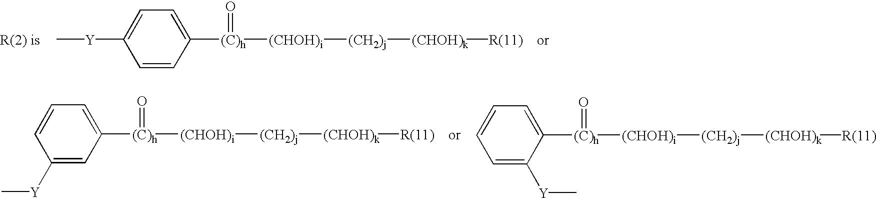
Y is oxygen, —S— or —NR(12)—;
R(11) and R(12)
are hydrogen or (C₁-C₃)-alkyl;
h is zero or 1;
i, j and k
independently are zero, 1, 2, 3 or 4;
but where h, i and k are not simultaneously zero,
R(3) is defined as R(1), or is (C₁-C₆)-alkyl or —X—R(13);
X is oxygen, S or NR(14);
R(14) is H or (C₁-C₃)-alkyl;
R(13) is H, (C₁-C₆)-alkyl, (C₃-C₈)-cycloalkyl or —C_{b}H_{2b}—R(15);
b is zero, 1, 2, 3 or 4;
or
R(13) and R(14)
together are 4 or 5 methylene groups, where one CH₂ group can be replaced by oxygen, S, NH, N—CH₃ or N-benzyl;
R(15) is phenyl,
which is unsubstituted or substituted by 1-3 substituents selected from the group consisting of F, Cl, CF₃, methyl, methoxy and NR(9)R(10);
R(9) and R(10)
are H or (C₁-C₄)-alkyl;
R(4) is hydrogen, —OR(16) or —NR(16)R(17);
R(16) and R(17)
independently are hydrogen or (C₁-C₃)-alkyl;
or a pharmaceutically tolerable salt thereof;
p) a benzoylguanidine of the formula 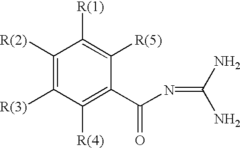
in which:
R(1) is R(6)—CO or R(7)R(8)N—CO;
R(6) is (C₁-C₆)-alkyl, (C₁-C₈)-perfluoroalkyl, (C₃-C₈)-alkenyl or —CₙH₂ₙ—R(9);
n is zero, 1, 2, 3 or 4;
R(9) is (C₃-C₈)-cycloalkyl, phenyl, biphenylyl or naphthyl,
where the aromatics are not substituted or are substituted by 1-3 substituents selected from the group consisting of F, Cl, CF₃, methyl, methoxy and NR(10)R(11);
R(10) and R(11)
are H, (C₁-C₄)-alkyl or (C₁-C₄)-perfluoro-alkyl;
R(7) is H, (C₁-C₈)-alkyl, (C₁-C₈)-perfluoroalkyl, (C₃-C₈)-alkenyl or —CₙH₂ₙ—R(12);
n is zero, 1, 2, 3 or 4;
R(12) is (C₃-C₈)-cycloalkyl, phenyl, biphenylyl or naphthyl,
where the aromatics are not substituted or are substituted by 1-3 substituents selected from the group consisting of F, Cl, CF₃, methyl, methoxy and NR(13)R(14);
R(13) and R(14)
are H, (C₁-C₄)-alkyl or (C₁-C₄)-perfluoro-alkyl;
R(8) is H, (C₁-C₄)-alkyl or (C₁-C₄)-perfluoroalkyl;
or
R(7) and R(8)
together are 4 or 5 methylene groups, of which one CH₂ group can be replaced by oxygen, S, NH, N—CH₃ or N-benzyl;
R(2) is defined as R(1), or is H, F, Cl, Br, I, CN, NO₂, (C₁-C₈)-alkyl, (C₁-C₈)-perfluoroalkyl, (C₃-C₈)-alkenyl or —CₙH₂ₙ—R(15);
n is zero,1, 2, 3 or 4;
R(15) is (C₃-C₈)-cycloalkyl, phenyl, biphenylyl or naphthyl,
where the aromatics are not substituted or are substituted by 1-3 substituents selected from the group consisting of F, Cl, CF₃, methyl, methoxy and
NR(16)R(17);
R(16) and R(17)
are H, (C₁-C₄)-alkyl or (C₁-C₄)-perfluoro-alkyl;
or
R(2) is (C₁-C₉)-heteroaryl,
which is linked via C or N and which is unsubstituted or substituted by 1-3 substituents selected from the group consisting of F, Cl, CF₃, CH₃, methoxy, hydroxyl, amino, methylamino and dimethylamino;
or
R(2) is SR(18), —OR(18), —NR(18)R(19) or —CR(18)R(19)R(20);
R(18) is —CₐH₂ₐ—(C₁-C₉)-heteroaryl,
which is unsubstituted or substituted by 1-3 substituents selected from the group consisting of F, Cl, CF₃, CH₃, methoxy, hydroxyl, amino, methylamino and dimethylamino;
a is zero, 1 or 2;
R(19) and R(20)
independently of one another are defined as R(18) or are hydrogen, (C₁-C₄)-alkyl or (C₁-C₄)-perfluoroalkyl;
or
R(2) is R(21)—SOₘ or R(22)R(23)N—SO₂—;
m is 1 or 2;
R(21) is (C₁-C₈)-alkyl, (C₁-C₈)-perfluoroalkyl, (C₃-C₉)-alkenyl or —CₙH₂ₙ—R(24),
n is zero, 1, 2, 3 or 4;
R(24) is (C₃-C₈)-cycloalkyl, phenyl, biphenylyl or naphthyl,
where the aromatics are not substituted or are substituted by 1-3 substituents selected from the group consisting of F, Cl, CF₃, methyl, methoxy and NR(27)R(28);
R(27) and R(28)
are H, (C₁-C₄)-alkyl or (C₁-C₄)-perfluoro-alkyl;
R(22) is H, (C₁-C₈)-alkyl, (C₁-C₈)-perfluoroalkyl, (C₃-C₈)-alkenyl or —CₙH₂ₙ—R(29);
n is zero, 1, 2, 3 or 4;
R(29) is (C₃-C₈)-cycloalkyl, phenyl, biphenylyl or naphthyl,
where the aromatics are not substituted or are substituted by 1-3 substituents selected from the group consisting of F, Cl, CF₃, methyl, methoxy and NR(30)R(31);
R(30) and R(31)
are H, (C₁-C₄)-alkyl or (C₁-C₄)-perfluoro-alkyl;
R(23) is H, (C₁-C₄)-alkyl or (C₁-C₄)-perfluoroalkyl;
or
R(22) and R(23)
together are 4 or 5 methylene groups, of which one CH₂ group can be replaced by oxygen, S, NH, N—CH₃ or N-benzyl;
or
R(2) is R(33)X—;
X is oxygen, S, NR(34), (D═O)A—, NR(34)C═MN^{(*)}R(35)—;
M is oxygen or S;
A is oxygen or NR(34);
D is C or SO;
R(33) is (C₁-C₈)-alkyl, (C₃-C₈)-alkenyl, (CH₂)_{b}C_{d}F_{2d+1} or —CₙH₂ₙ—R(36),
b is zero or 1;
d is 1, 2, 3, 4, 5, 6 or 7;
n is zero, 1, 2, 3 or 4;
R(36) is (C₃-C₈)-cycloalkyl, phenyl, biphenylyl or naphthyl,
where the aromatics are not substituted or are substituted by 1-3 substituents selected from the group consisting of F, Cl, CF₃, methyl, methoxy and NR(37)R(38);
R(37) and R(38)
are H, (C₁-C₄)-alkyl or (C₁-C₄)-perfluoro-alkyl;
R(34) is H, (C₁-C₄)-alkyl or (C₁-C₄)-perfluoroalkyl;
R(35) is defined as R(33);
or
R(33) and R(34)
together are 4 or 5 methylene groups, of which one CH₂ group can be replaced by oxygen, S, NH, N—CH₃ or N-benzyl;
where A and N^{(*)} are bonded to the phenyl nucleus of the benzoylguanidine parent structure;
or
R(2) is —SR(40), —OR(40), —NHR(40), —NR(40)R(41), —CHR(40)R(42), —C[R(42)R(43)OH], —C≡CR(45), —CR(46)═CHR(45) or —[CR(47)R(48)]ᵤ—(CO)—[CR(49)R(50)]ᵥ—R(44);
R(40), R(41)
identically or differently are —(CH₂)ₚ—(CHOH)_{q}—(CH₂)ᵣ—(CHOH)ₜ—R(51) or —(CH₂)ₚ—O—(CH₂-CH₂O)_{q}—R(51);
R(51) is hydrogen or methyl;
u is 1, 2, 3 or 4;
v is zero, 1, 2, 3 or 4;
p, q, r
identically or differently are zero, 1, 2, 3 or 4;
t is 1, 2, 3 or 4;
R(42) and R(43)
identically or differently are hydrogen or (C₁-C₆)-alkyl;
or
R(42) and R(43)
together with the carbon atom carrying them form a (C₃-C₈)-cycloalkyl;
R(44) is H, (C₁-C₆)-alkyl, (C₃-C₈)-cycloalkyl or —CₑH₂ₑ—R(45);
e is zero, 1, 2, 3 or 4; R(45) is phenyl,
which is unsubstituted or substituted by 1-3 substituents from the group consisting of F, Cl, CF₃, methyl, methoxy and NR(52)R(53) where
R(52) and R(53) are H or (C₁-C₄)-alkyl, or
R(45) is (C₁-C₉)-heteroaryl,
which is unsubstituted or substituted as phenyl;
or
R(45) is (C₁-C₆)-alkyl,
which is unsubstituted or substituted by 1-3 OH;
R(46), R(47), R(48), R(49) and R(50)
are hydrogen or methyl;
or
R(2) is R(55)—NH—SO₂—;
R(55) is R(56)R(57)N—(C═Y)—;
Y is oxygen, S or N—R(58);
R(56) and R(57)
identically or differently are H, (C₁-C₈)-alkyl, (C₃-C₆)-alkenyl or —C_{f}H_{2f}—R(59);
f is zero,1, 2, 3 or 4;
R(59) is (C₅-C₇)-cycloalkyl or phenyl,
which is unsubstituted or substituted by 1-3 substituents selected from the group consisting of F, Cl, CF₃, methoxy and (C₁-C₄)-alkyl;
or
R(56) and R(57)
together are 4 or 5 methylene groups, of which one CH₂ group can be replaced by oxygen, S, NH, N—CH₃ or N-benzyl;
R(58) is defined as R(56) or is amidine;
R(3), R(4) and R(5)
independently of one another are defined as R(1) or R(2);
or a pharmaceutically tolerable salt thereof;
q) a benzoylguanidine of the formula 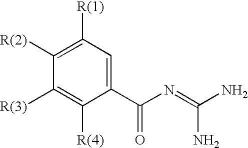
in which:
R(1) is hydrogen, F, Cl, Br, I, —NO₂, —C≡N, —Xₒ—(CH₂)ₚ—(CF₂)_{q}—CF₃, R(5)—SOₘ—, R(6)—CO—, R(6)R(7)N—CO— or R(6)R(7)N—SO₂—;
X is oxygen, —S—or NR(14);
m is zero, 1 or 2;
o is zero or 1;
p is zero, 1 or 2;
q is zero, 1, 2, 3, 4, 5 or 6;
R(5) and R(6)
are (C₁-C₈)-alkyl, (C₃-C₆)-alkenyl, —CₙH₂ₙ—R(8) or CF₃;
n is zero, 1, 2, 3 or 4;
R(8) is (C₃-C₇)-cycloalkyl, phenyl,
which is not substituted or is substituted by 1 to 3 substituents selected from the group consisting of F, Cl, CF₃, methyl, methoxy and NR(9)R(10);
R(9) and R(10)
are H or (C₁-C₄)-alkyl;
or
R(6) is hydrogen;
R(7) is hydrogen or (C₁-C₄)-alkyl;
or
R(6) and R(7)
together are 4 or 5 methylene groups, of which one CH₂ group can be replaced by oxygen, S, NH, N—CH₃ or N-benzyl;
R(2) is 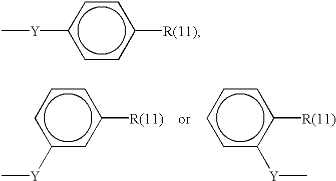
R(11) is (C₁-C₉)-heteroaryl,
which is linked via C or N and which is unsubstituted or substituted by 1 to 3 substituents selected from the group consisting of F, Cl, CF₃, CH₃, methoxy, hydroxyl, amino, methylamino, dimethylamino and benzyl;
Y is oxygen, —S—or NR(12);
R(12) is H or (C₁-C₄)-alkyl;
R(3) is defined as R(1);
or
R(3) is (C₁-C₆)-alkyl or —X—R(13);
X is oxygen, —S— or NR(14);
R(14) is H or (C₁-C₃)-alkyl;
R(13) is H, (C₁-C₆)-alkyl, (C₃-C₈)-cycloalkyl or —C_{b}H_{2b}—R(15);
b is zero, 1, 2, 3 or 4;
or
R(13) and R(14)
together are 4 or 5 methylene groups, of which one CH₂ group can be replaced by oxygen, S, NH, N—CH₃ or N-benzyl;
R(15) is phenyl,
which is unsubstituted or substituted by 1-3 substituents selected from the group consisting of F, Cl, CF₃, methyl, methoxy and NR(9)R(10);
R(9) and R(10)
are H or (C₁-C₄)-alkyl;
R(4) is hydrogen, —OR(16), —NR(16)R(17) or CᵣF₂ᵣ₊₁;
R(16) and R(17)
independently are hydrogen or (C₁-C₃)-alkyl;
r is 1, 2, 3 or 4;
or a pharmaceutically tolerable salt thereof;
r) a benzo-fused 5-membered ring heterocycle of the formula 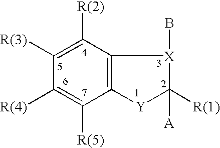
in which:
X is N or CR(6);
Y is oxygen, S or NR(7);
A, B together are a bond
or
A, B are both hydrogen, if X is CR(6) and Y is NR(7) simultaneously; one of the substituents R(1) to R(6) is a —CO—N═C(NH₂)₂ group;
the other substituents R(1) to R(6) in each case
are hydrogen, F, Cl, Br, I or (C₁-C₉)-alkyl;
up to two of the other substituents R(1) to R(6)
are CN, NO₂, N₃, (C₁-C₄)-alkyloxy or CF₃;
up to one of the other substituents
is R(8)—CₙH₂ₙ—Z—;
n is zero to 10;
where the alkylene chain —CₙH₂ₙ— is straight-chain or branched and where one carbon atom can be replaced by an oxygen or sulfur atom or by a nitrogen atom;
R(8) is hydrogen, (C₂-C₆)-alkenyl or (C₃-C₁₀)-cycloalkyl,
which is unsubstituted or substituted by 1 to 4 methyl groups or an OH group, or can contain an ethylene group —CH═CH—, and in which one methylene group can be replaced by an oxygen or sulfur atom or by a nitrogen atom;
or
R(8) is phenyl,
which is unsubstituted or substituted by 1 to 3 substituents selected from the group consisting of F, Cl, Br, I, CF₃, CH₃—S(O)ₛ— or R(9)—W_{y}—;
s is zero, 1 or 2;
R(9) is H, methyl, ethyl,
W is oxygen or NR(10);
R(10) is H or methyl;
y is zero or 1;
or
R(8) is CₘF₂ₘ₊₁;
m is 1 to 3;
or
R(8) is 1- or 2-naphthyl, pyridyl, quinolyl or isoquinolyl;
Z is —CO—, —CH₂— or —[CR(11)(OH)]_{q}—;
q is 1, 2 or 3;
R(11) is H or methyl;
or
Z is oxygen or —NR(12)—;
R(12) is H or methyl;
or
Z is —S(O)ₛ—;
s is zero, 1 or 2;
or
Z is —SO₂—NR(13)—;
R(13) is H or (C₁-C₄)-alkyl;
R(7) is hydrogen, (C₁-C₁₀)-alkyl, (C₂-C₁₀)-alkenyl or R(8)—CₙH₂ₙ—;
or a pharmaceutically tolerable salt thereof;
s) a benzoylguanidine of the formula 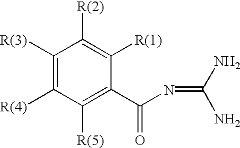
in which:
R(1), R(3) or R(4)
is —NR(6) C═X NR(7)R(8);
X is oxygen or S;
R(6) is hydrogen, (C₁-C₈)-alkyl, (C₁-C₈)-perfluoroalkyl, (C₃-C₈)-alkenyl or —CₙH₂ₙ—R(9);
n is zero, 1, 2, 3 or 4;
R(9) is (C₃-C₈)-cycloalkyl, phenyl, biphenylyl or naphthyl,
where the aromatics are not substituted or are substituted by 1-3 substituents selected from the group consisting of F, Cl, CF₃, methyl, methoxy and NR(10)R(11);
R(10) and R(11)
are H, (C₁-C₄)-alkyl or (C₁-C₄)-perfluoroalkyl;
R(7) is hydrogen, (C₁-C₈)-alkyl, (C₁-C₈)-perfluoroalkyl, (C₃-C₈)-alkenyl or —CₒH₂ₒ—R(12);
o is zero, 1, 2, 3 or 4;
R(12) is (C₃-C₈)-cycloalkyl, phenyl, biphenylyl or naphthyl,
where the aromatics are not substituted or are substituted by 1-3 substituents selected from the group consisting of F, Cl, CF₃, methyl, methoxy and NR(13)R(14);
R(13) and R(14)
are H, (C₁-C₄)-alkyl or (C₁-C₄)-perfluoroalkyl;
R(8) is defined as R(7);
or
R(7) and R(8)
together are 4 or 5 methylene groups, of which one CH₂ group can be replaced by oxygen, S, NH, N—CH₃ or N-benzyl;
the remaining substituents R(2), R(3), R(4), R(5) or R(1), R(2), R(4), R(5) or R(1), R(2), R(3), R(5) in each case
independently of one another are hydrogen, F, Cl, Br, I, —Oₜₐ(C₁-C₈)-alkyl,
—O_{tb}(C₃-C₈)-alkenyl,
—O_{tc}(CH₂)_{b}C_{d}F_{2d+1}, —O_{td}CₚH₂ₚR(18),
or up to 2 groups CN, NO₂, NR(16)R(17),
b is zero or 1;
d is 1, 2, 3, 4, 5, 6 or 7;
ta is zero or 1;
tb is zero or 1;
tc is zero or 1;
td is zero or 1;
p is zero, 1, 2, 3 or 4;
R(18) is (C₃-C₈)-cycloalkyl, phenyl, biphenylyl or naphthyl,
where the aromatics are not substituted or are substituted by 1-3 substituents selected from the group consisting of F, Cl, CF₃, methyl, methoxy and
NR(19)R(20);
R(19) and R(20)
are hydrogen or (C₁-C₄)-alkyl or (C₁-C₄)-perfluoroalkyl;
R(16) is hydrogen, (C₁-C₈)-alkyl, (C₁-C₈)-perfluoroalkyl, (C₃-C₈)-alkenyl or —C_{q}H₂q-R(21),
q is zero,1, 2, 3 or 4;
R(21) is (C₃-C₈)-cycloalkyl, phenyl, biphenylyl or naphthyl,
where the aromatics are not substituted or are substituted by 1-3 substituents from the group F, Cl, CF₃, methyl, methoxy or NR(22)R(23),
R(22) and R(23) are hydrogen, (C₁-C₄)-alkyl or (C₁-C₄)-perfluoroalkyl;
R(17) is hydrogen, (C₁-C₈)-alkyl, (C₁-C₈)-perfluoroalkyl, (C₃-C₈)-alkenyl or —CᵣH₂ᵣ—R(24);
r is zero, 1, 2, 3 or 4;
R(24) is (C₃-C₈)-cycloalkyl, phenyl, biphenylyl or naphthyl,
where the aromatics are not substituted or are substituted by 1-3 substituents selected from the group consisting of F, Cl, CF₃, methyl, methoxy and NR(25)R(26);
R(25) and R(26)
are hydrogen, (C₁-C₄)-alkyl or (C₁-C₄)-perfluoroalkyl;
or
R(16) and R(17)
together are 4 or 5 methylene groups, of which one CH₂ group can be replaced by oxygen, S, NH, N—CH₃ or N-benzyl;
or a pharmaceutically tolerable salt thereof;
t) a diacyl-substituted guanidine of the formula 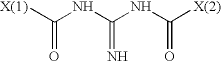
in which:
X(1) and X(2) are 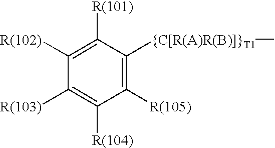
T1 is zero, 1, 2, 3 or 4;
R(A) and R(B)
independently of one another are hydrogen, F, Cl, Br, I, CN, OR(106), (C₁-C₈)-alkyl, (C₃-C₈)-cycloalkyl, O_{zk}(CH₂)_{zl}C_{zm}F_{2zm+1}, NR(107)R(108), phenyl or benzyl,
where the aromatics are not substituted or are substituted by 1-3 substituents selected from the group consisting of F, Cl, CF₃, methyl, methoxy and NR(109)R(10);
R(109) and R(110)
are hydrogen, (C₁-C₄)-alkyl or (C₁-C₄)-perfluoroalkyl;
zl is zero, 1, 2, 3 or 4;
zk is zero or 1;
zm is 1, 2, 3,4, 5, 6, 7 or 8;
R(106)
is hydrogen, (C₁-C₈)-alkyl, (C₁-C₈)-perfluoroalkyl, (C₃-C₈)-alkenyl, (C₃-C₈)-cycloalkyl, phenyl or benzyl,
where the aromatics are not substituted or are substituted by 1-3 substituents selected from the group consisting of F, Cl, CF₃, methyl, methoxy and NR(111)R(112);
R(11) and R(112)
are hydrogen, (C₁-C₄)-alkyl or (C₁-C₄)-perfluoroalkyl;
R(107) and R(108)
independently of one another are defined as R(106),
or
R(107) and R(108)
together are 4 or 5 methylene groups, of which one CH₂ group can be replaced by oxygen, S, NH, N—CH₃ or N-benzyl;
or
X(1) and X(2) are 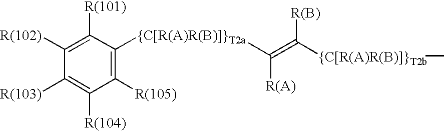
T2a and T2b
independently of one another are zero, 1 or 2;
where the double bond can have the (E)- or (Z)-configuration;
or
X(1) and X(2) are 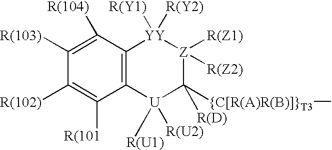
T3 is zero, 1 or 2;
U, YY and Z
independently of one another are C or N, where U, YY, Z can carry the following number of substituents:

| | Bonded in the ring to | Number of permitted |
|---|---|---|
| U, YY or Z | a double bond | substituents |
| C | yes | 1 |
| C | no | 2 |
| N | yes | 0 |
| N | no | 1 |

R(D) is hydrogen, (C₁-C₈)-alkyl or (C₁-C₈)-perfluoroalkyl,
R(U1), R(U2), R(Y1), R(Y2), R(Z1), R(Z2)
independently of one another are hydrogen, F, Cl, Br, I, CN, OR(114), (C₁-C₈)-alkyl, (C₃-C₈)-cycloalkyl, O_{zka}(CH₂)_{zla}C_{zma}F_{2zma+1}, NR(115)R(116), phenyl or benzyl,
where the aromatics are not substituted or are substituted by 1-3 substituents -selected from the group consisting of F, Cl, CF₃, methyl, methoxy, and NR(117)R(118),
R(117) and R(118)
are hydrogen, (C₁-C₄)-alkyl or (C₁-C₄)-perfluoroalkyl,
zka is zero or 1;
zla is zero, 1, 2, 3 or 4;
zma is 1, 2, 3, 4, 5, 6, 7 or 8;
R(114)
is hydrogen, (C₁-C₈)-alkyl, (C₁-C₈)-perfluoroalkyl, (C₃-C₈)-alkenyl, (C₃-C₈)-cycloalkyl, phenyl or benzyl,
where the aromatics are not substituted or are substituted by 1-3 substituents selected from the group consisting of F, Cl, CF₃, methyl, methoxy and NR(119)R(120);
R(119) and R(120)
are hydrogen, (C₁-C₄)-alkyl or (C₁-C₄)-perfluoro-alkyl;
R(115) and R(116) independently of one another are defined as
R(114);
or
R(115) and R(116)
together are 4 or 5 methylene groups, of which one CH₂ group can be replaced by oxygen, S, NH, N—CH₃ or N-benzyl;
but where the constitution of U is nitrogen (N), YY is nitrogen (N) and Z is carbon (C) is excluded,
R(101), R(102), R(103), R(104) and R(105)
independently of one another are hydrogen, F, Cl, Br, I, —C≡N,
X_{zoa}-(CH₂)_{zqa}-(C_{zqa}F₂₊₁), R(110a)—SO_{zbm}, R(110b)R(110c)N—CO,
R(111a)—CO— or R(112a)R(113a)N—SO₂—,
where the perfluoroalkyl group is straight-chain or branched,
X is oxygen, S or NR(114a);
R(114a)
is H or (C₁-C₃)-alkyl;
zoa is zero or 1;
zbm is zero, 1 or 2;
zpa is zero, 1, 2, 3 or 4;
zqa is 1, 2, 3,4, 5, 6, 7 or 8;
R(110a), R(110b), R(111a) and R(112a)
independently of one another are (C₁-C₈)-alkyl, (C₃-C₈)-alkenyl, —C_{zn}H_{2zn}—R(115a) or (C₁-C₈)-perfluoroalkyl;
zn is zero, 1, 2, 3 or 4;
R(115a)
is (C₃-C₈)-cycloalkyl, phenyl, biphenylyl or naphthyl, where the aromatics are not substituted or are substituted by 1-3 substituents selected from the group consisting of F, Cl, CF₃, methyl, methoxy and NR(116a)R(117a);
R(116a) and R(117a)
are hydrogen, (C₁-C₄)-perfluoroalkyl or (C₁-C₄)-alkyl;
or
R(110b), R(111a) and R(112a)
are hydrogen;
R(110c) and R(113a)
independently are hydrogen, (C₁-C₄)-perfluoroalkyl or (C₁-C₄)-alkyl;
or
R(110b) and R(110c) and R(112a) and R(113a)
together are 4 or 5 methylene groups, of which one CH₂ group can be replaced by oxygen, sulfur, NH, N—CH₃ or N-benzyl;
or
R(101), R(102), R(103), R(104), R(105)
independently of one another are (C₁-C₈)-alkyl, —C_{zal}H_{2zal}R(118a) or (C₃-C₈)-alkenyl,
zal is zero, 1, 2, 3 or 4;
R(118a)
is (C₃-C₈)-cycloalkyl, phenyl, biphenylyl or naphthyl,
where the aromatics are not substituted or are substituted by 1-3 substituents from the group consisting of F, Cl, CF₃, methyl, methoxy or NR(119a)R(119b);
R(119a) and R(119b)
are hydrogen, (C₁-C₄)-alkyl or (C₁-C₄)-perfluoroalkyl;
or
R(101), R(102), R(103), R(104), R(105)
independently of one another are (C₁-C₉)-heteroaryl, which is linked via C or N and which is unsubstituted or substituted by 1-3 substituents from the group consisting of F, Cl, CF₃, CH₃, methoxy, hydroxyl, amino, methylamino and dimethylamino;
or
R(101), R(102), R(103), R(104), R(105)
independently of one another are —C≡C—R(193);
R(193)
is phenyl which is not substituted or is substituted by 1-3 substituents from the group consisting of F, Cl, CF₃, methyl, methoxy or NR(194)R(195);
R(194) and R(195)
are hydrogen or CH₃;
or
R(101), R(102), R(103), R(104), R(105)
independently of one another are
—Y-para-C₆H₄—(CO)_{zh}—(CHOH)_{zi}—(CH₂)_{zj}—(CHOH)_{zk}—R(123),
—Y-meta-C₆H₄—(CO)_{zad}—(CHOH)_{zae}—(CH₂)_{zaf}—(CHOH)_{zag}—R(124)
or
—Y-Ortho-C₆H₄—(CO)_{zah}—(CHOH)_{zao}—(CH₂)_{zap}—(CHOH)_{zak}—R(125);
Y is oxygen, —S— or —NR(122d)—;
zh, zad, zah
independently are zero or 1;
zi, zj, zk, zae, zaf, zag, zao, zap and zak
independently are zero, 1, 2, 3 or 4;
but where in each case
zh, zi and zk are not simultaneously zero,
zad, zae and zag are not simultaneously zero, and
zah, zao and zak are not simultaneously zero,
R(123), R(124), R(125) and R(122d)
independently are hydrogen or (C₁-C₃)-alkyl;
or
R(101), R(102), R(103), R(104) and R(105)
independently of one another are SR(129), —OR(130), —NR(131)R(132) or —CR(133)R(134)R(135);
R(129), R(130), R(131) and R(133)
independently are —C_{zab}H_{2zab}—(C₁-C₉)-heteroaryl,
which is unsubstituted or substituted by 1-3 substituents from the group consisting of F, Cl, CF₃, CH₃, methoxy, hydroxyl, amino, methylamino and dimethylamino;
zab is zero, 1 or 2;
R(132), R(134) and R(135)
independently of one another are defined as R(129) or are hydrogen, (C₁-C₄)-alkyl or (C₁-C₄)-perfluoroalkyl;
or
R(101), R(102), R(103), R(104) and R(105)
independently of one another are —W-para-(C₆H₄)—R(196), —W-meta-(C₆H₄)—R(197) or —W-ortho-(C₆H₄)—R(198);
R(196), R(197) and R(198)
independently are (C₁-C₉)-heteroaryl,
which is linked via C or N and which is unsubstituted or substituted by 1 to 3 substituents from the group consisting of F, Cl, CF₃, CH₃, methoxy, hydroxyl, amino, methylamino, dimethylamino and benzyl;
W is oxygen, S or NR(136)—;
R(136)
is hydrogen or (C₁-C₄)-alkyl;
or
R(101), R(102), R(103), R(104) and R(105)
independently of one another are R(146)X(1a)—;
X(1a)
is oxygen, S, NR(147), (D═O)A—, NR(148)C═MN^{(*)}R(149)—;
M is oxygen or sulfur;
A is oxygen or NR(150);
D is C or SO;
R(146)
is (C₁-C₈)-alkyl, (C₃-C₈)-alkenyl, (CH₂)_{zbz}C_{zdz}F_{2dz+1} or —C_{zxa}H_{2zxa}—R(151);
zbz is zero or 1;
zdz is 1, 2, 3, 4, 5, 6 or 7;
zxa is zero, 1, 2, 3 or 4;
R(151)
is (C₃-C₈)-cycloalkyl, phenyl, biphenylyl or naphthyl,
where the aromatics are not substituted or are substituted by 1-3 substituents from the group consisting of F, Cl, CF₃, methyl, methoxy and NR(152)R(153);
R(152) and R(153)
are hydrogen, (C₁-C₄)-alkyl or (C₁-C₄)-perfluoroalkyl;
R(147), R(148) and R(150)
independently are hydrogen, (C₁-C₄)-alkyl, (C₁-C₄)-perfluoroalkyl;
R(149) is defined as R(146),
or
R(146) and R(147), or R(146) and R(148)
together are 4 or 5 methylene groups, of which one CH₂ group can be replaced by oxygen, sulfur, NH, N—CH₃ or N-benzyl;
where A and N^{(*)} are bonded to the phenyl nucleus of the alkanoyl parent structure;
or
R(101), R(102), R(103), R(104) and R(105)
independently of one another are —SR(164), —OR(165), —NHR(166), —NR(167)R(168), —CHR(169)R(170), —CR(154)R(155)OH, —C≡CR(156), —CR(158)═CR(157) or —[CR(159)R(160)]_{zu}—(C═O)—[CR(161)R(162)]_{zv}—R(163);
R(164), R(165), R(166), R(167), R(169)
identically or differently are —(CH₂)_{zy}—(CHOH)_{zz}—(CH₂)_{zaa}—(CHOH)_{zt}—R(171) or —(CH₂)_{zab}—O—(CH₂-CH₂O)_{zac—R(}172);
R(171) and R(172)
are hydrogen or methyl;
zu is 1, 2, 3 or 4;
zv is zero, 1, 2, 3 or 4;
zy, zz, zaa, zab, zac
identically or differently are zero, 1, 2, 3 or 4;
zt is 1, 2, 3 or 4;
R(168), R(170), R(154), R(155)
identically or differently are hydrogen or (C₁-C₆)-alkyl,
or
R(169) and R(170), or R(154) and R(155)
together with the carbon.atom carrying them are a (C₃-C₈)-cycloalkyl;
R(163)
is hydrogen, (C₁-C₆)-alkyl, (C₃-C₈)-cycloalkyl or —C_{zeb}H_{2zeb}—R(173);
zeb is zero, 1, 2, 3 or 4;
R(156), R(157) and R(173)
independently are phenyl which is unsubstituted or is substituted by 1-3 substituents from the group consisting of F, Cl, CF₃, methyl, methoxy and NR(174)R(175);
R(174) and R(175)
hydrogen or (C₁-C₄)-alkyl;
or
R(156), R(157) and R(173)
independently are (C₁-C₉)-heteroaryl,
which is unsubstituted or substituted as phenyl;
R(158), R(159), R(160), R(161) and R(162)
are hydrogen or methyl,
or
R(101), R(102), R(103), R(104), R(105)
independently of one another are R(176)—NH—SO₂—;
R(176)
is R(177)R(178)N—(C═Y′)—;
Y′ is oxygen, S or N—R(179);
R(177) and R(178)
identically or differently are hydrogen, (C₁-C₈)-alkyl, (C₃-C₆)-alkenyl or —C_{zfa}H_{2zfa}—R(180);
zfa is zero, 1, 2, 3 or 4;
R(180)
is (C₅-C₇)-cycloalkyl or phenyl,
which is unsubstituted or substituted by 1-3 substituents selected from the group consisting of F, Cl, CF₃, methoxy and (C₁-C₄)-alkyl;
or
R(177) and R(178)
together are 4 or 5 methylene groups, of which one CH₂ group can be replaced by oxygen, sulfur, NH, N—CH₃ or N-benzyl;
R(179)
is defined as R(177) or is amidine,
or
R(101), R(102), R(103), R(104), R(105)
independently of one another are NR(184a)R(185), OR(184b), SR(184c) or —C_{znx}H_{2znx}—R(184d);
znx is zero, 1, 2, 3 or 4;
R(184d)
is (C₃-C₇)-cycloalkyl or phenyl,
which is not substituted or is substituted by 1-3 substituents from the group consisting of F, Cl, CF₃, methyl, methoxy and NR(116k)R(117k);
R(116k) and R(117k)
are hydrogen or C₁-C₄-alkyl;
R(184a), R(184b), R(184c), R(185)
independently of one another are hydrogen, (C₁-C₈)-alkyl, (C₁-C₈)-perfluoroalkyl or (CH₂)_{zao}—R(184g);
zao is zero, 1, 2, 3 or 4;
R(184g)
is (C₃-C₇)-cycloalkyl or phenyl,
which is not substituted or is substituted by 1-3 substituents from the group consisting of F, Cl, CF₃, methyl, methoxy and NR(184u)R(184v);
R(184u) and R(184v)
are hydrogen or C₁-C₄-alkyl;
or
R(184a) and R(185)
together are 4 or 5 methylene groups, of
which one CH₂ group can be replaced by oxygen, sulfur, NH, N—CH₃ or N-benzyl;
or a pharmaceutically tolerable salt thereof;
u) a benzoylguanidine of the formula 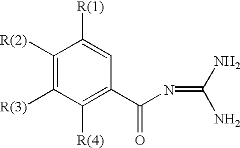
in which:
R(1) is H, F, Cl, Br, I, CN, NO₂, (C₁-C₈)-alkyl, (C₃-C₈)-cycloalkyl or Xₐ—(CH₂)_{b}—(CF₂)_{c}—CF₃;
X is oxygen, S or NR(5);
a is zero or 1;
b is zero, 1 or 2;
c is zero, 1, 2 or 3;
R(5) is H, (C₁-C₄)-alkyl or —C_{d}H_{2d}R(6);
d is zero, 1, 2, 3 or 4;
R(6) is (C₃-C₈)-cycloalkyl, phenyl, biphenylyl or naphthyl,
where the aromatics are not substituted or are substituted by 1 to 3 substituents selected from the group consisting of F, Cl, CF₃, methyl, methoxy and NR(7)R(8);
R(7) and R(8)
independently are H or (C₁-C₄)-alkyl;
or
R(1) is —SR(10), —OR(10) or —CR(10)R(11)R(12);
R(10) is —C_{f}H_{2f}—(C₃-C₈)-cycloalkyl, —(C₁-C₉)-heteroaryl or phenyl,
where the aromatic systems are unsubstituted or substituted by 1 to 3 substituents selected from the group consisting of F, Cl, CF₃, CH₃, methoxy, hydroxyl, amino, methylamino and dimethylamino;
f is zero, 1 or 2;
R(11) and R(12)
independently of one another are defined as R(10) or are hydrogen or (C₁-C₄)-alkyl;
or
R(1) is phenyl, naphthyl, biphenylyl or (C₁-C₉)-heteroaryl,
the latter linked via C or N, and which are unsubstituted or substituted by 1 to 3 substituents selected from the group consisting of F, Cl, CF₃, CH₃, methoxy, hydroxyl, amino, methylamino and dimethylamino;
or
R(1) is —SR(13), —OR(13), —NHR(13), —NR(13)R(14), —CHR(13)R(15), —C[R(15)R(16)]OH, —C≡CR(18), —C[R(19)]=CR(18) or —[CR(20)R(21)]ₖ—(CO)—[CR(22)R(23)R(24)]ₗ
R(13) and R(14)
identically or differently are —(CH₂)_{g}—(CHOH)ₕ—(CH₂)ⱼ—(CHOH)ⱼ—R(17),
R(17) is hydrogen or methyl; or
—(CH₂)_{g}—O—(CH₂-CH₂O )ₕ—R(24),
g, h, i
identically or differently are zero, 1, 2, 3 or 4;
J is 1, 2, 3 or 4;
R(15) and R(16)
identically or differently are hydrogen, (C₁-C₆)-alkyl or together with the carbon atom carrying them are a (C₃-C₈)-cycloalkyl;
R(18) is phenyl,
which is unsubstituted or substituted by 1 to 3 substituents selected from the group consisting of F, Cl, CF₃, methyl, methoxy and NR(25)R(26);
R(25) and R(26)
are H or (C₁-C₄)-alkyl;
or
R(18) is (C₁-C₉)-heteroaryl,
which is unsubstituted or substituted as phenyl;
or
R(18) is (C₁-C₆)-alkyl,
which is unsubstituted or substituted by 1 to 3 OH;
or
R(18) is (C₃-C₈)-cycloalkyl;
R(19), R(20), R(21), R(22) and R(23)
are hydrogen or methyl;
k is zero, 1, 2, 3 or 4;
l is zero, 1, 2, 3 or 4;
R(24) is H, (C₁-C₆)-alkyl, (C₃-C₈)-cycloalkyl or —CₘH₂ₘ—R(18);
m is 1, 2, 3 or 4;
R(2) and R(3)
independently of one another are defined as R(1);
R(4) is (C₁-C₃)-alkyl, F, Cl, Br, I, CN or —(CH₂)ₙ—(CF₂)ₒ—CF₃;
n is zero or 1;
o is zero, 1 or 2;
or a pharmaceutically tolerable salt thereof;
v) an acylguanidine of the formula 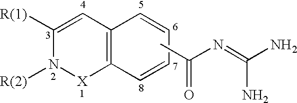
in which:
X is carbonyl or sulfonyl,
R(1) is H or (C₁-C₈)-alkyl,
unsubstituted or substituted by hydroxyl, (C₃-C₈)-cycloalkyl or phenyl,
which is unsubstituted or substituted by 1-3 substituents from the group F, Cl, CF₃, CH₃, methoxy, hydroxyl, amino, methylamino and dimethylamino,
R(2) is H or (C₁-C₄)-alkyl,
or a pharmaceutically tolerable salt thereof;
w) a phenyl-substituted alkylcarboxylic acid guanidide of the formula 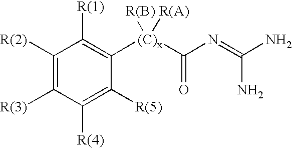
in which:
R(A) is hydrogen, F, Cl, Br, I, CN, OR(6), (C₁-C₈)-alkyl, (C₃-C₈)-cycloalkyl, Oᵣ(CH₂)ₐC_{b}F_{2b+1} or NR(7)R(8);
r is zero or 1;
a is zero, 1, 2, 3 or 4;
b is 1, 2, 3,4, 5, 6, 7 or 8;
R(6) is hydrogen, (C₁-C₈)-alkyl, (C₃-C₈)-alkenyl, (C₃-C₈)-cycloalkyl, phenyl or benzyl,
where the aromatics are not substituted or are substituted by 1-3 substituents selected from the group consisting of F, Cl, CF₃, methyl, methoxy and NR(9)R(10);
R(9) and R(10)
are H, (C₁-C₄)-alkyl or (C₁-C₄)-perfluoroalkyl;
R(7) and R(8)
independently of one another are defined as R(6);
R(B) independently is defined as R(A);
X is 1, 2 or 3;
R(1) is hydrogen, (C₁-C₈)-alkyl, (C₃-C₈)-cycloalkyl, —Oₜ(CH₂)_{d}CₑF₂ₑ₊₁, F,
Cl, Br, I or CN;
t is zero or 1;
d is zero, 1, 2, 3 or 4;
e is 1, 2, 3, 4, 5, 6, 7 or 8;
R(2), R(3), R(4) and R(5)
independently of one another are defined as R(1);
but with the condition
that at least one of the substituents R(1), R(2), R(3), R(4), R(5), R(A) and R(B) is an —Oₜ(CH₂)_{d}CₑF₂ₑ₊₁ or an Oᵣ(CH₂)ₐC_{b}F_{2b+1} group,
or a pharmaceutically tolerable salt thereof;
x) a heteroaroylguanidine of the formula 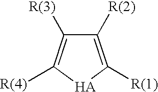
in which:
HA is SOₘ, O or NR(5);
m is zero, 1 or 2;
R(5) is hydrogen, (C₁-C₈)-alkyl or —CₐₘH₂ₐₘR(81);
am is zero, 1 or 2;
R(81) is (C₃-C₈)-cycloalkyl or phenyl,
which is not substituted or is substituted by 1-3 substituents selected from the group consisting of F, Cl, CF₃, methyl, methoxy and NR(82)R(83);
(82) and R(83)
are H or CH₃;
or
R(81) is (C₁-C₉)-heteroaryl,
which is linked via C or N and which is unsubstituted or substituted by 1-3 substituents selected from the group consisting of F, Cl, CF₃, CH₃, methoxy, hydroxyl, amino, methylamino and dimethylamino;
one of the two substituents R(1) and R(2)
is —CO—N═C(NH₂)₂;
and the other in each case
is hydrogen, F, Cl, Br, I, (C₁-C₃)-alkyl, —OR(6), CᵣF₂ᵣ₊₁, —CO—N═C(NH₂)₂ or —NR(6)R(7);
R(6) and R(7)
independently are hydrogen or (C₁-C₃)-alkyl;
r is 1, 2, 3 or 4;
R(3) and R(4)
independently of one another are hydrogen, F, Cl, Br, I, —C≡N, X—(CH₂)ₚ—(C_{q}—F_{2q+1}), R(8)—SO_{bm}, R(9)R(10)N—CO, R(11)—CO— or R(12)R(13)N—SO₂—,
where the perfluoroalkyl group is straight-chain or branched,
X is oxygen, S or NR(14);
R(14) is H or (C₁-C₃)-alkyl;
bm is zero, 1 or 2;
p is zero, 1 or 2;
q is zero, 1, 2, 3, 4, 5 or 6;
R(8), R(9), R(11) and R(12)
independently are (C₁-C₈)-alkyl, (C₃-C₆)-alkenyl, —CₙH₂ₙ—R(15) or CF₃;
n is zero, 1, 2, 3 or 4;
R(15) is (C₃-C₇)-cycloalkyl or phenyl,
which is not substituted or is substituted by 1-3 substituents selected from the group consisting of F, Cl, CF₃, methyl, methoxy and NR(16)R(17);
R(16) and R(17)
are H or C₁-C₄-alkyl;
or
R(9), R(11) and R(12)
are H;
R(10) and R(13) independently are H or (C₁-C₄)-alkyl;
or
R(9) and R(10), and R(12) and R(13)
together are 4 or 5 methylene groups, of which one CH₂ group can be replaced by oxygen, S, NH, N—CH₃ or N-benzyl,
or
R(3) and R(4)
independently of one another are (C₁-C₈)-alkyl or —CₐₗH₂ₐₗR(18);
al is zero, 1 or 2;
R(18) is (C₃-C₈)-cycloalkyl or phenyl;
which is not substituted or is substituted by 1-3 substituents selected from the group consisting of F, Cl, CF₃, methyl, methoxy and NR(19)R(20);
R(19) and R(20)
are H or CH₃;
or
R(3) and R(4)
independently of one another are (C₁-C₉)-heteroaryl,
which is linked via C or N and which is unsubstituted or substituted by 1-3 substituents selected from the group consisting of F, Cl, CF₃, CH₃, methoxy, hydroxyl, amino, methylamino and dimethylamino;
or
R(3) and R(4)
independently of one another are 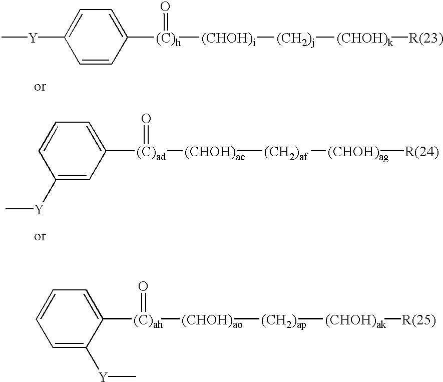
or
Y is oxygen, —S— or —NR(22)—;
h, ad, ah independently are zero or 1;
i, j, k, ae, af, ag, ao, ap and ak independently are zero, 1, 2, 3 or 4,
but where in each case
h, i and k are not simultaneously zero,
ad, ae and ag are not simultaneously zero,
ah, ao and ak are not simultaneously zero,
R(23), R(24), R(25) and R(22)
independently are hydrogen or (C₁-C₃)-alkyl;
or
R(3) and R(4)
independently of one another are hydrogen, F, Cl, Br, I, CN, (C₁-C₈)-alkyl, (C₁-C₈)-perfluoroalkyl, (C₃-C₈)-alkenyl or —C_{g}H_{2g}R(26);
g is zero, 1, 2, 3 or 4;
R(26) is (C₃-C₈)-cycloalkyl, phenyl, biphenylyl or naphthyl,
where the aromatics are not substituted or are substituted by 1-3 substituents selected from the group consisting of F, Cl, CF₃, methyl, methoxy and
NR(27)R(28);
R(27) and R(28)
are H, (C₁-C₄)-alkyl or (C₁-C₄)-pefluoroalkyl;
or
R(3) and R(4)
independently of one another are SR(29), —OR(30), —NR(31)R(32) or —CR(33)R(34)R(35);
R(29), R(30), R(31) and R(33)
independently are —CₐH₂ₐ—(C₁-C₉)-heteroaryl,
which is unsubstituted or substituted by 1-3 substituents selected from the group consisting of F, Cl, CF₃, CH₃, methoxy, hydroxyl, amino, methylamino and dimethylamino;
a is zero, 1 or 2;
R(32), R(34) and R(35)
independently of one another are defined as R(29) or are hydrogen, (C₁-C₄)-alkyl or (C₁-C₄)-perfluoroalkyl;
or
R(3) and R(4)
independently of one another are 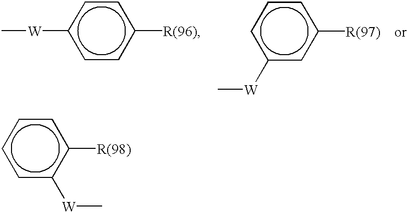
R(96), R(97) and R(98)
independently are (C₁-C₉)-heteroaryl,
which is linked via C or N and which is unsubstituted or substituted by 1 to 3 substituents selected from the group consisting of F, Cl, CF₃, CH₃, methoxy, hydroxyl, amino, methylamino, dimethylamino or benzyl;
W is oxygen, S or NR(36)—;
R(36) is H or (C₁-C₄)-alkyl;
or
R(3) and R(4)
independently of one another are R(37)—SO_{cm} or R(38)R(39)N—SO₂—;
cm is 1 or 2;
R(37) is (C₁-C₈)-alkyl, (C₁-C₈)-perfluoroalkyl, (C₃-C₈)-alkenyl or —CₛH₂ₛR(40);
s is zero, 1, 2, 3 or 4;
R(40) is (C₃-C₈)-cycloalkyl, phenyl, biphenylyl or naphthyl,
where the aromatics are not substituted or are substituted by 1-3 substituents selected from the group consisting of F, Cl, CF₃, methyl, methoxy and NR(41)R(42);
R(41) and R(42)
are H, (C₁-C₄)-alkyl or (C₁-C₄)-perfluoroalkyl;
R(38) is H, (C₁-C₈)-alkyl, (C₁-C₈)-perfluoroalkyl, (C₃-C₈)-alkenyl or —C_{w}H_{2w}—R(43);
w is zero, 1, 2, 3 or 4;
R(43) is (C₃-C₈)-cycloalkyl, phenyl, biphenylyl or naphthyl,
where the aromatics are not substituted or are substituted by 1-3 substituents selected from the group consisting of F, Cl, CF₃, methyl, methoxy and NR(44)R(45);
R(44) and R(45)
are H, (C₁-C₄)-alkyl or (C₁-C₄)-perfluoroalkyl;
R(39) is H, (C₁-C₄)-alkyl or (C₁-C₄)-perfluoroalkyl;
or
R(38) and R(39)
together are 4 or 5 methylene groups, of which one CH₂ group can be replaced by oxygen, S, NH, N—CH₃ or N-benzyl;
or
R(3) and R(4)
independently of one another are R(46)X(1)—;
X(1) is oxygen, S, NR(47), (D═O)A— or NR(48)C═MN^{(*)}R(49)—,
M is oxygen or S;
A is oxygen or NR(50);
D is C or SO;
R(46) is (C₁-C₈)-alkyl, (C₃-C₈)-alkenyl, (CH₂)_{b}C_{d}F_{2d+1} or —CₓH₂ₓ—R(51);
b is zero or 1;
d is 1, 2, 3, 4, 5, 6 or 7;
x is zero, 1, 2, 3 or 4;
R(51) is (C₃-C₈)-cycloalkyl, phenyl, biphenylyl or naphthyl,
where the aromatics are not substituted or are substituted by 1-3 substituents selected from the group consisting of F, Cl, CF₃, methyl, methoxy and NR(52)R(53);
R(52); and R(53)
are H, (C₁-C₄)-alkyl or (C₁-C₄)-perfluoroalkyl;
R(47), R(48) and R(50)
independently are hydrogen, (C₁-C₄)-alkyl or (C₁-C₄)-perfluoroalkyl;
R(49) is defined as R(46);
or
R(46) and R(47), or R(46) and R(48)
together are 4 or 5 methylene groups, of which one CH₂ group can be replaced by oxygen, S, NH, N—CH₃ or N-benzyl,
where A and N^{(*)} are bonded to the phenyl nucleus of the benzoylguanidine parent structure;
or
R(3) and R(4)
independently of one another are —SR(64), —OR(65), —NHR(66), —NR(67)R(68), —CHR(69)R(70), —C(OH)R(54)R(55), —C≡CR(56), —CR(58)═CH R(57) or —[CR(59)R(60)]ᵤ—(CO)—[CR(61)R(62)]ᵥ—R(63);
R(64), R(65), R(66), R(67) and R(69)
identically or differently are —(CH₂)_{y}—(CHOH)_{z}—(CH₂)ₐₐ—(CH₂OH)ₜ—R(71) or —(CH₂)_{ab}—O—(CH₂—CH₂O)_{ac}—R(72),
R(71) and R(72)
are hydrogen or methyl;
u is 1, 2, 3 or 4;
v is zero, 1, 2, 3 or 4;
y, z, aa, ab and ac
identically or differently are zero, 1, 2, 3 or 4;
t is 1, 2, 3 or 4;
R(68), R(70), R(54) and R(55)
identically or differently are hydrogen, (C₁-C₆)-alkyl;
or
R(69) and R(70), or R(54) and R(55)
together with the carbon atom carrying them are a (C₃-C₈)-cycloalkyl;
R(63)
is H, (C₁-C₆)-alkyl, (C₃-C₈)-cycloalkyl or —CₑH₂ₑ—R(73);
e is zero, 1, 2, 3 or 4;
R(56), R(57) and R(73)
independently are phenyl,
which is unsubstituted or substituted by 1-3 substituents selected from the group consisting of F, Cl, CF₃, methyl, methoxy and NR(74)R(75);
R(74) and R(75)
are H or (C₁-C₄)-alkyl;
or
R(56), R(57) and R(73)
independently are (C₁-C₉)-heteroaryl,
which is unsubstituted or substitued as phenyl;
R(58), R(59), R(60), R(61) and R(62)
are hydrogen or methyl,
or
R(3) and R(4)
independently of one another are R(76)—NH—SO₂—;
R(76) is R(77)R(78)N—(C═Y′)—;
Y′ is oxygen, S or N—R(79);
R(77) and R(78)
identically or differently are H, (C₁-C₈)-alkyl, (C₃-C₆)-alkenyl,
—C_{f}H_{2f}—R(80);
f is zero, 1, 2, 3 or 4;
R(80) is (C₅-C₇)-cycloalkyl or phenyl, which is unsubstituted or substituted by 1-3 substituents selected from the group consisting of F, Cl, CF₃, methoxy and (C₁-C₄)-alkyl;
or
R(77) and R(78)
together are 4 or 5 methylene groups, of which one CH₂ group can be replaced by oxygen, S, NH, N—CH₃ or N-benzyl,
R(79)
is defined as R(77) or is amidine;
or
R(3) and R(4)
independently of one another are NR(84)R(85);
R(84) and R(85)
independently of one another are H, (C₁-C₄)-alkyl, or together are 4 or 5 methylene groups,
of which one CH₂ group can be replaced by oxygen, S, NH, N—CH₃ or N-benzyl,
or a pharmaceutically tolerable salt thereof;
y) a bicyclic heteroaroylguanidine of the formula 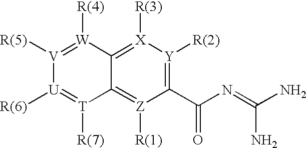
in which:
T, U, V, W, X, Y and Z
independently of one another are nitrogen or carbon;
but with the restriction
that X and Z are not simultaneously nitrogen,
and that T, U, V, W, X, Y and Z carry no substituents if they are nitrogen,
and that no more than four of them are simultaneously nitrogen,
R(1) and R(2)
independently of one another are hydrogen, F, Cl, Br, I, (C₁-C₃)-alkyl, (C₁-C₃)-perfluoroalkyl, OR(8), NR(8)R(9) or C(═O)N═C(NH₂)₂;
R(8) and R(9)
independently of one another are hydrogen or (C₁-C₃)-alkyl,
or
R(8) and R(9)
together are 4 or 5 methylene groups, of which one CH₂ group can be replaced by oxygen, S, NH, N—CH₃ or N-benzyl;
R(3), R(4), R(5), R(6) and R(7)
independently of one another are hydrogen, F, Cl, Br, I, —C≡N,
Xₖ—(CH₂)ₚ—(C_{q}F_{2q+1}), R(10a)—SO_{bm}, R(10b)R(10c)N—CO, R(11)—CO— or
R(12)R(13)N—SO₂—,
where the perfluoroalkyl group is straight-chain or branched;
X is oxygen, S or NR(14);
R(14) is H or (C₁-C₃)-alkyl;
bm is zero, 1 or 2;
p is zero, 1 or 2;
k is zero or 1;
q 1, 2, 3, 4, 5 or 6;
R(10a), R(10b), R(11) and R(12)
independently of one another are (C₁-C₈)-alkyl, (C₃-C₆)-alkenyl, —CₙH₂ₙ—R(15) or (C₁-C₈)-perfluoroalkyl;
n is zero, 1, 2, 3 or 4;
R(15) is (C₃-C₇)-cycloalkyl or phenyl, which is not substituted
or is substituted by 1-3 substituents selected from the group consisting of F, Cl, CF₃, methyl, methoxy and NR(16)R(17);
R(16) and R(17)
are H or C₁-C₄-alkyl;
or
R(10b), R(11) and R(12)
are hydrogen;
R(10c) and R(13)
independently are hydrogen or (C₁-C₄)-alkyl;
or
R(10b) and R(10c) and R(12) and R(13)
together are 4 or 5 methylene groups, of which one CH₂ group can be replaced by oxygen, sulfur, NH, N—CH₃ or N-benzyl;
or
R(3), R(4), R(5), R(6) and R(7)
independently of one another are (C₁-C₉)-alkyl, —CₐₗH₂ₐₗR(18) or (C₃-C₈)-alkenyl;
al is zero, 1 or 2;
R(18) is (C₃-C₈)-cycloalkyl, phenyl, biphenylyl or naphthyl, where the aromatics are not substituted or are substituted by 1-3 substituents selected from the group consisting of F, Cl, CF₃, methyl, methoxy and NR(19a)R(19b);
R(19a) and R(19b)
are hydrogen, (C₁-C₄)-alkyl or (C₁-C₄)-perfluoroalkyl;
or
R(3), R(4), R(5), R(6) and R(7)
independently of one another are (C₁-C₉)-heteroaryl, which is linked via C or N and which is unsubstituted or substituted by 1-3 substituents from the group consisting of F, Cl, CF₃, CH₃, methoxy, hydroxyl, amino, methylamino and dimethylamino;
or
R(3), R(4), R(5), R(6) and R(7)
independently of one another are 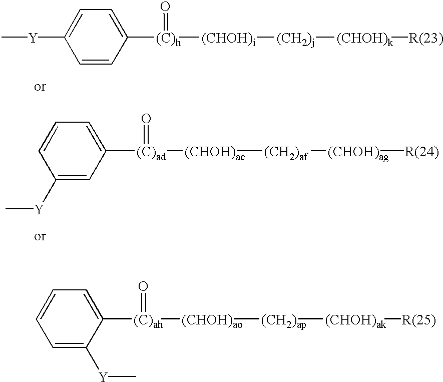
Y is oxygen, —S— or —NR(22)—;
h, ad, ah
independently of one another are zero or 1;
i, j, k, ae, af, ag, ao, ap and ak
independently of one another are zero, 1, 2, 3 or 4;
but where in each case
h, i and k are not simultaneously zero,
ad, ae and ag are not simultaneously zero, and
ah, ao and ak are not simultaneously zero,
R(23), R(24), R(25) and R(22)
independently of one another are hydrogen or (C₁-C₃)-alkyl;
or
R(3), R(4), R(5), R(6) and R(7)
independently of one another are SR(29), —OR(30), —NR(31)R(32) or —CR(33)R(34)R(35);
R(29), R(30), R(31) and R(33)
independently of one another are —CₐH₂ₐ—(C₁-C₉)-heteroaryl, which is unsubstituted or substituted by 1-3 substituents selected from the group consisting of F, Cl, CF₃, CH₃, methoxy, hydroxyl, amino, methylamino and dimethylamino;
a is zero, 1 or 2;
R(32), R(34) and R(35)
independently of one another are defined as R(29) or are hydrogen, (C₁-C₄)-alkyl or (C₁-C₄)-perfluoroalkyl;
or
R(3), R(4), R(5), R(6) and R(7)
independently of one another are 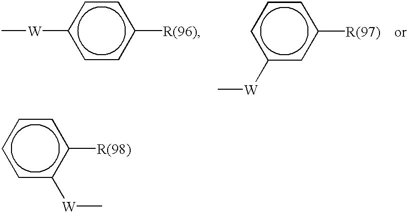
R(96), R(97) and R(98)
independently of one another are (C₁-C₉)-heteroaryl, which is linked via C or N and which is unsubstitued or substituted by 1 to 3 substituents from the group consisting of F, Cl, CF₃, CH₃, methoxy, hydroxyl, amino, methylamino, dimethylamino and benzyl;
W is oxygen, S or NR(36)—;
R(36) is H or (C₁-C₄)-alkyl;
or
R(3), R(4), R(5), R(6) and R(7)
independently of one another are R(46)X(1)—;
X(1) is oxygen, S, NR(47), (D═O)A— or NR(48)C═MN^{(*)}R(49)—;
M is oxygen or sulfur;
A is oxygen or NR(50);
D is C or SO;
R(46) is (C₁-C₈)-alkyl, (C₃-C₈)-alkenyl, (CH₂)_{b}C_{d}F_{2d+1} or —CₓH₂ₓ—R(51);
b is zero or 1;
d is 1, 2, 3, 4, 5, 6 or 7;
x is zero, 1, 2, 3 or 4;
R(51) is (C₃-C₈)-cycloalkyl, phenyl, biphenylyl or naphthyl,
where the aromatics are not substituted or are substituted by 1-3 substituents selected from the group consisting of F, Cl, CF₃, methyl, methoxy and
NR(52)R(53);
R(52) and R(53)
are hydrogen, (C₁-C₄)-alkyl or (C₁-C₄)-perfluoroalkyl;
R(47), R(48) and R(50) independently
are hydrogen, (C₁-C₄)-alkyl or (C₁-C₄)-perfluoroalkyl;
R(49) is defined as R(46);
or
R(46) and R(47), or R(46) and R(48)
together are 4 or 5 methylene groups, of which one CH₂ group can be replaced by oxygen, sulfur, NH, N—CH₃ or N-benzyl;
where A and N^{(*)} are bonded to the phenyl nucleus of the heteroaroylguanidine parent structure;
or
R(3), R(4), R(5), R(6) and R(7)
independently of one another are —SR(64), —OR(65), —NHR(66), —NR(67)R(68), —CHR(69)R(70) or —CR(54)R(55)OH, —C≡CR(56), —CR(58)═CR(57) or —[CR(59)R(60)]ᵤ—CO—[CR(61)R(62)]ᵥ—R(63);
R(64), R(65), R(66), R(67) and R(69)
identically or differently are —(CH₂)_{y}—(CHOH)_{z}—(CH₂)ₐₐ—(CHOH)ₜ—R(71) or —(CH₂)_{ab}—O—(CH₂—CH₂O)_{ac}—R(72);
R(71) and R(72)
independently of one another are hydrogen or methyl;
u is 1, 2, 3 or 4;
v is zero, 1, 2, 3 or 4;
y, z, aa, ab and ac identically or differently
are zero, 1, 2, 3 or 4;
t is 1, 2, 3 or 4;
R(68), R(70), R(54) and R(55)
identically or differently are hydrogen or (C₁-C₆)-alkyl;
or
R(69) and R(70), or R(54) and R(55)
together with the carbon atom carrying them are (C₃-C₈)-cycloalkyl;
R(63)
is hydrogen, (C₁-C₆)-alkyl, (C₃-C₈)-cycloalkyl or —CₑH₂ₑ—R(73);
e is zero, 1, 2, 3 or 4;
R(56), R(57) and R(73) independently
are phenyl, which is unsubstituted or substituted by 1-3 substituents selected from the group consisting of F, Cl, CF₃, methyl, methoxy and NR(74)R(75);
R(74) and R(75)
are hydrogen or (C₁-C₄)-alkyl;
or
R(56), R(57) and R(73) independently
are (C₁-C₉)-heteroaryl, which is unsubstituted or substituted as phenyl;
R(58), R(59), R(60), R(61) and R(62)
are hydrogen or methyl;
or
R(3), R(4), R(5), R(6) and R(7)
independently of one another are R(76)—NH—SO₂—;
R(76) is R(77)R(78)N—(C═Y′)—;
Y′ is oxygen, S or N—R(79);
R(77) and R(78)
identically or differently are hydrogen, (C₁-C₈)-alkyl, (C₃-C₆)-alkenyl or —C_{f}H_{2f}R(80);
f is zero, 1, 2, 3 or 4;
R(80) is (C₅-C₇)-cycloalkyl or phenyl, which is
unsubstituted or substituted by 1-3 substituents selected from the group consisting of F, Cl, CF₃, methoxy and (C₁-C₄)-alkyl;
or
R(77) and R(78)
together are 4 or 5 methylene groups, of which one CH₂ group can be replaced by oxygen, sulfur, NH, N—CH₃ or N-benzyl;
R(79) is defined as R(77) or is amidine;
or
R(3), R(4), R(5), R(6) and R(7)
independently of one another are NR(84a)R(85), OR(84b), SR(84c) or —CₙH₂ₙ—R(84d);
n is zero, 1, 2, 3 or 4;
R(84d) is (C₃-C₇)-cycloalkyl or phenyl, which is not substituted or is substituted by 1-3 substituents from the group consisting of F, Cl, CF₃, methyl, methoxy and NR(16)R(17);
R(16) and R(17)
are hydrogen, or C₁-C₄-alkyl;
R(84a), R(84b), R(84c) and R(85)
independently of one another are hydrogen, (C₁-C₈)-alkyl, (C₁-C₈)-perfluoroalkyl or (CH₂)ₐₓ—R(84g);
ax is zero, 1, 2, 3 or 4;
R(84g) is (C₃-C₇)-cycloalkyl or phenyl, which is not
substituted or is substituted by 1-3 substituents selected from the group consisting of F, Cl, CF₃, methyl, methoxy and NR(84u)R(84v);
R(84u) and R(84v)
are hydrogen or C₁-C₄-alkyl;
or
R(84a) and R(85)
together are 4 or 5 methylene groups, of which one CH₂ group can be replaced by oxygen, sulfur, NH, N—CH₃ or N-benzyl,
or a pharmaceutically tolerable salt thereof;
z) a benzoylguanidine of the formula 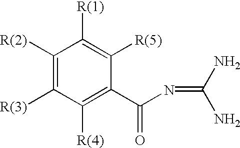
in which:
R(1) is R(6)—SOₘ;
m is zero, 1 or 2;
R(6) is perfluoroalkyl having 1, 2, 3, 4, 5 or 6 carbon atoms, which is straight-chain or branched;
R(2) and R(3)
independently of one another are hydrogen, F, Cl, Br, I, alkyl having 1, 2, 3 or 4 carbon atoms, alkoxy having 1, 2, 3 or 4 carbon atoms or phenoxy,
which is unsubstituted or substituted by 1-3 substituents selected from the group consisting of F, Cl, methyl and methoxy;
or
R(2) and R(3)
independently of one another are pyrrol-1-yl, pyrrol-2-yl- or pyrrol-3-yl,
which is not substituted or is substituted by 1 to 4 substituents selected from the group consisting of F, Cl, Br, I, CN, alkanoyl having 2, 3, 4, 5, 6, 7 or 8 carbon atoms, alkoxycarbonyl having 2, 3, 4, 5, 6, 7 or 8 carbon atoms, formyl, carboxyl, CF₃, methyl and methoxy;
R(4) and R(5)
independently of one another are hydrogen, alkyl having 1, 2 or 3 carbon atoms, F, Cl, Br, I, CN, OR(7), NR(8)R(9) or —(CH₂)ₙ—(CF₂)ₒ—CF₃;
R(7), R(8) and R(9)
independently of one another are hydrogen or alkyl having 1, 2, 3 or 4 carbon atoms;
n is zero or 1;
o is zero, 1 or 2;
or a pharmacologically acceptable salt thereof;
ab) a phenyl-substituted alkenylcarboxylic acid guanidide of the formula 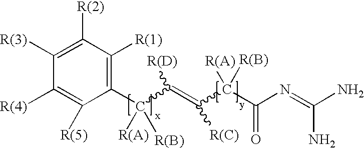
in which:
R(A) is hydrogen, F, Cl, Br, I, CN, OH, OR(6), (C₁-C₈)-alkyl, Oᵣ(CH₂)ₐC_{b}F_{2b+1}, (C₃-C₈)-cycloalkyl or NR(7)R(8);
r is zero or 1;
a is zero, 1, 2, 3 or 4;
b is 1, 2, 3, 4, 5, 6, 7 or 8;
R(6) is (C₁-C₈)-alkyl, (C₁-C₄)-perfluoroalkyl, (C₃-C₈)-alkenyl, (C₃-C₈)-cycloalkyl, phenyl or benzyl;
where the aromatics are not substituted or are substituted by 1-3 substituents from the group consisting of F, Cl, CF₃, methyl, methoxy and NR(9)R(10);
R(9) and R(10)
are H, (C₁-C₄)-alkyl or (C₁-C₄)-perfluoroalkyl;
R(7) and R(8)
independently of one another are defined as R(6);
or
R(7) and R(8)
together are 4 or 5 methylene groups, of which one CH₂ group can be replaced by oxygen, sulfur, NH, N—CH₃ or N-benzyl;
R(B) independently is defined as R(A);
X is zero, 1 or 2;
Y is zero, 1 or 2;
R(C) is hydrogen, F, Cl, Br, I, CN, OR(12), (C₁-C₈)-alkyl, Oₚ(CH₂)_{f}C_{g}F_{2g+1} or (C₃-C₈)-cycloalkyl;
p is zero or 1;
f is zero, 1, 2, 3 or 4;
g is 1, 2, 3, 4, 5, 6, 7 or 8;
R(12)
is (C₁-C₈)-alkyl, (C₁-C₄)-perfluoroalkyl, (C₃-C₈)-alkenyl, (C₃-C₈)-cycloalkyl, phenyl or benzyl;
where the aromatics phenyl or benzyl are not substituted or are substituted by 1-3 substituents from the group consisting of F, Cl, CF₃, methyl, methoxy and NR(13)R(14);
R(13) and R(14)
independently of one another are H, (C₁-C₄)-alkyl or (C₁-C₄)-perfluoroalkyl;
R(D) independently is defined as R(C),
R(1) is hydrogen, (C₁-C₈)-alkyl, —Oₜ(CH₂)_{d}CₑF₂ₑ₊₁, (C₃-C₈)-cycloalkyl, F, Cl, Br, I or CN;
t is zero or 1;
d is zero, 1, 2, 3 or 4;
e is 1, 2, 3, 4, 5, 6, 7 or 8;
R(2), R(3), R(4) and R(5)
independently of one another are defined as R(1);
but with the condition
that at least one of the substituents R(A), R(B), R(C), R(D), R(1), R(2), R(4) or R(5) is an Oᵣ(CH₂)ₐC_{b}F_{2b+1}, Oₚ(CH₂)_{f}C_{g}F_{2g+1} or Oₜ(CH₂)_{d}CₑF₂ₑ₊₁ group and R(3) is not an Oₜ(CH₂)_{d}CₑF₂ₑ₊₁ group;
or pharmaceutically tolerable salt thereof;
ac) an ortho-amino-substituted benzoylguanidine of the formula 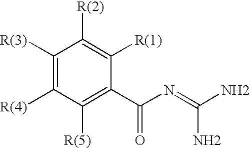
in which:
R(1) is NR(50)R(6),
R(50) and R(6)
independently of one another are hydrogen, (C₁-C₈)-alkyl or (C₁-C₈)-perfluoroalkyl;
R(2), R(3), R(4) and R(5)
independently of one another are R(10)—SOₐ—, R(11)R(12)N—CO—, R(13)—CO— or R(14)R(15)N—SO₂—;
a is zero, 1 or 2,
R(10), R(11), R(12), R(13), R(14) and R(15)
independently of one another are (C₁-C₈)-alkyl, (C₁-C₈)-perfluoroalkyl, (C₃-C₆)-alkenyl or —C_{ab}H_{2ab}—R(16);
ab is zero, 1, 2, 3 or 4;
R(16) is (C₃-C₇)-cycloalkyl, phenyl,
which is not substituted or is substituted by 1-3 substituents selected from the group consisting of F, Cl, CF₃, methyl, methoxy or NR(17)R(18);
R(17) and R(18)
independently of one another are H, CF₃ or (C₁-C₄)-alkyl;
or
R(11), R(12), and also R(14) and R(15)
together are 4 or 5 methylene groups, of which one CH₂ group can be replaced by oxygen, S, NH, N—CH₃ or N-benzyl;
or
R(11), R(12), R(14) and R(15)
independently of one another are hydrogen;
or
R(2), R(3), R(4) and R(5)
independently of one another are SR(21), —OR(22), —NR(23)R(24) or —CR(25)R(26)R(27);
R(21), R(22), R(23) and R(25)
independently of one another are —C_{b}H_{2b}—(C₁-C₉)-heteroaryl, which is unsubstituted or substituted by 1-3 substituents selected from the group consisting of F, Cl, CF₃, CH₃, methoxy, hydroxyl, amino, methylamino and dimethylamino;
b is zero, 1 or 2;
R(24), R(26) and R(27)
independently of one another are hydrogen, (C₁-C₄)-alkyl or (C₁-C₄)-perfluoroalkyl;
or
R(2), R(3), R(4) and R(5)
independently of one another are hydrogen, F, Cl, Br, I, CN, —(Xa)_{dg}—C_{da}H_{2da+1}, —(Xb)_{dh}—(CH₂)_{db}—C_{de}F_{2de+1}, (C₃-C₈)-alkenyl or —C_{df}H_{2df}R(30);
(Xa) is O, S or NR(33);
R(33)
is H, (C₁-C₄)-alkyl or (C₁-C₄)-perfluoroalkyl;
dg is zero or 1;
(Xb) is O, S or NR(34);
R(34)
is H, (C₁-C₄)-alkyl or (C₁-C₄)-perfluoroalkyl;
dh is zero or 1;
da is zero, 1, 2, 3, 4, 5, 6, 7, 8;
db is zero, 1, 2, 3, 4;
de is zero, 1, 2, 3, 4, 5, 6, 7;
df is zero, 1, 2, 3, 4;
R(30)
is (C₃-C₈)-cycloalkyl, phenyl, biphenylyl or naphthyl, where the aromatics phenyl, biphenylyl or naphthyl are not substituted or are substituted by 1-3 substituents selected from the group consisting of F, Cl, CF₃, methyl, methoxy and NR(31)R(32);
R(31) and R(32)
are H, (C₁-C₄)-alkyl or (C₁-C₄)-perfluoroalkyl;
or
R(2), R(3), R(4) and R(5)
independently of one another are NR(40)R(41) or —(Xe)—(CH₂)_{eb}R(45);
R(40) and R(41)
independently of one another are hydrogen, (C₁-C₈)-alkyl, (C₁-C₈)-perfluoroalkyl or (CH₂)ₑ—R(42);
e is zero, 1, 2, 3 or 4;
R(42)
is (C₃-C₇)-cycloalkyl, phenyl, which is not substituted or is substituted by 1-3 substituents selected from the group consisting of F, Cl, CF₃, methyl, methoxy and NR(43)R(44);
R(43) and R(44)
independently of one another are H, CF₃ or (C₁-C₄)-alkyl;
or
R(40) and R(41)
together are 4 or 5 methylene groups, of which one CH₂ group can be replaced by oxygen, sulfur, NH, N—CH₃ or N-benzyl;
(Xe) is O, S or NR(47);
R(47)
is H, (C₁-C₄)-alkyl or (C₁-C₄)-perfluoroalkyl;
eb is zero, 1, 2, 3 or 4;
R(45) is (C₃-C₇)-cycloalkyl, phenyl, which is not substituted or is substituted by 1-3 substituents selected from the group consisting of F, Cl, CF₃, methyl, methoxy, NR(50)R(51) and —(Xfa)—(CH₂)_{ed}—(Xfb)R(46);
Xfa is CH₂, O S or NR(48);
Xfb is O, S or NR(49);
ed is 1, 2, 3 or 4;
R(46)
is H, (C₁-C₄)-alkyl or (C₁-C₄)-perfluoroalkyl;
R(48), R(49), R(50) and R(51)
independently of one another are H or (C₁-C₄)-alkyl or (C₁-C₄)-perfluoroalkyl;
where R(3) and R(4), however, cannot be hydrogen,
or pharmaceutically tolerable salt thereof;
ad) a benzoylguanidine of the formula 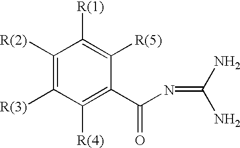
in which:
one of the three substituents R(1), R(2) and R(3)
is (C₁-C₉)-heteroaryl-N-oxide,
which is linked via C or N and which is unsubstituted or substituted by 1-3 substituents selected from the group consisting of F, Cl, CF₃, CH₃, methoxy, hydroxyl, amino, methylamino and dimethylamino;
or
one of the three substituents R(1), R(2) and R(3)
is —SR(10), —OR(10), —NR(10)R(11) or —CR(10)R(11)R(12);
R(10)
is —CₐH₂ₐ(C₁-C₉)-heteroaryl-N-oxide,
which is unsubstituted or substituted by 1-3 substituents selected from the group consisting of F, Cl, CF₃, CH₃, methoxy, hydroxyl, amino, methylamino and dimethylamino;
a is zero, 1 or 2;
R(11) and R(12)
independently of one another are defined as R(10), are hydrogen or (C₁-C₄)-alkyl;
and the other substituents R(1), R(2) and R(3) in each case
independently of one another are (C₁-C₈)-alkyl, (C₂-C₈)-alkenyl or —CₘH₂ₘR(14);
m is zero, 1 or 2;
R(14) is (C₃-C₈)-cycloalkyl or phenyl,
which is not substituted or is substituted by 1-3 substituents selected from the group consisting of F, Cl, CF₃, methyl, methoxy and NR(15)R(16),
R(15) and R(16)
are hydrogen or CH₃;
or
the other substituents R(1), R(2) and R(3) in each case
independently of one another are hydrogen, F, Cl, Br, I, —C≡N, X—(CH₂)ₚ—(C_{q}F_{2q+1}), R(22)—SOᵤ, R(23)R(24)N—CO, R(25)—CO— or R(26)R(27)N—SO₂—,
where the perfluoroalkyl group is straight-chain or branched;
X is a bond, oxygen, S or NR(28);
u is zero, 1 or 2;
p is zero, 1 or 2;
q is zero, 1, 2, 3, 4, 5 or 6;
R(22), R(23), R(25) and R(26)
independently are (C₁-C₈)-alkyl, (C₂-C₆)-alkenyl, —CₙH₂ₙ—R(29) or CF₃;
n is zero, 1, 2, 3 or 4;
R(28) is hydrogen or (C₁-C₃)-alkyl;
R(29) is (C₃-C₇)-cycloalkyl or phenyl;
which is not substituted or is substituted by 1-3 substituents selected from the group consisting of F, Cl, CF₃, methyl, methoxy and NR(30)R(31);
R(30) and R(31)
are hydrogen or C₁-C₄-alkyl,
or
R(23), R(25) and R(26)
are also hydrogen;
R(24) and R(27)
independently of one another are hydrogen or (C₁-C₄)-alkyl;
or
R(23) and R(24), and also R(26) and R(27) together are 4 or 5 methylene groups, of which one CH₂ group can be replaced by oxygen, S, NH, N—CH₃ or N-benzyl;
or
the other substituents R(1), R(2) and R(3) in each case
independently of one another are OR(35) or NR(35)R(36);
R(35) and R(36)
independently of one another are hydrogen or (C₁-C₆)-alkyl;
or
R(35) and R(36)
together are 4-7 methylene groups, of which one CH₂ group can be replaced by oxygen, S, NH, N—CH₃ or N-benzyl,
R(4) and R(5)
independently of one another are hydrogen, (C₁-C₄)-alkyl, F, Cl, —OR(32), —NR(33)R(34) or CᵣF₂ᵣ₊₁;
R(32), R(33) and R(34)
independently of one another are hydrogen or (C₁-C₃)-alkyl;
r is 1, 2, 3 or 4;
or pharmaceutically tolerable salt thereof;
ae) a benzoylguanidine of the formula 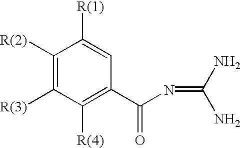
in which:
R(1) is hydrogen, F, Cl, Br, I, CN, NO₂, OH, (C₁-C₈)-alkyl, (C₃-C₈)-cycloalkyl or Oₐ—(CH₂)_{b}—(CF₂)_{c}—CF₃;
a is zero or 1;
b is zero, 1 or 2;
c is zero, 1, 2 or 3;
or
R(1) is R(5)—SOₘ or R(6)R(7)N—SO₂—;
m is zero, 1 or 2;
R(5) and R(6) independently of one another
are (C₁-C₈)-alkyl, (C₃-C₆)-alkenyl, CF₃ or —CₙH₂ₙ—R(8);
n is zero, 1, 2, 3 or 4;
R(7) is hydrogen or (C₁-C₄)-alkyl;
R(8) is (C₃-C₇)-cycloalkyl or phenyl,
which is not substituted or is substituted by 1-3 substituents selected from the group consisting of F, Cl, CF₃, methyl, methoxy and NR(9)R(10);
R(9) and R(10) independently of one another are hydrogen or (C₁-C₄)-alkyl;
or
R(6) is H;
or R(6) and R(7)
together are 4 or 5 methylene groups, of which one CH₂ group can be replaced by oxygen, S, NH, N—CH₃ or N-benzyl,
or
R(1) is —SR(11), —OR(11) or —CR(11)R(12)R(13);
R(11) is —CₚCₚH₂ₚ —(C₃-C₈)-cycloalkyl, —(C₁-C₉)-heteroaryl or phenyl,
where the aromatic systems are unsubstituted or substituted by 1-3 substituents selected from the group consisting of F, Cl, CF₃, CH₃, methoxy, hydroxyl, amino, methylamino and dimethylamino;
R(12), R(13) independently of one another
are defined as R(11) or are hydrogen or (C₁-C₄)-alkyl;
p is zero, 1 or 2;
or
R(1) is phenyl, naphthyl, biphenylyl or (C₁-C₉)-heteroaryl, the latter linked via C or N,
which are unsubstituted or substituted by 1-3 substituents selected from the group consisting of F, Cl, CF₃, CH₃, methoxy, hydroxyl, amino, methylamino and dimethylamino;
R(2) is —CF₂R(14), —CF[R(15)][R(16)], —CF[(CF₂)_{q}—CF₃)][R(15)] or —C[(CF₂)ᵣ—CF₃]═CR(15)R(16);
R(14) is (C₁-C₄)-alkyl or (C₃-C₆)-cycloalkyl;
R(15) and R(16) independently of one another
are hydrogen or (C₁-C₄)-alkyl;
q is zero, 1 or 2;
r is zero, 1 or 2;
R(3) is defined as R(1);
R(4) is hydrogen, (C₁-C₃)-alkyl, F, Cl, Br, I, CN or —(CH₂)ₛ—(CF₂)ₜ—CF₃;
s is zero or 1;
t is zero, 1 or 2;
or pharmaceutically tolerable salt thereof;
af) a benzoylguanidine of the formula 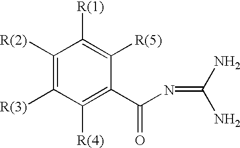
in which:
one of the three substituents R(1), R(2) and R(3)
is —Y-4-[(CH₂)ₖ—CHR(7)—(C═O)R(8)]-phenyl,
—Y-3—(CH₂)ₖ—CHR(7)—(C═O)R(8)]-phenyl or
—Y-2-[(CH₂)ₖ—CHR(7)—(C═O)R(8)]-phenyl,
where the phenyl in each case is unsubstituted or substituted by 1-2 substituents from the group F, Cl, —CF₃, methyl, hydroxyl, methoxy, and —NR(37)R(38);
R(37) and R(38)
independently of one another are hydrogen or —CH₃;
Y is a bond, oxygen, —S— or —NR(9);
R(9) is hydrogen or —(C₁-C₄)-alkyl;
R(7) is —OR(10) or —NR(10)R(11);
R(10) and R(11)
independently of one another are hydrogen, —(C₁-C₈)-alkyl, —(C₁-C₈)-alkanoyl, —(C₁-C₈)-alkoxycarbonyl, benzyl, benzyloxycarbonyl;
or
R(10) is trityl;
R(8) is —OR(12) or —NR(12)R(13);
R(12) and R(13)
independently of one another are hydrogen, —(C₁-C₈)-alkyl or benzyl;
k is zero, 1, 2, 3 or 4;
and the other radicals R(1), R(2) and R(3) in each case
independently of one another are —(C₁-C₈)-alkyl, —(C₂-C₈)-alkenyl or —(CH₂)ₘR(14);
m is zero, 1 or 2;
R(14) is —(C₃-C₈)-cycloalkyl or phenyl,
which is not substituted or is substituted by 1-3 substituents selected from the group consisting of F, Cl, —CF₃, methyl, methoxy and —NR(15)R(16);
R(15) and R(16)
are hydrogen or —CH₃;
or
the other radicals R(1), R(2) and R(3) in each case
independently of one another are R(18)R(19)N—(C═Y′)—NH—SO₂—;
Y′ is oxygen, —S— or —N—R(20);
R(18) and R(19)
independently of one another are hydrogen, —(C₁-C₈)-alkyl, —(C₃-C₆)-alkenyl or —(CH₂)ₜ—R(21);
t is zero, 1, 2, 3 or 4;
R(21) is —(C₅-C₇)-cycloalkyl or phenyl,
which is unsubstituted or substituted by 1-3 substituents selected from the group consisting of F, Cl, —CF₃, methoxy and —(C₁-C₄)-alkyl;
or
R(18) and R(19)
together are 4 or 5 methylene groups, of which one CH₂ group can be replaced by oxygen, —S—, —NH—, —N—CH₃ or —N-benzyl;
R(20)
is defined as R(18) or is amidine;
or
the other radicals R(1), R(2) and R(3) in each case
independently of one another are hydrogen, F, Cl, Br, I, —C≡N, X—(CH₂)ₚ—(C_{q}F_{2q+1}), R(22)—SOᵤ—, R(23)R(24)N—CO—, R(25)—CO— or R(26)R(27)N—SO₂—, where the perfluoroalkyl group is straight-chain or branched;
X is a bond, oxygen, —S— or —NR(28);
u is zero, 1 or 2;
p is zero, 1 or 2;
q is 1, 2, 3, 4, 5 or 6;
R(22), R(23), R(25) and R(26)
independently of one another are —(C₁-C₈)-alkyl, —(C₃-C₆)-alkenyl, —(CH₂)ₙ—R(29) or —CF₃;
n is zero, 1, 2, 3 or 4;
R(28) is hydrogen or —(C₁-C₃)-alkyl;
R(29) is —(C₃-C₇)-cycloalkyl or phenyl,
which is not substituted or is substituted by 1-3 substituents selected from the group consisting of F, Cl, —CF₃, methyl, methoxy and —NR(30)R(31);
R(30) and R(31)
are hydrogen or —(C₁-C₄)-alkyl;
or
R(23), R(25) and R(26)
are hydrogen;
R(24) and R(27)
independently of one another are hydrogen or —(C₁-C₄)-alkyl;
or
R(23) and R(24), and also R(26) and R(27)
together are 4 or 5 methylene groups, of which one CH₂ group can be replaced by oxygen, —S—, —NH—, —N—CH₃ or —N-benzyl;
or
the other radicals R(1), R(2) and R(3) in each case
independently of one another are —OR(35) or —NR(35)R(36);
R(35) and R(36)
independently of one another are hydrogen or —(C₁-C₆)-alkyl;
or
R(35) and R(36)
together are 4-7 methylene groups, of which one CH₂ group can be replaced by oxygen, —S—, —NH—, —N—CH₃ or —N-benzyl;
R(4) and R(5)
independently of one another are hydrogen, —(C₁-C₄)-alkyl, F, Cl, —OR(32), —NR(33)R(34) or —CᵣF₂ᵣ₊₁;
R(32), R(33) and R(34)
independently of one another are hydrogen or —(C₁-C₃)-alkyl;
r is 1, 2, 3 or 4;
or pharmaceutically tolerable salt thereof;
ag) a benzoylguanidine of the formula 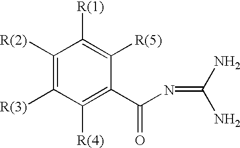
in which:
R(1) is R(6)—CO or R(7)R(8)N—CO;
R(6) is (C₁-C₈)-alkyl, (C₁-C₈)-perfluoroalkyl, (C₃-C₈)-alkenyl or —CₙH₂ₙ—R(9),
n is zero, 1, 2, 3 or 4;
R(9) is (C₃-C₉)-cycloalkyl, phenyl, biphenylyl or naphthyl,
where the aromatics are not substituted or are substituted by 1-3 substituents selected from the group consisting of F, Cl, CF₃, methyl, methoxy and NR(10)R(11),
R(10) and R(11)
are H, (C₁-C₄)-alkyl or (C₁-C₄)-perfluoroalkyl;
R(7) is H, (C₁-C₈)-alkyl, (C₁-C₈)-perfluoroalkyl, (C₃-C₈)-alkenyl or —CₙH₂ₙ—R(12);
n is zero, 1, 2, 3 or 4;
R(12) is (C₃-C₈)-cycloalkyl, phenyl, biphenylyl or naphthyl,
where the aromatics are not substituted or are substituted by 1-3 substituents selected from the group consisting of F, Cl, CF₃, methyl, methoxy and NR(13)R(14);
R(13) and R(14)
are H, (C₁-C₄)-alkyl or (C₁-C₄)-perfluoroalkyl;
R(8) is H, (C₁-C₄)-alkyl or (C₁-C₄)-perfluoroalkyl;
or
R(7) and R(8) together
are 4 or 5 methylene groups, of which one CH₂ group can be replaced by oxygen, S, NH, N—CH₃ or N-benzyl;
R(2) is defined as R(1), or is H, OH, F, Cl, Br, I, CN, NO₂, (C₁-C₈)-alkyl, (C₁-C₈)-perfluoroalkyl, (C₃-C₈)-alkenyl or —CₙH₂ₙR(15);
n is zero, 1, 2, 3 or 4;
R(15) is (C₃-C₈)-cycloalkyl, phenyl, biphenylyl or naphthyl,
where the aromatics are not substituted or are substituted by 1-3 substituents selected from the group consisting of F, Cl, CF₃, methyl, methoxy and NR(16)R(17);
R(16) and R(17)
are H, (C₁-C₄)-alkyl or (C₁-C₄)-perfluoroalkyl;
or
R(2) is (C₁-C₉)-heteroaryl,
which is linked via C or N and which is unsubstituted or substituted by 1-3 substituents selected from the group consisting of F, Cl, CF₃, CH₃, methoxy, hydroxyl, amino, methylamino and dimethylamino;
or
R(2) is SR(18), —OR(18), —NR(18)R(19) or —CR(18)R(19)R(20);
R(18) is —CₐH₂ₐ—(C₁-C₉)-heteroaryl,
which is unsubstituted or substituted by 1-3 substituents selected from the group consisting of F, Cl, CF₃, CH₃, methoxy, hydroxyl, amino, methylamino, and dimethylamino;
a is zero, 1 or 2;
R(19) and R(20)
independently of one another are defined as R(18) or are hydrogen, (C₁-C₄)-alkyl or (C₁-C₄)-perfluoroalkyl;
or
R(2) is R(21)—SOₘ or R(22)R(23)N—SO₂—;
m is 1 or 2;
R(21) is (C₁-C₈)-alkyl, (C₁-C₈)-perfluoroalkyl, (C₃-C₈)-alkenyl or —CₙH₂ₙ—R(24);
n is zero, 1, 2, 3 or 4;
R(24) is (C₃-C₈)-cycloalkyl, phenyl, biphenylyl or naphthyl,
where the aromatics are not substituted or are substituted by 1-3 substituents selected from the group consisting of F, Cl, CF₃, methyl, methoxy and NR(27)R(28);
R(27) and R(28)
are H, (C₁-C₄)-alkyl or (C₁-C₄)-perfluoroalkyl;
R(22) is H, (C₁-C₈)-alkyl, (C₁-C₈)-perfluoroalkyl, (C₃-C₈)-alkenyl or —CₙH₂ₙ—R(29);
n is zero, 1, 2, 3 or 4;
R(29) is (C₃-C₈)-cycloalkyl, phenyl, biphenylyl or naphthyl,
where the aromatics are not substituted or are substituted by 1-3 substituents selected from the group consisting of F, Cl, CF₃, methyl, methoxy and NR(30)R(31);
R(30) and R(31)
are H, (C₁-C₄)-alkyl or (C₁-C₄)-perfluoroalkyl;
R(23) is hydrogen, (C₁-C₄)-alkyl or (C₁-C₄)-perfluoroalkyl;
or
R(22) and R(23)
together are 4 or 5 methylene groups, of which one CH₂ group can be replaced by oxygen, S, NH, N—CH₃ or N-benzyl;
or
R(2) is R(33)X—;
X is oxygen, S, NR(34), (D═O)A— or NR(34)C═MN^{(*)}R(35)—;
M is oxygen or S;
A is oxygen or NR(34);
D is C or SO;
R(33) is (C₁-C₈)-alkyl, (C₃-C₈)-alkenyl, (CH₂)_{b}C_{d}F_{2d+1} or —CₙH₂ₙ—R(36);
b is zero or 1;
d is 1, 2, 3, 4, 5, 6 or 7;
n is zero, 1, 2, 3, or 4;
R(36) is (C₃-C₈)-cycloalkyl, phenyl, biphenylyl or naphthyl,
where the aromatics are not substituted or are substituted by 1-3 substituents selected from the group consisting of F, Cl, CF₃, methyl, methoxy and NR(37)R(38);
R(37) and R(38)
are H, (C₁-C₄)-alkyl or (C₁-C₄)-perfluoroalkyl;
R(34) is hydrogen, (C₁-C₄)-alkyl or (C₁-C₄)-perfluoroalkyl;
R(35) is defined as R(33);
or
R(33) and R(34)
together are 4 or 5 methylene groups, of which one CH₂ group can be replaced by oxygen, S, NH, N—CH₃ or N-benzyl;
where A and N^{(*)} are bonded to the phenyl nucleus of the benzoylguanidine parent structure;
or
R(2) is —SR(40), —OR(40), —NHR(40), —NR(40)R(41), —CHR(40)R(42),
—CR(42)R(43)OH, —C≡CR(45), —CR(46)═CR(45) or
—[CR(47)R(48)]ᵤ—CO—[C(R49)R(50)]ᵥ—R(44);
R(40) and R(41)
independently of one another are —(CH₂)ₚ—(CHOH)_{q}—(CH₂)ᵣ—(CHOH)ₜ—R(51) or —(CH₂)ₚ—O—(CH₂—CH₂O)_{q}—R(51);
R(51) is hydrogen or methyl;
u is 1, 2, 3 or 4;
v is zero, 1, 2, 3 or 4;
p, q and r
independently of one another are zero, 1, 2, 3 or 4;
t is 1, 2, 3 or 4;
R(42) and R(43)
independently of one another are hydrogen or (C₁-C₆)-alkyl;
or
R(42) and R(43)
together with the carbon atom carrying them are a (C₃-C₈)-cycloalkyl;
R(44) is hydrogen, (C₁-C₆)-alkyl, (C₃-C₈)-cycloalkyl, —CₑH₂ₑ—R(45);
e is zero, 1, 2, 3 or 4;
R(45) is phenyl,
which is unsubstituted or substituted by 1-3 substituents selected from the group consisting of F, Cl, CF₃, methyl, methoxy and NR(52)R(53);
R(52) and R(53)
are H or (C₁-C₄)-alkyl;
or
R(45) is (C₁-C₉)-heteroaryl,
which is unsubstituted or substituted as phenyl;
or
R(45) is (C₁-C₆)-alkyl,
which is unsubstituted or substituted by 1-3 OH;
R(46), R(47), R(48), R(49) and R(50)
independently of one another are hydrogen or methyl;
or
R(2) is R(55)—NH—SO₂—;
R(55) is R(56)R(57)N—(C═Y)—;
Y is oxygen, S or N—R(58);
R(56) and R(57)
independently of one another are hydrogen, (C₁-C₈)-alkyl, (C₃-C₆)-alkenyl or —C_{f}H_{2f}—R(59);
f is zero, 1, 2, 3 or 4;
R(59) is (C₅-C₇)-cycloalkyl or phenyl,
which is unsubstituted or substituted by 1-3 substituents selected from the group consisting of F, Cl, CF₃, methoxy and (C₁-C₄)-alkyl;
or
R(56) and R(57)
together are 4 or 5 methylene groups, of which one CH₂ group can be replaced by oxygen, S, NH, N—CH₃ or N-benzyl;
R(58)
is defined as R(56) or is amidine;
R(3), R(4) and R(5) are independently of one another defined as R(1) or R(2), but where at least one of the substituents R(2), R(3), R(4) and R(5) must be OH;
or pharmaceutically tolerable salt thereof;
ah) a benzoylguanidine of the formula 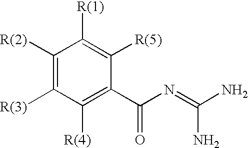
in which:
one of the three substituents R(1), R(2) and R(3)
is R(6)—A-B-D-;
R(6) is an amino group —NR(7)R(8), an amidino group R(7)R(8)N—C[═N—R(9)]— or a guanidino group 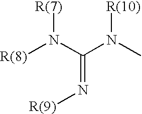
R(7), R(8), R(9) and R(10)
independently of one another are hydrogen or alkyl having 1, 2, 3 or 4 carbon atoms;
or
R(7) and R(8)
together are CₐH₂ₐ;
a is 4, 5,6 or 7;
where if a=5, 6 or 7 a methylene group of the group CₐH₂ₐ can be replaced by a heteroatom group O, SOₘ or NR(11),
or
R(8) and R(9) or R(9) and R(10) or R(7) and R(10)
are a group CₐH₂ₐ;
a is 2, 3, 4 or 5;
where if a=3, 4 or 5 a methylene group of the group CₐH₂ₐ can be replaced by a heteroatom group O, SOₘ or NR(11);
m is zero, 1 or 2;
R(11) is hydrogen or methyl;
or
R(6) is a basic heteroaromatic ring system having 1-9 carbon atoms;
A is C_{b}H_{2b};
b is 1, 2, 3, 4, 5, 6, 7, 8, 9 or 10;
where in the group C_{b}H_{2b} one or two methylene groups can be replaced by one of the groupings selected from the group consisting of —O—, —CO—, —CH[OR(20)]—, —SOₘ—, —NR(20)—, —NR(20)—CO—, —NR(20)—CO—NH—, —NR(20)—CO—NH—SO₂— 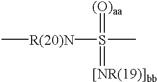
and —SOₐₐ[NR(19)]_{bb}—;
and where in the group C_{b}H_{2b} a methylene group can be replaced by —CH—R(99), where R(99) together with R(7) forms a pyrrolidine or piperidine ring;
aa is 1 or 2;
bb is 0 or 1;
aa+bb=2;
R(19) is hydrogen or alkyl having 1, 2, 3 or 4 carbon atoms;
R(20) is hydrogen or methyl;
B is a phenylene or naphthylene radical 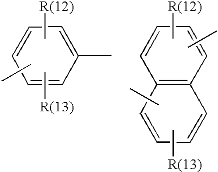
R(12) and R(13)
independently of one another are hydrogen, methyl, F, Cl, Br, I, CF₃ or —SO_{w}—R(14);
R(14) is methyl or NR(15)R(16);
R(15) and R(16)
independently of one another are hydrogen or alkyl having 1, 2, 3 or 4 carbon atoms;
w is zero, 1 or 2;
D is —C_{d}H_{2d}—X_{f}—;
d is zero, 1, 2, 3 or 4;
X is —O—, —CO—, —CH[OR(21)]—, —SOₘ— or —NR(21)—;
f is zero or 1;
R(21) is hydrogen or methyl;
m is zero, 1 or 2;
and the other substituents R(1) and R(2) and R(3) in each case
independently of one another are hydrogen, F, Cl, Br, I, —CN, —(C₁-C₈)-alkyl, —(C₂-C₈)-alkenyl, —NR(35)R(36) or R(17)—C_{g}H_{2g}—Zₕ—;
g is zero, 1, 2, 3 or 4;
h is zero or 1;
R(35) and R(36)
independently of one another are hydrogen or alkyl having 1, 2, 3, 4, 5 or 6 carbon atoms;
or
R(35) and R(36)
together are 4-7 methylene groups, of which one CH₂ group can be replaced by oxygen, —S—, —NH—, —NCH₃ or —N-benzyl;
Z is —O—, —CO—, —SOᵥ—, —NR(18)—, —NR(18)—CO—, —NR(18)—CO—NH— or —NR(18)—SO₂—;
R(18) is hydrogen or methyl;
v is zero, 1 or 2;
R(17) is hydrogen, cycloalkyl having 3, 5 or 6 carbon atoms or CₖF₂ₖ₊₁—;
k is 1, 2 or 3,
or
R(17) is pyrrol-1-yl, pyrrol-2-yl or pyrrol-3-yl,
which is not substituted or is substituted by 1-4 substituents selected from the group consisting of F, Cl, Br, I, —CN, (C₂-C₈)-alkanoyl, (C₂-C₈)-alkoxycarbonyl, formyl, carboxyl, —CF₃, methyl and methoxy;
or
R(17) —is (C₃-C₈)-cycloalkyl or phenyl, which is not substituted or is substituted by 1-3 substituents selected from the group consisting of F and Cl, —CF₃, methyl, hydroxyl, methoxy, —NR(37)R(38), CH₃SO₂— and H₂NO₂S—;
R(37) and R(38)
are hydrogen or —CH₃;
R(4) and R(5)
independently of one another are hydrogen, alkyl having 1, 2, 3 or 4 carbon atoms, F, Cl, —OR(32), —NR(33)R(34) or —CᵣF₂ᵣ₊₁;
R(32), R(33) and R(34)
independently of one another are hydrogen or alkyl having 1, 2 or 3 carbon atoms;
r is 1, 2, 3 or 4;
or pharmacologically tolerable salt thereof;
ai) an indenoylguanidine of the formula 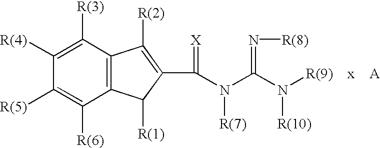
in which:
R(1) and R(2)
independently of one another are hydrogen, alkyl having 1, 2, 3, 4, 5, 6, 7, 8, 9 or 10 carbon atoms, cycloalkyl having 3, 4, 5 or 6 carbon atoms, O-alkyl having 1, 2, 3 or 4 carbon atoms, O—C(═O)-alkyl having 1, 2, 3 or 4 carbon atoms or CₘH₂ₘ—NR(12)R(13);
R(12) and R(13)
independently of one another are hydrogen or alkyl having 1, 2, 3 or 4 carbon atoms;
m is zero, 1, 2, 3 or 4; or
R(1) and R(2) independently of one another are NH—C(═O)—NH₂, C(═O)—O—alkyl having 1, 2, 3 or 4 carbon atoms, C(═O)—NH₂, C(═O)—NH-alkyl having 1, 2, 3 or 4 carbon atoms, C(═O)—N(alkyl)₂ having 1, 2, 3 or 4 carbon atoms in each alkyl group, alkenyl having 2, 3, 4, 5, 6, 7, 8, 9 or 10 carbon atoms, alkynyl having 2, 3, 4, 5, 6, 7, 8, 9 or 10 carbon atoms, alkylaryl having 1, 2, 3 or 4 carbon atoms in the alkyl group, alkenylaryl having 2, 3, 4, 5, 6, 7, 8, 9 or 10 carbon atoms in the alkenyl group, alkynylaryl having 2, 3, 4, 5, 6, 7, 8, 9 or 10 carbon atoms in the alkynyl group, C₁-C₄-alkyl-substituted aryl, C₁-C₄-alkylheteroaryl, C₁-C₄-alkenylheteroaryl, aminoalkylaryl having 1, 2, 3 or 4 carbon atoms in the alkyl group, substituted aryl, heteroaryl and substituted heteroaryl;
R(3), R(4), R(5) and R(6)
independently of one another are hydrogen, alkyl having 1, 2, 3, 4, 5, 6, 7, 8, 9 or 10 carbon atoms, O-alkyl having 1, 2, 3, 4, 5, 6, 7, 8, 9 or 10 carbon atoms, halogen, OH, aryl, substituted aryl, heteroaryl, substituted heteroaryl, O-lower alkyl, O-aryl, O-lower alkylaryl, O-substituted aryl, O-lower alkyl-substituted aryl, O—C(═O)—C₁-C₄-alkylaryl, O—C(═O)—NH—C₁-C₄-alkyl, O—C(═O)—N(C₁-C₄-alkyl)₂, NO₂, CN, CF₃, NH₂, NH—C(═O)—C₁-C₄-alkyl, NH—C(═O)—NH₂, COOH, C(═O)—O—C₁-C₄-alkyl, C(═O)—NH₂, C(═O)—NH—C₁-C₄-alkyl, C(═O)—N(C₁—C₄-alkyl)₂, C₁-C₄—COOH, C₁-C₄-alkyl-C(═O)—O—C₁-C₄-alkyl, SO₃H, SO₂-alkyl, SO₂-alkylaryl, SO₂—N-(alkyl)₂, SO₂—N(alkyl)(alkylaryl), C(═O)—R(11), C₁-C₁₀-alkyl-C(═O)—R(11), C₂-C₁₀-alkenyl-C(═O)—R(11), C₂-C₁₀-alkynyl-C(═O)—R(11), NH—C(═O)—C₁-C₁₀-alkyl-C(═O)—R(11) or O—C₁-C₄-alkyl-C(═O)—R(11);
R(11) is C₁-C₄-alkyl, C₁-C₄-alkynyl, aryl, substituted aryl, NH₂, NH—C₁-C₄-alkyl, N—(C₁-C₄-alkyl)₂, SO₃H, SO₂-alkyl, SO₂-alkylaryl, SO₂—N-(alkyl)₂ or SO₂—N(alkyl)(alkylaryl);
X is O, S or NH;
R(7), R(8), R(9) and R(10)
independently of one another are hydrogen, alkyl, cycloalkyl, aryl, alkylaryl;
or
R(8) and R(9)
together are part of a 5-, 6- or 7-membered heterocyclic ring;
A is absent or is a nontoxic organic or inorganic acid;
ak) a benzyloxycarbonylguanidine of the formula 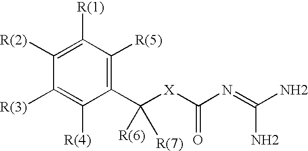
in which:
R(1), R(2) and R(3)
independently of one another are —Y-[4—R(8)-phenyl], —Y-[3—R(8)-phenyl] or —Y-[2—R(8)-phenyl],
where the phenyl is in each case unsubstituted or substituted by 1-2 substituents from the group consisting of F, Cl, —CF₃, methyl, hydroxyl, methoxy and —NR(96)R(97);
R(96) and R(97)
independently of one another are hydrogen or —CH₃;
Y is a bond, CH₂, oxygen, —S— or —NR(9);
R(9) is hydrogen or alkyl having 1, 2, 3 or 4 carbon atoms;
R(8) is SOₐ[NR(98)]_{b}NR(99)R(10);
a is 1 or 2;
b is 0 or 1;
a+b=2;
R(98), R(99) and R(10)
independently of one another are hydrogen, —(C₁-C₈)-alkyl, benzyl, —(C₂-C₈)-alkylene-NR(11)R(12), (C₂-C₈)-alkylene-NR(13)—(C₂-C₈)-alkylene-NR(37)R(38) or (C₀-C₈)-alkylene-CR(39)R(40)CR(41)R(42)(C₀-C₈)-alkylene-NR(43)R(44);
R(11), R(12), R(13), R(37), R(38), R(43) and R(44)
independently of one another are hydrogen, —(C₁-C₈)-alkyl or benzyl;
R(39), R(40), R(41) and R(42)
independently of one another are hydrogen, —(C₁-C₈)-alkyl or —(C₀-C₃)-alkylenephenyl,
where the phenyl is not substituted or is substituted by 1-3 substituents selected from the group consisting of F, Cl, —CF₃, methyl and methoxy;
or
R(99) and R(10)
together are 4-6 methylene groups, of which one CH₂ group can be replaced by oxygen, —S—, —NH—, —N—CH₃ or —N-benzyl;
or
R(8) is SOₐ[NR(98)]_{b}NR(95)—C[═N—R(94)]—NR(93)R(92);
R(92), R(93), R(94) and R(95)
independently of one another are hydrogen or alkyl having 1, 2, 3 or 4 carbon atoms;
or
R(1), R(2) and R(3)
independently of one another are pyrrol-1-yl, pyrrol-2-yl or pyrrol-3-yl,
which is not substituted or is substituted by 1-4 substituents selected from the group consisting of F, Cl, Br, I, —CN, (C₂-C₈)-alkanoyl, (C₂-C₈)-alkoxycarbonyl, formyl, carboxyl, —CF₃, methyl, and methoxy;
or
R(1), R(2) and R(3)
independently of one another are hydrogen, —(C₁-C₈)-alkyl, —(C₂-C₈)-alkenyl or —(CH₂)ₘR(14);
m is zero, 1 or 2;
R(14) is —(C₃-C₈)-cycloalkyl or phenyl,
which is not substituted or is substituted by 1-3 substituents selected from the group consisting of F and Cl, —CF₃, methyl, methoxy and —NR(15)R(16);
R(15) and R(16)
are hydrogen or —CH₃;
or
R(1), R(2) and R(3)
independently of one another are —Q4-[(CH₂)ₖ—CHR(17)—(C═O)R(20)]-phenyl, —Q-3—(CH₂)ₖ—CHR(17)—(C═O)R(20)]-phenyl or —Q-2-[(CH₂)ₖ—CHR(17)—(C═O)R(20)]-phenyl,
where the phenyl in each case is unsubstituted or substituted by 1-2 substituents from the group F, Cl, —CF₃, methyl, hydroxyl, methoxy and —NR(35)R(36);
R(35) and R(36)
independently of one another are hydrogen or —CH₃;
Q is a bond, oxygen, —S— or —NR(18);
R(18) is hydrogen or —(C₁-C₄)-alkyl;
R(17) is —OR(21) or —NR(21)R(22);
R(21) and R(22)
independently of one another are hydrogen, —(C₁-C₈)-alkyl, —(C₁-C₈)-alkanoyl, —(C₁-C₈)-alkoxycarbonyl, benzyl or benzyloxycarbonyl;
or
R(21) is trityl;
R(20) is —OR(23) or —NR(23)R(24);
R(23), R(24) independently of one another are
hydrogen, —(C₁-C₈)-alkyl or benzyl;
k is zero, 1, 2, 3 or 4;
or
R(1), R(2) and R(3)
independently of one another are (C₁-C₉)-heteroaryl,
which is linked via C or N and which is unsubstituted or substituted by 1-3 substituents from the group F, Cl, CF₃, CH₃, methoxy, hydroxyl, amino, methylamino and dimethylamino;
or
R(1), R(2) and R(3)
are —SR(25), —OR(25), —NR(25)R(26) or —CR(25)R(26)R(27);
R(25) is —C_{f}H_{2f}—(C₁-C₉)-heteroaryl,
which is unsubstituted or substituted by 1-3 substituents from the group F, Cl, CF₃, CH₃, methoxy, hydroxyl, amino, methylamino and dimethylamino;
f is zero, 1 or 2;
R(26) and R(27)
independently of one another are defined as R(25) or are hydrogen or (C₁-C₄)-alkyl,
or
R(1), R(2) and R(3)
independently of one another are (C₁-C₉)-heteroaryl-N-oxide, which is linked via C or N and which is unsubstituted or substituted by 1-3 substituents selected from the group consisting of F, Cl, CF₃, CH₃, methoxy, hydroxyl, amino, methylamino and dimethylamino;
or
R(1), R(2) and R(3)
independently of one another are —SR(28), —OR(28), —NR(28)R(29) or —CR(28)R(29)R(30);
R(28) is —C_{g}H_{2g}—(C₁-C₉)-heteroaryl-N-oxide,
which is unsubstituted or substituted by 1-3 substituents selected from the group consisting of F, Cl, CF₃, CH₃, methoxy, hydroxyl, amino, methylamino and dimethylamino;
g is zero, 1 or 2;
R(29), R(30)
independently of one another are defined as R(28), hydrogen or (C₁-C₄)-alkyl;
or
R(1), R(2) and R(3)
independently of one another are hydrogen, F, Cl, Br, I, —C≡N, T—(CH₂)ₕ—(CᵢF₂ᵢ₊₁), R(31)SO_{I}—, R(32)R(33)N—CO—, R(34)—CO— or R(45)R(46)N—SO₂, where the perfluoroalkyl group is straight-chain or branched;
T is a bond, oxygen, —S— or —NR(47);
I is zero, 1 or 2;
h is zero, 1 or 2;
i is 1, 2, 3, 4, 5 or 6;
R(31), R(32), R(34) and R(45)
independently of one another are —(C₁-C₈)-alkyl, —(C₃-C₆)-alkenyl, (CH₂)ₙR(48) or —CF₃;
n is zero, 1, 2, 3 or 4;
R(47) is hydrogen or alkyl with 1, 2 or 3 carbon atoms;
R(48) is —(C₃-C₇)-cycloalkyl or phenyl,
which is not substituted or is substituted by 1-3 substituents selected from the group consisting of F, Cl, —CF₃, methyl, methoxy and
—NR(49)R(50);
R(49) and R(50)
are hydrogen or alkyl having 1, 2, 3 or 4 carbon atoms;
or
R(32), R(34) and R(45)
are hydrogen;
R(33) and R(46)
independently of one another are hydrogen or alkyl having 1, 2, 3 or 4 carbon atoms;
or
R(32) and R(33), and R(45) and R(46)
together are 5 or 6 methylene groups, of which one CH₂ group can be replaced by oxygen, —S—, —NH—, —NCH₃ or —N-benzyl;
or
R(1), R(2) and R(3)
independently of one another are R(51)—A-G-D-;
R(51) is an amino group —NR(52)R(53), an amidino group
R(52)R(53)N—C[═N—R(54)]— or a guanidino group
R(52)R(53)N—C[═N—R(54)]—NR(55)—;
R(52), R(53), R(54) and R(55)
independently of one another are hydrogen or alkyl having 1, 2, 3 or 4 carbon atoms;
or
R(52) and R(53) are
a group C_{γ}H_{2γ};
γ is 4, 5, 6 or 7;
where if γ=5, 6 or 7 a carbon atom of the group C_{γ}H_{2γ} can be replaced by a heteroatom group O, SO_{d} or NR(56),
or
R(53) and R(54) or R(54) and R(55) or R(52) and R(55) are
a group C_{y}H_{2y};
y is 2, 3, 4 or 5;
where if y=3, 4 or 5 a carbon atom of the group C_{y}H_{2y} can be replaced by a heteroatom group O, SO_{d} or NR(56);
d is zero, 1 or 2;
R(56) is hydrogen or methyl;
or
R(51) is a basic heteroaromatic ring system having 1-9 carbon atoms;
A is a group CₑH₂ₑ;
e is zero, 1, 2, 3, 4, 5, 6, 7, 8, 9 or 1;
where in the group CₑH₂ₑ a carbon atom can be replaced by one of the groupings —O—, —CO—, or —SOᵣ—;
r is zero, 1 or 2;
G is a phenylene radical 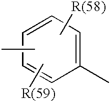
R(58) and R(59)
independently of one another are hydrogen, methyl, methoxy, F, Cl, Br, I, CF₃ or —Soₛ—R(60);
s is zero, 1 or 2;
R(60) is methyl or NR(61)R(62);
R(61) and R(62)
independently of one another are hydrogen or alkyl having 1, 2, 3 or 4 carbon atoms;
D is —CᵥH₂ᵥ—E_{w}—;
v is zero, 1, 2, 3 or 4;
E is —O—, —CO—, —CH[OR(63)]—, —SOₐₐ— or —NR(63)—;
w is zero or 1;
aa is zero, 1 or 2
R(63) is hydrogen or methyl,
or
R(1), R(2) and R(3)
independently of one another are —CF₂R(64), —CF[R(65)][R(66)], —CF[(CF₂)_{q}—CF₃)][R(65)] or —C[(CF₂)ₚ—CF₃]═CR(65)R(66);
R(64) is alkyl having 1, 2, 3 or 4 carbon atoms or cycloalkyl having 3, 4, 5, 6 or 7 carbon atoms;
R(65) and R(66) independently of one another are hydrogen or alkyl having 1, 2, 3 or 4 carbon atoms;
q is zero, 1 or 2;
p is zero, 1 or 2;
or
R(1), R(2) and R(3)
independently of one another are —OR(67) or —NR(67)R(68);
R(67) and R(68)
independently of one another are hydrogen or alkyl having 1, 2, 3, 4, 5 or 6 carbon atoms;
or
R(67) and R(68)
together are 4, 5, 6 or 7 methylene groups, of which one CH₂ group can be replaced by oxygen, —S—, SO₂, —NH—, —NCH₃ or —N-benzyl;
R(4) and R(5)
independently of one another are hydrogen, alkyl having 1, 2, 3 or 4 carbon atoms, F, Cl, —OR(69), —NR(70)R(71) or —C_{z}F_{2z+1};
R(69), R(70) and R(71)
independently of one another are hydrogen or alkyl having 1, 2 or 3 carbon atoms;
z is 1, 2, 3 or 4;
R(6) and R(7)
independently of one another are hydrogen or alkyl having 1, 2, 3 or 4 carbon atoms;
X is oxygen or NR(72);
R(72) is hydrogen or alkyl having 1, 2, 3 or 4 carbon atoms;
or pharmaceutically tolerable salt thereof;
al) an alkenylcarboxylic acid guanidide of the formula 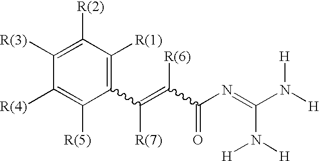
in which:
R(6) is hydrogen, (C₁-C₈)-alkyl, (C₃-C₈)-cycloalkyl or phenyl,
where the phenyl group is not substituted or is substituted by 1-3 substituents selected from the group consisting of F, Cl, CF₃, methyl, methoxy and NR(9)R(10);
R(9) and R(10)
are hydrogen, (C₁-C₄)-alkyl or (C₁-C₄)-perfluoroalkyl;
R(7) independently is defined as R(6);
R(1), R(2), R(3), R(4) and R(5)
independently of one another are hydrogen or F;
where, however, at least one of the radicals R(1), R(2), R(3), R(4) and R(5) must be fluorine;
or pharmaceutically tolerable salt thereof;
am) a benzoylguanidine of the formula 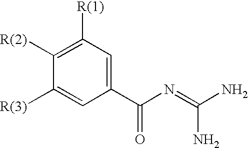
in which:
R(1) is R(4)—SOₘ or R(5)R(6)N—SO₂—;
m is 1 or 2;
R(4) and R(5)
independently of one another are alkyl having 1, 2, 3, 4, 5, 6, 7 or 8 carbon atoms, alkenyl having 3, 4, 5 or 6 carbon atoms, CF₃ or —CₙH₂ₙ—R(7);
n is zero, 1, 2, 3 or 4;
R(6) is H or alkyl having 1, 2, 3 or 4 carbon atoms;
R(7) is cycloalkyl having 3, 4, 5, 6, 7 or 8 carbon atoms or phenyl,
which is not substituted or is substituted by 1-3 substituents selected from the group consisting of F, Cl, CF₃, methyl, methoxy and NR(8)R(9);
R(8) and R(9)
are H or alkyl having 1, 2, 3 or 4 carbon atoms;
or
R(5) is also hydrogen;
or
R(5) and R(6)
together are 4 or 5 methylene groups, of which a CH₂ group can be replaced by oxygen, S, NH, N—CH₃ or N-benzyl;
or
R(1) is —Oₚ—(CH₂)_{q}—(CF₂)ᵣ—CF₃;
p is zero or 1;
q is zero, 1 or 2;
r is zero, 1, 2 or 3;
or
R(1) is —SR(10), —OR(10) or —CR(10)R(11)R(12);
R(10), R(11) and R(12)
independently of one another are hydrogen, alkyl having 1, 2, 3, 4, 5, 6, 7 or 8 carbon atoms, —CₛH₂ₛ—(C₃-C₈)-cycloalkyl or an aromatic system selected from the group consisting of pyridyl, pyrrolyl, quinolyl, isoquinolyl, imidazolyl and phenyl;
s is zero, 1 or 2;
where the aromatic systems pyridyl, pyrrolyl, quinolyl, isoquinolyl, imidazolyl and phenyl are unsubstituted or substituted by 1-3 substituents selected from the group consisting of F, Cl, CF₃, CH₃, methoxy, hydroxyl, amino, methylamino and dimethylamino;
R(2) is —(CH₂)ᵤ—(CF₂)ₜ—CF₃;
t is zero, 1, 2 or 3;
u is zero or 1;
R(3) is hydrogen or independently is defined as R(1);
or pharmaceutically tolerable salt thereof;
an) a substituted cinnamic acid guanidide of the formula 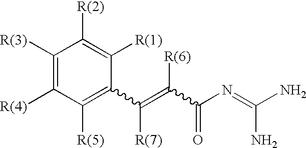
in which:
at least one of the substituents R(1), R(2), R(3), R(4) and R(5) is
—Xₐ—Y_{b}—Lₙ—U;
X is CR(16)R(17), O, S or NR(18);
R(16), R(17) and R(18)
independently of one another are H, alkyl having 1, 2, 3 or 4 carbon atoms or perfluoroalkyl having 1, 2, 3 or 4 carbon atoms;
a is zero or 1;
Y is alkylene having 1, 2, 3, 4, 5, 6, 7 or 8 carbon atoms, alkylene-T having 1, 2, 3, 4, 5, 6, 7 or 8 carbon atoms in the alkylene group, T, T-alkylene having 1, 2, 3, 4, 5, 6, 7 or 8 carbon atoms in the alkylene group;
T is NR(20), O, S or phenylene,
where the phenylene is not substituted or is substituted by 1-3 substituents selected from the group consisting of F, Cl, CF₃, methyl, methoxy and NR(21)R(22);
R(20), R(21) and R(22)
independently of one another are H, alkyl having 1, 2, 3 or 4 carbon atoms or perfluoroalkyl having 1, 2, 3 or 4 carbon atoms;
b is zero or 1;
L is O, S, NR(23) or CₖH₂ₖ;
k is 1, 2, 3, 4, 5, 6, 7 or 8;
n is zero or 1;
U is NR(24)R(25) or an N-containing heterocycle having 1, 2, 3, 4, 5, 6, 7, 8 or 9 carbon atoms;
R(24) and R(25)
independently of one another are hydrogen, alkyl having 1, 2, 3, 4, 5, 6, 7 or 8 carbon atoms or perfluoroalkyl having 1, 2, 3, 4, 5, 6, 7 or 8 carbon atoms;
or
R(24) and R(25)
together are 4 or 5 methylene groups, of which one CH₂ group can be replaced by oxygen, S, NH, N—CH₃ or N-benzyl;
where the N-containing heterocycles are N- or C-bridged and are not substituted or are substituted by 1-3 substituents selected from the group consisting of F, Cl, CF₃, methyl, methoxy and NR(27)R(28);
R(23), R(27) and R(28)
independently of one another are H, alkyl having 1, 2, 3 or 4 carbon atoms or perfluoroalkyl having 1, 2, 3 or 4 carbon atoms;
and the other substituents R(1), R(2), R(3), R(4) and R(5) in each case
independently of one another are H, F, Cl, Br, I, CN, —Oₙ—CₘH₂ₘ₊₁, —Oₚ—(CH₂)ₛ—C_{q}F_{2q+1} or —CᵣH₂ᵣR(10);
n is zero or 1;
m is zero, 1, 2, 3, 4, 5, 6, 7 or 8;
p is zero or 1;
q is 1, 2, 3, 4, 5, 6, 7 or 8;
s is zero, 1, 2, 3 or 4;
r is zero, 1, 2, 3 or 4;
R(10)
is cycloalkyl having 3, 4, 5, 6, 7 or 8 carbon atoms, or phenyl,
where the phenyl is not substituted or is substituted by 1-3 substituents selected from the group consisting of F, Cl, CF₃, methyl, methoxy and NR(11)R(12);
R(11) and R(12)
independently of one another are H, alkyl having 1, 2, 3 or 4 carbon atoms or perfluoroalkyl having 1, 2, 3 or 4 carbon atoms;
R(6) and R(7)
independently of one another are hydrogen, F, Cl, Br, I, CN, alkyl having 1, 2, 3, 4, 5, 6, 7 or 8 carbon atoms, perfluoroalkyl having 1, 2, 3, 4, 5, 6, 7 or 8 carbon atoms, cycloalkyl having 3, 4, 5, 6, 7 or 8 carbon atoms, or phenyl,
which is not substituted or is substituted by 1-3 substituents selected from the group consisting of F, Cl, CF₃, methyl, methoxy and NR(14)R(15);
R(14) and R(15)
independently of one another are H, alkyl having 1, 2, 3 or 4 carbon atoms or perfluoroalkyl having 1, 2, 3 or 4 carbon atoms;
or pharmaceutically tolerable salt thereof;
ao) a benzoylguanidine of the formula 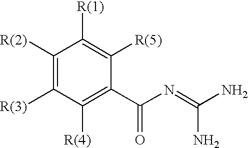
in which:
at least one of the substituents R(1), R(2) and R(3)
is R(6)—C(OH)₂—;
R(6) is perfluoroalkyl having 1, 2 or 3 carbon atoms, which is straight-chain or branched;
and the other substituents R(1), R(2) and R(3)
independently of one another are hydrogen, OH, F, Cl, Br, I, alkyl having 1, 2, 3, 4, 5 or 6 carbon atoms, cycloalkyl having 3, 4, 5 or 6 carbon atoms, alkoxy having 1, 2, 3 or 4 carbon atoms, or phenoxy,
which is unsubstituted or is substituted by 1-3 substituents selected from the group consisting of F, Cl, methyl and methoxy;
or
the other substituents R(1), R(2) and R(3)
independently of one another are alkyl-SOₓ, —CR(7)═CR(8)R(9) or —C≡CR(9);
x is zero, 1 or 2;
R(7) is hydrogen or methyl;
R(8) and R(9)
independently of one another are hydrogen, alkyl having 1, 2, 3 or 4 carbon atoms, cycloalkyl having 3, 4, 5, 6, 7 or 8 carbon atoms, or phenyl,
which is unsubstituted or is substituted by 1-3 substituents selected from the group consisting of F, Cl, CF₃, methyl and methoxy;
or
the other substituents R(1), R(2) and R(3)
independently of one another are phenyl, C₆H₅—(C₁-C₄)-alkyl, naphthyl, biphenylyl, quinolinyl, isoquinolinyl or imidazolyl,
where quinolinyl, isoquinolinyl or imidazolyl are bonded via C or N and where phenyl, C₆H₅—(C₁-C₄)-alkyl, naphthyl, biphenylyl, quinolinyl, isoquinolinyl and imidazolyl are unsubstituted or are substituted by 1-3 substituents selected from the group consisting of F, Cl, CF₃, CH₃, methoxy, hydroxyl, amino, methylamino and dimethylamino;
or
the other substituents R(1), R(2) and R(3)
independently of one another are SR(10), —OR(10), —CR(10)R(11)R(12);
R(10)
is —C_{f}H_{2f}—(C₃-C₈)-cycloalkyl, quinolinyl, isoquinolinyl, pyridinyl, imidazolyl or phenyl,
where the aromatic systems quinolinyl, isoquinolinyl, pyridinyl, imidazolyl and phenyl are unsubstituted or are substituted by 1-3 substituents selected from the group consisting of F, Cl, CF₃, CH₃, methoxy, hydroxyl, amino, methylamino and dimethylamino;
f is zero, 1 or 2;
R(11) and R(12)
independently of one another are defined as R(10), hydrogen or alkyl having 1, 2, 3 or 4 carbon atoms;
R(4) and R(5)
independently of one another are hydrogen, alkyl having 1, 2 or 3 carbon atoms, F, Cl, Br, I, CN, OR(13), NR(14)R(15) or —(CH₂)ₙ—(CF₂)ₒ—CF₃;
R(13), R(14) and R(15)
independently of one another are hydrogen or alkyl having 1, 2, 3 or 4 carbon atoms;
n is zero or 1;
o is zero, 1 or 2;
or pharmacologically acceptable salt thereof;
ap) a sulfonimidamide of the formula 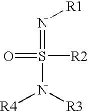
in which:
at least one of the three substituents R(1), R(2) and R(3)
is a benzoylguanidine, 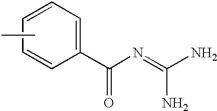
which is unsubstituted or substituted in the phenyl moiety by 1-4 radicals selected from the group consisting of alkyl having 1, 2, 3, 4, 5, 6, 7 or 8 carbon atoms, alkenyl having 2, 3, 4, 5, 6, 7 or 8 carbon atoms, —(CH₂)ₘ—R(14), F, Cl, Br, I, —C≡N, CF₃, R(22)SO₂—, R(23)R(24)N—CO—, R(25)—CO—, R(26)R(27)N—SO₂, —OR(35), —SR(35) or —NR(35)R(36);
m is zero, 1 or 2;
R(14)
is —(C₃-C₈)-cycloalkyl or phenyl,
which is not substituted or is substituted by 1-3 substituents selected from the group consisting of F and Cl, —CF₃, methyl, methoxy and —NR(15)R(16);
R(15) and R(16)
independently of one another are hydrogen or —CH₃;
R(22), R(23), R(25) and R(26)
independently of one another are alkyl having 1, 2, 3, 4, 5, 6, 7 or 8 carbon atoms, alkenyl having 2, 3, 4, 5, 6, 7 or 8 carbon atoms, (CH₂)ₙR(29) or —CF₃;
n is zero, 1, 2, 3 or 4;
R(29) is —(C₃-C₇)-cycloalkyl or phenyl,
which is not substituted or is substituted by 1-3 substituents selected from the group consisting of F, Cl, —CF₃, methyl, methoxy and —NR(30)R(31);
R(30) and R(31)
are hydrogen or alkyl having 1, 2, 3 or 4 carbon atoms;
or
R(23), R(25) and R(26)
are hydrogen;
R(24) and R(27)
independently of one another are hydrogen or alkyl having 1, 2, 3 or 4 carbon atoms;
or
R(23) and R(24), and also R(26) and R(27)
together are 5 or 6 methylene groups, of which one CH₂ group can be replaced by oxygen, —S—, —NH—, —NCH₃ or —N-benzyl;
R(35) and R(36)
independently of one another are hydrogen or alkyl having 1, 2, 3, 4, 5 or 6 carbon atoms;
or
R(35) and R(36)
together are 4-7 methylene groups, of which one CH₂ group can be replaced by oxygen, —S—, —NH—, —NCH₃ or —N-benzyl;
or
R(35) is phenyl,
which is not substituted or is substituted by 1-3 substituents selected from the group consisting of F, Cl, —CF₃, methyl, methoxy, SO₂R(5), SO₂NR(6)R(7) and —NR(32)R(33);
R(5) is alkyl having 1, 2, 3, 4, 5 or 6 carbon atoms;
R(6) and R(7)
independently of one another are hydrogen or alkyl having 1, 2, 3 or 4 carbon atoms;
R(32) and R(33)
independently of one another are hydrogen or alkyl having 1, 2, 3 or 4 carbon atoms;
or
R(35) is C₁-C₉-heteroaryl,
which is unsubstituted or substituted by 1-3 substituents selected from the group consisting of F, Cl, CF₃, CH₃, methoxy, hydroxyl, amino, methylamino and dimethylamino;
and the other substituents R(1), R(2) and R(3) in each case
independently of one another are alkyl having 1, 2, 3, 4, 5, 6, 7 or 8 carbon atoms, (CH₂)ₚR(10);
p is zero, 1, 2, 3 or 4;
R(10) is phenyl,
which is not substituted or is substituted by 1-3 substituents selected from the group consisting of F, Cl, —CF₃, methyl, methoxy, —SO₂NR(17)R(8) and —SO₂R(9);
R(17) and R(8)
independently of one another are hydrogen or alkyl having 1, 2, 3 or 4 carbon atoms;
R(9) is alkyl having 1, 2, 3 or 4 carbon atoms;
or
the other radical R(1) or R(3) in each case
is hydrogen,
R(4) is hydrogen or alkyl having 1, 2, 3 or 4 carbon atoms;
or pharmaceutically tolerable salt thereof;
aq) a benzoylguanidine of the formula 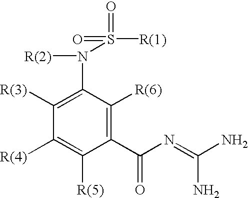
in which:
R(1) is alkyl having 1, 2, 3, 4, 5, 6, 7 or 8 carbon atoms or NR(7)R(8);
R(7) and R(8)
independently of one another are hydrogen or alkyl having 1, 2, 3, 4, 5, 6, 7 or 8 carbon atoms;
R(2) is hydrogen, alkyl having 1, 2, 3, 4, 5, 6, 7 or 8 carbon atoms or —SO₂R(9);
R(9) independently is defined as R(1);
R(3) is hydrogen, —SR(25), —OR(25), —NR(25)R(26) or —CR(25)R(26)R(27);
R(25) is hydrogen, alkyl having 1, 2, 3, 4, 5, 6, 7 or 8 carbon atoms, or phenyl,
which is unsubstituted or substituted by 1-3 substituents selected from the group consisting of F, Cl, CF₃, CH₃, methoxy, hydroxyl, amino, methylamino and dimethylamino;
or
R(25)
is —(C₁-C₉)-heteroaryl,
which is unsubstituted or substituted by 1-3 substituents selected from the group consisting of F, Cl, CF₃, CH₃, methoxy, hydroxyl, amino, methylamino and dimethylamino;
R(26) and R(27)
independently of one another are defined as R(25) or are hydrogen or alkyl having 1, 2, 3, 4, 5, 6, 7 or 8 carbon atoms;
R(4) is hydrogen, F, Cl, Br, I, OH, —C≡N, CF₃, alkyl having 1, 2, 3, 4, 5, 6, 7 or 8 carbon atoms, alkenyl having 2, 3, 4, 5, 6, 7 or 8 carbon atoms or —(CH₂)ₘR(14);
m is zero, 1 or 2;
R(14) is —(C₃-C₈)-cycloalkyl or phenyl,
which is not substituted or is substituted by 1-3 substituents selected from the group consisting of F and Cl, —CF₃, methyl, methoxy and —NR(15)R(16);
R(15) and R(16)
independently of one another are hydrogen or —CH₃;
R(5) and R(6)
independently of one another are hydrogen, alkyl having 1, 2, 3 or 4 carbon atoms, F, Cl, —OR(32), —NR(33)R(34) or CF₃;
R(32), R(33) and R(34)
independently of one another are hydrogen or alkyl having 1, 2, 3 or 4 carbon atoms;
or pharmaceutically tolerable salt thereof;
ar) a benzenedicarboxylic acid diguanidide of the formula 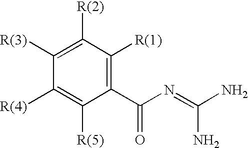
in which:
one of the radicals R(1), R(2), R(3) and R(4)
is —CO—N═C(NH₂)₂;
and of the other radicals R(1), R(2), R(3) and R(4) in each case:
R(1) is hydrogen, alkyl having 1, 2, 3 or 4 carbon atoms, F, Cl, Br, I, —OR(32), —NR(33)R(34) or CF₃;
R(32), R(33) and R(34)
independently of one another are hydrogen or alkyl having 1, 2, 3 or 4 carbon atoms;
R(2) and R(4)
independently of one another are hydrogen, F, Cl, Br, I, OH, —CN, CF₃, —CO—N═C(NH₂)₂, alkyl having 1, 2, 3, 4, 5, 6, 7 or 8 carbon atoms, alkenyl having 2, 3, 4, 5, 6, 7 or 8 carbon atoms or —(CH₂)ₘR(14);
m is zero, 1 or 2;
R(14) is —(C₃-C₈)-cycloalkyl or phenyl,
which is not substituted or is substituted by 1-3 substituents selected from the group consisting of F and Cl, —CF₃, methyl, methoxy and —NR(15)R(16);
R(15) and R(16)
are hydrogen or —CH₃;
or
R(2) and R(4)
independently of one another are pyrrol-1-yl, pyrrol-2-yl or pyrrol-3-yl,
which is not substituted or is substituted by 1-4 substituents selected from the group consisting of F, Cl, Br, I, —CN, (C₂-C₈)-alkanoyl, (C₂-C₈)-alkoxycarbonyl, formyl, carboxyl, —CF₃, methyl and methoxy;
or
R(2) and R(4)
independently of one another are R(22)—SO₂—, R(23)R(24)N—CO—, R(28)—CO— or R(29)R(30)N—SO₂;
R(22) and R(28)
independently of one another are methyl or —CF₃;
R(23), R(24), R(29) and R(30)
independently of one another are hydrogen or methyl;
or
R(2) and R(4)
independently of one another are —OR(35) or —NR(35)R(36);
R(35) and R(36)
independently of one another are hydrogen or alkyl having 1, 2, 3, 4, 5 or 6 carbon atoms;
or
R(35) and R(36)
together are 4-7 methylene groups, of which one CH₂ group can be replaced by oxygen, —S—, —NH—, —NCH₃ or —N-benzyl;
R(3) is hydrogen, —SR(25), —OR(25), —NR(25)R(26) or —CR(25)R(26)R(27);
R(25) is hydrogen, alkyl having 1, 2, 3, 4, 5, 6, 7 or 8 carbon atoms or phenyl,
which is unsubstituted or substituted by 1-3 substituents selected from the group consisting of F, Cl, CF₃, CH₃, methoxy, hydroxyl, amino, methylamino and dimethylamino;
or
R(25) is —(C₁-C₉)-heteroaryl,
which is unsubstituted or substituted by 1-3 substituents selected from the group consisting of F, Cl, CF₃, CH₃, methoxy, hydroxyl, amino, methylamino and dimethylamino;
R(26) and R(27)
independently of one another are defined as R(25) or are hydrogen or alkyl having 1, 2, 3, 4, 5, 6, 7 or 8 carbon atoms;
R(5) is alkyl having 1, 2, 3 or 4 carbon atoms, F, Cl, Br, I, X—(CH₂)_{y}—CF₃ or phenyl,
which is not substituted or is substituted by 1-3 substituents selected from the group consisting of F and Cl, —CF₃, methyl, methoxy and —NR(6)R(7);
R(6) and R(7)
independently of one another are hydrogen or —CH₃;
X is a bond or oxygen;
y is zero, 1 or 2;
or pharmaceutically tolerable salt thereof;
as) a benzenedicarboxylic acid diguanidide of the formula 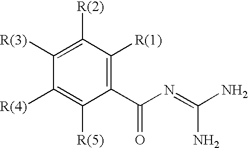
in which:
one of the radicals R(1), R(2), R(3) and R(5)
is —CO—N═C(NH₂)₂;
and of the other radicals R(1), R(2), R(3) and R(5) in each case:
R(1) and R(5)
independently of one another are hydrogen, alkyl having 1, 2, 3 or 4 carbon atoms, F, Cl, —OR(32), —NR(33)R(34) or CF₃;
R(32), R(33) and R(34)
independently of one another are hydrogen or alkyl having 1, 2, 3 or 4 carbon atoms;
R(2) is hydrogen, F, Cl, Br, I, OH, —CN, CF₃, —CO—N═C(NH₂)₂, alkyl having 1, 2, 3, 4, 5, 6, 7 or 8 carbon atoms, alkenyl having 2, 3, 4, 5, 6, 7 or 8 carbon atoms or —(CH₂)ₘR(14);
m is zero, 1 or 2;
R(14) is —(C₃-C₈)-cycloalkyl or phenyl,
which is not substituted or is substituted by 1-3 substituents selected from the group consisting of F and Cl, —CF₃, methyl, methoxy and —NR(15)R(16);
R(15) and R(16)
independently of one another are hydrogen or —CH₃;
or
R(2) is R(22)—SO₂—, R(23)R(24)N—CO—, R(28)—CO— or R(29)R(30)N—SO₂;
R(22) and R(28)
independently of one another are methyl or —CF₃;
R(23), R(24), R(29) and R(30)
independently of one another are hydrogen or methyl;
or
R(2) is —OR(35) or —NR(35)R(36);
R(35) and R(36)
independently of one another are hydrogen or alkyl having 1, 2, 3, 4, 5 or 6 carbon atoms;
or
R(35) and R(36)
together are 4-7 methylene groups, of which one CH₂ group can be replaced by oxygen, —S—, —NH—, —NCH₃ or —N-benzyl;
R(3) is hydrogen, —SR(25), —OR(25), —NR(25)R(26), —CR(25)R(26)R(27);
R(25) is hydrogen, alkyl having 1, 2, 3, 4, 5, 6, 7 or 8 carbon atoms, or phenyl,
which is unsubstituted or substituted by 1-3 substituents selected from the group consisting of F, Cl, CF₃, CH₃, methoxy, hydroxyl, amino, methylamino and dimethylamino;
or
R(25) is —(C₁-C₉)-heteroaryl, which is unsubstituted or substituted by 1-3 substituents selected from the group consisting of F, Cl, CF₃, CH₃, methoxy, hydroxyl, amino, methylamino and dimethylamino;
R(26) and R(27)
independently of one another are defined as R(25) or are hydrogen or alkyl having 1, 2, 3, 4, 5, 6, 7 or 8 carbon atoms;
R(4) is CF₃, alkyl having 1, 2, 3, 4, 5, 6, 7 or 8 carbon atoms, alkenyl having 2, 3, 4, 5, 6, 7 or 8 carbon atoms, —(C₃-C₈)-cycloalkyl or —(CH₂)ₘR(14);
m is 1 or 2;
R(14) is —(C₃-C₈)-cycloalkyl or phenyl,
which is not substituted or is substituted by 1-3 substituents selected from the group consisting of F and Cl, —CF₃, methyl, methoxy and —NR(15)R(16);
R(15) and R(16)
independently of one another are hydrogen or —CH₃;
or
R(4) is phenyl,
which is substituted by 2, 3, 4 or five substituents selected from the group consisting of F, Cl, CF₃, methyl, methoxy and —NR(15)R(16);
R(15) and R(16)
independently of one another are hydrogen or CH₃;
or pharmaceutically tolerable salt thereof;
at) a diaryldicarboxylic acid diguanidide of the formula 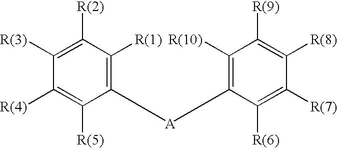
in which:
one of the radicals R(1), R(2), R(3), R(4) and R(5)
is —CO—N═C(NH₂)₂;
the other radicals R(1) and R(5) in each case
independently of one another are hydrogen, alkyl having 1, 2, 3 or 4 carbon atoms, F, Cl, —OR(32), —NR(33)R(34) or CF₃;
R(32), R(33) and R(34)
independently of one another are hydrogen or alkyl having 1, 2, 3 or 4 carbon atoms;
the other radicals R(2) and R(4) in each case
independently of one another are hydrogen, F, Cl, Br, I, OH, —CN, CF₃, —CO—N═C(NH₂)₂, alkyl having 1, 2, 3, 4, 5, 6, 7 or 8 carbon atoms, alkenyl having 2, 3, 4, 5, 6, 7 or 8 carbon atoms or —(CH₂)ₘR(14);
m is zero, 1 or 2;
R(14) is —(C₃-C₈)-cycloalkyl or phenyl,
which is not substituted or is substituted by 1-3 substituents selected from the group consisting of F and Cl, —CF₃, methyl, methoxy and —NR(15)R(16);
R(15) and R(16)
are hydrogen or —CH₃;
or
the other radicals R(2) and R(4) in each case
independently of one another are pyrrol-1-yl, pyrrol-2-yl or pyrrol-3-yl,
which is not substituted or is substituted by 1-4 substituents selected from the group consisting of F, Cl, Br, I, —CN, (C₂-C₈)-alkanoyl, (C₂-C₈)-alkoxycarbonyl, formyl, carboxyl, —CF₃, methyl and methoxy;
or
the other radicals R(2) and R(4) in each case
are R(22)—SO₂—, R(23)R(24)N—CO—, R(28)—CO— or R(29)R(30)N—SO₂;
R(22) and R(28)
independently of one another are methyl or —CF₃;
R(23), R(24), R(29) and R(30)
independently of one another are hydrogen or methyl;
or
the other radicals R(2) and R(4) in each case
independently of one another are —OR(35) or —NR(35)R(36);
R(35) and R(36)
independently of one another are hydrogen or alkyl having 1, 2, 3, 4, 5 or 6 carbon atoms;
or
R(35) and R(36)
together are 4-7 methylene groups, of which one CH₂ group can be replaced by oxygen, —S—, —NH—, —NCH₃ or —N-benzyl;
the other radical R(3) in each case
is hydrogen, —SR(25), —OR(25), —NR(25)R(26), —CR(25)R(26)R(27);
R(25) is hydrogen, alkyl having 1, 2, 3, 4, 5, 6, 7 or 8 carbon atoms, or phenyl,
which is unsubstituted or substituted by 1-3 substituents selected from the group consisting of F, Cl, CF₃, CH₃, methoxy, hydroxyl, amino, methylamino and dimethylamino;
or
R(25) is —(C₁-C₉)-heteroaryl,
which is unsubstituted or substituted by 1-3 substituents selected from the group consisting of F, Cl, CF₃, CH₃, methoxy, hydroxyl, amino, methylamino and dimethylamino;
R(26) and R(27)
independently of one another are defined as R(25) or are hydrogen or alkyl having 1, 2, 3, 4, 5, 6, 7 or 8 carbon atoms;
one of the radicals R(6), R(7), R(8), R(9) and R(10)
is —CO—N═C(NH₂)₂;
the other radicals R(6) and R(10) in each case
independently of one another are hydrogen, alkyl having 1, 2, 3 or 4 carbon atoms, F, Cl, —OR(132), —NR(133)R(134) or CF₃;
R(132), R(133) and R(134)
independently of one another are hydrogen or alkyl having 1, 2, 3 or 4 carbon atoms;
the other radicals R(7) and R(9) in each case
independently of one another are hydrogen, F, Cl, Br, I, OH, —CN, CF₃, —CO—N═C(NH₂)₂, alkyl having 1, 2, 3, 4, 5, 6, 7 or 8 carbon atoms, alkenyl having 2, 3, 4, 5, 6, 7 or 8 carbon atoms or —(CH₂)ₘₘR(114);
mm is zero, 1 or 2;
R(114)
is —(C₃-C₈)-cycloalkyl or phenyl,
which is not substituted or is substituted by 1-3 substituents selected from the group consisting of F and Cl, —CF₃, methyl, methoxy and —NR(115)R(116);
R(115) and R(116)
are hydrogen or —CH₃;
or
the other radicals R(7) and R(9) in each case
independently of one another are pyrrol-1-yl, pyrrol-2-yl or pyrrol-3-yl,
which is not substituted or is substituted by 1-4 substituents selected from the group consisting of F, Cl, Br, I, —CN, (C₂-C₈)-alkanoyl, (C₂-C₈)-alkoxycarbonyl, formyl, carboxyl, —CF₃, methyl and methoxy;
or
the other radicals R(7) and R(9) in each case
are R(122)—SO₂—, R(123)R(124)N—CO—, R(128)—CO— or R(129)R(130)N—SO₂;
R(122) and R(128)
independently of one another are methyl or —CF₃;
R(123), R(124), R(129) and R(130)
independently of one another are hydrogen or methyl;
or
the other radicals R(7) and R(9) in each case
independently of one another are —OR(135) or —NR(135)R(136);
R(135) and R(136)
independently of one another are hydrogen or alkyl having 1, 2, 3, 4, 5 or 6 carbon atoms;
or
R(135) and R(136)
together are 4-7 methylene groups, of which one CH₂ group can be replaced by oxygen, —S—, —NH—, —NCH₃ or —N-benzyl;
the other radical R(8) in each case
is hydrogen, —SR(125), —OR(125), —NR(125)R(126) or —CR(125)R(126)R(127);
R(125)
is hydrogen, alkyl having 1, 2, 3, 4, 5, 6, 7 or 8 carbon atoms or phenyl,
which is unsubstituted or substituted by 1-3 substituents selected from the group consisting of F, Cl, CF₃, CH₃, methoxy, hydroxyl, amino, methylamino and dimethylamino;
or
R(125)
is —(C₁-C₉)-heteroaryl,
which is unsubstituted or substituted by 1-3 substituents selected from the group consisting of F, Cl, CF₃, CH₃, methoxy, hydroxyl, amino, methylamino and dimethylamino;
R(126) and R(127)
independently of one another are defined as R(125) or are hydrogen or alkyl having 1, 2, 3, 4, 5, 6, 7 or 8 carbon atoms;
A is absent or is —NR(11)—CO—, —NR(12)—CO—NR(13)—, —NR(17)—CO—NR(18)—SO₂—, —NR(19)—SO₂—, —SO₂—NR(19)—SO₂—, —SO₂—NR(19)—CO—, —O—CO—NR(19)—SO₂— or —CR(20)═CR(21)—;
R(11), R(12), R(13), R(17), R(18), R(19), R(20) and R(21)
independently of one another are hydrogen or alkyl having 1, 2, 3, 4, 5, 6, 7 or 8 carbon atoms
or pharmaceutically tolerable salt thereof;
au) a substituted thiophenylalkenylcarboxylic acid guanidide of the formula 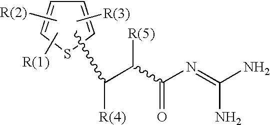
in which:
at least one of the substituents R(1), R(2) and R(3)
is —Oₚ—(CH₂)ₛ—C_{q}F_{2q+1}, R(40)CO— or R(31)SOₖ—;
p is zero or 1;
s is zero, 1, 2, 3 or 4;
q is 1, 2, 3, 4, 5, 6, 7 or 8;
k is zero, 1 or 2;
R(40) is alkyl having 1, 2, 3, 4, 5, 6, 7 or 8 carbon atoms, perfluoroalkyl having 1, 2, 3, 4, 5, 6, 7 or 8 carbon atoms, cycloalkyl having 3, 4, 5, 6, 7 or 8 carbon atoms, or phenyl,
which is not substituted or is substituted by 1-3 substituents selected from the group consisting of F, Cl, CF₃, methyl and methoxy;
R(31) is alkyl having 1, 2, 3, 4, 5, 6, 7 or 8 carbon atoms, perfluoroalkyl having 1, 2, 3, 4, 5, 6, 7 or 8 carbon atoms, cycloalkyl having 3, 4, 5, 6, 7 or 8 carbon atoms, or phenyl, which is not substituted or is substituted by 1-3 substituents selected from the group consisting of F, Cl, CF₃, methyl and methoxy;
or
R(31) is NR(41)R(42);
R(41) and R(42)
independently of one another are hydrogen, alkyl having 1, 2, 3 or 4 carbon atoms or perfluoroalkyl having 1, 2, 3 or 4 carbon atoms,
or
R(41) and R(42)
together are 4 or 5 methylene groups, of which one CH₂ group can be replaced by oxygen, S, NH, N—CH₃ or N-benzyl;
and the other substituents R(1), R(2) and R(3) in each case
independently of one another are H, F, Cl, Br, I, CN, —Oₙₐ—CₘₐH₂ₘₐ₊₁ or —O_{ga}CᵣₐH₂ᵣₐR(10);
na is zero or 1;
ma is zero, 1, 2, 3, 4, 5, 6, 7 or 8;
ga is zero or 1;
ra is zero, 1, 2, 3 or 4;
R(10) is cycloalkyl having 3, 4, 5, 6, 7 or 8 carbon atoms or phenyl,
where the phenyl is not substituted or is substituted by 1-3 substituents selected from the group consisting of F, Cl, CF₃, methyl and methoxy;
R(4) and R(5)
independently of one another are hydrogen, F, Cl, Br, I, CN, alkyl having 1, 2, 3, 4, 5, 6, 7 or 8 carbon atoms, perfluoroalkyl having 1, 2, 3, 4, 5, 6, 7 or 8 carbon atoms, cycloalkyl having 3, 4, 5, 6, 7 or 8 carbon atoms or phenyl,
which is not substituted or is substituted by 1-3 substituents selected from the group consisting of F, Cl, CF₃, methyl, methoxy and NR(14)R(15);
R(14) and R(15)
independently of one another are H, alkyl having 1, 2, 3 or 4 carbon atoms or perfluoroalkyl having 1, 2, 3 or 4 carbon atoms;
or pharmaceutically tolerable salt thereof;
av) an ortho-substituted benzoylguanidine of the formula 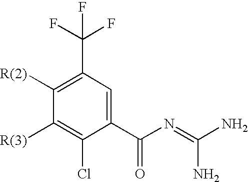
in which:
R(2) and R(3)
independently of one another are hydrogen, Cl, Br, I, (C₁-C₈)-alkyl, (C₃-C₈)-cycloalkyl or —OR(5);
R(5) is (C₁-C₈)-alkyl or —C_{d}H_{2d}—(C₃-C₈)-cycloalkyl;
d is zero, 1 or 2;
where one of the two substituents R(2) and R(3) is always hydrogen but both substituents R(2) and R(3) are not simultaneously hydrogen,
or pharmaceutically tolerable salt thereof;
aw) a benzoylguanidine of the formula 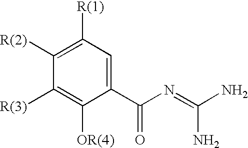
in which:
R(1) is H, F, Cl, Br, I, CN, NO₂, alkyl having 1, 2, 3, 4, 5, 6, 7 or 8 carbon atoms, alkoxy having 1, 2, 3, 4, 5, 6, 7 or 8 carbon atoms, cycloalkyl having 3, 4, 5, 6, 7 or 8 carbon atoms, cycloalkoxy having 3, 4, 5, 6, 7 or 8 carbon atoms or Xₐ—(CH₂)_{b}—(CF₂)_{c}—CF₃;
X is oxygen, S, NR(5),
a is zero or 1;
b is zero, 1 or 2;
c is zero, 1, 2 or 3;
R(5) is H, alkyl having 1, 2, 3 or 4 carbon atoms or —C_{d}H_{2d}R(6);
d is zero, 1, 2, 3 or 4;
R(6) is cycloalkyl having 3, 4, 5, 6, 7 or 8 carbon atoms, phenyl, biphenylyl or naphthyl,
where the aromatics phenyl, biphenylyl or naphthyl are not substituted or are substituted by 1-3 substituents selected from the group consisting of F, Cl, CF₃, methyl, methoxy and NR(7)R(8);
R(7) and R(8)
independently are H or alkyl having 1, 2, 3 or 4 carbon atoms;
or
R(1) is —SR(10), —OR(10) or —CR(10)R(11)R(12);
R(10) is —C_{f}H_{2f}-cycloalkyl having 3, 4, 5, 6, 7 or 8 carbon atoms in the cycloalkyl ring, or phenyl,
where phenyl is unsubstituted or substituted by 1-3 substituents selected from the group consisting of F, Cl, CF₃, CH₃, methoxy, hydroxyl, amino, methylamino and dimethylamino;
f is zero, 1 or 2;
R(11) and R(12)
independently of one another are defined as R(10) or are hydrogen or alkyl having 1, 2, 3 or 4 carbon atoms;
or
R(1) is phenyl, naphthyl, biphenylyl or heteroaryl having 1, 2, 3, 4, 5, 6, 7, 8 or 9 carbon atoms, with the latter being linked via a carbon atom or a nitrogen atom of the ring,
which are in each case unsubstituted or substituted by 1-3 substituents selected from the group consisting of F, Cl, CF₃, CH₃, methoxy, hydroxyl, amino, methylamino and dimethylamino,
or
R(1) is —SR(13), —OR(13), —NHR(13), —NR(13)R(14), —CHR(13)R(15),
—C[R(15)R(16)OH], —C≡CR(18), —C[R(19)]═CHR(18) or
—C[R(20) R(2 1)]ₖ—(CO)—[CR(22)R(23)]ₗ—R(24),
k is zero, 1, 2, 3 or 4;
l is zero, 1, 2, 3 or 4;
R(13) and R(14)
identically or differently are —(CH₂)_{g}—(CHOH)ₕ—(CH₂)ᵢ—(CHOH)_{j—R(}17) or —(CH₂)_{g}—O—(CH₂—CH₂O)ₕ—R(24);
R(17) is hydrogen or methyl,
g, h and i
identically or differently are zero, 1, 2, 3 or 4;
j is 1, 2, 3 or 4;
R(15) and R(16)
identically or differently are hydrogen, alkyl having 1, 2, 3, 4, 5 or 6 carbon atoms or, together with the carbon atom carrying them, are cycloalkyl having 3, 4, 5, 6, 7 or 8 carbon atoms;
R(18)
is phenyl,
which is unsubstituted or substituted by 1-3 substituents selected from the group consisting of F, Cl, CF₃, methyl, methoxy and NR(25)R(26);
R(25) and R(26)
are H or alkyl having 1, 2, 3 or 4 carbon atoms;
or
R(18) is heteroaryl having 1, 2, 3, 4, 5, 6, 7, 8 or 9 carbon atoms,
which is unsubstituted or substituted as phenyl;
or
R(18) is alkyl having 1, 2, 3, 4, 5 or 6 carbon atoms,
which is unsubstituted or substituted by 1-3 OH;
or
R(18)
is cycloalkyl having 3, 4, 5, 6, 7 or 8 carbon atoms;
R(19), R(20), R(21), R(22) and R(23)
identically or differently are hydrogen or methyl;
R(24) is H, alkyl having 1, 2, 3, 4, 5 or 6 carbon atoms, cycloalkyl having 3, 4, 5, 6, 7 or 8 carbon atoms or CₘH₂ₘ—R(18);
m is 1, 2, 3 or 4;
R(2) and R(3)
are defined as R(1);
R(4) is alkyl having 1, 2, 3 or 4 carbon atoms;
or pharmaceutically tolerable salt thereof;
ax) an ortho-substituted benzoylguanidine of the formula 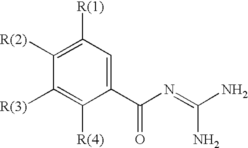
in which:
R(1) is H, F, Cl, Br, I, CN, NO₂, alkyl having 1, 2, 3, 4, 5, 6, 7 or 8 carbon atoms, alkoxy having 1, 2, 3, 4, 5, 6, 7 or 8 carbon atoms, cycloalkyl having 3, 4, 5, 6, 7 or 8 carbon atoms, cycloalkoxy having 3, 4, 5, 6, 7 or 8 carbon atoms or Xₐ—(CH₂)_{b}—(CF₂)_{c}—CF₃;
X is oxygen, S, NR(5),
a is zero or 1;
b is zero, 1 or 2;
c is zero, 1, 2 or 3;
R(5) is H, alkyl having 1, 2, 3 or 4 carbon atoms or —C_{d}H_{2d}R(6);
d is zero, 1, 2, 3 or 4;
R(6) is cycloalkyl having 3, 4, 5, 6, 7 or 8 carbon atoms, phenyl, biphenylyl or naphthyl,
where the aromatics phenyl, biphenylyl or naphthyl are not substituted or are substituted by 1-3 substituents selected from the group consisting of F, Cl, CF₃, methyl, methoxy and NR(7)R(8);
R(7) and R(8)
independently are H or alkyl having 1, 2, 3 or 4 carbon atoms;
or
R(1) is —SR(10), —OR(10) or —CR(10)R(11)R(12);
R(10) is —C_{f}H_{2f}-cycloalkyl having 3, 4, 5, 6, 7 or 8 carbon atoms in the cycloalkyl ring, or phenyl,
where phenyl is unsubstituted or substituted by 1-3 substituents selected from the group consisting of F, Cl, CF₃, CH₃, methoxy, hydroxyl, amino, methylamino and dimethylamino;
f is zero, 1 or 2;
R(11) and R(12)
independently of one another are defined as R(10), or are hydrogen or alkyl having 1, 2, 3 or 4 carbon atoms;
or
R(1) is phenyl, naphthyl, biphenylyl or heteroaryl having 1, 2, 3, 4, 5, 6, 7, 8 or 9 carbon atoms, with the latter being linked via a carbon atom or a nitrogen atom of the ring,
which are in each case unsubstituted or substituted by 1-3 substituents selected from the group consisting of F, Cl, CF₃, CH₃, methoxy, hydroxyl, amino, methylamino and dimethylamino,
or
R(1) is —SR(13), —OR(13), —NHR(13), —NR(13)R(14), —CHR(13)R(15),
—C[R(15)R(16)OH], —C≡CR(18), —C[R(19)]═CHR(18) or
—C[R(20)R(21)]ₖ—(CO)—[CR(22)R(23)]ₗ—R(24),
k is zero, 1, 2, 3 or 4;
l is zero, 1, 2, 3 or 4;
R(13) and R(14)
identically or differently are —(CH₂)_{g}—(CHOH)ₕ—(CH₂)ᵢ—(CHOH)ⱼ—R(17) or —(CH₂)_{g}—O—(CH₂—CH₂O)ₕ—R(24);
R(17) is hydrogen or methyl,
g, h and i
identically or differently are zero, 1, 2, 3 or 4;
j is 1, 2, 3 or 4;
R(15) and R(16)
identically or differently are hydrogen, alkyl having 1, 2, 3, 4, 5 or 6 carbon atoms or, together with the carbon atom carrying them, are cycloalkyl having 3, 4, 5, 6, 7 or 8 carbon atoms;
R(18)
is phenyl,
which is unsubstituted or substituted by 1-3 substituents selected from the group consisting of F, Cl, CF₃, methyl, methoxy and NR(25)R(26);
R(25) and R(26)
are H or alkyl having 1, 2, 3 or 4 carbon atoms;
or
R(18) is heteroaryl having 1, 2, 3, 4, 5, 6, 7, 8 or 9 carbon atoms,
which is unsubstituted or substituted as phenyl;
or
R(18) is alkyl having 1, 2, 3, 4, 5 or 6 carbon atoms,
which is unsubstituted or substituted by 1-3 OH;
or
R(18)
is cycloalkyl having 3, 4, 5, 6, 7 or 8 carbon atoms;
R(19), R(20), R(21), R(22) and R(23)
identically or differently are hydrogen or methyl;
R(24) is H, alkyl having 1, 2, 3, 4, 5 or 6 carbon atoms, cycloalkyl having 3, 4, 5, 6, 7 or 8 carbon atoms or —CₘH₂ₘ—R(18);
m is 1, 2, 3 or 4;
one of the two substituents R(2) and R(3) is hydroxyl;
and
the other of the substituents R(2) and R(3) in each case is defined as R(1);
R(4) is alkyl having 1, 2, 3 or 4 carbon atoms; alkoxy having 1, 2, 3 or 4 carbon atoms, F, Cl, Br, I or —(CH₂)ₙ—(CF₂)ₒ—CF₃;
n is zero or 1;
o is zero or 1;
or pharmaceutically tolerable salt thereof;
ay) a bis-ortho-substituted benzoylguanidine of the formula 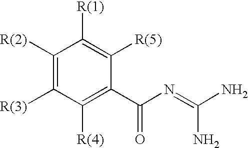
in which:
R(1), R(2) and R(3)
independently of one another are R(10)—SOₐ— or R(14)R(15)N—SO₂—;
a is zero, 1 or 2,
R(10), R(14) and R(15)
independently of one another are alkyl having 1, 2, 3, 4, 5, 6, 7 or 8 carbon atoms, perfluoroalkyl having 1, 2, 3, 4, 5, 6, 7 or 8 carbon atoms, alkenyl having 3, 4, 5 or 6 carbon atoms or —C_{ab}H_{2ab}—R(16);
ab is zero, 1, 2, 3 or 4;
R(16) is cycloalkyl having 3, 4, 5, 6 or 7 carbon atoms, or phenyl,
which is not substituted or is substituted by 1-3 substituents selected from the group consisting of F, Cl, CF₃, methyl, methoxy and NR(17)R(18);
R(17) and R(18)
independently of one another are hydrogen, CF₃ or alkyl having 1, 2, 3 or 4 carbon atoms;
or
R(14) and R(15)
together are 4 or 5 methylene groups, of which one CH₂ group can be replaced by oxygen, sulfur, NH, N—CH₃ or N-benzyl;
or
R(14) and R(15)
are hydrogen;
or
R(1), R(2) and R(3)
independently of one another are SR(21), —OR(22), —NR(23)R(24) or —CR(25)R(26)R(27);
R(21), R(22), R(23) and R(25)
independently of one another are —C_{b}H_{2b}—(C₁-C₉)-heteroaryl,
which is unsubstituted or substituted by 1-3 substituents selected from the group consisting of F, Cl, CF₃, CH₃, methoxy, hydroxyl, amino, methylamino and dimethylamino;
b is zero, 1 or 2;
R(24), R(26) and R(27)
independently of one another are hydrogen, alkyl having 1, 2, 3 or 4 carbon atoms or perfluoroalkyl having 1, 2, 3 or 4 carbon atoms;
or
R(1), R(2) and R(3)
independently of one another are hydrogen, F, Cl, Br, I, CN, (Xₐ)_{dg}—C_{da}H_{2da+1}, —(Xb)_{dh}—(CH₂)_{db}—C_{de}F_{2de+1}, alkenyl having 3, 4, 5, 6, 7 or 8 carbon atoms or —C_{df}H_{2df}R(30);
(Xₐ) is oxygen, sulfur or NR(33);
R(33) is hydrogen, alkyl having 1, 2, 3 or 4 carbon atoms or perfluoroalkyl having 1, 2, 3 or 4 carbon atoms;
dg is zero or 1;
(Xb) is oxygen, sulfur or NR(34);
R(34) is hydrogen, alkyl having 1, 2, 3 or 4 carbon atoms or perfluoroalkyl having 1, 2, 3 or 4 carbon atoms;
dh is zero or 1;
da is zero, 1, 2, 3, 4, 5, 6, 7 or 8;
db is zero, 1, 2, 3 or 4;
de is zero, 1, 2, 3, 4, 5, 6 or 7;
df is zero, 1, 2, 3 or 4;
R(30) is cycloalkyl having 3, 4, 5, 6, 7 or 8 carbon atoms, phenyl, biphenylyl or naphthyl,
where the aromatics phenyl, biphenylyl and naphthyl are not substituted or are substituted by 1-3 substituents selected from the group consisting of F, Cl, CF₃, methyl, methoxy and NR(31)R(32);
R(31) and R(32)
are hydrogen, alkyl having 1, 2, 3 or 4 carbon atoms or perfluoroalkyl having 1, 2, 3 or 4 carbon atoms;
or
R(1), R(2) and R(3)
independently of one another are NR(40)R(41) or —(Xe)—(CH₂)_{eb}R(45);
R(40) and R(41)
independently of one another are hydrogen, alkyl having 1, 2, 3, 4, 5, 6, 7 or 8 carbon atoms, perfluoroalkyl having 1, 2, 3, 4, 5, 6, 7 or 8 carbon atoms or (CH₂)ₑ—R(42);
e is zero, 1, 2, 3 or 4;
R(42) is cycloalkyl having 3, 4, 5, 6 or 7 carbon atoms or phenyl,
which is not substituted or is substituted by 1-3 substituents selected from the group consisting of F, Cl, CF₃, methyl, methoxy and NR(43)R(44);
R(43) and R(44)
independently of one another are hydrogen, CF₃ or alkyl having 1, 2, 3 or 4 carbon atoms;
or
R(40) and R(41) together are 4 or 5 methylene groups, of which one CH₂ group can be replaced by oxygen, sulfur, NH, N—CH₃ or N-benzyl;
(Xe) is oxygen, sulfur or NR(47);
R(47) is hydrogen, alkyl having 1, 2, 3 or 4 carbon atoms or perfluoroalkyl having 1, 2, 3 or 4 carbon atoms;
eb is zero, 1, 2, 3 or 4;
R(45) is cycloalkyl having 3, 4, 5, 6 or 7 carbon atoms, or phenyl,
which is not substituted or is substituted by 1-3 substituents selected from the group consisting of F, Cl, CF₃, methyl, methoxy, NR(50)R(51) and —(Xfa)—(CH₂)_{d}—(Xfb)R(46);
Xfa is CH₂, oxygen, sulfur or NR(48);
Xfb is oxygen, sulfur or NR(49);
R(48), R(49), R(50) and R(51)
independently of one another are hydrogen, alkyl having 1, 2, 3 or 4 carbon atoms or perfluoroalkyl having 1, 2, 3 or 4 carbon atoms;
ed is 1, 2, 3 or 4;
R(46) is hydrogen, alkyl having 1, 2, 3 or 4 carbon atoms or perfluoroalkyl having 1, 2, 3 or 4 carbon atoms;
or
R(1), R(2) and R(3)
independently of one another are —CHR(52)R(53); R(52) is —(CH₂)_{g}—(CHOH)ₕ—(CH)ᵢ—(CHOH)ₖ—R(54) or —(CH₂)_{g}—O—(CH₂—CH₂O)ₕ—R(54);
R(54) is hydrogen or methyl;
g, h, i
identically or differently are zero, 1, 2, 3 or 4;
k is 1, 2, 3 or 4;
R(53) is hydrogen or alkyl having 1, 2, 3 or 4 carbon atoms;
or
R(1), R(2) and R(3)
independently of one another are —C(OH)R(55)R(56);
R(55) and R(56)
identically or differently are hydrogen or alkyl having 1, 2, 3 or 4 carbon atoms;
or
R(55) and R(56)
together are cycloalkyl having 3, 4, 5 or 6 carbon atoms;
or
R(55) is —CH₂OH;
and
R(4) and R(5)
independently of one another are alkyl having 1, 2, 3 or 4 carbon atoms, alkoxy having 1, 2, 3 or 4 carbon atoms, OH, F, Cl, Br, I, CN or —Oₙ—(CH₂)ₒ—(CF₂)ₚ—CF₃;
n is zero or 1;
o is zero, 1 or 2;
p is zero, 1 or 2;
or pharmaceutically tolerable salt thereof;
az) a substituted 1-naphthoylguanidine of the formula 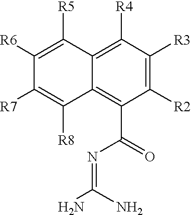
in which:
R2, R3, R4, R5, R6, R7 and RB
independently of one another are H, F, Cl, Br, I, CN, NO₂, CF₃, C₂F₅
or XₐY_{b}Z;
X is O, S, NR(10), CR(11)R(12), C═O, C(═O)NR(10), C(═O)O, SO, SO₂, SO₂NR(10), OC═O, NR(10)C═O or NR(10)SO₂,
where the linkage with the naphthalene ring in each case takes place via the left atom;
R(10), R(11) and R(12)
independently of one another are H, alkyl having 1, 2, 3, 4, 5 or 6 carbon atoms, perfluoroalkyl having 1, 2, 3 or 4 carbon atoms or cycloalkyl having 3, 4, 5, 6 or 7 carbon atoms;
a is zero or 1;
Y is alkylene having 1, 2, 3, 4, 5, 6, 7 or 8 CH₂ groups,
where one of these CH₂ groups can be replaced by O, S, NR(13) or o-, p- or m-phenylene;
R(13) is H, alkyl having 1, 2, 3, 4, 5 or 6 carbon atoms, perfluoroalkyl having 1, 2, 3 or 4 carbon atoms or cycloalkyl having 3, 4, 5 or 6 carbon atoms;
b is zero or 1;
Z is H, alkyl having 1, 2, 3, 4, 5, 6 or 7 carbon atoms, cycloalkyl having 3, 4, 5, 6 or 7 carbon atoms, C(═O)R(15), SO₂R(15), NR(16)R(17) or phenyl,
which is not substituted or is substituted by 1-3 substituents selected from the group consisting of F, Cl, Br, CF₃, methyl, methoxy and NR(21)R(22);
R(21) and R(22)
independently of one another are H or alkyl having 1, 2, 3 or 4 carbon atoms or perfluoroalkyl having 1, 2, 3 or 4 carbon atoms;
R(15) is N═C(NH₂)₂, NR(18)R(19), N(CH₂)_{c}NR(18)R(19) or OR(20);
c is 2 or 3;
R(18) and R(19)
independently of one another are H, alkyl having 1, 2, 3, 4, 5, 6, 7 or 8 carbon atoms or perfluoroalkyl having 1, 2, 3 or 4 carbon atoms;
or
R(18) and R(19)
together are 4 or 5 methylene groups,
of which one CH₂ group can be replaced by oxygen, S, NH, N—CH₃, N-benzyl or N-(p-chlorophenyl);
R(20) is H, alkyl having 1, 2, 3, 4, 5 or 6 carbon atoms, perfluoroalkyl having 1, 2, 3 or 4 carbon atoms or cycloalkyl having 3, 4, 5, 6 or 7 carbon atoms;
R(16) and R(17)
independently of one another are H, alkyl having 1, 2, 3, 4, 5, 6, 7 or 8 carbon atoms or perfluoroalkyl having 1, 2, 3 or 4 carbon atoms;
or
R(16) and R(17)
together are 4 or 5 methylene groups, of which one CH₂ group can be replaced by oxygen, S, NH, N—CH₃, N-benzyl or N-(p-chlorophenyl);
or
Z is an N-containing heterocycle having 1, 2, 3, 4, 5, 6, 7, 8 or 9 carbon atoms,
where the N-containing heterocycle is linked via N or C and is not substituted or is substituted by 1-3 substituents selected from the group consisting of F, Cl, Br, CF₃, methyl, methoxy and NR(21)R(22);
but where, in the case where R(4) is an alkoxy radical, at least one of the substituents R(2), R(3), R(5), R(6), R(7) and R(8) is not hydrogen;
or pharmaceutically tolerable salt thereof;
ba) a substituted 2-naphthoylguanidine of the formula 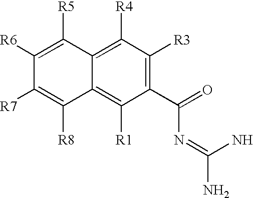
in which:
at least one of the substituents R1, R3, R4, R5, R6, R7 and R8 is
XYₐWZ or X′YₐWZ′;
X is O, S, NR(10) or CR(11)R(12);
R(10), R(11) and R(12)
independently of one another are H, alkyl having 1, 2, 3, 4, 5 or 6 carbon atoms, perfluoroalkyl having 1, 2, 3 or 4 carbon atoms or cycloalkyl having 3, 4, 5, 6 or 7 carbon atoms;
Y is alkylene having 1, 2, 3, 4, 5, 6, 7 or 8 CH₂ groups,
where one of these CH₂ groups can be replaced by O, S, NR(13) or o-, p- or m-phenylene;
R(13) is H, alkyl having 1, 2, 3, 4, 5 or 6 carbon atoms, perfluoroalkyl having 1, 2, 3 or 4 carbon atoms or cycloalkyl having 3, 4, 5 or 6 carbon atoms;
a is zero or 1;
W is CH₂, SO₂, S(═O)(═NH) or, if W does not immediately follow a heteroatom of the group Xyₐ, alternatively O or NR(14);
R(14) is H, alkyl having 1, 2, 3, 4, 5 or 6 carbon atoms, perfluoroalkyl having 1, 2, 3 or 4 carbon atoms or cycloalkyl having 3, 4, 5 or 6 carbon atoms;
Z is C(═O)R(15), SO₂R(15) or, if W is not O or NR(14), alternatively NR(16)R(17);
R(15) is N═C(NH₂)₂, NR(18)R(19), N(CH₂)_{b}NR(18)R(19) or OR(20);
b is 2 or 3;
R(18) and R(19)
independently of one another are H, alkyl having 1, 2, 3, 4, 5, 6, 7 or 8 carbon atoms or perfluoroalkyl having 1, 2, 3 or 4 carbon atoms;
or
R(18) and R(19)
together are 4 or 5 methylene groups, of which one CH₂ group can be replaced by oxygen, S, NH, N—CH₃, N-benzyl or N-(p-chlorophenyl);
R(20) is
H, alkyl having 1, 2, 3, 4, 5 or 6 carbon atoms, perfluoroalkyl having 1, 2, 3 or 4 carbon atoms or cycloalkyl having 3, 4, 5, 6 or 7 carbon atoms;
R(16) and R(17)
independently of one another are H, alkyl having 1, 2, 3, 4, 5, 6, 7 or 8 carbon atoms or perfluoroalkyl having 1, 2, 3 or 4 carbon atoms;
or,
R(16) and R(17)
together are 4 or 5 methylene groups,
of which one CH₂ group can be replaced by oxygen, S, NH, N—CH₃, N-benzyl or N-(p-chlorophenyl);
X′ is C═O, C(═O)NR(30), C(═O)O, SO, SO₂, SO₂NR(30), OC═O, NR(30)C═O or NR(30)SO₂,
where the linkage with the naphthalene ring in each case takes place via the left atom;
R(30) is H, alkyl having 1, 2, 3, 4, 5 or 6 carbon atoms, perfluoroalkyl having 1, 2, 3 or 4 carbon atoms or cycloalkyl having 3, 4, 5, 6 or 7 carbon atoms;
Z is C(═O)R(15), SO₂R(15), an N-containing heterocycle having 1, 2, 3, 4, 5, 6, 7, 8 or 9 carbon atoms,
where the N-containing heterocycle is linked via N or C and is not substituted or is substituted by 1-3 substituents selected from the group consisting of F, Cl, Br, CF₃, methyl, methoxy and NR(21)R(22);
R(21) and R(22)
independently of one another are H, alkyl having 1, 2, 3 or 4 carbon atoms or perfluoroalkyl having 1, 2, 3 or 4 carbon atoms;
R(15) is N═C(NH₂)₂, NR(18)R(19), N(CH₂)_{b}NR(18)R(19) or OR(20);
R(18) and R(19)
independently of one another are H, alkyl having 1, 2, 3, 4, 5, 6, 7 or 8 carbon atoms or perfluoroalkyl having 1, 2, 3 or 4 carbon atoms;
or
R(18) and R(19)
together are 4 or 5 methylene groups,
of which one CH₂ group can be replaced by oxygen, S, NH, N—CH₃, N-benzyl or N-(p-chlorophenyl);
b is 2 or 3;
R(20) is H, alkyl having 1, 2, 3, 4, 5 or 6 carbon atoms, perfluoroalkyl having 1, 2, 3 or 4 carbon atoms or cycloalkyl having 3, 4, 5, 6 or 7 carbon atoms;
or
Z′ if W is not O or NR(14), is NR(16)R(17);
R(16) and R(17)
independently of one another are H, alkyl having 1, 2, 3, 4, 5, 6, 7 or 8 carbon atoms or perfluoroalkyl having 1, 2, 3 or 4 carbon atoms;
or
R(16) and R(17)
together are 4 or 5 methylene groups,
of which one CH₂ group can be replaced by oxygen, S, NH, N—CH₃, N-benzyl or N-(p-chlorophenyl);
and the other substituents R1, R3, R4, R5, R6, R7 and R8 in each case, which are still not allocated by the definitions given above,
independently of one another are H, F, Cl, Br, I, CN, NO₂, CF₃, C₂F₅ or VₚQ_{q}U;
V is O, S, SO, SO₂, NR(60), OC═O, C═O, C(═O)NR(60), C(═O)O or CR(66)R(67);
R(60), R(66) and R(67)
independently of one another are H, alkyl having 1, 2, 3, 4, 5 or 6 carbon atoms, perfluoroalkyl having 1, 2, 3 or 4 carbon atoms or cycloalkyl having 3, 4, 5, 6 or 7 carbon atoms;
p is zero or 1;
Q is alkylene having 1, 2, 3, 4, 5, 6, 7 or 8 CH₂ groups,
where one of these CH₂ groups can be replaced by O, S, NR(68) or o-, p- or m-phenylene;
R(68) is H, alkyl having 1, 2, 3, 4, 5 or 6 carbon atoms, perfluoroalkyl having 1, 2, 3 or 4 carbon atoms or cycloalkyl having 3, 4, 5 or 6 carbon atoms;
q is zero or 1;
U is H, alkyl having 1, 2, 3, 4, 5, 6 or 7 carbon atoms, cycloalkyl having 3, 4, 5, 6 or 7 carbon atoms, C(═O)R(65), SO₂R(65), NR(61)R(62) or phenyl,
which is not substituted or is substituted by 1-3 substituents selected from the group consisting of F, Cl, Br, CF₃, methyl, methoxy and NR(63)R(64);
R(63) and R(64)
independently of one another are H, alkyl having 1, 2, 3 or 4 carbon atoms or perfluoroalkyl having 1, 2, 3 or 4 carbon atoms;
R(65) is N═C(NH₂)₂, NR(61)R(62) or OR(60);
R(61) and R(62)
independently of one another are H, alkyl having 1, 2, 3, 4, 5, 6, 7 or 8 carbon atoms or perfluoroalkyl having 1, 2, 3 or 4 carbon atoms;
or
R(61) and R(62)
together are 4 or 5 methylene groups,
of which one CH₂ group can be replaced by oxygen, S, NH, N—CH₃, N-benzyl or N-(p-chlorophenyl);
or
U is an N-containing heterocycle having 1, 2, 3, 4, 5, 6, 7, 8 or 9 carbon atoms,
where the N-containing heterocycle is linked via N or C and is not substituted or is substituted by 1-3 substituents selected from the group consisting of F, Cl, Br, CF₃, methyl, methoxy and NR(63)R(64);
but where at least one of the substituents R5, R6, R7 and R8 is not hydrogen;
or pharmaceutically tolerable salt thereof;
bb) an ortho-substituted benzoylguanidine of the formula 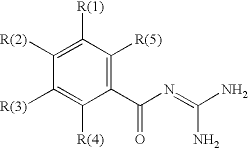
in which:
R(1) is H, F, Cl, Br, I, CN, NO₂, alkyl having 1, 2, 3, 4, 5, 6, 7 or 8 carbon atoms, alkoxy having 1, 2, 3, 4, 5, 6, 7 or 8 carbon atoms, cycloalkyl having 3, 4, 5, 6, 7 or 8 carbon atoms, cycloalkoxy having 3, 4, 5, 6, 7 or 8 carbon atoms or Xₐ—(CH₂)_{b}—(CF₂)_{c}—CF₃;
X is oxygen, sulfur or NR(9);
a is zero or 1;
b is zero, 1 or 2;
c is zero, 1, 2 or 3;
R(9) is H, alkyl having 1, 2, 3 or 4 carbon atoms or —C_{d}H_{2d}R(6);
d is zero, 1, 2, 3 or 4;
R(6) is cycloalkyl having 3, 4, 5, 6, 7 or 8 carbon atoms, phenyl, biphenylyl or naphthyl,
where the aromatics phenyl, biphenylyl or naphthyl are not substituted or are substituted by 1-3 substituents selected from the group consisting of F, Cl, CF₃, methyl, methoxy and NR(7)R(8);
R(7) and R(8) are
independently, H or alkyl having 1, 2, 3 or 4 carbon atoms;
or
R(1) is —SR(10), —OR(10) or —CR(10)R(11)R(12);
R(10) is —C_{f}H_{2f}-cycloalkyl having 3, 4, 5, 6, 7 or 8 carbon atoms in the cycloalkyl ring, heteroaryl having 1, 2, 3, 4, 5, 6, 7, 8 or 9 carbon atoms or phenyl,
where heteroaryl and phenyl are unsubstituted or are substituted by 1-3 substituents selected from the group consisting of F, Cl, CF₃, CH₃, methoxy, hydroxyl, amino, methylamino and dimethylamino;
f is zero, 1 or 2;
R(11) and (R12),
independently of each other, are defined as R(10) or are hydrogen or alkyl having 1, 2, 3 or 4 carbon atoms;
or
R(1) is phenyl, naphthyl, biphenylyl or heteroaryl having 1, 2, 3, 4, 5, 6, 7, 8, or 9 carbon atoms, the latter linked via a carbon or nitrogen atom of the ring,
each of which is unsubstituted or is substituted by 1-3 substituents selected from the group consisting of F, Cl, CF₃, CH₃, methoxy, hydroxyl, amino, methylamino and dimethyl-amino,
or
R(1) is —SR(13), —OR(13), —NHR(13), —NR(13)R(14), —CHR(13)R(15),
—C[R(15)R(16)OH], —C≡CR(18), —C[R(19)]═CHR(18) or
—C[R(20)R(21)]ₖ—(CO)—[CR(22)R(23)]ₗ—R(24),
k is zero, 1, 2, 3 or 4;
l is zero, 1, 2, 3 or 4;
R(13) and R(14),
identically or differently, are
—(CH₂)_{g}—(CHOH)ₕ—(CH₂)ᵢ—(CHOH)ₖₖ—R(17)
or —(CH₂)_{g}—O—(CH₂—CH₂O)ₕ—R(24);
R(17) is hydrogen or methyl,
g, h and i,
identically or differently, are zero, 1, 2, 3 or 4;
kk is 1, 2, 3 or 4;
R(15) and R(16),
identically or differently, are hydrogen, alkyl having 1, 2, 3, 4, 5 or 6 carbon atoms, or, together with the carbon atom carrying them, cycloalkyl having 3, 4, 5, 6, 7 or 8 carbon atoms;
R(18)
is phenyl,
which is unsubstituted or is substituted by 1-3 substituents selected from the group consisting of F, Cl, CF₃, methyl, methoxy and NR(25)R(26);
R(25) and R(26) are
H or alkyl having 1, 2, 3 or 4 carbon atoms;
or
R(18) is heteroaryl having 1, 2, 3, 4, 5, 6, 7, 8 or 9 carbon atoms,
which is unsubstituted or is substituted as phenyl;
or
R(18) is alkyl having 1, 2, 3, 4, 5 or 6 carbon atoms,
which is unsubstituted or is substituted by 1-3 OH;
or
R(18)
is cycloalkyl having 3, 4, 5, 6, 7 or 8 carbon atoms;
R(19), R(20), R(21), R(22) and R(23),
identically or differently, are hydrogen or methyl;
R(24) is H, alkyl having 1, 2, 3, 4, 5 or 6 carbon atoms, cycloalkyl
having 3, 4, 5, 6, 7 or 8 carbon atorms or —CₘH₂ₘ—R(18);
m is 1, 2, 3 or 4;
one of the two substituents R(2) and R(3) is
—O—CO—R(27);
R(27) is alkyl having 1, 2, 3, 4, 5, 6, 7 or 8 carbon atoms, cycloalkyl having 3, 4, 5, 6, 7 or 8 carbon atoms, phenyl, biphenylyl, naphthyl, pyridyl or quinolinyl,
where phenyl, biphenylyl, naphthyl, pyridyl or quinolyl are unsubstituted or substituted by 1-3 substituents selected from the group consisting of F, Cl, CF₃, methyl, methoxy and NR(7)R(8);
R(7) and R(8)
independently of one another are hydrogen or alkyl having 1, 2, 3 or 4 carbon atoms;
where one of the substituents R(2) and R(3)
is always defined as R(1);
R(4) and R(5)
independently of one another are hydrogen, alkyl having 1, 2, 3 or 4 carbon atoms, alkoxy having 1, 2, 3 or 4 carbon atoms, F, Cl, Br, I, CN or —(CH₂)ₙ—(CF₂)ₒ—CF₃,
n is zero or 1,
o is zero or 1,
or pharmaceutically tolerable salt thereof;
bc) a benzoylguanidine of the formula 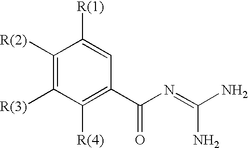
in which:
R(1) is R(13)—SOₘ or R(14)R(15)N—SO₂—;
m is 1 or 2;
R(13) is alkyl having 1, 2, 3, 4, 5, 6, 7 or 8 carbon atoms, perfluoroalkyl having 1, 2, 3, 4, 5, 6, 7 or 8 carbon atoms, alkenyl having 3, 4, 5, 6, 7 or 8 carbon atoms or —CₙH₂ₙ—R(16),
n is zero, 1, 2, 3 or 4;
R(16) is cycloalkyl having 3, 4, 5, 6, 7 or 8 carbon atoms, phenyl, biphenylyl or naphthyl,
where phenyl, biphenylyl and naphthyl are not substituted or are substituted by 1-3 substituents selected from the group consisting of F, Cl, CF₃, methyl, methoxy and NR(25)R(26);
R(25) and R(26) are,
independently of each other, hydrogen, alkyl having 1, 2, 3 or 4 carbon atoms or perfluoroalkyl having 1, 2, 3 or 4 carbon atoms;
R(14) is hydrogen, alkyl having 1, 2, 3, 4, 5, 6, 7 or 8 carbon atoms, perfluoroalkyl having 1, 2, 3, 4, 5, 6, 7 or 8 carbon atoms, alkenyl having 3, 4, 5, 6, 7 or 8 carbon atoms or —CₙH₂ₙ—R(27),
n is zero, 1, 2, 3 or 4;
R(27) is cycloalkyl having 3, 4, 5, 6, 7 or 8 carbon atoms, phenyl, biphenylyl or naphthyl,
where phenyl, biphenylyl and naphthyl are not substituted or are substituted by 1-3 substituents selected from the group consisting of F, Cl, CF₃, methyl, methoxy and NR(28)R(29);
R(28) and R(29) are,
independently of each other, hydrogen, alkyl having 1, 2, 3 or 4 carbon atoms or perfluoroalkyl having 1, 2, 3 or 4 carbon atoms;
R(15) is hydrogen, alkyl having 1, 2, 3 or 4 carbon atoms or perfluoroalkyl having 1, 2, 3 or 4 carbon atoms;
or
R(14) and R(15) are,
together, 4 or 5 methylene groups of which one CH₂ group can be replaced by oxygen, S, NH, N—CH₃ or N-benzyl;
one of the substituents R(2) and R(3)
is hydrogen;
and the other of the substituents R(2) and R(3) in each case is
—CHR(30)R(31);
R(30) is
—(CH₂)_{g}—(CHOH)ₕ—(CH₂)ᵢ—(CHOH)ₖ—R(32) or
—(CH₂)_{g}—O—(CH₂—CH₂O)ₕ—R(24);
R(24) and R(32) are,
independently of each other, hydrogen or methyl;
g, h and i are,
identically or differently, zero, 1, 2, 3 or 4;
k is 1, 2, 3 or 4;
or the other of the substituents R(2) and R(3) in each case is
—C(OH)R(33)R(34);
R(31), R(33) and R(34) are,
identically or differently, hydrogen or alkyl having 1, 2, 3 or 4 carbon atoms,
or
R(33) and R(34) are,
together, cycloalkyl having 3, 4, 5 or 6 carbon atoms;
or
R(33) is —CH₂OH;
R(4) is alkyl having 1, 2, 3 or 4 carbon atoms, alkoxy having 1, 2, 3 or 4 carbon atoms, F, Cl, Br, I, CN or —(CH₂)ₙ—(CF₂)ₒ—CF₃;
n is zero or 1;
o is zero, 1 or 2;
or pharmaceutically tolerable salt thereof;
bd) an indanylidineacetylguanidine of the formula 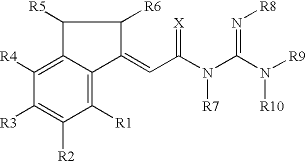
in which:
R1, R2, R3, R4, R5 and R6
independently of one another are H, C₁-C₁₀-alkyl; haloalkyl having 1-6 carbon atoms, O—C₁-C₁₀-alkyl, haloalkoxy having 1-6 carbon atoms, F, Cl, Br, I, aryl, substituted aryl, heteroaryl, substituted heteroaryl, OH, O-lower alkyl, O-aryl, O-lower alkylaryl, O-substituted aryl, O-lower alkyl-substituted aryl, O—C(═O)—C₁-C₄-alkylaryl, O—C(═O)—NH—C₁-C₄-alkyl, O—C(═O)—N(C₁-C₄-alkyl)₂, NO₂, CN, CF₃, NH₂, NH—C(═O)—C₁-C₄-alkyl, NH—C(═O)—NH₂, COOH, C(═O)—O—C₁-C₄-alkyl, C(═O)—NH₂, C(═O)—NH—C₁-C₄-alkyl, C(═O)—N(C₁-C₄-alkyl)₂, C₁-C₄—COOH, C₁-C₄-alkyl-C(═O)—O—C₁-C₄-alkyl, SO₃H, SO₂-alkyl; SO₂-alkylaryl, SO₂—N-(alkyl)₂, SO₂—N(alkyl)(alkylaryl), C(═O)—R11, C₁-C₁₀-alkyl-C(═O)—R11, C₂-C₁₀-alkenyl-C(═O)—R11, C₂-C₁₀-alkynyl-C(═O)—R11, NH—C(═O)—C₁-C₁₀-alkyl-C(═O)—R11 or O—C₁-C₁₁-alkyl-C(═O)—R11;
R11 is C₁-C₄-alkyl, C₁-C₄-alkynyl, aryl, substituted aryl, NH₂, NH—C₁-C₄-alkyl, N-(C₁-C₄-alkyl)₂, SO₃H, SO₂-alkyl, SO₂-alkylaryl, SO₂—N-(alkyl)₂ or SO₂—N(alkyl)(alkylaryl);
X is O, S or NH;
R7, R8, R9 and R10
independently of one another are H, alkyl, cycloalkyl, aryl, alkylaryl,
or
R8 and R9
together are part of a 5-, 6- or 7-membered heterocyclic ring;
or pharmaceutically acceptable salt thereof;
be) a phenyl-substituted alkenylcarboxylic acid guanidide of the formula 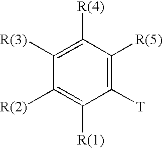
in which:
T is 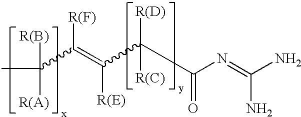
R(A) is hydrogen, F, Cl, Br, I, CN, OH, OR(6), (C₁-C₄)-alkyl,
Oᵣ(CH₂)ₐC_{b}F_{2b+1}, (C₃-C₈)-cycloalkyl or NR(7)R(8)
r is zero or 1;
a is zero, 1, 2, 3 or 4;
b is 1, 2, 3 or 4;
R(6) is (C₁-C₄)-alkyl, (C₁-C₄)-perfluoroalkyl, (C₃-C₆)-alkenyl, (C₃-C₈)-cycloalkyl, phenyl or benzyl,
the phenyl nucleus not being substituted or being substituted by 1-3 substituents selected from the group consisting of F, Cl, CF₃, methyl, methoxy and NR(9)R(10);
R(9) and R(10)
are H, (C₁-C₄)-alkyl or (C₁-C₄)-perfluoro-alkyl;
R(7) and R(8)
independently of one another are defined as R(6);
or
R(7) and R(8)
together are 4 or 5 methylene groups, of which one CH₂ group can be replaced by oxygen, sulfur, NH, N—CH₃ or N-benzyl;
R(B), R(C) and R(D)
independently are defined as R(A);
x is zero, 1 or 2;
y is zero, 1 or 2;
R(F) is hydrogen, F, Cl, Br, I, CN, OR(12), (C₁-C₈)-alkyl,
Oₚ(CH₂)_{f}C_{g}F_{2g+1}, (C₃-C₈)-cycloalkyl or (C₁-C₉)-heteroaryl;
p is zero or 1;
f is zero, 1, 2, 3 or 4;
g is 1, 2, 3, 4, 5, 6, 7 or 8;
R(12) is (C₁-C₈)-alkyl, (C₁-C₄)-perfluoroalkyl, (C₃-C₈)-alkenyl, (C₃-C₈)-cycloalkyl, phenyl or benzyl,
the phenyl nucleus not being substituted or being substituted by 1-3 substituents selected from the group consisting of F, Cl, CF₃, methyl, methoxy and NR(13)R(14);
R(13) and R(14)
are H, (C₁-C₄)-alkyl or (C₁-C₄)-perfluoroalkyl;
R(E) is defined independently as R(F);
R(1) is defined independently as T;
or
R(1) is hydrogen, —OₖCₘH₂ₘ₊₁, —Oₙ(CH₂)ₚC_{q}F_{2q+1}, F, Cl, Br, I, CN, —(C═O)—N═C(NH₂)₂, —SoᵣR(17), —Soᵣ₂NR(31)R(32), —Oᵤ(CH₂)ᵥC₆H₅, —Oᵤ₂—(C₁-C₉)-heteroaryl or —Sᵤ₂—(C₁-C₉)-heteroaryl;
k is zero or 1;
m is zero, 1, 2, 3, 4, 5, 6, 7 or 8;
n is zero or 1;
p is zero, 1, 2, 3 or 4;
q is 1, 2, 3, 4, 5, 6, 7 or 8;
r is zero, 1 or 2;
r2 is zero, 1 or 2;
R(31) and R(32)
independently of one another are hydrogen, (C₁-C₈)-alkyl or (C₁-C₈)-pernluoroalkyl;
or
R(31) and R(32)
are, together, 4 or 5 methylene groups of which one CH₂ group can be replaced by oxygen, S, NH, N—CH₃ or N-benzyl;
R(17) is (C₁-C₈)-alkyl;
u is zero or 1;
u2 is zero or 1;
v is zero, 1, 2, 3 or 4;
where the phenyl nucleus is not substituted or is substituted by 1-3 substituents selected from the group consisting of F, Cl, CF₃, methyl, methoxy, —(CH₂),NR(21)R(22), NR(18)R(19) and (C₁-C₉)-heteroaryl;
R(18), R(19), R(21) and R(22)
independently of one another are (C₁-C₄)-alkyl or (C₁-C₄)-perfluoroalkyl;
w is 1, 2, 3 or 4;
where the heterocycle of the (C₁-C₉)-heteroaryl is unsubstituted or substituted by 1-3 substituents selected from the group consisting of F, Cl, CF₃, methyl and methoxy;
R(2), R(3), R(4) and R(5)
independently of one another are defined as R(1),
or
R(1) and R(2) or R(2) and R(3)
in each case together are —CH—CH═CH—CH—,
which is not substituted or is substituted by 1-3 substituents selected from the group consisting of F, Cl, CF₃, methyl, methoxy, —(CH₂)_{w2}NR(24)R(25) and NR(26)R(27);
R(24), R(25), R(26) and R(27)
are H, (C₁-C₄)-alkyl or (C₁-C₄)-perfluoroalkyl;
w2 is 1, 2, 3 or 4;
the radical T being present in the molecule at least twice, but only three times at most;
or pharmaceutically tolerable salt thereof; or
bf) a benzoylguanidine of the formula 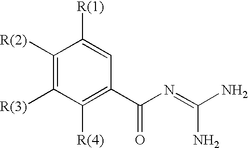
in which:
R(1) is CF₃;
one of the substituents R(2) and R(3)
is hydrogen;
and the other substituent R(2) or R(3) in each case
is —C(OH)(CH₃)—CH₂OH, —CH(CH₃)—CH₂OH or —C(OH)(CH₃)₂;
R(4) is methyl, methoxy, Cl or CF₃;
or pharmaceutically tolerable salt thereof.

3. A method as claimed in claim 1, wherein the inhibitor of the Na⁺/H⁺ exchanger is:
A. a compound of the formula 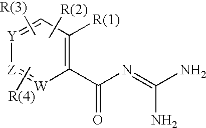
in which:
W, Y and Z
are a nitrogen atom or a carbon atom substituted by R(2) or R(3) or R(4);
R(1) is hydrogen, A, Hal, —CF₃, —CH₂F, —CHF₂, —CH₂CF₃, —C₂F₅, —CN, —NO₂, -ethynyl, or an X—R′;
A is alkyl having 1 to 6 carbon atoms;
Hal is F, Cl, Br or I;
X is oxygen, S or NR″;
R″ is hydrogen, A or a cyclic methylene chain having 3 to 7 carbon atoms;
R′ is H, A, HO—A—, HOOC—A—, (C₃-C₇)-cycloalkyl, (C₆-C₈)-cycloalkylalkyl, CF₃, CH₂F, CHF₂, CH₂—CF₃, Ph, —CH₂—Ph or Het;
Ph is phenyl, naphthyl or biphenylyl which is unsubstituted or mono-, di- or trisubstituted by A, OA, NR′R″, Hal or CF₃;
Het is a mono- or binuclear saturated, unsaturated or aromatic heterocycle having 1 to 4 nitrogen, oxygen or sulfur atoms,
which is unsubstituted or mono-, di- or trisubstituted by Hal, CF₃, A, OH, OA, —X—R′, —CN, —NO₂, or carbonyl oxygen,
where Het is bonded via N or an alkylene chain CₘH₂ₘ where m=zero to 6;
or
R′ and R″
together are alkylene having 4-5 carbon atoms, in which one CH₂ group can also be replaced by oxygen, S, NH, N—A, N—Ph and N—CH₂—Ph;
R(2) and R(3)
independently of one another are hydrogen, Hal, A, HO—A—, X—R′, —C(═N—OH)—A, A—O—CO—(C₁-C₄)-alkyl-, CN, NO₂, COOH, CF₃, CH₂F, CHF₂, C₂F₅, CH₂CF₃, or S(O)ₙR′″;
R′″ is A, Ph or —Het;
n is zero, 1 or 2;
or
R(2) and R(3)
independently of one another are SO₂NR′R″, Ph or —O—Ph, —O—CH₂—Ph, —CO—A, —CHO, —COOA, —CSNR′R″, CONR′R″, —CH═CH—COOH, —CH═CH—COOA, indenyl, indanyl, decahydronaphthyl, cyclopentenyl, dihydrothienyl, dihydrofuryl, heterobicyclyl, alkylthienyl, halothienyl, haloalkylthienyl, acylthienyl, halofuryl, haloalkylfuryl or pyrrolyl;
or
R(2) and R(3)
independently of one another are R(5)—O—;
R(5) is hydrogen, A, (C₁-C₆)-alkenyl or (C₃-C₇)-cycloalkyl;
R(4) is Ph, Het, —O-Het; CF₃, S(O)ᵣR′″, —SO₂NR′R″, alk; 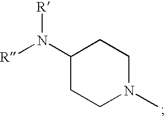
or
two of the substituents R(1) to R(4)
together are a group —O—CR(6)R(7)—CO—NR(8)—, 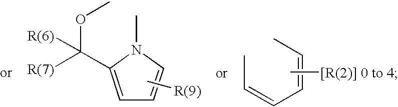
where R(2) has the meaning indicated;
R(6), R(7), R(8) and R(9)
independently of one another are H or A;
or
R(8) is (C₅-C₇)-cycloalkyl;
or
R(9) is cyano;
alk is straight-chain or branched (C₁-C₈)-alkyl or (C₃-C₈)-cycloalkyl, which is unsubstituted or mono-, di- or trisubstituted by A;
or
alk is an ethenyl or ethynyl radical which is substituted by H, A, Ph or Het;
B. a compound of the formula 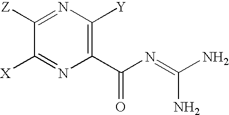
in which:
X is H, Hal, (Hal)₃C—, (C₁-C₆)-alkyl, (C₃-C₆)-cycloalkyl, substituted phenyl, (C₁-C₅)-alkyl-S— or (C₁-C₅)-alkyl-SO₂—;
Y is NH₂ or substituted amino;
or
X and Z
together are a —(CH₂)₄— or a 1,3-butadienylene chain;
or
Z is H, Hal, OH, HS, (C₁-C₅)-alkyl, (C₃-C₆)-cycloalkyl or substituted phenyl;
or
Z is an amino group —NR(1)R(2);
R(1) is H, straight- and branched-chain, optionally substituted (C₁-C₈)-alkyl,
which can be interrupted by oxygen;
or
R(1) is (C₃-C₈)-alkenyl, (C₃-C₈)-alkynyl, (C₃-C₇)-cycloalkyl or OH-substituted phenyl or OH-substituted phenyl-(C₁-C₄)-alkyl or OH-substituted (C₃-C₇)-cycloalkyl;
R(2) is 1-morpholino, hydrogen or a straight or branched (C₁-C₈)-alkyl chain,
which can be interrupted by oxygen or an amino group,
which straight or branched (C₁-C₈)-alkyl chain is unsubstituted or substituted by
a substituted or unsubstituted mono- or polynuclear heterocycle which contains a nitrogen, oxygen or sulfur atom;
or
which alkyl chain is substituted by phenyl,
optionally mono- or polysubstituted by (C₁-C₄) alkoxy, optionally substituted by OH, alkylamino, alkyl or phenyl;
or
by an aminocarbonyl group
or
by hydroxyl or (C₁-C₄)-alkoxy group,
or
R(2) is phenyl,
unsubstituted or substituted by alkyl, alkoxy or an amino group, which as substituents carries:
H, a mono- or polynuclear heterocycle which contains a nitrogen, oxygen or sulfur atom,
which is unsubstituted or substituted by H, Hal or (C₁-C₄)-alkyl; or
a phenyl radical,
unsubstituted or substituted by a substituent selected from the group consisting of (C₁-C₄)-alkyl, (C₁-C₄)-alkoxy, Hal and OH;
or
R(2) is 1-piperidino,
unsubstituted or substituted in the 4-position by an acyl radical of an aliphatic, alicyclic, aromatic or heteroaromatic carboxylic acid, (C₁-C₈) alkyl, which for its part can be substituted by OH or (C₁-C₄)-alkoxy or a (C₁-C₄)-alkoxy-substituted phenyl radical;
or
R(2) is amidino,
which is unsubstituted or substituted by phenyl,
which is unsubstituted or substituted by Hal or alkyl;
or
R(2) is an acyl radical of an aliphatic, alicyclic, aromatic or heteroaromatic carboxylic acid,
or
R(2) is a (C₁-C₈) alkyl chain, which can be substituted by a phenyl radical carrying an OH, alkoxy or alkyl radical;
or
R(1) and R(2)
together with the nitrogen atom to which they are bonded, are a piperazine ring,
which is unsubstituted or, via a (C₁-C₆)-methylene chain, carries a mono- or polynuclear heterocycle,
which contains nitrogen, oxygen or sulfur
Hal is F, Cl, Br or I;
C. an indoloylguanidine derivative of the formula 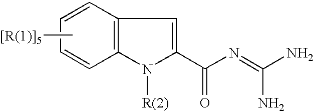
in which
R(2) is hydrogen, unsubstituted or substituted (C₁-C₈)-alkyl, (C₃-C₇)-cycloalkyl, OH, (C₁-C₆)-alkyl-O—, an aromatic radical or a group —CH₂—R(20);
R(20) is (C₂-C₆)-alkenyl or (C₂-C₆)-alkynyl;
R(1) is 1 to 5 identical or different substituents, which are: hydrogen, unsubstituted or substituted (C₁-C₈)-alkyl, (C₂-C₆)-alkenyl, (C₂-C₆)-alkynyl, (C₃-C₇)-cycloalkyl, halogen, —NO₂, (C₂-C₈)-alkanoyl, arylalkanoyl having up to 10 carbon atoms, aroyl having up to 11 carbon atoms, —COOH, (C₂-C₆)-alkoxycarbonyl, an aromatic group or one of the following mentioned groups: —OR(3), —NR(6)R(7) or —S(O)ₙR(40);
R(3) is hydrogen, (C₁-C₈)-alkyl, substituted (C₁-C₈)-alkyl, (C₃-C₇)-cycloalkyl, an aromatic radical or a group —CH₂—R(30); R(30) is alkenyl or alkynyl;
R(6) and R(7)
independently of one another are hydrogen, unsubstituted or substituted (C₁-C₈)-alkyl, (C₃-C₇)-cycloalkyl, (C₂-C₈)-alkanoyl, an arylalkanoyl group having up to 10 carbon atoms, an aroyl group having up to 11 carbon atoms, an aromatic group or —CH₂—R(60);
R(60) is (C₂-C₆)-alkenyl or (C₂-C₆)-alkynyl;
or
R(6) and R(7)
together with the nitrogen atom are a 5-7-membered cyclic amine, which can additionally contain further heteroatoms in the ring;
n is zero, 1 or 2;
R(40) is unsubstituted or substituted (C₁-C₈)-alkyl, or an aromatic group, or a group 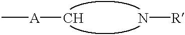
A is oxygen, —S(O)ₙ— or —NR(50)—;
R(50) is hydrogen or (C₁-C₈)-alkyl;
R′ is hydrogen, unsubstituted or substituted (C₁-C₈)-alkyl, in which the ring represents a saturated 3-8-membered heterocycle having a nitrogen atom,
said substituted alkyl carries one or more groups selected from the group consisting of halogen, —OH, (C₁-C₆)-alkoxy, —CN, —COOH, (C₂-C₆)-alkoxycarbonyl, (C₂-C₈)-alkanoyl, arylalkanoyl having up to 10 carbon atoms, aroyl having up to 11 carbon atoms, an aromatic group and —CONR(4)R(5),
R(4) and R(5)
identically or differently are hydrogen or (C₁-C₈)-alkyl;
or
R(4) and R(5)
are connected to one another and together form a 5-7-membered cyclic amine which can additionally contain further heteroatoms in the ring,
or said substituted alkyl carries a group 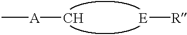
in which:
E is a nitrogen atom or a CH group;
R″ is hydrogen, (C₁-C₈)-alkyl which is unsubstituted or substituted by OH or substituted (C₁-C₈)-alkyl, (C₁-C₆)-alkoxy, —CN, —COOH, (C₂-C₆)-alkoxycarbonyl, (C₂-C₈)-alkanoyl, aralkanoyl having up to 10 carbon atoms, aroyl having up to 11 carbon atoms, an aromatic group, —NR(6)R(7) or —CONR(4)R(5);
R(4) and R(5)
independently of one another are hydrogen or (C₁-C₈)-alkyl;
where the cyclic system of the formula 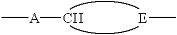
is a 3-8-membered saturated aliphatic or heterocyclic ring system having a nitrogen atom,
and where the aromatic groups mentioned are an aryl radical having up to 10 carbon atoms, a 5- or 6-membered heteroaryl radical having 1-4 nitrogen atoms, a 5- or 6-membered heteroaryl group containing 1 or 2 nitrogen atoms and a heteroatom which is oxygen or sulfur, or furyl, and where the aryl radicals mentioned can be unsubstituted or substituted by unsubstituted (C₁-C₈)-alkyl or substituted (C₁-C₈)-alkyl, halogen, —NO₂, (C₂-C₆)-alkoxycarbonyl, COOH, —OR(3), NR(6)R(7), —CONR(4)R(5), —SO₂NR(6)R(7) or S(O)ₙR(40),
where R(1) and the guanidinocarbonyl radical can be in any desired position of the 5- or 6-membered ring of the indole system,
or pharmaceutically tolerable salt thereof;
D. a heterocyclic guanidine derivative of the formula 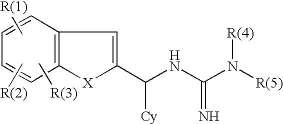
in which:
X is —O—, —S—, —NH—, —N[(C₁-C₄)-alkyl]- or —N(phenyl)-;
R(1), R(2) and R(3)
are hydrogen, halogen, (C₁-C₄)-alkyl, (C₁-C₄)-alkyl-O—, phenyl or benzyl;
or
two of the substituents R(1), R(2) and R(3)
together with one side of the benzo system are a 4-6-membered carbocyclic ring;
R(4) and R(5)
independently of one another are hydrogen, (C₁-C₁₂)-alkyl, benzhydryl, aralkyl,
which is unsubstituted or substituted by one or more substituents from the groups halogen, (C₁-C₄)-alkyl, (C₁-C₄)-alkyl-O—, —CF₃ and —(CH₂)ₘ—CH₂—T,
m is zero to 3;
T is —CO—O—T(1);
T(1) is hydrogen or (C₁-C₄)-alkyl;
Cy is a benzo-fused unsaturated or dihydro-5-membered ring heterocycle 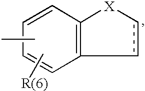
a pyrazole or imidazole ring of the formula 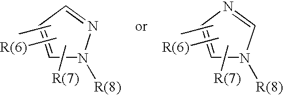
a naphthyl radical or a dihydro- or tetrahydronaphthyl radical 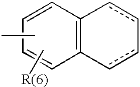
a 2-, 3- or 4-pyridyl radical 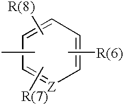
Z is N— or CH;
a thienyl radical 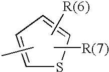
R(6) is hydrogen, halogen, hydroxyl, (C₁-C₁₀)-alkyl, (C₁-C₁₀)-alkyl-O—, phenoxy, (C₁-C₁₀)-alkyloxymethyloxy- or —(O)ₙS—R(9);
n is zero, 1 or 2;
R(9) is (C₁-C₁₀)-alkyl, thienyl, pyridyl, thiazolyl, thiadiazolyl, imidazolyl, pyrazolyl or phenyl,
each of which is unsubstituted or mono- or disubstituted by halogen, (C₁-C₄)-alkyl or (C₁-C₄)-alkyl-O—;
R(7) and R(8)
are hydrogen, halogen, hydroxyl, (C₁-C₁₀)-alkyl, (C₁-C₁₀)-alkyl-O—, phenyl, phenoxy or (C₁-C₁₀)-alkoxymethyloxy;
or
Cy is phenyl,
which is unsubstituted or is mono- or disubstituted by halogen, (C₁-C₄)-alkyl or (C₁-C₄)-alkyl-O—;
or
Cy is —Gr—Am;
Gr is —R(13)—R(12)—(CH₂)_{q}—C[W][W(1)]—(CH₂)_{q′}—; R(13)R(14)— or
—R(15)—;
R(12) is a single bond, —O—, —(O)ₙS—, —CO— or —CONH—;
n is zero, 1 or 2;
R(13) is a single bond, phenyl, thienyl, pyridyl, thiazolyl,
thiadiazolyl, imidazolyl or pyrazolyl;
R(14) is a single bond or SO₂—;
R(15) is (C₂-C₁₀)-alkenyl or (C₂-C₁₀)-alkynyl;
W and W(1)
independently of one another are hydrogen, (C₁-C₄)-alkyl;
or
W and W(1)
cyclically connected to one another are a (C₃-C₈)-hydrocarbon ring;
q and q′
are zero to 9;
Am is —NR(10)R(11);
R(10) is hydrogen, (C₁-C₄)-alkyl or benzyl,
R(11) is (C₁-C₄)-alkyl, phenyl or benzyl;
or
R(10) and R(11)
together are a (C₃-C₁₀)-alkylene group,
which is unsubstituted or substituted by —COOH, (C₁-C₅)-alkoxycarbonyl, (C₂-C₄)-hydroxylalkylene or benzyl;
or
Am is pyrrolyl, pyridyl, pyrazolyl, morpholinyl, dihydropyridyl, tetrahydropyridyl, quinuclidinyl, imidazolyl or 3-azabicyclo[3.2.1 ]octyl,
which is unsubstituted or substituted by (C₁-C₄)-alkyl,
or
Am is azabicyclo[3.2.2]nonyl;
or
Am is a piperazine group of the formula 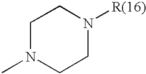
R(16) is hydrogen, (C₁-C₄)-alkyl, (C₃-C₆)-cycloalkyl,phenyl, tolyl, methoxyphenyl, halophenyl, diphenylmethylene, benzyl or pyridyl;
or
Am is an azido group —(O)ₜ—(CH₂)_{q}—C[W][W(1)]—(CH₂)_{q}—N₃;
t is zero or 1;
where W and W(1) have the previously indicated meaning; or an optical enantiomer thereof, or pharmacologically tolerable salt thereof; or
E. a guanidine compound of the formula 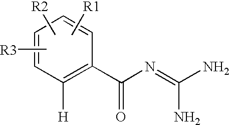
where R1=R2 is H, halo, alkyl, CN, NO₂, perfluoroalkyl, or SOₙCF₃; R3 is CH═CH₂, CH₂—CH═CH₂, CH₂—CH₂—CH═CH₂, cycloalkenyl, or cycloalkenylalkyl; n is zero, 1 or 2; and R4 is alkyl, substituted phenyl, or unsubstituted phenyl.

4. A method as claimed in claim 1, wherein the inhibitor of the Na⁺/H⁺ exchanger is a guanidine compound.

5. A method as claimed in claim 1, wherein the inhibitor of the Na⁺/H⁺ exchanger is a cariporide.

6. A method as claimed in claim 1, wherein the disturbance of the central nervous system is hyperexcitability of neurons occurring under a nonischemic condition.

7. A method as claimed in claim 2, wherein the disturbance of the central nervous system is hyperexcitability of neurons occurring under a nbnischemic condition.

8. A method as claimed in claim 3, wherein the disturbance of the central nervous system is hyperexcitability of neurons occurring under a nonischemic condition.

9. A method as claimed in claim 4, wherein the disturbance of the central nervous system is hyperexcitability of neurons occurring under a nonischemic condition.

10. A method as claimed in claim 5, wherein the disturbance of the central nervous system is hyperexcitability of neurons occurring under a nonischemic condition.

11. A method as claimed in claim 1, wherein the disturbance of the central nervous system is an epilepsy occurring under a nonischemic condition.

12. A method as claimed in claim 2, wherein the disturbance of the central nervous system is an epilepsy occurring under a nonischemic condition.

13. A method as claimed in claim 3, wherein the disturbance of the central nervous system is an epilepsy occurring under a nonischemic condition.

14. A method as claimed in claim 4, wherein the disturbance of the central nervous system is an epilepsy occurring under a nonischemic condition.

15. A method as claimed in claim 5, wherein the disturbance of the central nervous system is an epilepsy occurring under a nonischemic condition.

16. A method as claimed in claim 1, wherein the disburbance of the central nervous system is an affective psychosis or anxiety disorder occurring under a nonischemic condition.

17. A method as claimed in claim 2, wherein the disburbance of the central nervous system is an affective psychosis or anxiety disorder occurring under a nonischemic condition.

18. A method as claimed in claim 3, wherein the disburbance of the central nervous system is an affective psychosis or anxiety disorder occurring under a nonischemic condition.

19. A method as claimed in claim 4, wherein the disburbance of the central nervous system is an affective psychosis or anxiety disorder occurring under a nonischemic condition.

20. A method as claimed in claim 5, wherein the disburbance of the central nervous system is an affective psychosis or anxiety disorder occurring under a nonischemic condition.
